SEQUENCE LISTING (253 sequences)

SEQ ID NO: 1 - protein, 510 aa, Homo sapiens
MEDTKESNVKTFCSKNILAILGFSSIIAVIALLAVGLTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTYVFLMVLFSLVLFTVAIIGLLIFHKPSYFWKDMV

SEQ ID NO: 2 - protein, 511 aa, Rattus norvegicus
MEDIKDSKVKRFCSKNILIILGFSSVLAVIALIAVGLTHNKPLPENVKYGIVLDAGSSHTNLYIYKWPAEKENDTGVVQLLEECQVKGPGISKYAQKTDEIAAYLAECMKMSTERIPASKQHQTPVYLGATAGMRLLRMESKQSADEVLAAVSRSLKSYPFDFQGAKIITGQEEGAYGWITINYLLGRFTQEQSWLNFISDSQKQATFGALDLGGSSTQVTFVPLNQTLEAPETSLQFRLYGTDYTVYTHSFLCYGKDQALWQKLAQDIQVSSGGILKDPCFYPGYKKVVNVSELYGTPCTKRFEKKLPFNQFQVQGTGDYEQCHQSILKFFNNSHCPYSQCAFNGVFLPPLQGSFGAFSAFYFVMDFFKKMANDSVSSQEKMTEITKNFCSKPWEEVKASYPTVKEKYLSEYCFSGTYILSLLLQGYNFTGTSWDQIHFMGKIKDSNAGWTLGYMLNLTNMIPAEQPLSPPLPHSTYISLMVLFSLVLVAMVITGLFIFSKPSYFWKEAV

SEQ ID NO: 3 - protein, 441 aa, Homo sapiens
QIHKQEVLPPGLKYGIVLDAGSSRTTVYVYQWPAEKENNTGVVSQTFKCSVKGSGISSYGNNPQDVPRAFEECMQKVKGQVPSHLHGSTPIHLGATAGMRLLRLQNETAANEVLESIQSYFKSQPFDFRGAQIISGQEEGVYGWITANYLMGNFLEKNLWHMWVHPHGVETTGALDLGGASTQISFVAGEKMDLNTSDIMQVSLYGYVYTLYTHSFQCYGRNEAEKKFLAMLLQNSPTKNHLTNPCYPRDYSISFTMGHVFDSLCTVDQRPESYNPNDVITFEGTGDPSLCKEKVASIFDFKACHDQETCSFDGVYQPKIKGPFVAFAGFYYTASALNLSGSFSLDTFNSSTWNFCSQNWSQLPLLLPKFDEVYARSYCFSANYIYHLFVNGYKFTEETWPQIHFEKEVGNSSIAWSLGYMLSLTNQIPAESPLIRLPIEP

SEQ ID NO: 4 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 5 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 6 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 7 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccatggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 8 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVMDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 9 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccatggaagtgatccccagaagccagcaccaggaaacccccgtgtatctgggagccaccgccggcatgagactgctgagaatggaaagcgaggaactggccgaccgggtgatggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccaaggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 10 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKGFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLRTPLSHST

SEQ ID NO: 11 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccatggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagggattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgcgcacacctctgagccacagcacc

SEQ ID NO: 12 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFAQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLRTPLSHST

SEQ ID NO: 13 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgcgcagaaagtgaacgagatcggcatctacctgaccgactgcatggaacgggccatggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgaggacacctctgagccacagcacc

SEQ ID NO: 14 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVMDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIDKIQGSDAGWTLGYMLNLTNMIPAEQPLSTPLSHST

SEQ ID NO: 15 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgatggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgctgcagggctaccacttcaccgccgatagctgggagcacatccacttcatcgacaagattcagggcagcgacgccggctggacactgggctacatgctgaatctgaccaacatgatccccgccgagcagcccctgagcacacctctgagccacagcacc

SEQ ID NO: 16 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFVQKVNEIGISLTDCMERAREVIPRSQHQETPVYLGATAGMRLLRMESEELADRVMDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALRQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 17 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctccctgaccgactgcatggaacgggccagggaagtgatccccagaagccagcaccaggaaacccccgtgtatctgggagccaccgccggcatgagactgctgagaatggaaagcgaggaactggccgaccgggtgatggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgcggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 18 - protein, 427 aa, synthetic construct
  REGION 1..427: note = Description of Artificial Sequence: Synthetic polypeptide
TQNKALPENVKYGIVLDAGSSHTSLYIYKWPAEEENDTGVVHQVEECRVKGPGISKFVQKVNEIGIYLSDCMERAREVIPRSQHQETPVYLGATAGMRLLRMESEELADRVLDAVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGTGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 19 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgaggaagaaaacgacaccggcgtggtgcatcaggtggaagagtgcagagtgaagggccctggcatcagcaagttcgtgcagaaagtgaacgagatcggcatctacctgtccgactgcatggaacgggccagggaagtgatccccagaagccagcaccaggaaacccccgtgtatctgggagccaccgccggcatgagactgctgagaatggaaagcgaggaactggccgaccgggtgctggacgcggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcaccggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 20 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDAVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 21 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgattcccagaagccagcaccaggaaacccccgtgtatctgggagccaccgccggcatgagactgctgagaatggaaagcgaggaactggccgaccgggtgctggacgcggtggaaagaagcctgagcaactacccattcgattttcaaggcgccagaatcatcaccggccaggaagaaggcgcctacggctggatcaccatcaactacctgctgggtaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtagtgaacgtgtccgacctgtacaagaccccctgcaccaagagattcgagatgaccctgccattccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 22 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALRQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 23 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgaggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 24 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGTGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 25 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctatggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcaccggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcctttgagcacacctctgagccacagcacc

SEQ ID NO: 26 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGTGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 27 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccatggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcaccggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgactagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 28 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNDILRDPCFHPGYKKVVNVSDLYKTPCTKRFEMTLPFQQFEIQGTGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 29 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgatatcctgcgggacccttgcttccaccccggctacaagaaagtcgtgaacgtgtccgacctgtacaagaccccctgcaccaagagattcgagatgaccctgcccttccagcagttcgagatccagggcaccggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 30 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYSVMKFLNLTSEKVSQEKVTEMMKKFCAQPWEEIKTSYAGVKEKYLSEYCFSGTYILSLLPQGYHFTADSWEHIHFIGKIQGSDAGWTLGYMLNLTNMIPAEQPLSTPLSHST

SEQ ID NO: 31 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctactccgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgccgcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 32 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYSVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 33 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccttggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctactccgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgctgcagggctaccacttcaccgccgatagctgggagcacatccacttcatcggcaagattcagggcagcgacgccggctggacactgggttacatgctgaatctgaccaacatgatccccgccgagcagcccctgagcacacctctgagccacagcacc

SEQ ID NO: 34 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVKVSDLYKTPCTKRFEMTLPFQQFEIQGIGNYQQCHQSILELFNTSYCPYSQCAFNGIFLPPLQGDFGAFSAFYSVMKFLNLTSEKVSQEKVTEMMKKFCAQPWEEIKTSYAGVKEKYLSEYCFSGTYILSLLPQGYHFTADSWEHIHFIGKIQGSDAGWTLGYMLNLTNMISAEQPLSTPLSHST

SEQ ID NO: 35 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggcgctgtggcagaagctggccaaggacatccaggtggccagcaacgagatcctgcgggacccttgcttccaccccggctacaagaaagtcgtgaaggtgtccgacctgtacaagaccccctgcaccaagagattcgagatgaccctgcccttccagcagttcgagatccagggcatcggcaattaccagcagtgccaccagagcatcctggaactgttcaacaccagctactgcccatacagccagtgcgccttcaacggcatcttcctgccacctctgcagggggatttcggcgccttcagcgccttctactccgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgccgcagggctaccacttcaccgccgatagctgggagcacatccacttcatcggcaagattcagggcagcgacgccggctggacactgggctacatgctgaatctgaccaacatgatctccgccgagcagcccctgagcacacctctgagccacagcacc

SEQ ID NO: 36 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVNEKYLSEFCFSGTYILSLLLQGYHFTADSWEHIHFIGKIQGSDAGWTLGYMLNLTNMIPAEQPLSTPLSHST

SEQ ID NO: 37 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccaaccctgggaggaaatcaagacctcctacgctggcgtgaacgagaagtacctgagcgagttttgcttcagcggcacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 38 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEFCFSGTYILSLLLQGYHFTADSWEHIHFIGKIQGSDAGWTLGYMLNLTNMIPAEQPLSTPLSHST

SEQ ID NO: 39 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagttttgcttcagcggcacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 40 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFVQKVNEIDIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLQQGYHFTADSWEHIHFIGKIQGSDAGWTLGYMLNLTNMIPAEQPLSTPLSHST

SEQ ID NO: 41 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcgacatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgcagcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 42 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLQQGYHFTADSWEDIHFIGKIQGSDAGWTLGYMLNLTNMIPAEQPLSTPLSHST

SEQ ID NO: 43 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgcagcagggctaccacttcaccgccgatagctgggaggacatccacttcatcggcaagattcagggcagcgacgccggctggacactgggctacatgctgaatctgaccaacatgatccccgccgagcagcccctgagcacacctctgagccacagcacc

SEQ ID NO: 44 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNGILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHSIGKIQGSDAGWTLGYMLNLTNMIPAEQPLSTPLSHST

SEQ ID NO: 45 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgggatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgctgcagggctaccacttcaccgccgatagctgggagcacatccactccatcggcaagattcagggcagcgacgccggctggacactgggctacatgctgaatctgaccaacatgatccccgccgagcagcccctgagcacacctctgagccacagcacc

SEQ ID NO: 46 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLRTPLSHST

SEQ ID NO: 47 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccatggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgcgcacacctctgagccacagcacc

SEQ ID NO: 48 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNGILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLGTPLSHST

SEQ ID NO: 49 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgggatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgctgcagggctaccacttcaccgccgatagctgggagcacatccacttcatcggcaagattcagggcagcgacgccggctggacactgggctacatgttgaatctgaccaacatgatccccgccgagcagcccctgggcacacctctgagccacagcacc

SEQ ID NO: 50 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFMCYGKDQALRQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHSIGKIQGSDAGWTLGYMLNLTNMIPAEQPLRTPLSHST

SEQ ID NO: 51 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttatgtgctacggaaaggaccaggctctgaggcagaagctggccaaggacatccaggtggccagcaacgagatcctgcgggacccttgcttccaccccggctacaagaaagtcgtgaacgtgtccgacctgtacaagaccccctgtaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgctgcagggctaccacttcaccgccgatagctgggagcacatccactccatcggcaagattcagggcagcgacgccggctggacactgggctacatgctgaatctgaccaacatgatccccgccgagcagcccctgagaacacctctgagccacagcacc

SEQ ID NO: 52 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAKEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLQQGYHFTADSWEHNHFIGKIQGSDAGWTLGYMLNLTNMIPAEQPLSTPLSHST

SEQ ID NO: 53 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccaaggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgcagcagggctaccacttcaccgccgatagctgggagcacaaccacttcatcggcaagattcagggcagcgacgccggctggacactgggctacatgctgaatctgaccaacatgatccccgccgagcagcccctgagcacacctctgagccacagcacc

SEQ ID NO: 54 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVNEKYLSEFCFSGTYILSLLLQGYHFTADSWEHIHFIGKIQGSDAGWTLGYMLNLTNMIPAEQPLSTPLSHST

SEQ ID NO: 55 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccatggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccaaccctgggaggaaatcaagacctcctacgctggcgtgaacgagaagtacctgagcgagttttgcttcagcggcacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 56 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEFCFSGTYILSLLLQGYHFTADSWEHIHFIGKIQGSDAGWTLGYMLNLTNMIPAEQPLSTPLSHST

SEQ ID NO: 57 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccatggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagttttgcttcagcggcacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 58 - protein, 430 aa, synthetic construct
  REGION 1..430: note = Description of Artificial Sequence: Synthetic polypeptide
APTTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 59 - DNA, 1290 bp, synthetic construct
  misc_feature 1..1290: note = Description of Artificial Sequence: Synthetic polynucleotide
  source 1..1290: mol_type = other DNA
gcccctaccacccagaacaaggccctgcccgagaacgtgaagtacggcatcgtgctggacgccggctcctcccacacctccctgtacatctacaagtggcctgccgagaaagaaaacgacaccggcgtggtgcaccaagtggaagagtgcagagtgaagggccccggcatctccaagttcgtgcagaaagtgaacgagatcggcatctacctgaccgactgcatggaacgggccagagaagtgatccctcggtcccagcaccaggaaacccctgtctacctgggcgccaccgccggcatgcggctgctgcggatggaatccgaggaactggccgaccgggtgctggacgtggtggaacggtccctgtccaactacccattcgattttcaaggcgccagaatcatcaccggccaggaagagggcgcctacggctggatcaccatcaactacctgctgggcaagttctcccagaagaatcaggaaaccttcggcgccctggacctgggcggagccagcacccaagtcacattcgtgccccagaaccagaccatcgagagccccgacaacgccctgcagttccggctgtacggcaaggactacaacgtgtacacccacagctttctgtgctacggcaaggaccaggccctgtggcagaagctggccaaggacatccaagtggcctccaacgagatcctgcgggacccctgcttccaccccggctacaagaaagtggtcaacgtgtccgacctgtacaagaccccttgcaccaagagattcgagatgaccctgcccttccagcagttcgagatccagggcatcggcaactaccagcagtgccaccagtccatcctggaactgttcaacacctcctactgcccctactcccagtgcgccttcaacggcatcttcctgcctccactgcagggcgacttcggcgccttctccgccttctacttcgtgatgaagttcctgaacctgacctccgagaaagtgtcccaggaaaaagtgaccgagatgatgaagaagttctgcgcccagccctgggaggaaatcaagacctcctacgctggcgtgaaagagaagtacctgtccgagtactgcttctccggcacctacatcctgtccctgctgctgcagggctaccacttcaccgccgacagctgggagcacatccacttcatcggcaagatccagggatccgacgctggctggaccctgggctacatgctgaatctgaccaacatgatccccgccgagcagcccctgtccacccctctgtctcactccacc

SEQ ID NO: 60 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYSVMKFLNLTSEKVSQEKVTEMMKKFCAQPWEEIKTSYAGVKEKYLSEFCFSGTYILSLLLQGYHFTADSWEHIHFIGKIQGSDAGWTLGYMLNLTNMIPAEQPLSTPLSHST

SEQ ID NO: 61 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccttggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctactccgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagttttgcttcagcggcacctacatcctgagcctgctgctgcagggctaccacttcaccgccgatagctgggagcacatccacttcatcggcaagattcagggcagcgacgccggctggacactgggttacatgctgaatctgaccaacatgatccccgccgagcagcccctgagcacacctctgagccacagcacc

SEQ ID NO: 62 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGTGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLQQGYHFTADSWEDIHFIGKIQGSDAGWTLGYMLNLTNMIPAEQPLSTPLSHST

SEQ ID NO: 63 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcaccggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgcagcagggctaccacttcaccgccgatagctgggaggacatccacttcatcggcaagattcagggcagcgacgccggctggacactgggctacatgctgaatctgaccaacatgatccccgccgagcagcccctgagcacacctctgagccacagcacc

SEQ ID NO: 64 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYSVMKFLNLTSEKVSQEKVTEMMKKFCAQPWEEIKTSYAGVKEKYLSEYCFSGTYILSLLQQGYHFTADSWEHIHFIGKIQGSDAGWTLGYMLNLTNMIPAEQPLSTPLSHST

SEQ ID NO: 65 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccttggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctactccgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgcagcagggctaccacttcaccgccgatagctgggagcacatccacttcatcggcaagattcagggcagcgacgccggctggacactgggttacatgctgaatctgaccaacatgatccccgccgagcagcccctgagcacacctctgagccacagcacc

SEQ ID NO: 66 - protein, 442 aa, synthetic construct
  REGION 1..442: note = Description of Artificial Sequence: Synthetic polypeptide
APTSSSTKKTQLTSSTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 67 - DNA, 1326 bp, synthetic construct
  misc_feature 1..1326: note = Description of Artificial Sequence: Synthetic polynucleotide
  source 1..1326: mol_type = other DNA
gcccctaccagcagcagcaccaagaaaacccagctgaccagcagcacccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 68 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEFCFSGTYILSLLQQGYHFTADSWEDIHFIGKIQGSDAGWTLGYMLNLTNMIPAEQPLSTPLSHST

SEQ ID NO: 69 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagttttgcttcagcggcacctacatcctgagcctgctgcagcagggctaccacttcaccgccgatagctgggaggacatccacttcatcggcaagattcagggcagcgacgccggctggacactgggctacatgctgaatctgaccaacatgatccccgccgagcagcccctgagcacacctctgagccacagcacc

SEQ ID NO: 70 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEFCFSGTYILSLLQQGYHFTADSWEHIHFIGKIQGSDAGWTLGYMLNLTNMIPAEQPLSTPLSHST

SEQ ID NO: 71 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagttttgcttcagcggcacctacatcctgagcctgctgcagcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 72 - protein, 443 aa, synthetic construct
  REGION 1..443: note = Description of Artificial Sequence: Synthetic polypeptide
APTSSSTKKTQLTSSGTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 73 - DNA, 1329 bp, synthetic construct
  misc_feature 1..1329: note = Description of Artificial Sequence: Synthetic polynucleotide
  source 1..1329: mol_type = other DNA
gcccctacctcctccagcaccaagaaaacccagctgacctccagcggcacccagaacaaggccctgcccgagaacgtgaagtacggcatcgtgctggacgccggctcctcccacacctccctgtacatctacaagtggcctgccgagaaagaaaacgacaccggcgtggtgcaccaagtggaagagtgcagagtgaagggccccggcatctccaagttcgtgcagaaagtgaacgagatcggcatctacctgaccgactgcatggaacgggccagagaagtgatccctcggtcccagcaccaggaaacccctgtctacctgggcgccaccgccggcatgcggctgctgcggatggaatccgaggaactggccgaccgggtgctggacgtggtggaacggtccctgtccaactacccattcgattttcaaggcgccagaatcatcaccggccaggaagagggcgcctacggctggatcaccatcaactacctgctgggcaagttctcccagaagaatcaggaaaccttcggcgccctggacctgggcggagccagcacccaagtcacattcgtgccccagaaccagaccatcgagagccccgacaacgccctgcagttccggctgtacggcaaggactacaacgtgtacacccacagctttctgtgctacggcaaggaccaggccctgtggcagaagctggccaaggacatccaagtggcctccaacgagatcctgcgggacccctgcttccaccccggctacaagaaagtggtcaacgtgtccgacctgtacaagaccccttgcaccaagagattcgagatgaccctgcccttccagcagttcgagatccagggcatcggcaactaccagcagtgccaccagtccatcctggaactgttcaacacctcctactgcccctactcccagtgcgccttcaacggcatcttcctgcctccactgcagggcgacttcggcgccttctccgccttctacttcgtgatgaagttcctgaacctgacctccgagaaagtgtcccaggaaaaagtgaccgagatgatgaagaagttctgcgcccagccctgggaggaaatcaagacctcctacgctggcgtgaaagagaagtacctgtccgagtactgcttctccggcacctacatcctgtccctgctgctgcagggctaccacttcaccgccgacagctgggagcacatccacttcatcggcaagatccagggatccgacgctggctggaccctgggctacatgctgaatctgaccaacatgatccccgccgagcagcccctgtccacccctctgtctcactccacc

SEQ ID NO: 74 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYSVMKFLNLTSEKVSQEKVTEMMKKFCAQPWEEIKTSYAGVKEKYLSEYCFSGTYILSLLQQGYHFTADSWEHIHFIGKIQGSDAGWTLGYMLNLTNMIPAEQPLSTPLSHST

SEQ ID NO: 75 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccatggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctactccgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgcagcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 76 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEFCFSGTYILSLLQQGYHFTADSWEHIHFIGKIQGSDAGWTLGYMLNLTNMIPAEQPLSTPLSHST

SEQ ID NO: 77 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccatggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagttctgcttcagcggcacctacatcctgagcctgctgcagcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 78 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYSVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 79 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccatggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccttggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctactccgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgctgcagggctaccacttcaccgccgatagctgggagcacatccacttcatcggcaagattcagggcagcgacgccggctggacactgggttacatgctgaatctgaccaacatgatccccgccgagcagcccctgagcacacctctgagccacagcacc

SEQ ID NO: 80 - protein, 433 aa, synthetic construct
  REGION 1..433: note = Description of Artificial Sequence: Synthetic polypeptide
APTSSSTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 81 - DNA, 1299 bp, synthetic construct
  misc_feature 1..1299: note = Description of Artificial Sequence: Synthetic polynucleotide
  source 1..1299: mol_type = other DNA
gcccctaccagcagcagcacccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 82 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEAGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 83 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagccggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 84 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEAGAYGWITINYLLGKFSQKNQETFGALDLGGAATQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 85 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagccggcgcctacggctggatcaccatcaactacctgctgggcaagttcagccagaagaatcaggaaaccttcggcgccctggacctgggcggagctgctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 86 - protein, 427 aa, synthetic construct
  REGION 1..427: note = Description of Artificial Sequence: Synthetic polypeptide
TQNKALPENVKYGIVLDAGSSHTSLYIYKWPAEKEQDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYSVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 87 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaacaggacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctactccgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 88 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQAIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYSVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 89 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcaggccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctactccgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 90 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVQVSDLYKTPCTKRFEMTLPFQQFEIQGIGNYQQCHQSILELFNTSYCPYSQCAFNGIFLPPLQGDFGAFSAFYSVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 91 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgcaggtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctactccgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 92 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFQTSYCPYSQCAFNGIFLPPLQGDFGAFSAFYSVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 93 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttccagaccagctactgcccctacagccagtgcgccttcaacggcatcttcctgccacctctgcagggggatttcggcgccttcagcgccttctactccgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 94 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYSVMKFLQLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 95 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctactccgtgatgaagttcctgcagctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 96 - protein, 427 aa, synthetic construct
  REGION 1..427: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYSVMKFLNLTSEKVSQEKVTEMMKKFCAQPWEEIKTSYAGVKEKYLSEYCFSGTYILSLLLQGYHFTADSWEHIHFIGKIQGSDAGWTLGYMLQLTNMIPAEQPLSTPLSHST

SEQ ID NO: 97 - DNA, 1281 bp, synthetic construct
  misc_feature 1..1281: note = Description of Artificial Sequence: Synthetic polynucleotide
  source 1..1281: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctactccgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgctgcagggctaccacttcaccgccgatagctgggagcacatccacttcatcggcaagattcagggcagcgacgccggctggacactgggctacatgctgcagctgaccaacatgatccccgccgagcagcccctgagcacacctctgagccacagcacc

SEQ ID NO: 98 - DNA, 69 bp, synthetic construct
  misc_feature 1..69: note = Description of Artificial Sequence: Synthetic primer
  source 1..69: mol_type = other DNA
tgccctacgagacaaacaatcaggaaaccttcggcgccctggacctgggcggagcttctacccaagtga

SEQ ID NO: 99 - protein, 431 aa, Homo sapiens
QNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLS

SEQ ID NO: 100 - DNA, 1293 bp, Homo sapiens
  source 1..1293: mol_type = unassigned DNA
cagaacaaagccctgcccgagaacgtgaagtacggcatcgtgctggatgccggcagcagccacaccagcctgtacatctacaagtggcctgccgagaaagaaaacgataccggtgtcgtgcaccaggtggaagagtgcagagtgaagggccctggcatcagcaagttcgtgcagaaagtgaacgagatcggcatctacctgaccgactgcatggaacgggccagagaagtgatccccagaagccagcaccaggaaacccccgtgtacctgggagccacagccggcatgagactgctgcggatggaaagcgaggaactggccgacagagtgctggacgtggtggaaagaagcctgagcaactacccattcgattttcaaggggccagaatcatcaccggccaggaagagggcgcttacggctggatcaccatcaactacctgctgggcaagttcagccagaaaacccggtggttcagcatcgtgccctacgagacaaacaatcaggaaaccttcggagccctggacctgggcggagcctctacccaagtgaccttcgtgccccagaatcagaccatcgagagccccgacaacgccctgcagttccggctgtacggcaaggactacaatgtgtacacccacagctttctgtgctacggaaaggaccaggccctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaactaccagcagtgccaccagagcatcctggaactgttcaacaccagctactgcccctacagccagtgcgccttcaacggcatcttcctgccacctctgcagggggacttcggcgctttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggtacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgagc

SEQ ID NO: 101 - protein, 431 aa, Homo sapiens
NVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 102 - DNA, 1293 bp, Homo sapiens
  source 1..1293: mol_type = unassigned DNA
aacgtgaagtacggcatcgtgctggatgccggcagcagccacaccagcctgtacatctacaagtggcctgccgagaaagaaaacgataccggtgtcgtgcaccaggtggaagagtgcagagtgaagggccctggcatcagcaagttcgtgcagaaagtgaacgagatcggcatctacctgaccgactgcatggaacgggccagagaagtgatccccagaagccagcaccaggaaacccccgtgtacctgggagccacagccggcatgagactgctgcggatggaaagcgaggaactggccgacagagtgctggacgtggtggaaagaagcctgagcaactacccattcgattttcaaggggccagaatcatcaccggccaggaagagggcgcttacggctggatcaccatcaactacctgctgggcaagttcagccagaaaacccggtggttcagcatcgtgccctacgagacaaacaatcaggaaaccttcggagccctggacctgggcggagcctctacccaagtgaccttcgtgccccagaatcagaccatcgagagccccgacaacgccctgcagttccggctgtacggcaaggactacaatgtgtacacccacagctttctgtgctacggaaaggaccaggccctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaactaccagcagtgccaccagagcatcctggaactgttcaacaccagctactgcccctacagccagtgcgccttcaacggcatcttcctgccacctctgcagggggacttcggcgctttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggtacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgagcacacctctgtctcacagcacc

SEQ ID NO: 103 - protein, 416 aa, Homo sapiens
NVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNM

SEQ ID NO: 104 - DNA, 1248 bp, Homo sapiens
  source 1..1248: mol_type = unassigned DNA
aacgtgaagtacggcatcgtgctggatgccggcagcagccacaccagcctgtacatctacaagtggcctgccgagaaagaaaacgataccggtgtcgtgcaccaggtggaagagtgcagagtgaagggccctggcatcagcaagttcgtgcagaaagtgaacgagatcggcatctacctgaccgactgcatggaacgggccagagaagtgatccccagaagccagcaccaggaaacccccgtgtacctgggagccacagccggcatgagactgctgcggatggaaagcgaggaactggccgacagagtgctggacgtggtggaaagaagcctgagcaactacccattcgattttcaaggggccagaatcatcaccggccaggaagagggcgcttacggctggatcaccatcaactacctgctgggcaagttcagccagaaaacccggtggttcagcatcgtgccctacgagacaaacaatcaggaaaccttcggagccctggacctgggcggagcctctacccaagtgaccttcgtgccccagaatcagaccatcgagagccccgacaacgccctgcagttccggctgtacggcaaggactacaatgtgtacacccacagctttctgtgctacggaaaggaccaggccctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaactaccagcagtgccaccagagcatcctggaactgttcaacaccagctactgcccctacagccagtgcgccttcaacggcatcttcctgccacctctgcagggggacttcggcgctttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggtacctacatcctgagcctgctgctgcagggctaccacttcaccgccgatagctgggagcacatccacttcatcggcaagattcagggcagcgacgccggctggacactgggctacatgctgaatctgaccaacatg

SEQ ID NO: 105 - protein, 404 aa, synthetic construct
  REGION 1..404: note = Description of Artificial Sequence: Synthetic polypeptide
NVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNM

SEQ ID NO: 106 - DNA, 1212 bp, synthetic construct
  misc_feature 1..1212: note = Description of Artificial Sequence: Synthetic polynucleotide
  source 1..1212: mol_type = other DNA
aacgtgaagtacggcatcgtgctggatgccggcagcagccacaccagcctgtacatctacaagtggcctgccgagaaagaaaacgataccggtgtcgtgcaccaggtggaagagtgcagagtgaagggccctggcatcagcaagttcgtgcagaaagtgaacgagatcggcatctacctgaccgactgcatggaacgggccagagaagtgatccccagaagccagcaccaggaaacccccgtgtacctgggagccacagccggcatgagactgctgcggatggaaagcgaggaactggccgacagagtgctggacgtggtggaaagaagcctgagcaactacccattcgattttcaaggggccagaatcatcaccggccaggaagagggcgcttacggctggatcaccatcaactacctgctgggcaagttcagccagaaaaatcaggaaaccttcggagccctggacctgggcggagcctctacccaagtgaccttcgtgccccagaatcagaccatcgagagccccgacaacgccctgcagttccggctgtacggcaaggactacaatgtgtacacccacagctttctgtgctacggaaaggaccaggccctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaactaccagcagtgccaccagagcatcctggaactgttcaacaccagctactgcccctacagccagtgcgccttcaacggcatcttcctgccacctctgcagggggacttcggcgctttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggtacctacatcctgagcctgctgctgcagggctaccacttcaccgccgatagctgggagcacatccacttcatcggcaagattcagggcagcgacgccggctggacactgggctacatgctgaatctgaccaacatg

SEQ ID NO: 107 - protein, 416 aa, synthetic construct
  REGION 1..416: note = Description of Artificial Sequence: Synthetic polypeptide
NVKYGIVLDAGSSHTSLYIYKWPAEKENDTGVVHQVEEARVKGPGISKFVQKVNEIGIYLTDAMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNM

SEQ ID NO: 108 - DNA, 1248 bp, synthetic construct
  misc_feature 1..1248: note = Description of Artificial Sequence: Synthetic polynucleotide
  source 1..1248: mol_type = other DNA
aacgtgaagtacggcatcgtgctggatgccggcagcagccacaccagcctgtacatctacaagtggcctgccgagaaagaaaacgataccggtgtcgtgcaccaggtggaagaggccagagtgaagggccctggcatcagcaagttcgtgcagaaagtgaacgagatcggcatctacctgaccgacgccatggaacgggccagagaagtgatccccagaagccagcaccaggaaacccccgtgtacctgggagccacagccggcatgagactgctgcggatggaaagcgaggaactggccgacagagtgctggacgtggtggaaagaagcctgagcaactacccattcgattttcaaggggccagaatcatcaccggccaggaagagggcgcttacggctggatcaccatcaactacctgctgggcaagttcagccagaaaacccggtggttcagcatcgtgccctacgagacaaacaatcaggaaaccttcggagccctggacctgggcggagcctctacccaagtgaccttcgtgccccagaatcagaccatcgagagccccgacaacgccctgcagttccggctgtacggcaaggactacaatgtgtacacccacagctttctgtgctacggaaaggaccaggccctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaactaccagcagtgccaccagagcatcctggaactgttcaacaccagctactgcccctacagccagtgcgccttcaacggcatcttcctgccacctctgcagggggacttcggcgctttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggtacctacatcctgagcctgctgctgcagggctaccacttcaccgccgatagctgggagcacatccacttcatcggcaagattcagggcagcgacgccggctggacactgggctacatgctgaatctgaccaacatg

SEQ ID NO: 109 - protein, 416 aa, synthetic construct
  REGION 1..416: note = Description of Artificial Sequence: Synthetic polypeptide
NVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPAFHPGYKKVVNVSDLYKTPCTKRFEMTLPFQQFEIQGIGNYQQAHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNM

SEQ ID NO: 110 - DNA, 1248 bp, synthetic construct
  misc_feature 1..1248: note = Description of Artificial Sequence: Synthetic polynucleotide
  source 1..1248: mol_type = other DNA
aacgtgaagtacggcatcgtgctggatgccggcagcagccacaccagcctgtacatctacaagtggcctgccgagaaagaaaacgataccggtgtcgtgcaccaggtggaagagtgcagagtgaagggccctggcatcagcaagttcgtgcagaaagtgaacgagatcggcatctacctgaccgactgcatggaacgggccagagaagtgatccccagaagccagcaccaggaaacccccgtgtacctgggagccacagccggcatgagactgctgcggatggaaagcgaggaactggccgacagagtgctggacgtggtggaaagaagcctgagcaactacccattcgattttcaaggggccagaatcatcaccggccaggaagagggcgcttacggctggatcaccatcaactacctgctgggcaagttcagccagaaaacccggtggttcagcatcgtgccctacgagacaaacaatcaggaaaccttcggagccctggacctgggcggagcctctacccaagtgaccttcgtgccccagaatcagaccatcgagagccccgacaacgccctgcagttccggctgtacggcaaggactacaatgtgtacacccacagctttctgtgctacggaaaggaccaggccctgtggcagaagctggccaaggacatccaggtggccagcaacgagatcctgcgggaccctgccttccaccccggctacaagaaagtcgtgaacgtgtccgacctgtacaagaccccctgcaccaagagattcgagatgaccctgcccttccagcagttcgagatccagggcatcggcaactaccagcaggcccaccagagcatcctggaactgttcaacaccagctactgcccctacagccagtgcgccttcaacggcatcttcctgccacctctgcagggggacttcggcgctttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggtacctacatcctgagcctgctgctgcagggctaccacttcaccgccgatagctgggagcacatccacttcatcggcaagattcagggcagcgacgccggctggacactgggctacatgctgaatctgaccaacatg

SEQ ID NO: 111 - protein, 416 aa, synthetic construct
  REGION 1..416: note = Description of Artificial Sequence: Synthetic polypeptide
NVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLAYGKDQALWQKLAKDIQVASNEILRDPCFHPGYKKVVNVSDLYKTPATKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNM

SEQ ID NO: 112 - DNA, 1248 bp, synthetic construct
  misc_feature 1..1248: note = Description of Artificial Sequence: Synthetic polynucleotide
  source 1..1248: mol_type = other DNA
aacgtgaagtacggcatcgtgctggatgccggcagcagccacaccagcctgtacatctacaagtggcctgccgagaaagaaaacgataccggtgtcgtgcaccaggtggaagagtgcagagtgaagggccctggcatcagcaagttcgtgcagaaagtgaacgagatcggcatctacctgaccgactgcatggaacgggccagagaagtgatccccagaagccagcaccaggaaacccccgtgtacctgggagccacagccggcatgagactgctgcggatggaaagcgaggaactggccgacagagtgctggacgtggtggaaagaagcctgagcaactacccattcgattttcaaggggccagaatcatcaccggccaggaagagggcgcttacggctggatcaccatcaactacctgctgggcaagttcagccagaaaacccggtggttcagcatcgtgccctacgagacaaacaatcaggaaaccttcggagccctggacctgggcggagcctctacccaagtgaccttcgtgccccagaatcagaccatcgagagccccgacaacgccctgcagttccggctgtacggcaaggactacaatgtgtacacccacagctttctggcctacggaaaggaccaggccctgtggcagaagctggccaaggacatccaggtggccagcaacgagatcctgcgggacccttgcttccaccccggctacaagaaagtcgtgaacgtgtccgacctgtacaagacccccgccaccaagagattcgagatgaccctgcccttccagcagttcgagatccagggcatcggcaactaccagcagtgccaccagagcatcctggaactgttcaacaccagctactgcccctacagccagtgcgccttcaacggcatcttcctgccacctctgcagggggacttcggcgctttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggtacctacatcctgagcctgctgctgcagggctaccacttcaccgccgatagctgggagcacatccacttcatcggcaagattcagggcagcgacgccggctggacactgggctacatgctgaatctgaccaacatg

SEQ ID NO: 113 - protein, 416 aa, synthetic construct
  REGION 1..416: note = Description of Artificial Sequence: Synthetic polypeptide
NVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYAPYSQAAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNM

SEQ ID NO: 114 - DNA, 1248 bp, synthetic construct
  misc_feature 1..1248: note = Description of Artificial Sequence: Synthetic polynucleotide
  source 1..1248: mol_type = other DNA
aacgtgaagtacggcatcgtgctggatgccggcagcagccacaccagcctgtacatctacaagtggcctgccgagaaagaaaacgataccggtgtcgtgcaccaggtggaagagtgcagagtgaagggccctggcatcagcaagttcgtgcagaaagtgaacgagatcggcatctacctgaccgactgcatggaacgggccagagaagtgatccccagaagccagcaccaggaaacccccgtgtacctgggagccacagccggcatgagactgctgcggatggaaagcgaggaactggccgacagagtgctggacgtggtggaaagaagcctgagcaactacccattcgattttcaaggggccagaatcatcaccggccaggaagagggcgcttacggctggatcaccatcaactacctgctgggcaagttcagccagaaaacccggtggttcagcatcgtgccctacgagacaaacaatcaggaaaccttcggagccctggacctgggcggagcctctacccaagtgaccttcgtgccccagaatcagaccatcgagagccccgacaacgccctgcagttccggctgtacggcaaggactacaatgtgtacacccacagctttctgtgctacggaaaggaccaggccctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaactaccagcagtgccaccagagcatcctggaactgttcaacaccagctacgccccctacagccaggccgccttcaacggcatcttcctgccacctctgcagggggacttcggcgctttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggtacctacatcctgagcctgctgctgcagggctaccacttcaccgccgatagctgggagcacatccacttcatcggcaagattcagggcagcgacgccggctggacactgggctacatgctgaatctgaccaacatg

SEQ ID NO: 115 - protein, 416 aa, synthetic construct
  REGION 1..416: note = Description of Artificial Sequence: Synthetic polypeptide
NVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFAAQPWEEIKTSYAGVKEKYLSEYAFSGTYILSLLLQGYHFTADSWEHIHFIGKIQGSDAGWTLGYMLNLTNM

SEQ ID NO: 116 - DNA, 1248 bp, synthetic construct
  misc_feature 1..1248: note = Description of Artificial Sequence: Synthetic polynucleotide
  source 1..1248: mol_type = other DNA
aacgtgaagtacggcatcgtgctggatgccggcagcagccacaccagcctgtacatctacaagtggcctgccgagaaagaaaacgataccggtgtcgtgcaccaggtggaagagtgcagagtgaagggccctggcatcagcaagttcgtgcagaaagtgaacgagatcggcatctacctgaccgactgcatggaacgggccagagaagtgatccccagaagccagcaccaggaaacccccgtgtacctgggagccacagccggcatgagactgctgcggatggaaagcgaggaactggccgacagagtgctggacgtggtggaaagaagcctgagcaactacccattcgattttcaaggggccagaatcatcaccggccaggaagagggcgcttacggctggatcaccatcaactacctgctgggcaagttcagccagaaaacccggtggttcagcatcgtgccctacgagacaaacaatcaggaaaccttcggagccctggacctgggcggagcctctacccaagtgaccttcgtgccccagaatcagaccatcgagagccccgacaacgccctgcagttccggctgtacggcaaggactacaatgtgtacacccacagctttctgtgctacggaaaggaccaggccctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaactaccagcagtgccaccagagcatcctggaactgttcaacaccagctactgcccctacagccagtgcgccttcaacggcatcttcctgccacctctgcagggggacttcggcgctttcagcgccttctacttcgtgatgaagttcctgaacctgaccagcgagaaggtgtcccaggaaaaagtgacagagatgatgaagaagttcgccgcccagccctgggaggaaatcaagacctcctacgctggcgtgaaagagaagtacctgagcgagtacgccttcagcggtacctacatcctgagcctgctgctgcagggctaccacttcaccgccgatagctgggagcacatccacttcatcggcaagattcagggcagcgacgccggctggacactgggctacatgctgaatctgaccaacatg

SEQ ID NO: 117 - protein, 404 aa, synthetic construct
  REGION 1..404: note = Description of Artificial Sequence: Synthetic polypeptide
NVKYGIVLDAGSSHTSLYIYKWPAEKENDTGVVHQVEEARVKGPGISKFVQKVNEIGIYLTDAMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNM

SEQ ID NO: 118 - DNA, 1212 bp, synthetic construct
  misc_feature 1..1212: note = Description of Artificial Sequence: Synthetic polynucleotide
  source 1..1212: mol_type = other DNA
aacgtgaagtacggcatcgtgctggatgccggcagcagccacaccagcctgtacatctacaagtggcctgccgagaaagaaaacgataccggtgtcgtgcaccaggtggaagaggccagagtgaagggccctggcatcagcaagttcgtgcagaaagtgaacgagatcggcatctacctgaccgacgccatggaacgggccagagaagtgatccccagaagccagcaccaggaaacccccgtgtacctgggagccacagccggcatgagactgctgcggatggaaagcgaggaactggccgacagagtgctggacgtggtggaaagaagcctgagcaactacccattcgattttcaaggggccagaatcatcaccggccaggaagagggcgcttacggctggatcaccatcaactacctgctgggcaagttcagccagaaaaatcaggaaaccttcggagccctggacctgggcggagcctctacccaagtgaccttcgtgccccagaatcagaccatcgagagccccgacaacgccctgcagttccggctgtacggcaaggactacaatgtgtacacccacagctttctgtgctacggaaaggaccaggccctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaactaccagcagtgccaccagagcatcctggaactgttcaacaccagctactgcccctacagccagtgcgccttcaacggcatcttcctgccacctctgcagggggacttcggcgctttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggtacctacatcctgagcctgctgctgcagggctaccacttcaccgccgatagctgggagcacatccacttcatcggcaagattcagggcagcgacgccggctggacactgggctacatgctgaatctgaccaacatg

SEQ ID NO: 119 - protein, 404 aa, synthetic construct
  REGION 1..404: note = Description of Artificial Sequence: Synthetic polypeptide
NVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPAFHPGYKKVVNVSDLYKTPCTKRFEMTLPFQQFEIQGIGNYQQAHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNM

SEQ ID NO: 120 - DNA, 1212 bp, synthetic construct
  misc_feature 1..1212: note = Description of Artificial Sequence: Synthetic polynucleotide
  source 1..1212: mol_type = other DNA
aacgtgaagtacggcatcgtgctggatgccggcagcagccacaccagcctgtacatctacaagtggcctgccgagaaagaaaacgataccggtgtcgtgcaccaggtggaagagtgcagagtgaagggccctggcatcagcaagttcgtgcagaaagtgaacgagatcggcatctacctgaccgactgcatggaacgggccagagaagtgatccccagaagccagcaccaggaaacccccgtgtacctgggagccacagccggcatgagactgctgcggatggaaagcgaggaactggccgacagagtgctggacgtggtggaaagaagcctgagcaactacccattcgattttcaaggggccagaatcatcaccggccaggaagagggcgcttacggctggatcaccatcaactacctgctgggcaagttcagccagaaaaatcaggaaaccttcggagccctggacctgggcggagcctctacccaagtgaccttcgtgccccagaatcagaccatcgagagccccgacaacgccctgcagttccggctgtacggcaaggactacaatgtgtacacccacagctttctgtgctacggaaaggaccaggccctgtggcagaagctggccaaggacatccaggtggccagcaacgagatcctgcgggaccctgccttccaccccggctacaagaaagtcgtgaacgtgtccgacctgtacaagaccccctgcaccaagagattcgagatgaccctgcccttccagcagttcgagatccagggcatcggcaactaccagcaggcccaccagagcatcctggaactgttcaacaccagctactgcccctacagccagtgcgccttcaacggcatcttcctgccacctctgcagggggacttcggcgctttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggtacctacatcctgagcctgctgctgcagggctaccacttcaccgccgatagctgggagcacatccacttcatcggcaagattcagggcagcgacgccggctggacactgggctacatgctgaatctgaccaacatg

SEQ ID NO: 121 - protein, 404 aa, synthetic construct
  REGION 1..404: note = Description of Artificial Sequence: Synthetic polypeptide
NVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLAYGKDQALWQKLAKDIQVASNEILRDPCFHPGYKKVVNVSDLYKTPATKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNM

SEQ ID NO: 122 - DNA, 1212 bp, synthetic construct
  misc_feature 1..1212: note = Description of Artificial Sequence: Synthetic polynucleotide
  source 1..1212: mol_type = other DNA
aacgtgaagtacggcatcgtgctggatgccggcagcagccacaccagcctgtacatctacaagtggcctgccgagaaagaaaacgataccggtgtcgtgcaccaggtggaagagtgcagagtgaagggccctggcatcagcaagttcgtgcagaaagtgaacgagatcggcatctacctgaccgactgcatggaacgggccagagaagtgatccccagaagccagcaccaggaaacccccgtgtacctgggagccacagccggcatgagactgctgcggatggaaagcgaggaactggccgacagagtgctggacgtggtggaaagaagcctgagcaactacccattcgattttcaaggggccagaatcatcaccggccaggaagagggcgcttacggctggatcaccatcaactacctgctgggcaagttcagccagaaaaatcaggaaaccttcggagccctggacctgggcggagcctctacccaagtgaccttcgtgccccagaatcagaccatcgagagccccgacaacgccctgcagttccggctgtacggcaaggactacaatgtgtacacccacagctttctggcctacggaaaggaccaggccctgtggcagaagctggccaaggacatccaggtggccagcaacgagatcctgcgggacccttgcttccaccccggctacaagaaagtcgtgaacgtgtccgacctgtacaagacccccgccaccaagagattcgagatgaccctgcccttccagcagttcgagatccagggcatcggcaactaccagcagtgccaccagagcatcctggaactgttcaacaccagctactgcccctacagccagtgcgccttcaacggcatcttcctgccacctctgcagggggacttcggcgctttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggtacctacatcctgagcctgctgctgcagggctaccacttcaccgccgatagctgggagcacatccacttcatcggcaagattcagggcagcgacgccggctggacactgggctacatgctgaatctgaccaacatg

SEQ ID NO: 123 - protein, 404 aa, synthetic construct
  REGION 1..404: note = Description of Artificial Sequence: Synthetic polypeptide
NVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYAPYSQAAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNM

SEQ ID NO: 124 - DNA, 1212 bp, synthetic construct
  misc_feature 1..1212: note = Description of Artificial Sequence: Synthetic polynucleotide
  source 1..1212: mol_type = other DNA
aacgtgaagtacggcatcgtgctggatgccggcagcagccacaccagcctgtacatctacaagtggcctgccgagaaagaaaacgataccggtgtcgtgcaccaggtggaagagtgcagagtgaagggccctggcatcagcaagttcgtgcagaaagtgaacgagatcggcatctacctgaccgactgcatggaacgggccagagaagtgatccccagaagccagcaccaggaaacccccgtgtacctgggagccacagccggcatgagactgctgcggatggaaagcgaggaactggccgacagagtgctggacgtggtggaaagaagcctgagcaactacccattcgattttcaaggggccagaatcatcaccggccaggaagagggcgcttacggctggatcaccatcaactacctgctgggcaagttcagccagaaaaatcaggaaaccttcggagccctggacctgggcggagcctctacccaagtgaccttcgtgccccagaatcagaccatcgagagccccgacaacgccctgcagttccggctgtacggcaaggactacaatgtgtacacccacagctttctgtgctacggaaaggaccaggccctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaactaccagcagtgccaccagagcatcctggaactgttcaacaccagctacgccccctacagccaggccgccttcaacggcatcttcctgccacctctgcagggggacttcggcgctttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggtacctacatcctgagcctgctgctgcagggctaccacttcaccgccgatagctgggagcacatccacttcatcggcaagattcagggcagcgacgccggctggacactgggctacatgctgaatctgaccaacatg

SEQ ID NO: 125 - protein, 404 aa, synthetic construct
  REGION 1..404: note = Description of Artificial Sequence: Synthetic polypeptide
NVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFAAQPWEEIKTSYAGVKEKYLSEYAFSGTYILSLLLQGYHFTADSWEHIHFIGKIQGSDAGWTLGYMLNLTNM

SEQ ID NO: 126 - DNA, 1212 bp, synthetic construct
  misc_feature 1..1212: note = Description of Artificial Sequence: Synthetic polynucleotide
  source 1..1212: mol_type = other DNA
aacgtgaagtacggcatcgtgctggatgccggcagcagccacaccagcctgtacatctacaagtggcctgccgagaaagaaaacgataccggtgtcgtgcaccaggtggaagagtgcagagtgaagggccctggcatcagcaagttcgtgcagaaagtgaacgagatcggcatctacctgaccgactgcatggaacgggccagagaagtgatccccagaagccagcaccaggaaacccccgtgtacctgggagccacagccggcatgagactgctgcggatggaaagcgaggaactggccgacagagtgctggacgtggtggaaagaagcctgagcaactacccattcgattttcaaggggccagaatcatcaccggccaggaagagggcgcttacggctggatcaccatcaactacctgctgggcaagttcagccagaaaaatcaggaaaccttcggagccctggacctgggcggagcctctacccaagtgaccttcgtgccccagaatcagaccatcgagagccccgacaacgccctgcagttccggctgtacggcaaggactacaatgtgtacacccacagctttctgtgctacggaaaggaccaggccctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaactaccagcagtgccaccagagcatcctggaactgttcaacaccagctactgcccctacagccagtgcgccttcaacggcatcttcctgccacctctgcagggggacttcggcgctttcagcgccttctacttcgtgatgaagttcctgaacctgaccagcgagaaggtgtcccaggaaaaagtgacagagatgatgaagaagttcgccgcccagccctgggaggaaatcaagacctcctacgctggcgtgaaagagaagtacctgagcgagtacgccttcagcggtacctacatcctgagcctgctgctgcagggctaccacttcaccgccgatagctgggagcacatccacttcatcggcaagattcagggcagcgacgccggctggacactgggctacatgctgaatctgaccaacatg

SEQ ID NO: 127 - protein, 431 aa, synthetic construct
  REGION 1..431: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 128 - DNA, 1293 bp, synthetic construct
  misc_feature 1..1293: note = Description of Artificial Sequence: Synthetic polynucleotide
  source 1..1293: mol_type = other DNA
acacagaacaaagccctgcccgagaacgtgaagtacggcatcgtgctggatgccggcagcagccacaccagcctgtacatctacaagtggcctgccgagaaagaaaacgataccggtgtcgtgcaccaggtggaagagtgcagagtgaagggccctggcatcagcaagttcgtgcagaaagtgaacgagatcggcatctacctgaccgactgcatggaacgggccagagaagtgatccccagaagccagcaccaggaaacccccgtgtacctgggagccacagccggcatgagactgctgcggatggaaagcgaggaactggccgacagagtgctggacgtggtggaaagaagcctgagcaactacccattcgattttcaaggggccagaatcatcaccggccaggaagagggcgcttacggctggatcaccatcaactacctgctgggcaagttcagccagaaaacccggtggttcagcatcgtgccctacgagacaaacaatcaggaaaccttcggagccctggacctgggcggagcctctacccaagtgaccttcgtgccccagaatcagaccatcgagagccccgacaacgccctgcagttccggctgtacggcaaggactacaatgtgtacacccacagctttctgtgctacggaaaggaccaggccctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaactaccagcagtgccaccagagcatcctggaactgttcaacaccagcttcaacggcatcttcctgccacctctgcagggggacttcggcgctttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggtacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgagcacacctctgtctcacagcacc

SEQ ID NO: 129 - protein, 439 aa, synthetic construct
  REGION 1..439: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYAPYSQAAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 130 - DNA, 1317 bp, synthetic construct
  misc_feature 1..1317: note = Description of Artificial Sequence: Synthetic polynucleotide
  source 1..1317: mol_type = other DNA
acacagaacaaagccctgcccgagaacgtgaagtacggcatcgtgctggatgccggcagcagccacaccagcctgtacatctacaagtggcctgccgagaaagaaaacgataccggtgtcgtgcaccaggtggaagagtgcagagtgaagggccctggcatcagcaagttcgtgcagaaagtgaacgagatcggcatctacctgaccgactgcatggaacgggccagagaagtgatccccagaagccagcaccaggaaacccccgtgtacctgggagccacagccggcatgagactgctgcggatggaaagcgaggaactggccgacagagtgctggacgtggtggaaagaagcctgagcaactacccattcgattttcaaggggccagaatcatcaccggccaggaagagggcgcttacggctggatcaccatcaactacctgctgggcaagttcagccagaaaacccggtggttcagcatcgtgccctacgagacaaacaatcaggaaaccttcggagccctggacctgggcggagcctctacccaagtgaccttcgtgccccagaatcagaccatcgagagccccgacaacgccctgcagttccggctgtacggcaaggactacaatgtgtacacccacagctttctgtgctacggaaaggaccaggccctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaactaccagcagtgccaccagagcatcctggaactgttcaacaccagctacgccccctacagccaggccgccttcaacggcatcttcctgccacctctgcagggggacttcggcgctttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggtacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgagcacacctctgtctcacagcacc

SEQ ID NO: 131 - protein, 16 aa, synthetic construct
  REGION 1..16: note = Description of Artificial Sequence: Synthetic peptide
APTSSSTKKTQLTSSG

SEQ ID NO: 132 - DNA, 48 bp, synthetic construct
  misc_feature 1..48: note = Description of Artificial Sequence: Synthetic oligonucleotide
  source 1..48: mol_type = other DNA
gcccccaccagcagcagcaccaagaagacccagctgaccagcagcggc

SEQ ID NO: 133 - protein, 15 aa, synthetic construct
  REGION 1..15: note = Description of Artificial Sequence: Synthetic peptide
APTSSSTKKTQLTSS

SEQ ID NO: 134 - DNA, 45 bp, synthetic construct
  misc_feature 1..45: note = Description of Artificial Sequence: Synthetic oligonucleotide
  source 1..45: mol_type = other DNA
gcccctaccagcagcagcaccaagaaaacccagctgaccagcagc

SEQ ID NO: 135 - protein, 6 aa, synthetic construct
  REGION 1..6: note = Description of Artificial Sequence: Synthetic peptide
APTSSS

SEQ ID NO: 136 - DNA, 18 bp, synthetic construct
  misc_feature 1..18: note = Description of Artificial Sequence: Synthetic oligonucleotide
  source 1..18: mol_type = other DNA
gcccctaccagcagcagc

SEQ ID NO: 137-138 - skipped (2 entries)

SEQ ID NO: 139 - protein, 9 aa, synthetic construct
  REGION 1..9: note = Description of Artificial Sequence: Synthetic peptide
APTSSSTKK

SEQ ID NO: 140 - DNA, 27 bp, synthetic construct
  misc_feature 1..27: note = Description of Artificial Sequence: Synthetic oligonucleotide
  source 1..27: mol_type = other DNA
gcccctaccagcagcagcaccaagaaa

SEQ ID NO: 141 - protein, 12 aa, synthetic construct
  REGION 1..12: note = Description of Artificial Sequence: Synthetic peptide
APTSSSTKKTQL

SEQ ID NO: 142 - DNA, 36 bp, synthetic construct
  misc_feature 1..36: note = Description of Artificial Sequence: Synthetic oligonucleotide
  source 1..36: mol_type = other DNA
gcccctaccagcagcagcaccaagaaaacccagctg

SEQ ID NO: 143 - protein, 9 aa, synthetic construct
  REGION 1..9: note = Description of Artificial Sequence: Synthetic peptide
SSSTKKTQL

SEQ ID NO: 144 - DNA, 27 bp, synthetic construct
  misc_feature 1..27: note = Description of Artificial Sequence: Synthetic oligonucleotide
  source 1..27: mol_type = other DNA
agcagcagcaccaagaaaacccagctg

SEQ ID NO: 145 - protein, 426 aa, synthetic construct
  REGION 1..426: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQDEGAYGWITINYLLGKFSQKNQETFGALDLGGASTQVTFVPQNQTIESPDNALQFRLYGKDYNVYTHSFLCYGRDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSNFYYVMKFLNLTSEKVSQEKVTEMMKKFCAQPWEEIKTSYAGQERWLRDYCFSGTYILSLLLQGYHFTADSWEHIHFIGKIQGSDAGWTLGYMLNLTNMIPAEQPLSTPLSHST

SEQ ID NO: 146 - DNA, 1278 bp, synthetic construct
  misc_feature 1..1278: note = Description of Artificial Sequence: Synthetic polynucleotide
  source 1..1278: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagagaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggacgagggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggccgggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaactaccagcagtgccaccagagcatcctggaactgttcaacaccagctactgcccctacagccagtgcgccttcaacggcatcttcctgccacctctgcagggggatttcggcgccttcagcaacttctactacgtgatgaagttcctgaacctgaccagcgagaaggtgtcccaggaaaaagtgacagagatgatgaagaagttctgcgcccagccctgggaggaaatcaagacctcttacgccggacaggaacggtggctgcgggactactgtttcagcggcacctacatcctgtccctgctgctgcagggctaccacttcaccgccgatagctgggagcacatccacttcatcggcaagattcagggcagcgacgccggctggacactgggctacatgctgaatctgaccaacatgatccccgccgagcagcccctgagcacacctctgtctcacagcacc

SEQ ID NO: 147 - protein, 537 aa, synthetic construct
  REGION 1..537: note = Description of Artificial Sequence: Synthetic polypeptide
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEGGGGSTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 148 - DNA, 1611 bp, synthetic construct
  misc_feature 1..1611: note = Description of Artificial Sequence: Synthetic polynucleotide
  source 1..1611: mol_type = other DNA
acggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaggtgacccaccagggcctgtccagccccgtgaccaagagcttcaaccggggcgagggaggcggaggatctacccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 149 - protein, 508 aa, synthetic construct
  REGION 1..508: note = Description of Artificial Sequence: Synthetic polypeptide
MQIFVKTLTGKTITLEVEPSDTIENVKAKIQDKEGIPPDQQRLIFAGKQLEDGRTLSDYNIQKESTLHLVLRLRGGGGGGSTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 150 - DNA, 1524 bp, synthetic construct
  misc_feature 1..1524: note = Description of Artificial Sequence: Synthetic polynucleotide
  source 1..1524: mol_type = other DNA
atgcaaatcttcgtgaagaccctgactggtaagaccatcaccctcgaggtggagcccagtgacaccatcgagaatgtcaaggcaaagatccaagataaggaaggcatccctcctgatcagcagaggttgatctttgctgggaaacagctggaagatggacgcaccctgtctgactacaacatccagaaagagtccactctgcacttggtcctgcgcttgagggggggtggaggcggaggatctacccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 151 - protein, 618 aa, synthetic construct
  REGION 1..618: note = Description of Artificial Sequence: Synthetic polypeptide
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRGGGGSTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 152 - DNA, 1854 bp, synthetic construct
  misc_feature 1..1854: note = Description of Artificial Sequence: Synthetic polynucleotide
  source 1..1854: mol_type = other DNA
gacgcccacaagagcgaggtggcccaccggttcaaggacctgggcgaggaaaacttcaaggccctggtgctgatcgccttcgcccagtacctgcagcagagccccttcgaagatcacgtaaagttagtcaacgaggttacggaattcgcaaagacatgcgttgctgacgaatccgctgagaattgtgacaagagtttgcacactttattcggagataagttgtgtactgtagctactttgagagagacttacggtgaaatggctgactgctgtgcaaaacaggaaccagaacgtaacgaatgtttccttcagcataaggatgataaccctaaccttccaaggcttgttaggccagaagtcgacgtgatgtgcaccgccttccatgataatgaagagacttttcttaaaaagtacctatacgagattgcaaggcgtcatccatatttttacgccccagagctgttgtttttcgcaaagagatacaaagctgcatttactgagtgttgccaagctgccgacaaggccgcttgtttgctaccaaagttggacgaattgagaggaggcggaggatctacccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 153 - protein, 625 aa, synthetic construct
  REGION 1..625: note = Description of Artificial Sequence: Synthetic polypeptide
DEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPGGGGSTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 154 - DNA, 1875 bp, synthetic construct
  misc_feature 1..1875: note = Description of Artificial Sequence: Synthetic polynucleotide
  source 1..1875: mol_type = other DNA
gacgagggtaaggcatcatctgccaagcagagattaaaatgtgcatctttgcaaaaatttggagagagagcttttaaggcatgggctgttgcccgactaagccaaagattcccaaaagccgaatttgctgaagtatccaagctggtgactgatttgactaaagtacatacagaatgttgccatggcgaccttttagaatgtgctgatgacagagcagatttggctaagtatatctgcgaaaatcaagattcaatcagctctaagctgaaggaatgttgcgagaaaccactgttagaaaaatcgcattgtattgctgaagttgaaaatgatgagatgcctgctgacttgccttctcttgccgctgattttgttgagtcgaaggatgtctgtaagaattatgctgaagctaaagacgttttcctgggtatgttcttatatgagtacgcaagacgtcacccagattactctgtggttctgctactgagattggctaaaacatacgagacaacgctggagaagtgctgtgctgccgctgaccctcatgagtgctatgcaaaggtttttgatgaattcaaaccaggaggcggaggatctacccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 155 - protein, 439 aa, synthetic construct
  REGION 1..439: note = Description of Artificial Sequence: Synthetic polypeptide
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYSVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 156 - DNA, 1317 bp, synthetic construct
  misc_feature 1..1317: note = Description of Artificial Sequence: Synthetic polynucleotide
  source 1..1317: mol_type = other DNA
acccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaccagatggttcagcatcgtgccctacgagacaaacaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctactccgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 157 - DNA, 90 bp, synthetic construct
  misc_feature 1..90: note = Description of Artificial Sequence: Synthetic primer
  source 1..90: mol_type = other DNA
tcgcgatcctggaaggcgtgcactgcgcccctaccagcagcagcaccaagaaaacccagctgaccagcagcacccagaacaaggccctgc

SEQ ID NO: 158 - DNA, 18 bp, synthetic construct
  misc_feature 1..18: note = Description of Artificial Sequence: Synthetic primer
  source 1..18: mol_type = other DNA
tagaaggcacagtcgagg

SEQ ID NO: 159 - DNA, 66 bp, synthetic construct
  misc_feature 1..66: note = Description of Artificial Sequence: Synthetic primer
  source 1..66: mol_type = other DNA
ctggtcgcgatcctggaaggcgtgcactgcgcccctaccagcagcagcacccagaacaaggccctg

SEQ ID NO: 160 - DNA, 66 bp, synthetic construct; identical to SEQ ID NO: 159
  misc_feature 1..66: note = Description of Artificial Sequence: Synthetic primer
  source 1..66: mol_type = other DNA

SEQ ID NO: 161 - DNA, 57 bp, synthetic construct
  misc_feature 1..57: note = Description of Artificial Sequence: Synthetic primer
  source 1..57: mol_type = other DNA
ctggtcgcgatcctggaaggcgtgcactgcgcccctaccacccagaacaaggccctg

SEQ ID NO: 162 - DNA, 75 bp, synthetic construct
  misc_feature 1..75: note = Description of Artificial Sequence: Synthetic primer
  source 1..75: mol_type = other DNA
ctggtcgcgatcctggaaggcgtgcactgcgcccctaccagcagcagcaccaagaaaacccagaacaaggccctg

SEQ ID NO: 163 - DNA, 84 bp, synthetic construct
  misc_feature 1..84: note = Description of Artificial Sequence: Synthetic primer
  source 1..84: mol_type = other DNA
ctggtcgcgatcctggaaggcgtgcactgcgcccctaccagcagcagcaccaagaaaacccagctgacccagaacaaggccctg

SEQ ID NO: 164 - DNA, 75 bp, synthetic construct
  misc_feature 1..75: note = Description of Artificial Sequence: Synthetic primer
  source 1..75: mol_type = other DNA
ctggtcgcgatcctggaaggcgtgcactgcagcagcagcaccaagaaaacccagctgacccagaacaaggccctg

SEQ ID NO: 165 - DNA, 16 bp, synthetic construct
  misc_feature 1..16: note = Description of Artificial Sequence: Synthetic primer
  source 1..16: mol_type = other DNA
catacgatttaggtga

SEQ ID NO: 166 - DNA, 18 bp, synthetic construct
  misc_feature 1..18: note = Description of Artificial Sequence: Synthetic primer
  source 1..18: mol_type = other DNA
tagaaggcacagtcgagg

SEQ ID NO: 167 - protein, 76 aa, synthetic construct [sequence omitted]

SEQ ID NO: 168 - protein, 105 aa, synthetic construct
  REGION 1..105: note = Description of Artificial Sequence: Synthetic polypeptide
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 169 - protein, 186 aa, Homo sapiens
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELR

SEQ ID NO: 170 - protein, 193 aa, Homo sapiens
DEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKP

SEQ ID NO: 171 - DNA, 30 bp, synthetic construct
  misc_feature 1..30: note = Description of Artificial Sequence: Synthetic primer
  source 1..30: mol_type = other DNA
ctgccgagaaagaacaggacaccggcgtgg

SEQ ID NO: 172 - DNA, 30 bp, synthetic construct
  misc_feature 1..30: note = Description of Artificial Sequence: Synthetic primer
  source 1..30: mol_type = other DNA
ccacgccggtgtcctgttctttctcggcag

SEQ ID NO: 173 - DNA, 29 bp, synthetic construct
  misc_feature 1..29: note = Description of Artificial Sequence: Synthetic primer
  source 1..29: mol_type = other DNA
cgtgccccagaatcaggccatcgagagcc

SEQ ID NO: 174 - DNA, 29 bp, synthetic construct
  misc_feature 1..29: note = Description of Artificial Sequence: Synthetic primer
  source 1..29: mol_type = other DNA
ggctctcgatggcctgattctggggcacg

SEQ ID NO: 175 - DNA, 41 bp, synthetic construct
  misc_feature 1..41: note = Description of Artificial Sequence: Synthetic primer
  source 1..41: mol_type = other DNA
ggctacaagaaagtcgtgcaggtgtccgacctgtacaagac

SEQ ID NO: 176 - DNA, 41 bp, synthetic construct
  misc_feature 1..41: note = Description of Artificial Sequence: Synthetic primer
  source 1..41: mol_type = other DNA
gtcttgtacaggtcggacacctgcacgactttcttgtagcc

SEQ ID NO: 177 - DNA, 35 bp, synthetic construct
  misc_feature 1..35: note = Description of Artificial Sequence: Synthetic primer
  source 1..35: mol_type = other DNA
gcatcctggaactgttccagaccagctactgcccc

SEQ ID NO: 178 - DNA, 35 bp, synthetic construct
  misc_feature 1..35: note = Description of Artificial Sequence: Synthetic primer
  source 1..35: mol_type = other DNA
ggggcagtagctggtctggaacagttccaggatgc

SEQ ID NO: 179 - DNA, 35 bp, synthetic construct
  misc_feature 1..35: note = Description of Artificial Sequence: Synthetic primer
  source 1..35: mol_type = other DNA
ccgtgatgaagttcctgcagctgaccagcgagaag

SEQ ID NO: 180 - DNA, 35 bp, synthetic construct
  misc_feature 1..35: note = Description of Artificial Sequence: Synthetic primer
  source 1..35: mol_type = other DNA
cttctcgctggtcagctgcaggaacttcatcacgg

SEQ ID NO: 181 - DNA, 36 bp, synthetic construct
  misc_feature 1..36: note = Description of Artificial Sequence: Synthetic primer
  source 1..36: mol_type = other DNA
cactgggctacatgctgcagctgaccaacatgatcc

SEQ ID NO: 182 - DNA, 36 bp, synthetic construct
  misc_feature 1..36: note = Description of Artificial Sequence: Synthetic primer
  source 1..36: mol_type = other DNA
ggatcatgttggtcagctgcagcatgtagcccagtg

SEQ ID NO: 183 - DNA, 39 bp, synthetic construct
  misc_feature 1..39: note = Description of Artificial Sequence: Synthetic primer
  source 1..39: mol_type = other DNA
ctacatcctgagcctgctgcagcagggctaccacttcac

SEQ ID NO: 184 - DNA, 39 bp, synthetic construct
  misc_feature 1..39: note = Description of Artificial Sequence: Synthetic primer
  source 1..39: mol_type = other DNA
gtgaagtggtagccctgctgcagcaggctcaggatgtag

SEQ ID NO: 185 - DNA, 43 bp, synthetic construct
  misc_feature 1..43: note = Description of Artificial Sequence: Synthetic primer
  source 1..43: mol_type = other DNA
gagaagtacctgagcgagttttgcttcagcggcacctacatcc

SEQ ID NO: 186 - DNA, 43 bp, synthetic construct
  misc_feature 1..43: note = Description of Artificial Sequence: Synthetic primer
  source 1..43: mol_type = other DNA
ggatgtaggtgccgctgaagcaaaactcgctcaggtacttctc

SEQ ID NO: 187 - DNA, 36 bp, synthetic construct
  misc_feature 1..36: note = Description of Artificial Sequence: Synthetic primer
  source 1..36: mol_type = other DNA
gttcgagatccagggcaccggcaattaccagcagtg

SEQ ID NO: 188 - DNA, 36 bp, synthetic construct
  misc_feature 1..36: note = Description of Artificial Sequence: Synthetic primer
  source 1..36: mol_type = other DNA
cactgctggtaattgccggtgccctggatctcgaac

SEQ ID NO: 189 - DNA, 37 bp, synthetic construct
  misc_feature 1..37: note = Description of Artificial Sequence: Synthetic primer
  source 1..37: mol_type = other DNA
cgccgatagctgggagcacatccacttcatcggcaag

SEQ ID NO: 190 - DNA, 37 bp, synthetic construct
  misc_feature 1..37: note = Description of Artificial Sequence: Synthetic primer
  source 1..37: mol_type = other DNA
cttgccgatgaagtggatgtgctcccagctatcggcg

SEQ ID NO: 191 - DNA, 120 bp, synthetic construct
  misc_feature 1..120: note = Description of Artificial Sequence: Synthetic primer
  source 1..120: mol_type = other DNA
gagatcggcatctacctgaccgactgcatggaacgggccatggaagtgatccccagaagccagcaccaggaaacccccgtgtatctgggagccaccgccggcatgagactgctgagaatg

SEQ ID NO: 192 - DNA, 25 bp, synthetic construct
  misc_feature 1..25: note = Description of Artificial Sequence: Synthetic primer
  source 1..25: mol_type = other DNA
gagatcggcatctacctgaccgact

SEQ ID NO: 193 - DNA, 18 bp, synthetic construct
  misc_feature 1..18: note = Description of Artificial Sequence: Synthetic primer
  source 1..18: mol_type = other DNA
cattctcagcagtctcat

SEQ ID NO: 194 - DNA, 120 bp, synthetic construct
  misc_feature 1..120: note = Description of Artificial Sequence: Synthetic primer
  source 1..120: mol_type = other DNA
cttcagcgccttctactccgtgatgaagttcctgaacctgaccagcgagaaggtgtcccaggaaaaagtgacagagatgatgaagaagttctgcgcccagccctgggaggaaatcaagac

SEQ ID NO: 195 - DNA, 19 bp, synthetic construct
  misc_feature 1..19: note = Description of Artificial Sequence: Synthetic primer
  source 1..19: mol_type = other DNA
cttcagcgccttctactcc

SEQ ID NO: 196 - DNA, 19 bp, synthetic construct
  misc_feature 1..19: note = Description of Artificial Sequence: Synthetic primer
  source 1..19: mol_type = other DNA
ggtcttgatttcctcccag

SEQ ID NO: 197 - DNA, 119 bp, synthetic construct
  misc_feature 1..119: note = Description of Artificial Sequence: Synthetic primer
  source 1..119: mol_type = other DNA
ctgggaggaaatcaagacctcctacgctggcgtgaaagagaagtacctgagcgagttctgcttcagcggcacctacatcctgagcctgctgctgcagggctaccacttcaccgccgata

SEQ ID NO: 198 - DNA, 119 bp, synthetic construct
  misc_feature 1..119: note = Description of Artificial Sequence: Synthetic primer
  source 1..119: mol_type = other DNA
ctgggaggaaatcaagacctcctacgctggcgtgaaagagaagtacctgagcgagtactgcttcagcggcacctacatcctgagcctgctgcagcagggctaccacttcaccgccgata

SEQ ID NO: 199 - DNA, 119 bp, synthetic construct
  misc_feature 1..119: note = Description of Artificial Sequence: Synthetic primer
  source 1..119: mol_type = other DNA
ctgggaggaaatcaagacctcctacgctggcgtgaaagagaagtacctgagcgagttctgcttcagcggcacctacatcctgagcctgctgcagcagggctaccacttcaccgccgata

SEQ ID NO: 200 - DNA, 19 bp, synthetic construct
  misc_feature 1..19: note = Description of Artificial Sequence: Synthetic primer
  source 1..19: mol_type = other DNA
ctgggaggaaatcaagacc

SEQ ID NO: 201 - DNA, 19 bp, synthetic construct
  misc_feature 1..19: note = Description of Artificial Sequence: Synthetic primer
  source 1..19: mol_type = other DNA
tatcggcggtgaagtggta

SEQ ID NO: 202 - DNA, 119 bp, synthetic construct
  misc_feature 1..119: note = Description of Artificial Sequence: Synthetic primer
  source 1..119: mol_type = other DNA
ctgggaggaaatcaagacctcctacgctggcgtgaaagagaagtacctgagcgagtactgcttcagcggcacctacatcctgagcctgctgctgcagggctaccacttcaccgccgata

SEQ ID NO: 203 - DNA, 26 bp, synthetic construct
  misc_feature 1..26: note = Description of Artificial Sequence: Synthetic primer
  source 1..26: mol_type = other DNA
caccggccaggaagccggcgcctacg

SEQ ID NO: 204 - DNA, 26 bp, synthetic construct
  misc_feature 1..26: note = Description of Artificial Sequence: Synthetic primer
  source 1..26: mol_type = other DNA
cgtaggcgccggcttcctggccggtg

SEQ ID NO: 205 - DNA, 34 bp, synthetic construct
  misc_feature 1..34: note = Description of Artificial Sequence: Synthetic primer
  source 1..34: mol_type = other DNA
ggacctgggcggagctgctacccaagtgaccttc

SEQ ID NO: 206 - DNA, 34 bp, synthetic construct
  misc_feature 1..34: note = Description of Artificial Sequence: Synthetic primer
  source 1..34: mol_type = other DNA
gaaggtcacttgggtagcagctccgcccaggtcc

SEQ ID NO: 207 - DNA, 80 bp, synthetic construct
  misc_feature 1..80: note = Description of Artificial Sequence: Synthetic primer
  source 1..80: mol_type = other DNA
ctggatcaccatcaactacctgctgggcaagttcagccagaagaccagatggttcagcatcgtgccctacgagacaaaca

SEQ ID NO: 208 - DNA, 69 bp, synthetic construct
  misc_feature 1..69: note = Description of Artificial Sequence: Synthetic primer
  source 1..69: mol_type = other DNA
tcacttgggtagaagctccgcccaggtccagggcgccgaaggtttcctgattgtttgtctcgtagggca

SEQ ID NO: 209 - protein, 430 aa, synthetic construct
  REGION 1..430: note = Description of Artificial Sequence: Synthetic polypeptide
APTTQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 210 - DNA, 1290 bp, synthetic construct
  misc_feature 1..1290: note = Description of Artificial Sequence: Synthetic polynucleotide
  source 1..1290: mol_type = other DNA
gcccctaccacccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccatggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 211 - protein, 430 aa, synthetic construct
  REGION 1..430: note = Description of Artificial Sequence: Synthetic polypeptide
APTTQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEFCFSGTYILSLLLQGYHFTADSWEHIHFIGKIQGSDAGWTLGYMLNLTNMIPAEQPLSTPLSHST

SEQ ID NO: 212 - DNA, 1290 bp, synthetic construct
  misc_feature 1..1290: note = Description of Artificial Sequence: Synthetic polynucleotide
  source 1..1290: mol_type = other DNA
gcccctaccacccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccatggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagttttgcttcagcggcacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 213 - protein, 433 aa, synthetic construct
  REGION 1..433: note = Description of Artificial Sequence: Synthetic polypeptide
APTSSSTQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEFCFSGTYILSLLLQGYHFTADSWEHIHFIGKIQGSDAGWTLGYMLNLTNMIPAEQPLSTPLSHST

SEQ ID NO: 214 - DNA, 1299 bp, synthetic construct
  misc_feature 1..1299: note = Description of Artificial Sequence: Synthetic polynucleotide
  source 1..1299: mol_type = other DNA
gcccctaccagcagcagcacccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccatggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagttctgcttcagcggcacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 215 - protein, 442 aa, synthetic construct
  REGION 1..442: note = Description of Artificial Sequence: Synthetic polypeptide
APTSSSTKKTQLTSSTQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVNEKYLSEFCFSGTYILSLLLQGYHFTADSWEHIHFIGKIQGSDAGWTLGYMLNLTNMIPAEQPLSTPLSHST

SEQ ID NO: 216 - DNA, 1326 bp, synthetic construct
  misc_feature 1..1326: note = Description of Artificial Sequence: Synthetic polynucleotide
  source 1..1326: mol_type = other DNA
gcccctaccagcagcagcaccaagaaaacccagctgaccagcagcacccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccatggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccaaccctgggaggaaatcaagacctcctacgctggcgtgaacgagaagtacctgagcgagttttgcttcagcggcacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 217 - protein, 430 aa, synthetic construct
  REGION 1..430: note = Description of Artificial Sequence: Synthetic polypeptide
APTTQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEFCFSGTYILSLLQQGYHFTADSWEHIHFIGKIQGSDAGWTLGYMLNLTNMIPAEQPLSTPLSHST

SEQ ID NO: 218 - DNA, 1290 bp, synthetic construct
  misc_feature 1..1290: note = Description of Artificial Sequence: Synthetic polynucleotide
  source 1..1290: mol_type = other DNA
gcccctaccacccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccatggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagttctgcttcagcggcacctacatcctgagcctgctgcagcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 219 - protein, 433 aa, synthetic construct
  REGION 1..433: note = Description of Artificial Sequence: Synthetic polypeptide
APTSSSTQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEFCFSGTYILSLLQQGYHFTADSWEHIHFIGKIQGSDAGWTLGYMLNLTNMIPAEQPLSTPLSHST

SEQ ID NO: 220 - DNA, 1299 bp, synthetic construct
  misc_feature 1..1299: note = Description of Artificial Sequence: Synthetic polynucleotide
  source 1..1299: mol_type = other DNA
gcccctaccagcagcagcacccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccatggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagttctgcttcagcggcacctacatcctgagcctgctgcagcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 221 - protein, 430 aa, synthetic construct
  REGION 1..430: note = Description of Artificial Sequence: Synthetic polypeptide
APTTQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYSVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 222 - DNA, 1290 bp, synthetic construct
  misc_feature 1..1290: note = Description of Artificial Sequence: Synthetic polynucleotide
  source 1..1290: mol_type = other DNA
gcccctaccacccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccatggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccttggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctactccgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgctgcagggctaccacttcaccgccgatagctgggagcacatccacttcatcggcaagattcagggcagcgacgccggctggacactgggttacatgctgaatctgaccaacatgatccccgccgagcagcccctgagcacacctctgagccacagcacc

SEQ ID NO: 223 - protein, 433 aa, synthetic construct
  REGION 1..433: note = Description of Artificial Sequence: Synthetic polypeptide
APTSSSTQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYSVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 224 - DNA, 1299 bp, synthetic construct
  misc_feature 1..1299: note = Description of Artificial Sequence: Synthetic polynucleotide
  source 1..1299: mol_type = other DNA
gcccctaccagcagcagcacccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccatggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccttggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctactccgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgctgcagggctaccacttcaccgccgatagctgggagcacatccacttcatcggcaagattcagggcagcgacgccggctggacactgggttacatgctgaatctgaccaacatgatccccgccgagcagcccctgagcacacctctgagccacagcacc

SEQ ID NO: 225 - protein, 430 aa, synthetic construct
  REGION 1..430: note = Description of Artificial Sequence: Synthetic polypeptide
APTTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEFCFSGTYILSLLQQGYHFTADSWEHIHFIGKIQGSDAGWTLGYMLNLTNMIPAEQPLSTPLSHST

SEQ ID NO: 226 - DNA, 1290 bp, synthetic construct
  misc_feature 1..1290: note = Description of Artificial Sequence: Synthetic polynucleotide
  source 1..1290: mol_type = other DNA
gcccctaccacccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagttttgcttcagcggcacctacatcctgagcctgctgcagcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 227 - protein, 433 aa, synthetic construct
  REGION 1..433: note = Description of Artificial Sequence: Synthetic polypeptide
APTSSSTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEFCFSGTYILSLLQQGYHFTADSWEHIHFIGKIQGSDAGWTLGYMLNLTNMIPAEQPLSTPLSHST

SEQ ID NO: 228 - DNA, 1299 bp, synthetic construct
  misc_feature 1..1299: note = Description of Artificial Sequence: Synthetic polynucleotide
  source 1..1299: mol_type = other DNA
gcccctaccagcagcagcacccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagttttgcttcagcggcacctacatcctgagcctgctgcagcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 229 - protein, 430 aa, synthetic construct
  REGION 1..430: note = Description of Artificial Sequence: Synthetic polypeptide
APTTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYSVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 230 - DNA, 1290 bp, synthetic construct
  misc_feature 1..1290: note = Description of Artificial Sequence: Synthetic polynucleotide
  source 1..1290: mol_type = other DNA
gcccctaccacccagaacaaggccctgcccgagaacgtgaagtacggcatcgtgctggacgccggctcctcccacacctccctgtacatctacaagtggcctgccgagaaagaaaacgacaccggcgtggtgcaccaagtggaagagtgcagagtgaagggccccggcatctccaagttcgtgcagaaagtgaacgagatcggcatctacctgaccgactgcatggaacgggccagagaagtgatccctcggtcccagcaccaggaaacccctgtctacctgggcgccaccgccggcatgcggctgctgcggatggaatccgaggaactggccgaccgggtgctggacgtggtggaacggtccctgtccaactacccattcgattttcaaggcgccagaatcatcaccggccaggaagagggcgcctacggctggatcaccatcaactacctgctgggcaagttctcccagaagaatcaggaaaccttcggcgccctggacctgggcggagccagcacccaagtcacattcgtgccccagaaccagaccatcgagagccccgacaacgccctgcagttccggctgtacggcaaggactacaacgtgtacacccacagctttctgtgctacggcaaggaccaggccctgtggcagaagctggccaaggacatccaagtggcctccaacgagatcctgcgggacccctgcttccaccccggctacaagaaagtggtcaacgtgtccgacctgtacaagaccccttgcaccaagagattcgagatgaccctgcccttccagcagttcgagatccagggcatcggcaactaccagcagtgccaccagtccatcctggaactgttcaacacctcctactgcccctactcccagtgcgccttcaacggcatcttcctgcctccactgcagggcgacttcggcgccttctccgccttctactccgtgatgaagttcctgaacctgacctccgagaaagtgtcccaggaaaaagtgaccgagatgatgaagaagttctgcgcccagccctgggaggaaatcaagacctcctacgctggcgtgaaagagaagtacctgtccgagtactgcttctccggcacctacatcctgtccctgctgctgcagggctaccacttcaccgccgacagctgggagcacatccacttcatcggcaagatccagggatccgacgctggctggaccctgggctacatgctgaatctgaccaacatgatccccgccgagcagcccctgtccacccctctgtctcactccacc

SEQ ID NO: 231 - protein, 433 aa, synthetic construct
  REGION 1..433: note = Description of Artificial Sequence: Synthetic polypeptide
APTSSSTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYSVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 232 - DNA, 1299 bp, synthetic construct
  misc_feature 1..1299: note = Description of Artificial Sequence: Synthetic polynucleotide
  source 1..1299: mol_type = other DNA
gcccctaccagcagcagcacccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccttggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctactccgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgctgcagggctaccacttcaccgccgatagctgggagcacatccacttcatcggcaagattcagggcagcgacgccggctggacactgggttacatgctgaatctgaccaacatgatccccgccgagcagcccctgagcacacctctgagccacagcacc

SEQ ID NO: 233 - protein, 430 aa, synthetic construct
  REGION 1..430: note = Description of Artificial Sequence: Synthetic polypeptide
APTTQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYSVMKFLNLTSEKVSQEKVTEMMKKFCAQPWEEIKTSYAGVKEKYLSEYCFSGTYILSLLQQGYHFTADSWEHIHFIGKIQGSDAGWTLGYMLNLTNMIPAEQPLSTPLSHST

SEQ ID NO: 234 - DNA, 1290 bp, synthetic construct
  misc_feature 1..1290: note = Description of Artificial Sequence: Synthetic polynucleotide
  source 1..1290: mol_type = other DNA
gcccctaccacccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccatggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctactccgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgcagcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 235 - protein, 433 aa, synthetic construct
  REGION 1..433: note = Description of Artificial Sequence: Synthetic polypeptide
APTSSSTQNKALPENVKYGIVLDAGSSHTSLYIYKWPAEKENDTGVVHQVEECRVKGPGISKFVQKVNEIGIYLTDCMERAMEVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYSVMKFLNLTSEKVSQEKVTEMMKKFCAQPWEEIKTSYAGVKEKYLSEYCFSGTYILSLLQQGYHFTADSWEHIHFIGKIQGSDAGWTLGYMLNLTNMIPAEQPLSTPLSHST

SEQ ID NO: 236 - DNA, 1299 bp, synthetic construct
  misc_feature 1..1299: note = Description of Artificial Sequence: Synthetic polynucleotide
  source 1..1299: mol_type = other DNA
gcccctaccagcagcagcacccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccatggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctactccgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgcagcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 237 - protein, 430 aa, synthetic construct
  REGION 1..430: note = Description of Artificial Sequence: Synthetic polypeptide
APTTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVNEKYLSEFCFSGTYILSLLLQGYHFTADSWEHIHFIGKIQGSDAGWTLGYMLNLTNMIPAEQPLSTPLSHST

SEQ ID NO: 238 - DNA, 1290 bp, synthetic construct
  misc_feature 1..1290: note = Description of Artificial Sequence: Synthetic polynucleotide
  source 1..1290: mol_type = other DNA
gcccctaccacccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccaaccctgggaggaaatcaagacctcctacgctggcgtgaacgagaagtacctgagcgagttttgcttcagcggcacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 239 - protein, 433 aa, synthetic construct
  REGION 1..433: note = Description of Artificial Sequence: Synthetic polypeptide
APTSSSTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVNEKYLSEFCFSGTYILSLLLQGYHFTADSWEHIHFIGKIQGSDAGWTLGYMLNLTNMIPAEQPLSTPLSHST

SEQ ID NO: 240 - DNA, 1299 bp, synthetic construct
  misc_feature 1..1299: note = Description of Artificial Sequence: Synthetic polynucleotide
  source 1..1299: mol_type = other DNA
gcccctaccagcagcagcacccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccaaccctgggaggaaatcaagacctcctacgctggcgtgaacgagaagtacctgagcgagttttgcttcagcggcacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 241 - protein, 442 aa, synthetic construct
  REGION 1..442: note = Description of Artificial Sequence: Synthetic polypeptide
APTSSSTKKTQLTSSTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVNEKYLSEFCFSGTYILSLLLQGYHFTADSWEHIHFIGKIQGSDAGWTLGYMLNLTNMIPAEQPLSTPLSHST

SEQ ID NO: 242 - DNA, 1326 bp, synthetic construct
  misc_feature 1..1326: note = Description of Artificial Sequence: Synthetic polynucleotide
  source 1..1326: mol_type = other DNA
gcccctaccagcagcagcaccaagaaaacccagctgaccagcagcacccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccaaccctgggaggaaatcaagacctcctacgctggcgtgaacgagaagtacctgagcgagttttgcttcagcggcacctacatcctgagcctgctgctgcagggctaccacttcaccgccgatagctgggagcacatccacttcatcggcaagattcagggcagcgacgccggctggacactgggctacatgctgaatctgaccaacatgatccccgccgagcagcccctgagcacacctctgagccacagcacc

SEQ ID NO: 243 - protein, 430 aa, synthetic construct
  REGION 1..430: note = Description of Artificial Sequence: Synthetic polypeptide
APTTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLQQGYHFTADSWEDIHFIGKIQGSDAGWTLGYMLNLTNMIPAEQPLSTPLSHST

SEQ ID NO: 244 - DNA, 1290 bp, synthetic construct
  misc_feature 1..1290: note = Description of Artificial Sequence: Synthetic polynucleotide
  source 1..1290: mol_type = other DNA
gcccctaccacccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgcagcagggctaccacttcaccgccgatagctgggaggacatccacttcatcggcaagattcagggcagcgacgccggctggacactgggctacatgctgaatctgaccaacatgatccccgccgagcagcccctgagcacacctctgagccacagcacc

SEQ ID NO: 245 - protein, 433 aa, synthetic construct
  REGION 1..433: note = Description of Artificial Sequence: Synthetic polypeptide
APTSSSTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLQQGYHFTADSWEDIHFIGKIQGSDAGWTLGYMLNLTNMIPAEQPLSTPLSHST

SEQ ID NO: 246 - DNA, 1299 bp, synthetic construct
  misc_feature 1..1299: note = Description of Artificial Sequence: Synthetic polynucleotide
  source 1..1299: mol_type = other DNA
gcccctaccagcagcagcacccagaacaaggccctgcccgagaacgtgaagtacggcatcgtgctggatgccggcagcagccacaccagcctgtacatctacaagtggcctgccgagaaagaaaacgacaccggcgtggtgcatcaggtggaagagtgcagagtgaagggccctggcatcagcaagttcgtgcagaaagtgaacgagatcggcatctacctgaccgactgcatggaacgggccagggaagtgatccccagaagccagcaccaggaaacccccgtgtatctgggagccaccgccggcatgagactgctgagaatggaaagcgaggaactggccgaccgggtgctggacgtggtggaaagaagcctgagcaactacccattcgattttcaaggcgccagaatcatcaccggccaggaagaaggcgcctacggctggatcaccatcaactacctgctgggcaagttcagccagaagaatcaggaaaccttcggcgccctggacctgggcggagcttctacccaagtgaccttcgtgccccagaatcagaccatcgagagccccgacaacgccctgcagttccggctgtacggcaaggactacaatgtgtacacccacagctttctgtgctacggaaaggaccaggctctgtggcagaagctggccaaggacatccaggtggccagcaacgagatcctgcgggacccttgcttccaccccggctacaagaaagtcgtgaacgtgtccgacctgtacaagaccccctgcaccaagagattcgagatgaccctgcccttccagcagttcgagatccagggcatcggcaattaccagcagtgccaccagagcatcctggaactgttcaacaccagctactgcccctacagccagtgcgccttcaacggcatcttcctgccacctctgcagggggatttcggcgccttcagcgccttctacttcgtgatgaagttcctgaacctgaccagcgagaaggtgtcccaggaaaaagtgacagagatgatgaagaagttctgcgcccagccctgggaggaaatcaagacctcctacgctggcgtgaaagagaagtacctgagcgagtactgcttcagcggcacctacatcctgagcctgctgcagcagggctaccacttcaccgccgatagctgggaggacatccacttcatcggcaagattcagggcagcgacgccggctggacactgggctacatgctgaatctgaccaacatgatccccgccgagcagcccctgagcacacctctgagccacagcacc

SEQ ID NO: 247 - protein, 6 aa, synthetic construct
  REGION 1..6: note = Description of Artificial Sequence: Synthetic peptide
EFRHDS

SEQ ID NO: 248 - DNA, 18 bp, synthetic construct
  misc_feature 1..18: note = Description of Artificial Sequence: Synthetic oligonucleotide
  source 1..18: mol_type = other DNA
gaattccggcacgacagc

SEQ ID NO: 249 - protein, 6 aa, synthetic construct
  REGION 1..6: note = Description of Artificial Sequence: Synthetic 6xHis tag
HHHHHH

SEQ ID NO: 250 - DNA, 18 bp, synthetic construct
  misc_feature 1..18: note = Description of Artificial Sequence: Synthetic oligonucleotide
  source 1..18: mol_type = other DNA
catcatcatcatcatcac

SEQ ID NO: 251 - protein, 5 aa, synthetic construct
  REGION 1..5: note = Description of Artificial Sequence: Synthetic peptide
GGGGS

SEQ ID NO: 252 - protein, 8 aa, synthetic construct
  REGION 1..8: note = Description of Artificial Sequence: Synthetic peptide
VKEKYLSE

SEQ ID NO: 253 - protein, 7 aa, synthetic construct
  REGION 1..7: note = Description of Artificial Sequence: Synthetic peptide
QERWLRD

(19) United States
(12) Patent Application Publication
(10) Pub. No.: US 2025/0179447 A1
(43) Pub. Date: Jun. 5, 2025

(54) SOLUBILIZED APYRASES, METHODS AND USE

(71) Applicant: NOVARTIS AG, Basel (CH)

(72) Inventors: Agostino CIRILLO, Basel (CH); Hilmar EBERSBACH, wallisellen (CH); Boerje HARALDSSON, Basel (CH); Thomas HUBER, Allschwil (CH); Guido JUNGE, Basel (CH); Regina LINK, Münchenstein (CH); Max WARNCKE, Basel (CH); Chao ZOU, Allschwil (CH)

(21) Appl. No.: 18/897,352

(22) Filed: Sep. 26, 2024

Related U.S. Application Data
(62) Division of application No. 17/261,533, filed on Jan. 19, 2021, now abandoned.

(30) Foreign Application Priority Data
Jul. 18, 2018 (EP) 18184269.1

(51) Int. Cl. C12N 9/14; A61K 38/00; A61K 38/46; A61K 45/06

(52) U.S. Cl. CPC C12N 9/14; A61K 38/465; A61K 45/06; A61K 38/00; C12Y 306/01005

(57) ABSTRACT

The invention relates to design and therapeutic use of solubilized apyrase polypeptides, pharmaceuticals compositions, therapeutic uses and methods useful for preventing and treating tissue damage.

### RELATED APPLICATIONS

This application is a divisional of Ser. No. 17/261,533, filed Jan. 19, 2021 which is a 371 Application of PCT/IB2019/056117, filed Jul. 17, 2019, which claims priority to EP 18184269.1, filed Jul. 18, 2018, the disclosure of which is incorporated by reference herein in its entirety.

### SEQUENCE LISTING

The instant application contains a Sequence Listing which has been submitted electronically in XML file format and is hereby incorporated by reference in its entirety. Said XML copy, created on Oct. 15, 2024, is named PAT057510-US-DIV_SL.xml and is 476,018 bytes in size.

### TECHNICAL FIELD

The present invention relates to design and therapeutic use of solubilized apyrase polypeptides, pharmaceuticals compositions, and methods useful for preventing and treating tissue damage.

### BACKGROUND OF THE DISCLOSURE

Apyrase (ATP-diphosphatase, adenosine diphosphatase, ADPase, or ATP diphosphohydrolase) is a plasma membrane-bound enzyme group of enzymes active against both di- and triphosphate nucleotides (NDPs and NTPs) and hydrolyze NTPs to nucleotide monophosphates (NMPs) in two distinct successive phosphate-releasing steps, with NDPs as intermediates. Most of the ecto-ATPases that occur on the cell surface and hydrolyze extracellular nucleotides belong to this enzyme family. They differ from ATPases, which specifically hydrolyze ATP, by hydrolyzing both ATP and ADP.

The first known human apyrase, ectonucleoside triphosphate diphosphohydrolase-1 (gene: ENTPD1, protein: NTPDase1), also known as cluster of differentiation 39 (CD39, UniProt P49961, or SEQ ID NO: 1) is a cell surface-located enzymes with an extracellularly facing catalytic site.

Among the known human CD39 family, the member CD39L3 is known as an ecto-apyrase (ecto-ATPDase) with biochemical activity between CD39 and CD39L1 (ecto-ATPase). Specifically human CD39L3 has been solubilized and purified for therapeutic purposes, e.g. as disclosed in U.S. Pat. No. 7,247,300B1 (incorporated herein by reference) or included herein as SEQ ID NO: 3.

### SUMMARY OF THE DISCLOSURE

The present disclosure is inter alia based on the unexpected finding that certain modifications of solubilized human apyrase, such as human CD39 lead to a surprisingly active protein, which is still safe and easy to manufacture.

According to a first aspect of the invention, a solubilized human apyrase with at least two modifications selected from the list consisting of: N terminal deletion, C terminal deletion and central modification is provided.

In one embodiment the solubilized human apyrase comprises a N terminal deletion, a C terminal deletion and a modification deletion.

In one embodiment, the central modification comprises a deletion of one or more amino acids. In another embodiment, the central modification comprises a point mutation of one or more amino acids, such as a substitution mutation. In yet another embodiment, the central modification is a combination of a deletion of one or more amino acids and a point mutation, such as a substitution mutation, of one or more amino acids.

The N terminal deletion may be between 30 and 50 amino acids deleted from the N terminus of the wild type CD39 sequence according to SEQ ID NO: 1, such as a deletion of 30, 31, 32, 33, 34, 35, 36, 37, 38, 39, 40, 41, 42, 43, 44, 45, 46, 47, 48, 49 or 50 amino acids. In a preferred embodiment, the N terminal deletion is 34, 37, 38 or 45 amino acids.

The C terminal deletion may be between 20 and 40 amino acids deleted from the C terminus of the wild type CD39 sequence according to SEQ ID NO: 1, such as a deletion of 20, 21, 22, 23, 24, 25, 26, 27, 28, 29, 30, 31, 32, 33, 34, 35, 36, 37, 38, 39 or 40 amino acids. In a preferred embodiment, the C terminal deletion is 22, 29 of 37 amino acids.

The central deletion may be between 10 and 15 consecutive amino acids, deleted from the wild type CD39 sequence according to SEQ ID NO: 1, such as a deletion of 10, 11, 12, 13, 14 or 15 amino acids. In a preferred embodiment, the central deletion is 12 amino acids, such as amino acids number 193 to 204 in relation to the wild type CD39 sequence according to SEQ ID NO: 1.

In one embodiment, the solubilized human apyrase comprises one, two, three, four, or five point mutation(s) in relation to the wild type CD39 sequence according to SEQ ID NO: 1, selected from the group consisting of K71E, N73Q, V95A, G102D, Y104S, T106S, R113M, L149M, V151A, E173D, T229A, L254M, K258R, W263R, E276D, N292Q, R304G, 1319T, N327Q, A362N, F365S, N371Q, K405N, Y412F, L424Q, H436D, 1437N, F439S, G441D, N457Q, P463S, and S469R.

In one embodiment the solubilized human apyrase comprises a sequence selected from the group consisting of SEQ ID NO: 4, SEQ ID NO: 6, SEQ ID NO: 32, SEQ ID NO: 54, SEQ ID NO: 56, SEQ ID NO: 70, SEQ ID NO: 76, and SEQ ID NO: 78.

In one embodiment, the solubilized human apyrase comprises a sequence selected from the group consisting of SEQ ID NO: 131, SEQ ID NO: 133, SEQ ID NO: 135, SEQ ID NO: 137, SEQ ID NO: 139 and SEQ ID NO: 141.

In one specific embodiment, the solubilized human apyrase comprises of a sequence selected from the group consisting of SEQ ID NO: 213, SEQ ID NO: 227, SEQ ID NO: 219, SEQ ID NO: 227, SEQ ID NO: 217, SEQ ID NO: 209, SEQ ID NO: 221, SEQ ID NO: 72, SEQ ID NO: 215, SEQ ID NO: 223, SEQ ID NO: 211, SEQ ID NO: 58 and SEQ ID NO: 229.

In one specific embodiment, the solubilized human apyrase consists of a sequence selected from the group consisting of SEQ ID NO: 213, SEQ ID NO: 227, SEQ ID NO: 219, SEQ ID NO: 227, SEQ ID NO: 217, SEQ ID NO: 209, SEQ ID NO: 221, SEQ ID NO: 72, SEQ ID NO: 215, SEQ ID NO: 223, SEQ ID NO: 211, SEQ ID NO: 58 and SEQ ID NO: 229.

In a preferred embodiment, the solubilized human apyrase comprises a sequence selected from the group consisting of SEQ ID NO: 58, SEQ ID NO: 72 and SEQ ID NO: 229.

In one embodiment, the solubilized human apyrase comprises SEQ ID NO: 58. In one embodiment, the solubilized human apyrase comprises SEQ ID NO: 72. In one embodiment, the solubilized human apyrase comprises SEQ ID NO: 229.

In a preferred embodiment, the solubilized human apyrase consists of a sequence selected from the group consisting of SEQ ID NO: 58, SEQ ID NO: 72 and SEQ ID NO: 229.

In one embodiment, the solubilized human apyrase consists SEQ ID NO: 58. In one embodiment, the solubilized human apyrase consists SEQ ID NO: 72. In one embodiment, the solubilized human apyrase consists SEQ ID NO: 229.

According to a second aspect of the invention, the invention relates to a pharmaceutical composition comprising a therapeutically effective dose of the apyrase according to the first aspect of the invention, and one or more pharmaceutically acceptable carriers is provided.

In one embodiment, the pharmaceutical composition further comprises one or more additional active ingredients.

According to a third aspect of the invention, an isolated apyrase according to the first aspect for use as a medicament is provided.

According to a fourth aspect of the invention, an isolated apyrase according to the first aspect for use in the treatment of tissue damage is provided.

The tissue damage may be acute brain injury (stroke); acute multi-organ failure; delayed graft function after transplantation of kidney or other solid organs; burn damage; radiation damage; acute damage due to trauma and/or hypoxia, such as acute respiratory distress syndrome (ARDS) or lung injury; acute kidney injury, such as acute kidney injury secondary to thoracic surgery (e.g. aortic valve replacement, coronary artery bypass surgery) or sepsis or rhabdomyolysis or toxic effects of antibiotics or other medication; acute myocardial injury.

In another embodiment, the fourth aspect of the disclosure relates to an isolated apyrase according to the first aspect of the invention for use in the treatment of cardiac surgery associated acute kidney injury.

In another embodiment, the fourth aspect of the disclosure relates to an isolated apyrase according to the first aspect of the invention for use in the treatment of delayed graft function (DGF), acute respiratory distress syndrome (ARDS), acute myocardial infarction (AMI), traumatic brain injury (TBI)/acute ischemic stroke (AIS), ischemia-reperfusion injury (IRI), or combinations thereof often referred to as multi-organ failures (MOF).

In one embodiment, the solubilized human apyrase used for the treatment of cardiac surgery associated acute kidney injury comprises an amino acid sequence of SEQ ID NO: 58.

In one embodiment, the solubilized human apyrase used for the treatment of cardiac surgery associated acute kidney injury comprises an amino acid sequence of SEQ ID NO: 72. In one embodiment, the solubilized human apyrase used for the treatment of cardiac surgery associated acute kidney injury comprises an amino acid sequence of SEQ ID NO: 229.

In an additional preferred embodiment the disclosure relates to the use of an isolated apyrase according to the first aspect of the invention for the treatment of sepsis associated acute kidney injury.

In one embodiment of the fourth aspect, the solubilized human apyrase for use in the treatment of sepsis associated acute kidney injury comprises an amino acid sequence of SEQ ID NO: 58.

In one embodiment of the fourth aspect, the solubilized human apyrase for use in the treatment of sepsis associated acute kidney injury comprises an amino acid sequence of SEQ ID NO: 72.

In one embodiment of the fourth aspect, the solubilized human apyrase for use in the treatment of sepsis associated acute kidney injury comprises an amino acid sequence of SEQ ID NO: 229.

According to a fifth aspect of the invention, a method of treating tissue damage in a human subject is provided, comprising administering a therapeutically effective dose of solubilized human apyrase according to the first aspect to said subject. One embodiment of the fifth aspect of the invention relates to a method of treating cardiac surgery associated acute kidney injury comprising administering a therapeutically effective dose of an isolated apyrase according to the first aspect of the invention to a subject in need of such treatment. 10

Another embodiment of the fifth aspect of the invention relates to a method of treating delayed graft function (DGF), acute respiratory distress syndrome (ARDS), acute myocardial infarction (AMI), traumatic brain injury (TBI)/acute ischemic stroke (AIS) ischemia-reperfusion injury (IRI), or combinations thereof often referred to as multi-organ failures (MOF) comprising administering a therapeutically effective dose of an isolated apyrase according to the first aspect of the invention to a subject in need of such treatment.

In one embodiment of the fifth aspect, the solubilized human apyrase used in the method of treating cardiac surgery associated acute kidney injury comprises an amino acid sequence of SEQ ID NO: 58, SEQ ID NO: 72 or SEQ ID NO: 229.

One embodiment of the fifth aspect of the invention relates to a method of treating sepsis associated acute kidney injury comprising administering a therapeutically effective dose of an isolated apyrase according to the first aspect of the invention to a subject in need of such treatment.

In one embodiment of the fifth aspect, the solubilized human apyrase used in the method of treating sepsis associated acute kidney injury comprises an amino acid sequence of SEQ ID NO: 58, SEQ ID NO: 72 or SEQ ID NO: 229. The tissue damage may be acute brain injury (stroke); acute multi-organ failure; delayed graft function after transplantation of kidney or other solid organs; burn damage; radiation damage; acute damage due to trauma and/or hypoxia, such as acute respiratory distress syndrome (ARDS) or lung injury; acute kidney injury, such as acute kidney injury secondary to thoracic surgery (e.g. aortic valve replacement, coronary artery bypass surgery) or sepsis or rhabdomyolysis or toxic effects of antibiotics or other medication; acute myocardial injury.

According to a sixth aspect of the invention, an isolated nucleic acid molecule encoding any apyrase according to the first aspect is provided.

According to a seventh aspect of the invention, a cloning or expression vector comprising one or more nucleic acid sequences according to the sixth aspect is provided, wherein the vector is suitable for the recombinant production of isolated apyrase according to the first aspect.

According to an eight aspect of the invention, a host cell is provided comprising one or more cloning or expression vectors according the seventh aspect.

According to a ninth aspect of the invention, a process for the production of an apyrase according to the first aspect is provided, comprising culturing a host cell according to the eight aspect, purifying and recovering said apyrase.

### BRIEF DESCRIPTION OF THE FIGURES

FIG. 1 is a sequence alignment;

FIG. 2A is a representation of an expression level of supernatant containing human CD39 by anti-APP Western Blot according to an embodiment;

FIG. 2B is a representation of expression level of supernatant containing cysteine bridge deletion human CD39 variants by anti-APP Western Blot according to an embodiment;

FIG. 3 is a graph showing solid-phase ATPase assay results of CD39 variants;

FIG. 4 is a graph showing solid-phase ATP cleavage on HEK293 cells transformed with human CD39 variants according to an embodiment;

FIG. 5 is a schematic overview of a vector according to an embodiment;

FIG. 6 is an enzymatic model based on steady state approximation;

FIG. 7 is an overview of kinetic data and model fit for a protein according to an embodiment;

FIG. 8 is an overview of kinetic data and model fit for a protein according to an embodiment;

FIG. 9 is an overview of kinetic data and model fit for a protein according to an embodiment;

FIG. 10A-10B is a schematic representation of experimental conditions;

FIG. 11 is a graph showing AMP levels for proteins according to embodiments; and

FIG. 12A-12C are graphs showing in vivo results for proteins according to embodiments.

### DETAILED DESCRIPTION OF THE DISCLOSURE

The present disclosure is inter alia based on the unexpected finding that certain modifications of solubilized CD39 lead to a surprisingly active protein, which is safe and easy to manufacture.

As will be shown in the specific examples below, a preferred embodiment is a solubilized human apyrase with at least two modifications selected from the list consisting of: N terminal deletion, C terminal deletion and central deletion, such as a solubilized human apyrase comprising a sequence selected from the group consisting of SEQ ID NO: 4, SEQ ID NO: 6, SEQ ID NO: 32, SEQ ID NO: 54, SEQ ID NO: 56, SEQ ID NO: 70, SEQ ID NO: 76, and SEQ ID NO: 78.

The inventors tried several different sequence modification strategies to achieve solubilized human apyrase with both retained activity and ability to be expressed, while still not introducing too many modifications because of the risk of increased immunogenicity and thus increased safety risk. Surprisingly, one sequence modification which was found to both increase efficiency and ability to express human apyrase was a deletion of a central section, the so called delta MIL (ΔMIL) modification, at the same time as not adding too much immunogenicity risk.

To increase the expression of the solubilized human apyrase according to embodiments of the invention, N terminal expression tags were tested. Various N terminal expression tags are known in the art, but surprisingly not all tags worked. The inventors found that a few tags worked, which could not have been foreseen.

These N terminal tags were SEQ ID NO: 131, SEQ ID NO: 133, SEQ ID NO: 135, SEQ ID NO: 137, SEQ ID NO: 139 or SEQ ID NO: 141. As is shown herein, particularly preferred tags are SEQ ID NO: 133, SEQ ID NO: 135 or SEQ ID NO: 137.

Specific details are set forth in Examples 9 to 13 below. However, in order to illustrate the unpredictable nature of these Examples, a comparative summary is presented in Table 1.

TABLE 1
Summary of preferred embodiments.

| | Modifications | | Activity | | |
|---|---|---|---|---|---|
| | No of | | compared to | | |
| | N-terminal | Number of | parental EP28 | | |
| | aa from SEQ | point | In | In | Titer |
| Construct | ID NO: 131 | mutations | vitro | vivo | (g/L) |
| EP1xEP17 | 6 | 2 | 1.5x | | 0.6 |
| EP17xEP19 | 6 | 2 | 1.5x | | 0.26 |
| EP1xEP17xEP19 | 6 | 3 | 1.5x | | 0.46 |
| EP17xEP19 | 3 | 2 | 1.5x | | 0.40 |
| EP1xEP17xEP19 | 3 | 3 | 1.5x | | 0.58 |
| EP1 | 3 | 1 | 1x | | 0.29 |
| EP1xEP14 | 3 | 2 | 4x | | 0.50 |
| EP28 | 16 | 0 | 1x | 1x | 0.08 |
| EP1xEP17_K405N | 15 | 3 | 1.5x | 1x | 1.4 |
| EP1xEP14 | 6 | 2 | 4x | | 0.3 |
| EP1xEP17 | 3 | 2 | 1.5x | | 0.68 |
| EP28 | 3 | 0 | 1x | | 0.10 |
| EP14 | 3 | 1 | 4x | 4x | 0.27 |

### 1. Definitions

To facilitate for a person skilled in the art to practice the invention, the following terms are used throughout the description.

The terms “CD39” and “hCD39” are used synonymously throughout the disclosure and unless stated otherwise means human cluster of differentiation 39 (CD39) according to UniProt P49961 or SEQ ID NO: 1.

The term “apyrase” refers to human apyrase unless stated otherwise. A “solubilized apyrase” as used herein means that that the apyrase, which as a wild type protein exist bound to a cell membrane, has been modified so that it is no longer bound to the cell membrane but exists in a soluble state i.e. no longer anchored to the cell membrane.

The abbreviation “MIL” refers to membrane interaction loop, which is a central part of the wild type (human) CD39 protein which interacts with the cell membrane, in addition to the N terminal and C terminal parts which are physically anchored through the cell membrane. The term “delta MIL”, or “ΔMIL”, refers to the deletion of the MIL sequence from wild type (human) CD39.

The term “about” in relation to a numerical value x means, for example, +/−10%. When used in front of a numerical range or list of numbers, the term “about” applies to each number in the series, e.g., the phrase “about 1-5” should be interpreted as “about 1-about 5”, or, e.g., the phrase “about 1, 2, 3, 4” should be interpreted as “about 1, about 2, about 3, about 4, etc.”

The word “substantially” does not exclude “completely,” e.g., a composition which is “substantially free” from Y may be completely free from Y. Where necessary, the word “substantially” may be omitted from the definition of the disclosure.

The term “comprising” encompasses “including” as well as “consisting,” e.g., a composition “comprising” X may consist exclusively of X or may include something additional, e.g., X+Y.

“Identity” with respect to a native polypeptide and its functional derivative is defined herein as the percentage of amino acid residues in the candidate sequence that are identical with the residues of a corresponding native polypeptide, after aligning the sequences and introducing gaps, if necessary, to achieve the maximum percent identity, and not considering any conservative substitutions as part of the sequence identity. Neither N- or C-terminal extensions nor insertions shall be construed as reducing identity. Methods and computer programs for the alignment are well known. The percent identity can be determined by standard alignment algorithms, for example, the Basic Local Alignment Search Tool (BLAST) described by Altshul et al. ((1990) J. Mol. Biol., 215:403 410); the algorithm of Needleman et al. ((1970) J. Mol. Biol., 48:444 453); or the algorithm of Meyers et al. ((1988) Comput. Appl. Biosci., 4:11 17). A set of parameters may be the Blosum 62 scoring matrix with a gap penalty of 12, a gap extend penalty of 4, and a frameshift gap penalty of 5. The percent identity between two amino acid or nucleotide sequences can also be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

“Amino acid(s)” refer to all naturally occurring L-a-amino acids, e.g., and include D-amino acids. The phrase “amino acid sequence variant” refers to molecules with some differences in their amino acid sequences as compared to the sequences according to the present disclosure. Amino acid sequence variants of a protein according to the present disclosure, e.g., of a specified sequence, still have apyrase activity. Amino acid sequence variants include substitutional variants (those that have at least one amino acid residue removed and a different amino acid inserted in its place at the same position in a polypeptide according to the present disclosure), insertional variants (those with one or more amino acids inserted immediately adjacent to an amino acid at a particular position in a polypeptide according to the present disclosure) and deletional variants (those with one or more amino acids removed in a polypeptide according to the present disclosure).

The term “treatment” or “treat” is herein defined as the application or administration of apyrase according to the invention to a subject, or application or administration a pharmaceutical composition comprising said apyrase to a subject, or an isolated tissue or cell line from a subject, where the subject has tissue damage, a symptom associated with tissue damage, where the purpose is to alleviate, ameliorate, or improve the tissue damage or any associated symptoms of the tissue damage inter alia by reducing levels of extracellular ATP.

By “treatment” is also intended the application or administration of a pharmaceutical composition comprising an apyrase to a subject, or application or administration of a pharmaceutical composition comprising apyrase of the invention to an isolated tissue or cell line from a subject, where the subject has an tissue damage or a symptom associated with tissue damage, where the purpose is to alleviate, ameliorate, or improve the tissue damage or any associated symptoms of the tissue damage.

The term “prevent” or “preventing” refer to prophylactic or preventative treatment; it is concerned about delaying the onset of, or preventing the onset of the disease, disorders and/or symptoms associated thereto.

As used herein, a subject is “in need of” a treatment if such subject would benefit biologically, medically or in quality of life from such treatment.

The term “pharmaceutically acceptable” means a nontoxic material that does not interfere with the effectiveness of the biological activity of the active ingredient(s).

As used herein, the term “administration” or “administering” of the subject compound means providing a compound of the invention and prodrugs thereof to a subject in need of treatment. Administration “in combination with” one or more further therapeutic agents includes simultaneous (concurrent) and consecutive administration in any order, and in any route of administration.

As used herein, a “therapeutically effective dose” refers to a dose (an amount) of an apyrase that is effective, upon single or multiple dose administration to a patient (such as a human) for treating, preventing, preventing the onset of, curing, delaying, reducing the severity of, ameliorating at least one symptom of a disorder or recurring disorder, or prolonging the survival of the patient beyond that expected in the absence of such treatment. When applied to an individual active ingredient (e.g., apyrase) administered alone, the term refers to that ingredient alone. When applied to a combination, the term refers to combined doses or amounts of the active ingredients that result in the therapeutic effect, whether administered in combination, serially or simultaneously.

The phrase “dosage regimen” means the regimen used to treat an illness, e.g., the dosing protocol used during the treatment of tissue damage.

The phrase “means for administering” is used to indicate any available implement for systemically administering a drug to a patient, including, but not limited to, a pre-filled syringe, a vial and syringe, an injection pen, an autoinjector, an intraveneous (i.v.) drip and bag, a pump, a patch pump, etc. With such items, a patient may self-administer the drug (i.e., administer the drug on their own behalf) or a physician may administer the drug.

### 2. Example 1: Membrane Free CD39

Wild type human apyrase CD39 (hCD39, UniProt P49961, or SEQ ID NO: 1) is naturally anchored in the cell membrane by a transmembrane domain at the N-terminus (putative aa 17-37), a central putative membrane interaction loop (MIL putative aa 193-204) and a C-terminal transmembrane domain (putative aa 479-499). To enable expression of a soluble variant of CD39 using a mammalian host cell, several elements of the CD39 sequence have been modified to obtain a membrane free, or solubilized, protein. The natural leader sequence and N-terminal transmembrane region were substituted by a secretion leader and a purification tag (SEQ ID NO: 133). The boundaries of the extracellular domain of CD39 have been varied to optimize expression, purification and activity parameters (amino acids no. 38-476 of SEQ ID NO: 1, amino acids no. 39-469 of SEQ ID NO: 1, amino acids 46-461 of SEQ ID NO: 1, and amino acids 46-476 of SEQ ID NO: 1, respectively). The impact of the cysteines and disulfide bridges on aggregation propensity and enzymatic activity was systematically assessed by substituting the cysteines by alanine or by shortening the loop made up by the disulfide bridge (SEQ ID 107, 109, 111, 113, and 115). A stretch of hydrophobic amino acid was described in the structural work of the rat-CD39 (Zebisch et al, J.Mol. Biol. (2012), 415, 288-306, wild type rat CD39, Uniprot P97687, set forth in SEQ ID NO: 2) and it is thought that this loop may be interacting with the cell membrane (MIL). We translated the findings to the human CD39 sequence by sequence alignment and generated CD39 variants having a loop deletion (CD39ΔMIL or EP28, as set forth in SEQ ID NO: 4). The impact of the deletion (or delta/A) of the MIL on expression level of functional CD39 and thermal stability was assessed.

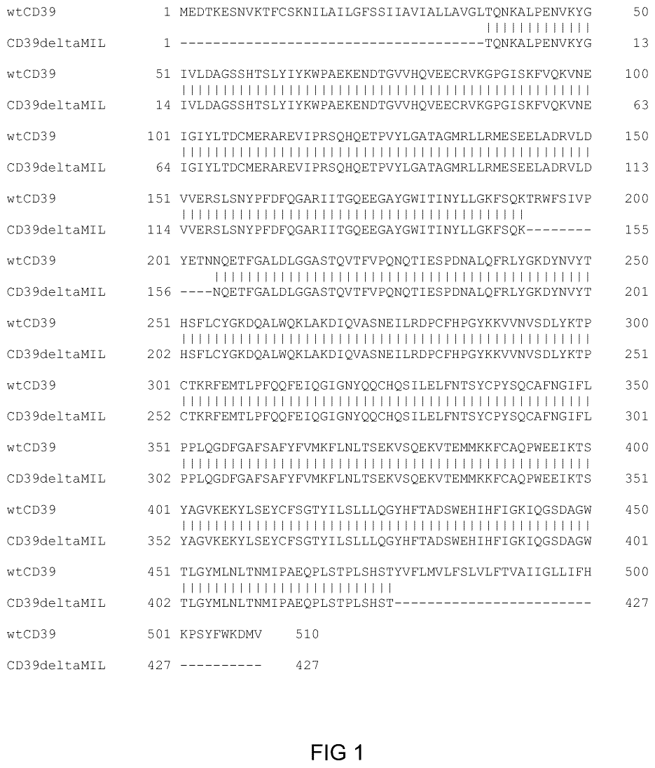

As can be seen from FIG. 1, showing a sequence alignment of SEQ ID NO: 1 and SEQ ID NO: 4, N terminal amino acids 1 to 27, C terminal amino acids 477 to 510, and the central membrane interaction loop (MIL) amino acids 193 to 204, were deleted from wtCD39 (SEQ ID NO: 1) to form CD39ΔMIL (SEQ ID NO: 4).

The impact of the different sequences modifications on thermal stability was studied. In addition, impact of the different sequences modifications on CHO cell expression yields and monomeric content was studied.

### (1) Methods

### (a) Generation of Expression Plasmids

DNA sequences coding for different hCD39 boundary variants and membrane interaction loop (MIL) deletion were ordered at GeneArt (Life Technologies Inc. Regensburg, Germany) including codon optimization for Homo sapiens. Sequences coding for hCD39 variants were sub cloned by standard molecular biology techniques from the GeneArt derived vectors or internally generated variants thereof into an expression vector suitable for secretion in mammalian cells. Cysteine to alanine mutations present in the cysteine bridge deleted variants were targeted by modified oligonucleotides and after a subsequent assembly PCR step the generated fragments were sub cloned into the same expression vector mentioned previously. Elements of the expression vector include a promoter (Cytomegalovirus (CMV) enhancer-promoter), a signal sequence to facilitate secretion, a polyadenylation signal and transcription terminator (Bovine Growth Hormone (BGH) gene), an element allowing episomal replication and replication in prokaryotes (e.g. SV40 origin and ColE1 or others known in the art) and elements to allow selection (ampicillin resistance gene and zeocin marker). A list of truncated, solubilized human CD39 versions is illustrated in Table 2, with amino acid modifications numbered with reference to SEQ ID NO: 1.

TABLE 2
Truncated, solubilized human CD39 versions.

| | Amino acid | |
|---|---|---|
| Reference | sequence ID | Modification(s) |
| CD39(aa39-469) | SEQ ID NO: 99 | N and C terminal truncations |
| CD39(aa46-476) | SEQ ID NO: 101 | N and C terminal truncations |
| CD39(aa46-461) | SEQ ID NO: 103 | N and C terminal truncations |
| CD39(aa46-461)_dMIL(193-204) | SEQ ID NO: 105 | N and C terminal truncations, |
| | | and central deletion |
| CD39(aa46-461)_delta cys1 | SEQ ID NO: 107 | N and C terminal truncations, |
| | | and cysteine deletion |
| CD39(aa46-461)_delta cys2 | SEQ ID NO: 109 | N and C terminal truncations, |
| | | and cysteine deletion |
| CD39(aa46-461)_delta cys3 | SEQ ID NO: 111 | N and C terminal truncations, |
| | | and cysteine deletion |
| CD39(aa46-461)_delta cys4 | SEQ ID NO: 113 | N and C terminal truncations, |
| | | and cysteine deletion |
| CD39(aa46-461)_delta cys5 | SEQ ID NO: 115 | N and C terminal truncations, |
| | | and cysteine deletion |
| CD39(aa46-461)_dMIL(193-204)_delta | SEQ ID NO: 117 | N and C terminal truncations, |
| cys1 | | central deletion and cysteine |
| | | deletion |
| CD39(aa46-461)_dMIL(193-204)_delta | SEQ ID NO: 119 | N and C terminal truncations, |
| cys2 | | central deletion and cysteine |
| | | deletion |
| CD39(aa46-461)_dMIL(193-204)_delta | SEQ ID NO: 121 | N and C terminal truncations, |
| cys3 | | central deletion and cysteine |
| | | deletion |
| CD39(aa46-461)_dMIL(193-204)_delta | SEQ ID NO: 123 | N and C terminal truncations, |
| cys4 | | central deletion and cysteine |
| | | deletion |
| CD39(aa46-461)_dMIL(193-204)_delta | SEQ ID NO: 125 | N and C terminal truncations, |
| cys5 | | central deletion and cysteine |
| | | deletion |
| CD39(aa38-476)_delta337-344 | SEQ ID NO: 127 | N and C terminal truncations, |
| | | and cysteine deletion |
| CD39(aa38-476)_C338A_C343A | SEQ ID NO: 129 | N and C terminal truncations, |
| | | and cysteine point mutations |

(b) Micro-Scale Expression of hCD39 Variants

293-6E cells (as disclosed in WO2006096989A2, incorporated herein by reference) were chosen for micro-scale experiment as one of the preferred host cell lines for transient expression of proteins in absence of serum. Transfection was performed using FuGene HD (Roche Applied Science, Cat. No. 04709705001) as transfection reagent. 293-6E cells were cultivated in suspension culture using V3 serum-free culture medium (Bioconcept, Cat. No. V3-K) for transfection and propagation of the cells. Cells were grown in Corning shake flasks (Corning, Tewksbury, MA) on an orbital shaker (100-120 rpm) in a humidified incubator at 5% CO₂ (seed flasks). Cells in the seed cultures should be maintained in the exponential growth phase (cell densities between 5×10⁵ and 3×10⁶/mL) and display a viability of >90% for transfection. Cell densities outside of this range will result in either a lag phase after dilution or reduced transfection efficiency.

For micro-scale (0.5 ml) transfections, an aliquot of cells was taken out of the seed cultures and adjusted to 0.5×10⁶ cells/mL in V3 serum-free culture medium. The DNA solution (called Solution 1) was prepared by diluting 0.5 μg of hCD39 expression plasmids in 14 μl of V3 serum-free culture medium, then 2.3 μl of FuGene HD solution was also diluted in 14 μl of V3 serum-free culture medium (Solution 2). Both solutions were incubated for 5-10 min at room temperature (RT). Thereafter solution 2 was added to solution 1 with gentle mixing and incubated for another 5-15 minutes at room temperature. The transfection mix was then added to 0.5 ml of cells at 0.5×10⁶ cells/mL seeded in a 48-well tissue culture plate (Corning, Tewksbury, MA) and plate placed on an orbital shaker (300 rpm) in a humidified incubator at 5% CO₂. The culture was harvested 3 days post transfection by centrifugation at 4000 rpm for 10 minutes at 4° C. (Heraeus, Multifuge 3 S-R, Thermo Scientific, Rockford, IL). The recovered cell supernatant was stored at 4° C. until further processing.

### (c) Western-Blot Analysis on Micro-Scale Expression Supernatant

Western-Blot analysis was performed on micro-scale expression supernatant in order to check expression and correct formation of recombinant hCD39 variants. 8 μl of supernatant was diluted in E-PAGE™ Loading Buffer (4×, Invitrogen, #EPBNF-01) and loaded on E-Page 48, 8% gel (Invitrogen, #EP04808) in non-reducing conditions. Gel was run on E-base mother device (Invitrogen) for 23 min and proteins were transferred to Nitrocellulose membrane (Invitrogen IB301001) using the iBlot system (Invitrogen) according to manufacturer's instructions (7 min run). After 3 times washing in TBS/0.05 Tween20 (TBST), membrane was incubated for 1 h with 5% Milk/TBST in gentle agitation followed by 1 hr incubation with 4 μg/ml solution of anti-APP mouse antibody (Novartis internally antibody raised against a peptide stretch of amyloid precursor protein (APP) used for protein tagging) diluted in 2% Milk/TBST. After an additional 3 washing steps, membrane was incubated with 1:1000 dilution of Anti-Mouse IgG-Alkaline Phosphatase (Sigma-Aldrich, A5153-1ML) diluted in 2% Milk/TBST and washed again 3 times in TBST followed by a rinsing step in TBS. Signal was developed for 1-5 minutes using SIGMAFAST™ BCIP®/NBT (Sigma-Aldrich, #B5655-25TAB) according to manufacturer's instructions and signal stopped by rinsing the membrane with water.

### (d) Solid-Phase AxPase Assay

ATPase, ADPase and AMPase activities were determined using Pi ColorLock Gold phosphate detection system (Innova Biosciences, cat n. 303-0030) on plate-captured hCD39 variants from micro-scale expression supernatant (Solid-phase Axpase assay). This method was found to be less sensitive compared to solution based assay (Liquid-phase Axpase assay) recommended by manufacturer, but would have the advantage to reduce AxPase activity mediated by host cell enzymes potentially present in the micro-scale expression supernatant. 20 μl of anti-APP mouse antibody 10 μg/ml solution antibody (Novartis internally antibody raised against a peptide stretch of amyloid precursor protein (APP) used for protein tagging) diluted in PBS was added to each well of a maxisorp 384 well clear plate (Nunc) and incubated over-night at 4° C. After three washing with TBST, wells were blocked for 1h using 100 μl of 5% Milk/TBST at room temperature in gentle agitation. After an additional three washing steps, 20 μl of serially diluted micro-scale expression supernatant in 2% Milk/TBST was added in triplicate to the wells and incubated for 2 hrs at room temperature with gentle agitation. Wells were then washed again four times with 100 μl of TBST and twice with 80 μl of 50 mM Tris-Cl/5 mM MgCl₂ PH 7.5. 30 μl of 80 μM Adenosine Phosphate solutions diluted in 50 mM Tris-Cl/5 mM MgCl₂ PH 7.5 (ATP: SIGMA A2383, ADP: SIGMA A2754) was added to each triplicate and incubated for 24 hrs at 37° C. Signal was developed using 7.5 μl of Gold reagent mix prepared according to manufacturer's instructions for 10 minutes and reaction stopped using 3 μl of Stabilizer. Absorbance at 620 nm read using TECAN Genios Pro instrument.

### (2) Results

(a) Effect of Boundaries, Membrane Interaction Loop (MIL) Deletion and Cysteine Bridge Deletion on hCD39 Expression Level

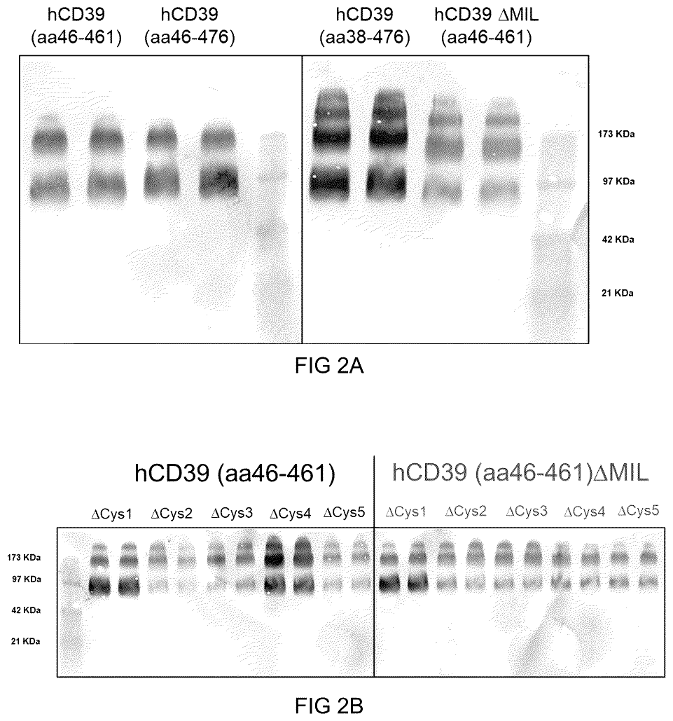

In order to evaluate expression level of different hCD39 variants, corresponding expression plasmids were transfected in duplicate in 0.5 ml of 293-6E cells and Western Blot (anti-APP detection Ab) performed on supernatant collected 3 days post transfection. Results are illustrated in FIG. 2A and FIG. 2B.

Results indicate a higher expression level of hCD39 starting at aa38 compared to aa46. N-terminal boundaries as well as MIL deletion seem to have no major impact on expression level. Higher expression level of hCD39 having the first or fourth cysteine-bridge deleted in the context of hCD39 (aa46-461) was also observed. Higher expression level of first cysteine bridge deletion was confirmed also using hCD39 (aa46-461) ΔMIL backbone.

(b) Effect of Boundaries, Membrane Interaction Loop (MIL) Deletion and Cysteine Bridge Deletion on hCD39 Activity

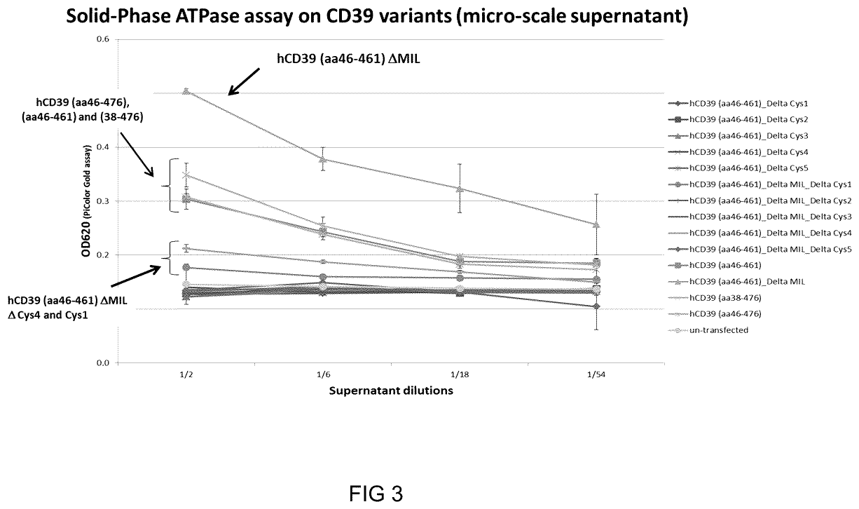

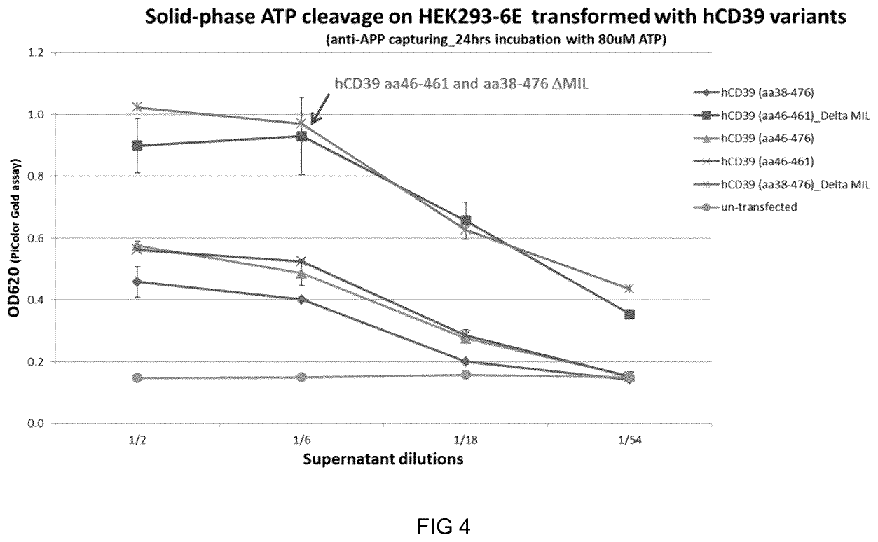

CD39 enzymatic activity was measured by solid-phase AxPase assay on the above described supernatant samples. Results are illustrated in FIG. 3 and FIG. 4, as well as Table 3.

TABLE 3
Solid-phase ATP assay on CD39 variants

| | | Average | | | |
|---|---|---|---|---|---|
| | | signal | | | |
| | | Dilution | Dilution | Dilution | Dilution |
| Sample | Sequence | 1/2 | 1/6 | 1/18 | 1/54 |
| FIG. 3 | | | | | |
| hCD39 | SEQ ID | 0.12755 | 0.12905 | 0.13155 | 0.13545 |
| (aa46-461)_Delta Cys1 | NO: 107 | | | | |
| hCD39 | SEQ ID | 0.1287 | 0.13615 | 0.12915 | 0.1375 |
| (aa46-461)_Delta Cys2 | NO: 109 | | | | |
| hCD39 | SEQ ID | 0.12265 | 0.1336 | 0.13485 | 0.1365 |
| (aa46-461)_Delta Cys3 | NO: 111 | | | | |
| hCD39 | SEQ ID | 0.14 | 0.1302 | 0.13535 | 0.1322 |
| (aa46-461)_Delta Cys4 | NO: 113 | | | | |
| hCD39 | SEQ ID | 0.1226 | 0.13865 | 0.13375 | 0.13225 |
| (aa46-461)_Delta Cys5 | NO: 115 | | | | |
| hCD39 | SEQ ID | 0.17695 | 0.16 | 0.15755 | 0.1551 |
| (aa46-461)_Delta | NO: 117 | | | | |
| MIL_Delta Cys1 | | | | | |
| hCD39 | SEQ ID | 0.1318 | 0.1419 | 0.13065 | 0.1299 |
| (aa46-461)_Delta | NO: 119 | | | | |
| MIL_Delta Cys2 | | | | | |
| hCD39 | SEQ ID | 0.13505 | 0.1376 | 0.1347 | 0.13775 |
| (aa46-461)_Delta | NO: 121 | | | | |
| MIL_Delta Cys3 | | | | | |
| hCD39 | SEQ ID | 0.21195 | 0.18745 | 0.16915 | 0.14975 |
| (aa46-461)_Delta | NO: 123 | | | | |
| MIL_Delta Cys4 | | | | | |
| hCD39 | SEQ ID | 0.1343 | 0.1486 | 0.13135 | 0.10465 |
| (aa46-461)_Delta | NO: 125 | | | | |
| MIL_Delta Cys5 | | | | | |
| hCD39 (aa46-461) | SEQ ID | 0.30345 | 0.2433 | 0.1879 | 0.1851 |
| | NO: 103 | | | | |
| hCD39 | SEQ ID | 0.5037 | 0.3779 | 0.32345 | 0.2567 |
| (aa46-461)_Delta MIL | NO: 105 | | | | |
| hCD39 (aa38-476) | Sequence not | 0.30745 | 0.23855 | 0.18315 | 0.17295 |
| | shown | | | | |
| hCD39 (aa46-476) | SEQ ID | 0.34815 | 0.254 | 0.1975 | 0.1817 |
| | NO: 101 | | | | |
| untransfected | | 0.1459 | 0.14155 | 0.13855 | 0.13675 |
| FIG. 4 | | | | | |
| hCD39 (aa38-476) | Sequence not | 0.45775 | 0.4007 | 0.1997 | 0.14215 |
| | shown | | | | |
| hCD39 | SEQ ID | 0.8983 | 0.9294 | 0.6561 | 0.35405 |
| (aa46-461)_Delta MIL | NO: 105 | | | | |
| hCD39 (aa46-476) | SEQ ID | 0.5756 | 0.4855 | 0.27525 | 0.153 |
| | NO: 101 | | | | |
| hCD39 (aa46-461) | SEQ ID | 0.562 | 0.52455 | 0.28625 | 0.15175 |
| | NO: 103 | | | | |
| hCD39 | SEQ ID | 1.0224 | 0.9696 | 0.6263 | 0.4354 |
| (aa38-476)_Delta MIL | NO: 4 | | | | |
| (EP28) | | | | | |
| untransfected | | 0.1483 | 0.1494 | 0.158 | 0.1502 |

Deletion of MIL seems to increase the fraction of functionally expressed CD39 recombinant proteins. Different boundaries do not show any major impact on active hCD39 activity. Results indicate strongly reduced or completely abolished ATPase activity of all the cysteine-bridge deleted variants. Similar results were obtained using Solid-Phase ADPase assay. Thus, surprisingly, the sequence modification which both increase efficiency and ability to express CD39 is the delta MIL (ΔMIL) modification.

### 3. Example 2: Expression Tags

In order to improve the expression properties of the candidates, different expression tags were tested.

Different expression tags based on the N-terminal portion of IL-2 (SEQ ID NO: 131) were tested, as set forth in Table 4. Expression tag 1-16 aa, according to SEQ ID NO: 131, was synthesized by Geneart.

TABLE 4
IL-2 expression tag variant overview

| | Amino acid | Forward primer | Reverse primer |
|---|---|---|---|
| Reference | sequence ID | sequence ID | sequence ID |
| Expression | SEQ ID NO: 131 | n/a | n/a |
| tag aa1-16 | | | |
| Expression | SEQ ID NO: 133 | SEQ ID NO: 157 | SEQ ID NO: 158 |
| tag aa1-15 | | | |
| Expression | SEQ ID NO: 135 | SEQ ID NO: 159 | SEQ ID NO: 158 |
| tag aa1-6 | | | |
| Expression | SEQ ID NO: 137 | SEQ ID NO: 161 | SEQ ID NO: 158 |
| tag aa1-3 | | | |
| Expression | SEQ ID NO: 139 | SEQ ID NO: 162 | SEQ ID NO: 158 |
| tag aa1-9 | | | |
| Expression | SEQ ID NO: 141 | SEQ ID NO: 163 | SEQ ID NO: 158 |
| tag aa1-12 | | | |
| Expression | SEQ ID NO: 143 | SEQ ID NO: 164 | SEQ ID NO: 158 |
| tag aa4-12 | | | |

All expression tags were tested in relation to CD39ΔMIL, as set forth in SEQ ID NO: 4. All the constructs included an APP tag and a His tag.

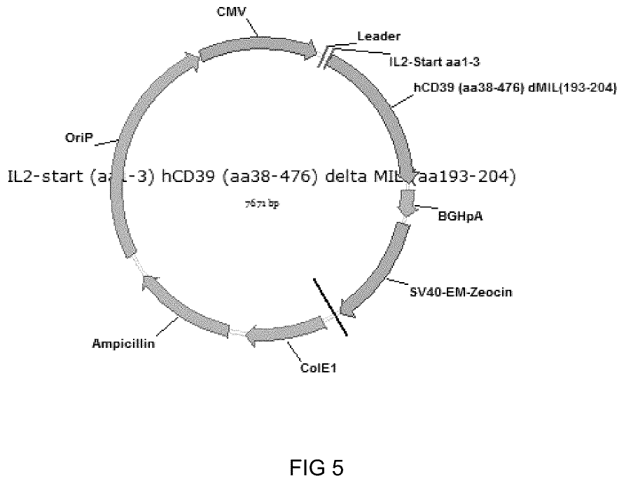

The vector pRS5a, as set forth in FIG. 5, was used for the expression. Primer pairs were as set forth in Table 4.

Annealing temperature was 64° C. in all cases.

PCR solution was prepared by mixing 1 μl Template DNA stock, 25 μl Kapa Hifi Hotstart polymerase (from kappa Biosystems/KK2602). 1.5 μl forward Primer, 1.5 μl reverse primer, and adjusting the final volume to 50 μl with H₂O.

The PCR reaction was run according to schedule in Table 5.

TABLE 5
PCR schedule

| | Temperature | Time | |
|---|---|---|---|
| | (C. °) | (min) | Cycles |
| Denaturation | 94 | 5 | 1 |
| Denaturation | 94 | 0.5 | 35 |
| Annealing | 64 | 1.5 | |
| Polymerization | 72 | 0.5 | |
| Final Polymerization | 72 | 5 | |

After completion of PCR reaction, DNA extraction was performed using Wizard® SV Gel and PCR Clean-Up Kit, Promega, No. 9282, 1 column, elution in 30 μl according to the instructions of the manufacturer.

Inserts and vector were cut with enzyme supplied by New England Biolabs (NEB), Nrul-HF (NEB #R3192) and Notl-HF (NEB #R3189), in CutSmart (R) buffer. Reaction time was 3 h at 37° C.

Ligation has been performed over night with dephosphorylated Vector with Rapid DNA Dephosphorylation and Ligation Kit, Fa. Roche, No. 04898117001 according to the valid protocol of the producer.

Next day, single colonies were picked for DNA-Miniprep and sequence analysis with forward Primer P270 (SEQ ID NO: 165) and reverse Primer P271 (SEQ ID NO: 166).

In addition, the a few protein sequences known in the art to increase expression were tested, according to Table 6.

TABLE 6
Prior art tags

| Reference | Sequence identifier |
|---|---|
| Ubiquitin | SEQ ID NO: 167 |
| CKappa | SEQ ID NO: 168 |
| HSA Domain I | SEQ ID NO: 169 |
| HSA Domain II | SEQ ID NO: 170 |

The resulting combinations tested are set forth in Table 7.

TABLE 7
Tested constructs

| | Resulting amino |
|---|---|
| Plasmid Name | acid sequence |
| pRS5a_IL2 leader-APP6-Flag-hCD39 | SEQ ID NO: 145 |
| (aa38-476)_delta MIL (aa193-204)_8M opt | |
| pRS5a_IL2 leader-hsUbiquitin-hCD39 | SEQ ID NO: 149 |
| (aa38-476)_delta MIL (aa193-204)_APP_His | |
| pRS5a_IL2 leader-hsCKappa-hCD39 | SEQ ID NO: 147 |
| (aa38-476)_delta MIL (aa193-204)_APP_His | |
| pRS5a_IL2 leader-HSA-Dom.I-hCD39 | SEQ ID NO: 151 |
| (aa38-476)_delta MIL (aa193-204)_APP_His | |
| pRS5a_IL2 leader-HSA-Dom.II-hCD39 | SEQ ID NO: 153 |
| (aa38-476)_delta MIL (aa193-204)_APP_His | |

None of the prior art tags from Table 6 gave expression of protein (data not shown). This was unexpected, since prior art teaches that these sequences should increase expression.

### 4. Example 3: Further Mutations

In order to improve the characteristics of soluble CD39, and make it suitable for pharmaceutical development, further modifications were introduced in the CD39ΔMIL, EP28, set forth in SEQ ID NO: 4. The different mutations and mutated variants are seen in Table 8 and are numbered according to the amino acid positions of the wild type CD39 as set forth in SEQ ID NO: 1.

TABLE 8
Point mutations

| Short | No. | Mutation | Mutation | Mutation | Mutation | Mutation | Amino acid |
|---|---|---|---|---|---|---|---|
| name | Mutations | Pos. 1 | Pos. 2 | Pos. 3 | Pos. 4 | Pos. 5 | sequence ID |
| EP1 | 1 | 113: R −> M | | | | | SEQ ID NO: 6 |
| EP2 | 2 | 113: R −> M | 149: L −> M | | | | SEQ ID NO: 8 |
| EP3 | 3 | 113: R −> M | 304: R −> G | 469: S −> R | | | SEQ ID NO: 10 |
| EP4 | 3 | 95: V −> A | 113: R −> M | 469: S −> R | | | SEQ ID NO: 12 |
| EP5 | 2 | 149: L −> M | 441: G −> D | | | | SEQ ID NO: 14 |
| EP6 | 3 | 104: Y −> S | 149: L −> M | 263: W −> R | | | SEQ ID NO: 16 |
| EP7 | 4 | 71: K −> E | 106: T −> S | 151: V −> A | 319: I −> T | | SEQ ID NO: 18 |
| EP8 | 1 | 151: V −> A | | | | | SEQ ID NO: 20 |
| EP9 | 1 | 263: W −> R | | | | | SEQ ID NO: 22 |
| EP10 | 1 | 319: I −> T | | | | | SEQ ID NO: 24 |
| EP11 | 2 | 113: R −> M | 319: I −> T | | | | SEQ ID NO: 26 |
| EP12 | 2 | 276: E −> D | 319: I −> T | | | | SEQ ID NO: 28 |
| EP13 | 2 | 365: F −> S | 424: L −> P | | | | SEQ ID NO: 30 |
| EP14 | 1 | 365: F −> S | | | | | SEQ ID NO: 32 |
| EP15 | 4 | 292: N −> K | 365: F −> S | 424: L −> P | 463: P −> S | | SEQ ID NO: 34 |
| EP17.1 | 1 | 412: Y −> F | | | | | SEQ ID NO: 38 |
| EP17 | 2 | 405: K −> N | 412: Y −> F | | | | SEQ ID No. 36 |
| EP18 | 2 | 102: G −> D | 424: L −> Q | | | | SEQ ID No. 40 |
| EP19 | 2 | 424: L −> Q | 436: H −> D | | | | SEQ ID No. 42 |
| EP20 | 2 | 276: E −> G | 439: F −> S | | | | SEQ ID No. 44 |
| EP21 | 2 | 113: R −> M | 469: S −> R | | | | SEQ ID No. 46 |
| EP22 | 2 | 276: E −> G | 469: S −> G | | | | SEQ ID No. 48 |
| EP23 | 4 | | 254: L −> M | 263: W −> R | 439: F −> S | 469: S −> R | SEQ ID No. 50 |
| EP24 | 3 | 113: R −> K | 424: L −> Q | 437: I −> N | | | SEQ ID No. 52 |
| EP28_8M | 8 | 173: E −> D | 258: K −> R | 362: A −> N | 365: F −> Y | 404-411: | SEQ ID No. 145 |
| | | | | | | VKEKYLSE −> | |
| | | | | | | QERWLRD | |

Two mutations in active site lead to higher activity (365 and 412).

### 5. Example 4: Glycosylation Site Removal

Based on the EP14 variant above, the effect of glycosylation sites was checked by introducing point mutations according to Table 9, numbered according to the amino acid positions of the wild type CD39 as set forth in SEQ ID NO: 1.

TABLE 9
Glycosylation site mutations

| Mutation | Glycosylation | |
|---|---|---|
| position | site | Primer |
| N73Q | NDT | P928/P929 |
| T229A | NQT | P930/P931 |
| N292Q | NVS | P932/P933 |
| N327Q | NTS | P934/P935 |
| N371Q | NLT | P936/P937 |
| N457Q | NLT | P938/P939 |

### (a) Materials and Methods

The expression vector pRS5a (FIG. 5) was used for the cloning. Primers were used as set forth in Table 10.

TABLE 10
Primer sequences

| Primers | Sequence | Description |
|---|---|---|
| P928 | SEQ ID NO: 171 | Forward primer for removal of |
| | | Glycosylation site N73Q |
| P929 | SEQ ID NO: 172 | Reverse primer for removal of |
| | | Glycosylation site N73Q |
| P930 | SEQ ID NO: 173 | Forward primer for removal of |
| | | Glycosylation site T229A |
| P931 | SEQ ID NO: 174 | Reverse primer for removal of |
| | | Glycosylation site T229A |
| P932 | SEQ ID NO: 175 | Forward primer for removal of |
| | | Glycosylation site N292Q |
| P933 | SEQ ID NO: 176 | Reverse primer for removal of |
| | | Glycosylation site N292Q |
| P934 | SEQ ID NO: 177 | Forward primer for removal of |
| | | Glycosylation site N327Q |
| P935 | SEQ ID NO: 178 | Reverse primer for removal of |
| | | Glycosylation site N327Q |
| P936 | SEQ ID NO: 179 | Forward primer for removal of |
| | | Glycosylation site N371Q |
| P937 | SEQ ID NO: 180 | Reverse primer for removal of |
| | | Glycosylation site N371Q |
| P938 | SEQ ID NO: 181 | Forward primer for removal of |
| | | Glycosylation site N457Q |
| P939 | SEQ ID NO: 182 | Reverse primer for removal of |
| | | Glycosylation site N457Q |

The QuikChange Lightning Site-directed Mutagenesis Kit (Agilent, No. 210519-5) was used for the PCR, according to the manufacturer's instructions.

Next day, single colonies were picked for DNA-Miniprep and sequence analysis was performed with forward Primer P270 (SEQ ID NO: 165) and reverse Primer P271 (SEQ ID NO: 166).

To ensure the correctness of the vector backbone as well (because of mutagenesis), the sequenced insert fragment was cloned into a new vector backbone of pRS5a (FIG. 5) using the following method.

The vector was prepared using the vector backbone of SEQ ID NO: 36 with expression tag SEQ ID NO: 135, containing an APP_HIS-Tag, stock conc. 3.3 μg/μl.

Vector was digested by mixing 10 μg vector-DNA, 0.4 μl HindIII (100U/μl, NEB), 2 μl EcoRI (20U/μl, NEB), 5 μl Cutsmart buffer 10× conc. (NEB), H₂O to final volume of 50 μl. Digestion was run for 3 h at 37° C.

Dephosphorylation was performed with Alkaline Phosphatase, Calf intestinal (CIP, NEB, No. M0290L), 10U/μl. Directly after digestion, 3 μl of CIP was added to digested vector and incubated for 30 min. at 37° C. Digested and dephosphorylated Vector was loaded on preparative 0.8% TAE Agarose gel, correct band size of vector with ˜6100 bp had been cut out. Cleanup was done with Wizard® SV Gel and PCR Clean-Up Kit, Promega, No. 9282, 1 column, elution in 100 μl. OD260 nm showed a concentration of 64 ng/μl.

Digestion of mutated Insert fragments was done by mixing 42.5 μl DNA (˜3-5 ug for each DNA), 5 μl Cutsmart buffer, 10× conc., NEB no B7204S, 0.4 μl HindIII-HF, 100 U/μl, NEB no. R3104S, 2 μl EcoRI-HF, 20 U/μl, NEB no. R3103L, and adjust volume to 50 μl with H₂O. Digestion was carried out for 3 h at 37° C. in PCR-machine. Digested inserts were loaded on preparative 0.8% TAE Agarose gel, correct band size of vector with ˜1400 bp had been cut out. Cleanup was done with Wizard® SV Gel and PCR Clean-Up Kit, Promega, No. 9282, 1 column, elution in 30 μl. OD260 nm showed a concentration of 1-25 ng/μl.

Ligation was done using (vector: insert ratio ˜1:10), with Rapid DNA Ligation Kit, No. K1423, Fa. Thermo Scientific. 4 μl 5× Ligation Buffer was mixed with 1 μl Ligase, 2 μl vector fragment, HindIII/EcoRI-digested, stock conc. 64 ng/μl, 13 μl insert fragment, HindIII/EcoRI-digested, stock conc. 1-25 ng/μl. Ligation was run for 10 minutes at RT.

Transformation was done by incubation of 10 μl of ligation with 80 μl chemical competent XL1Blue cells (Novartis, FS/RL) for 30 min on ice. Heat shock for 45 sec at 42° C. on Eppendorf incubator, followed by incubation for 2 min on ice. After that, 1 ml 2YT media was added, followed by incubation for 1.5h at 37° C. on Eppendorf shaker (800 rpm). Cells were centrifuged for 3 min at 7000 rpm and colonies plating on LB/Carb/Gluc. Plates, followed by incubation overnight at 37° C.

Next day, single colonies were picked for DNA-Miniprep and sequence analysis was performed with forward Primer P270 (SEQ ID NO: 165) and reverse Primer P271 (SEQ ID NO: 166).

Correct sequences were transfected into HEK293 cells according to 7 days of expression, 200 ml scale.

### The Following Material was Used:

Human Embryonic Kidney cells constitutively expressing the SV40 large T antigen (HEK293-T), e.g. ATCC11268

Polyethylenimine “MAX” MW 40.000 (PEI) (Polysciences, Cat. 24765), dissolved in H20 at RT, adjusted with NaOH to pH7.05.

M11V3 serum-free culture medium (Bioconcept, CH, Cat: V3-K)

DNA: prepared with Qiagen DNA Kit, Midiprep-Kit (No. 12943) according to protocol recommended by supplier

All cell culture work for the transient transfections is carried out using suspension adapted HEK293-Tcells growing in serum-free M11V3 medium.

Cells are grown in Corning shake flasks (Corning, USA) on an orbital shaker (115 rpm) in a humidified CO₂-incubator with 5% CO₂ (seed flasks).

Used Cells have been in exponential growth phase (cell density between 5×10⁵ and 3×10⁶/ml) and had a viability of >90%.

Transfection was performed in small scale (here 20/50 or 100 ml), using cells that have been counted and corresponding amount of cells has been adjusted to 1.4×10⁶ cells/ml with M11V3-media. 36% cell suspension of the final transfection volume is used.

The DNA solution (solution 1) is prepared by diluting 1 mg/L final volume DNA in 7% final volume M11V3 and gentle mixing. To prevent contamination of the cultures because of the not steril filtrated DNA, Penc./Strep has been added to the transfection after the feeding. Then 3 mg/L final volume PEI solution is diluted in 7% final volume M11V3 and mixed gently (solution 2). Both solutions are incubated for 5-10 min at room temperature (RT). Thereafter solution 2 is added to solution 1 with gentle mixing and incubated another 5-15 minutes at RT After the incubation the transfection mix is added to the cells and the culture is cultivated for four hours (115 rpm, 37° C., 5% CO2).

Supernatant was harvested after 7 days of expression.

Centrifugation 4500 rpm., 15 min., 4° C. (Heraeus, Multifuge 3 S-R)

Clarification through a sterile filter, 0.22 μm (Stericup filter, Thermo Scientific, Cat. 567-0020))

Deliver supernatant to purification for further steps. 1 ml sample of supernatant are used for IPC on Open Access APP-column

Sample vials were glass crimp vials, 2 ml Agilent, catalog number 5182-0543 and caps: crimp 11 mm, catalog number 5040-4667.

Protein was purified using immobilized metal affinity chromatography (IMAC) on Aekta Pure or Aekta Avant (GE Healthcare), according to the following protocol, using a 5 ml Histrap HP column (GE Life Sciences, Order No. 17-5248-02). The specifications are set forth in Table 11.

TABLE 11
IMAC protocol

| | | Flow | |
|---|---|---|---|
| | CV | (ml/min) | Buffer |
| Equilibration | 5 | 5 | IMAC A |
| Sample load | | 3 | Cell supernatant |
| Column Wash | 10 | 3 | IMAC A |
| Elution | 10 | 3 | Gradient 0-20% |
| | | | IMAC B |
| | 10 | 3 | 100% IMAC B |
| Fractionation | | | 2 ml fractions |

The buffers used were composed according to Table 12 and Table 13.

TABLE 12
IMAC A, equilibration- and wash buffer

| Concentration | | Chemical |
|---|---|---|
| 10 | mM | Na₂HPO₄ |
| 10 | mM | NaH₂PO₄ |
| 500 | mM | NaCl |
| 20 | mM | Imidazol (Merck) |

TABLE 13
IMAC B, elution buffer

| Concentration | | Chemical |
|---|---|---|
| 10 | mM | Na₂HPO₄ |
| 10 | mM | NaH₂PO₄ |
| 500 | mM | NaCl |
| 500 | mM | Imidazol (Merck) |

The resulting protein, according to Table 14, was stored.

TABLE 14
Constructs

| Construct | Mutation positions |
|---|---|
| C1140 | N73A/F365S |
| C1141 | T229A/F365S |
| C1142 | N292Q/F365S |
| C1143 | N334Q/F365S |
| C1144 | F365S/N371Q |
| C1145 | F365S/N457Q |
| C1058 | F365S |

### (b) Results and Interpretation

There was no improvement of mutants concerning yield and monomeric peak of analytical SEC. The parental protein (EP14) with expression tag according to SEQ ID NO: 137 gave best yield and highest monomeric peak in analytical. Lowest yield and as well worst monomeric peak achieved with mutant N371Q.

### 6. Example 5: Combinations

In order to try and further improve properties, some of the mutations introduced in Example 3 above were combined according to Table 15, below. Mutations are numbered according to the amino acid positions of the wild type CD39 as set forth in SEQ ID NO: 1.

TABLE 15
Combination of constructs

| Construct | Mutation 1 | Mutation 2 | Mutation 3 | Without | Primers | SEQ ID |
|---|---|---|---|---|---|---|
| EP14xEP19 | 365: F−>S | 424: L−>Q | | 436: H−>D | P878/P879 | SEQ ID NO: 64 |
| EP17xEP19 | 424: L−>Q | 436: H−>D | 412: Y−>F | 405: K−>N | P880/P881 | SEQ ID NO: 70 |
| EP14xEP17 | 365: F−>S | 412: Y−>F | | 405: K−>N | P880/P881 | SEQ ID NO: 60 |
| EP10xEP19_H436D | 424: L−>Q | 436: H−>D | 319: I−>T | | P882/P883 | SEQ ID NO: 62 |
| EP19 w/o H436D | 424: L−>Q | | | 436: H−>D | P884/P885 | Sequence not |
| | | | | | | included |

### (a) Materials and Methods

The primers according to Table 16 were used.

TABLE 16
Primers

| Primer | Sequence |
|---|---|
| P878 | SEQ ID NO: 183 |
| P879 | SEQ ID NO: 184 |
| P880 | SEQ ID NO: 185 |
| P881 | SEQ ID NO: 186 |
| P882 | SEQ ID NO: 187 |
| P883 | SEQ ID NO: 188 |
| P884 | SEQ ID NO: 189 |
| P885 | SEQ ID NO: 190 |

A PCR reaction was set up using the following pipetting scheme:
5 μl of 10× reaction buffer,
1 μl ds-DNA-template (stock conc. 100 ng/μl),
1.5 μl primer 1,
1.5 μl primer 2,
1 μl dNTP mix
1.5 μl QuickSolution reagent,
35.5 μl H₂O (for final volume of 50 μl), and
1 μl QuickChange Lightning Enzyme.

The PCR cycling parameters according to Table 17 were used.

TABLE 17
PCR cycling parameters

| | No. of | | | |
|---|---|---|---|---|
| | cycles | Temp | Time | |
| 1 | 1 | 96° C. | 2 | min |
| 2 | 18 | 96° C. | 20 | sec |
| | | 60° C. | 10 | sec |
| | | 68° C. | 3 | min |
| | | | (30 | sec/kb) |
| 3 | 1 | 68° C. | 6 | min |

Directly after reaction, 2 μl Dpnl-Enzyme was added to each reaction, mixed and incubated for 5 min at 37° C.

Transformation into XL10-gold ultra-competent cells was performed as follows. Cells were thawed on ice. 45 μl/transformation was used, and 2 μl B-ME was added to each vial. Then, 3 μl Dpnl-digested PCR product was added, and incubated for 30 min on ice in 15 ml BD tubes. Thereafter, the samples were heat shocked for 40 seconds and incubated on ice for 2 min. Next, 950 μl SOC media was added, followed by incubation for 1.5 h at 37° C. in a shaking incubator. Finally, cells were plated on LB-carb-plates and incubated over night at 37° C. Next day, single colonies were picked for DNA-miniprep and sequence analysis.

Correct sequences were transfected into HEK293 cells as described in Example 4.

Protein was purified using immobilized metal affinity chromatography (IMAC) according to the following. 95 ml Supernatant was used (˜4 ml of all is kept for analysis (IPC)).

### Used Material:

Nickel-NTA Agarose, Qiagen, Cat No./ID: 30230, Poly-Prep Chromatography Columns, empty, BioRad, No. 731-1550, IMAC A Buffer pH7.4 (containing 20 mM NaPO₄-buffer and 50 mM Imidazol). IMAC B Buffer pH7.4 (containing 20 mM NaPO₄-buffer and 300 mM Imidazol). TBS (10×-conc. diluted to 1x conc. With MilliQ-Water). Amicon Ultra-4 Centrifugal Filter Unit with Ultracel-10 membrane, 10K, UFC801096.

Process steps:
1. Columns were prepared with 1 ml Nickel-NTA-Agarose of Qiagen (=0.5 ml CV);
2. Equilibration with 10 CV IMAC A;
3. Loading of 15/45 ml SN on column (collect flow through);
4. Washing with 10 CV IMAC A (collect in 15 ml Falcon tube);
5. Elution in 6.5 CV of IMAC B;
6. Determination of concentration of eluate;
7. Concentration of 3.5 ml sample to ˜400 μl with Amicon Ultra-4 Centrifugal Filter Unit 10K;
8. Buffer exchange by adding TBS and centrifugation 5000;

Samples were analyzed using analytical SEC with 40 μl of each sample and using protein gel with 12 μl of each sample.

The resulting protein was stored.

### (b) Results and Interpretation

The results are shown in Table 18.

TABLE 18
Result overview

| | | | Aggregation |
|---|---|---|---|
| | | IPC | (% |
| | Yield | (app, | monomeric |
| Name | (mg/l) | mg/l) | peak) |
| EP14xEP19 | 6.85 | 4.5 | 75.1 |
| EP17xEP19 | 13.35 | 11.5 | 68.9 |
| EP14xEP17 | 17.27 | 11.5 | 69.2 |
| EP10xEP19_H436D | 7.42 | 4.3 | 74.3 |
| EP19 w/o H436D | 8.28 | 2.5 | 55.6 |
| EP1xEP17 | 13.46 | 13.2 | 77.4 |
| EP28 | 7.8 | 2.8 | 57.5 |

### Protease Sites:

There was no/very low yield when Matriptase was inserted. With Furin site, there was ˜40% yield (but for transfection only 50% of DNA has been used as well, as it was a co-transfection with Furin plasmid).

### IL2-Truncations:

All truncations where aa1-3 are included give a comparable result, aa1-3 only might be slightly lower compared to the others, but this might be a variation from sample to sample. Truncation aa4-12 lead to no protein expression. No difference could be found between EP28 that contains, like all other EP-variants, a TSS linker between IL2-start and the hCD39-protein.

### Combinations:

Combinations with EP19 (L424Q) did not lead to a significant improvement of protein expression.

Combination with EP1 (R113M) displayed a lower aggregation in analytical SEC. NEG726 was well expressed, but showed worst aggregation of all tested (˜37%). Combination of EP14×EP17 did not lead to any further improvement (F365S+Y412F).

### 7. Example 6: Cloning of Final Candidates

A selection of clinical candidates as set forth in Table 19 below, were expressed for further testing.

TABLE 19
Overview of final candidates.

| Construct | Combination of clones | IL2 leader | Main Sequence |
|---|---|---|---|
| 1 | EP1xEP14xEP19aa1-3 | SEQ ID NO: 137 | SEQ ID NO: 233 |
| 2 | EP1xEP17xEP19aa1-3 | SEQ ID NO: 137 | SEQ ID NO: 217 |
| 3 | EP14aa1-3 | SEQ ID NO: 137 | SEQ ID NO: 229 |
| 4 | EP17aa1-3 | SEQ ID NO: 137 | SEQ ID NO: 237 |
| 5 | EP19aa1-3 | SEQ ID NO: 137 | SEQ ID NO: 243 |
| 6 | EP28aa1-3 | SEQ ID NO: 137 | SEQ ID NO: 58 |
| | (lead/parental) | | |
| 7 | EP1xEP14xEP19aa1-6 | SEQ ID NO: 135 | SEQ ID NO: 235 |
| 8 | EP1xEP17xEP19aa1-6 | SEQ ID NO: 135 | SEQ ID NO: 219 |
| 9 | EP14aa1-6 | SEQ ID NO: 135 | SEQ ID NO: 231 |
| 10 | EP17aa1-6 | SEQ ID NO: 135 | SEQ ID NO: 239 |
| 11 | EP19aa1-6 | SEQ ID NO: 135 | SEQ ID NO: 245 |
| 12 | EP28aa1-6 | SEQ ID NO: 135 | SEQ ID NO: 80 |
| | (lead/parental) | | |

The following primers were used:

TABLE 20
Primers

| Primer | Sequence |
|---|---|
| R113Mtempl | SEQ ID NO: 191 |
| R113MFW | SEQ ID NO: 192 |
| R113MRev | SEQ ID NO: 193 |
| F330Stempl | SEQ ID NO: 194 |
| F330SFW | SEQ ID NO: 195 |
| F330SRev | SEQ ID NO: 196 |
| Y377Ftempl | SEQ ID NO: 197 |
| L389Ctempl | SEQ ID NO: 198 |
| Y377F/L389Qtempl | SEQ ID NO: 199 |
| FW | SEQ ID NO: 200 |
| Rev | SEQ ID NO: 201 |
| WT | SEQ ID NO: 202 |

A PCR reaction was set up using the following pipetting scheme:
0.25 μl DMSO,
20 ng vector
1.5 μl insert (45 ng/μl),
2 μl 5×HF buffer,
0.1 μl Phusion pol,
0.08 μl dNTP mix
10-X μl ddH₂O

The PCR cycling parameters according to Table 17 were used.

TABLE 21
PCR cycling parameters

| | No. of | | | |
|---|---|---|---|---|
| | cycles | Temp | Time | |
| 1 | 1 | 98° C. | 3 | min |
| 2 | 25 | 98° C. | 30 | sec |
| | | 60° C. | 30 | sec |
| | | 72° C. | 5 | min |
| | | | 46 | sec |
| 3 | 1 | 72° C. | 10 | min |

Directly after reaction, 0.5 μl Dpnl-Enzyme was added to each reaction, mixed and incubated for 2 h at 37° C.

Transformation into XL10-gold ultra-competent cells was performed as follows. Cells were thawed on ice. 45 μl/transformation was used, and 2 μl B-ME was added to each vial. Then, 3 μl Dpnl-digested PCR product was added, and incubated for 30 min on ice in 15 ml BD tubes. Thereafter, the samples were heat shocked for 40 seconds and incubated on ice for 2 min. Next, 950 μl SOC media was added, followed by incubation for 1.5 h at 37° C. in a shaking incubator. Finally, cells were plated on LB-carb-plates and incubated over night at 37° C. Next day, single colonies were picked for DNA-miniprep and sequence analysis.

All constructs were subcloned into new vector background to ensure that sequences were correct. For this, all constructs were amplified with PCR, with G4S-linkers inserted, followed by digestion with HindIII/EcoRI.

The resulting protein was stored.

### 8. Example 7: Generation of Comparator Proteins

### (1) Null Mutations

In order to generate negative control proteins for in vivo studies, one/two mutations were inserted into the parental human CD39ΔMIL protein (EP28). This mutations have been described in the literature to remove or lower the Enzyme activity of this protein. Mutation positions are E174A and S218A.

The following primers were used:

TABLE 22
Primers

| Primer | Sequence |
|---|---|
| P910 | SEQ ID NO: 203 |
| P911 | SEQ ID NO: 204 |
| P914 | SEQ ID NO: 205 |
| P915 | SEQ ID NO: 206 |

A PCR reaction was set up using the following pipetting scheme:
5 μl of 10× reaction buffer,
1 μl ds-DNA-template (stock conc. 100 ng/μl),
1.5 μl primer 1,
1.5 μl primer 2,
1 μl dNTP mix
1.5 μl QuickSolution reagent,
35.5 μl H₂O (for final volume of 50 μl), and
1 μl QuickChange Lightning Enzyme.

The PCR cycling parameters according to Table 17 were used.

TABLE 23
PCR cycling parameters

| | No. of | | | |
|---|---|---|---|---|
| | cycles | Temp | Time | |
| 1 | 1 | 96° C. | 2 | min |
| 2 | 18 | 96° C. | 20 | sec |
| | | 60° C. | 10 | sec |
| | | 68° C. | 3 | min |
| | | | (30 | sec/kb) |
| 3 | 1 | 68° C. | 6 | min |

Directly after reaction, 2 μl Dpnl-Enzyme was added to each reaction, mixed and incubated for 5 min at 37° C.

Transformation into XL10-gold ultra-competent cells was performed as follows. Cells were thawed on ice. 45 μl/transformation was used, and 2 μl B-ME was added to each vial. Then, 3 μl Dpnl-digested PCR product was added, and incubated for 30 min on ice in 15 ml BD tubes. Thereafter, the samples were heat shocked for 40 seconds and incubated on ice for 2 min. Next, 950 μl SOC media was added, followed by incubation for 1.5 h at 37° C. in a shaking incubator. Finally, cells were plated on LB-carb-plates and incubated over night at 37° C. Next day, single colonies were picked for DNA-miniprep and sequence analysis.

Correct sequences were transfected into HEK293 cells according to the following protocol.

A digestion buffer was prepared, using 10 μg vector-DNA, 0.4 μl HindIII (100 U/μl, NEB), 2 μl EcoRI (20 U/μl, NEB), 5 μl Cutsmart buffer 10× conc. (NEB), and H₂O to a final volume of 50 μl. The digestion reaction was run for 3 h at 37° C.

Immediately after digestion, a dephosphorylating reaction was run. Calf intestinal alkaline phosphatase (10U/μl, CIP, NEB, No. M0290L) was added (3 μl) to the digested vector mix and incubated for 30 min at 37° C.

The digested and dephosphorylated vector was sub cloned to check the sequence.

Correct sequences were transfected into HEK293 cells according to the following protocol.

7 days of expression was performed using the following material; 1. Human Embryonic Kidney cells constitutively expressing the SV40 large T antigen (HEK293-T, ATCC11268); 2. Polyethylenimine “MAX” MW 40.000 (PEI) (Polysciences, Cat. 24765).

The PEI solution is prepared by carefully dissolving 1 g PEI in 900 ml cell culture grade water at room temperature (RT). Then it is neutralized with NaOH for a final pH of 7.05. Finally the volume is adjusted to 1L and the solution filtered through a 0.22 μm filter, distributed in aliquots and frozen at −80° C. until further use. Once thawed, an aliquot can be re-frozen up to 3 times at −20° C. but should not be stored long term at −20° C.

M11V3 serum-free culture medium (Bioconcept, CH, Cat: V3-K).

All cell culture work for the transient transfections is carried out using suspension adapted HEK293-Tcells growing in serum-free M11V3 medium.

For small scale (<5L) transfections cells are grown in Corning shake flasks (Corning, USA) on an orbital shaker (100 rpm) in a humidified CO₂-incubator with 5% CO₂ (seed flasks).

In general, cells in the seed cultures should be in the exponential growth phase (cell density between 5×10⁵ and 3×10⁶/ml) and have a viability of >90%. Cell densities outside of this range will result in either a lag phase after splitting or reduced transfection efficacy.

For small scale (here 2L) transfection an aliquot of cells is taken out of the seed cultures and adjusted to 1.4×10⁶ cells/ml in 36% of the final volume with M11V3 medium.

The DNA solution (solution 1) is prepared by diluting 1 mg/L final volume DNA in 7% final volume M11V3 and gentle mixing. To prevent contamination of the cultures, this solution might be filtered using a 0.22 μm filter (e.g. Millipore Stericup). Here because of the small volume no sterile filtration has been done. Then 3 mg/L final volume PEI solution is diluted in 7% final volume M11V3 and mixed gently (solution 2). Both solutions are incubated for 5-10 min at room temperature (RT). Thereafter solution 2 is added to solution 1 with gentle mixing and incubated another 5-15 minutes at RT (do not mix again during incubation time, as PEI covers/condenses DNA into positively charged particles, which bind to anionic cell surface residues and are brought into the cell via endocytosis). After the incubation the transfection mix is added to the cells and the culture is cultivated for four hours (10 rpm, 37° C., 6% CO₂).

Finally the culture is fed with the remaining 50% final volume M11V3 medium according to the following example: Inoculation volume: 36 ml with 1.4×10⁶ cells/ml.

Solution 1:7 ml M11V3 medium with 100 μg plasmid DNA. Solution 2:7 ml M11V3 medium with 300 μg PEI (300 μl)

Feed: 50 ml M11V3, Total 100 ml.

Protein was purified using immobilized metal affinity chromatography (IMAC) according to the following. 95 ml Supernatant was used (˜4 ml of all is kept for analysis (IPC)).

### Used Material:

Nickel-NTA Agarose, Qiagen, Cat No./ID: 30230, Poly-Prep Chromatography Columns, empty, BioRad, No. 731-1550, IMAC A Buffer pH7.4 (containing 20 mM NaPO₄-buffer and 50 mM Imidazol). IMAC B Buffer pH7.4 (containing 20 mM NaPO₄-buffer and 300 mM Imidazol). TBS (10×-conc. diluted to 1× conc. With MilliQ-Water). Amicon Ultra-4 Centrifugal Filter Unit with Ultracel-10 membrane, 10K, UFC801096.

### Process Steps:

1. Columns were prepared with 1 ml Nickel-NTA-Agarose of Qiagen (=0.5 ml CV);
2. Equilibration with 10 CV IMAC A;
3. Loading of 15/45 ml SN on column (collect flow through);
4. Washing with 10 CV IMAC A (collect in 15 ml Falcon tube);
5. Elution in 6.5 CV of IMAC B;
6. Determination of concentration of eluate;
7. Concentration of 3.5 ml sample to ˜400 μl with Amicon Ultra-4 Centrifugal Filter Unit 10K;
8. Buffer exchange by adding TBS and centrifugation 5000;
Samples were analyzed using analytical SEC with 40 μl of each sample and using protein gel with 12 μl of each sample.

The resulting protein was stored.

(2) plusMIL

Cloning of EP14aa1-3 with Membrane Interaction Loop (aa193-204) with Overlap extension PCR was performed.

The following primers were used:

TABLE 24
Primers

| Primer | Sequence |
|---|---|
| FW | SEQ ID NO: 207 |
| REV | SEQ ID NO: 208 |
| Rev-sense | SEQ ID NO: 98 |

A PCR reaction was set up using the following pipetting scheme:
1.2 μl Phusion Hot Start Polymerase,
24 μl 5× HF-buffer,
0.96 μl 100 mM dNTPs (25 mM of each dNTP),
0.6 μl Fw primer,
0.6 μl Rev primer,
92.64 μl DEPC H₂O.

The PCR cycling parameters according to Table 17 were used.

TABLE 25
PCR cycling parameters

| | No. of | | | |
|---|---|---|---|---|
| | cycles | Temp | Time | |
| 1 | 1 | 98° C. | 30 | sec |
| 2 | 30 | 98° C. | 10 | sec |
| | | 50-70° C. | 30 | sec |
| | | gradient | | |
| | | 72° C. | 30 | sec |
| 3 | 1 | 72° C. | 10 | min |

Directly after reaction, 2 μl Dpnl-Enzyme was added to each reaction, mixed and incubated for 2 h at 37° C.

Transformation was performed by transferring 2 μl PCR product to a 96-well PCR plate and cool down on ice. 20 μl STELLAR chemical component bacteria was added and carefully mixed by pipetting once up-and-down. The samples were incubated 30 min on ice, and then 45 sec at 42° C. in a PCR machine, followed by another 60 sec incubation on ice. Finally, 90 μl SOC medium was added and incubated 1 hr at 37° C. The whole transfection mix was plated on LB-Ampicilin or LB-Carbencilin plates and grown over night at 37° C.

The resulting protein EP14_plusMIL, with an amino acid sequence according to SEQ ID NO: 155, was stored.

### 9. Example 8: Enzymatic Activity

The candidates generated in previous examples, were characterized using an enzymatic activity assay.

The following reagents were used: Pi free buffer, a phosphate-free physiological saline solution (140 mM NaCl, 5 mM KCl, 1 mM MgCl₂, 2 mM CaCl₂, 10 mM Hepes, pH7.4); and Pi free buffer+2% BSA, a phosphate-free physiological saline solution with 20 mg/ml BSA; CD39 protein (according to SEQ ID NO: 1); ATP.

Duplicate CD39 solution was prepared at 2 μg/ml. Duplicate ATP solution was prepared at 1000 μM from a 15 μl ATP stock+1185 μl buffer, total 1.2 ml.

The enzymatic reaction was studied by mixing the 60 μl ATP with 60 μl CD39 or 60 μl with Pi free buffer for the controls, in 48 well PCR plates filled with 120 μl final/well. The final concentration was 500 μM ATP and 1 μg/ml CD39.

Samples were incubated at 37° C. for 0, 5, 15, 30, 60, 90, and 150 minutes, respectively. Then, samples were evaluated either by Pi release assay or HPLC.

### (1) Pi Release Assay

### (a) Materials and Methods

Reagents were prepared from a standard Pi detection kit according the manufacturers instructions.

A standard curve with Pi was prepared by dilution in water. A 1:2 serial dilution of the Pi stock (100 μM) was prepared: 450 μl+450 μl water. The standard curve concentration was: 50 μM/25 μM/12.5 μM/6.25 μM/3.1 μM/1.5 μM/0 μM.

Gold reagent mix was prepared: 4 ml gold reagent+40 μl accelerator (for 3 plates). In a 96 well plate, the samples were diluted 1:10 in H₂O (Dilution in water: 10 μl sample+90 μl H2O). 50 μl, 1:10 diluted sample was distributed in each well of a 96 half-area well plate (Corning, 3690). 12.5 μl Gold reagent mix was added to each well (25% sample volume) and the samples were incubated 10 min at room temperature. Absorbance was read at 635 nm.

### (b) Results and Interpretation

Comparative results for candidates are shown in Table 26.

TABLE 26
Comparative results for candidates.

| | | | | | | | In vitro |
|---|---|---|---|---|---|---|---|
| | | | | | | | enzyme |
| | | | | Yield | | Melting | activity |
| | | | | after | Monomeric % | Temp | (relative |
| | Construct | IL-2 start | SEQ ID | ALC | after ALC | (° C.) | to EP28) |
| 1 | EP14aa1-3 | SEQ ID NO: 137 | SEQ ID NO: 229 | 33.1 | 83.8 | 62 | 4 |
| 2 | EP1xEP14aa1-3 | SEQ ID NO: 137 | SEQ ID NO: 221 | 3.7 | 50.6 | 65.5 | 4 |
| 3 | EP1xEP17aa1-3 | SEQ ID NO: 137 | SEQ ID NO: 211 | 95.8 | 91.9 | 61 | 1.5 |
| 4 | EP17xEP19 aa1-3 | SEQ ID NO: 137 | SEQ ID NO: 227 | 3.5 | 38.9 | 60.5 | 1.5 |
| 5 | EP1xEP17xEP19aa1-3 | SEQ ID NO: 137 | SEQ ID NO: 217 | 153.2 | 89.7 | 60.75 | 1.5 |
| 6 | EP1 aa1-3 | SEQ ID NO: 137 | SEQ ID NO: 209 | 6.0 | 9.2 | 59.75 | 1 |
| 7 | EP1xEP14 aa1-6 | SEQ ID NO: 135 | SEQ ID NO: 223 | 148.8 | 91.8 | 64 | 4 |
| 8 | EP1xEP17 aa1-6 | SEQ ID NO: 135 | SEQ ID NO: 213 | 59.4 | 86.1 | 59.5 | 1.5 |
| 9 | EP17xEP19 aa1-6 | SEQ ID NO: 135 | SEQ ID NO: 227 | 2.7 | 13.7 | 60 | 1.5 |
| 10 | EP1xEP17xEP19aa1-6 | SEQ ID NO: 135 | SEQ ID NO: 219 | 30.9 | 88.4 | 60.75 | 1.5 |
| 11 | EP1xEP17_K405Naa1-15 | SEQ ID NO: 133 | SEQ ID NO: 215 | 156.3 | 86.4 | 62.25 | 1.5 |
| 12 | EP28aa1-3 | SEQ ID NO: 137 | SEQ ID NO: 58 | 2.7 | 10 | 60.5 | 1 |
| 13 | EP28aa1-16 | SEQ ID NO: 131 | SEQ ID NO: 72 | 4.7 | 45 | 61.8 | 1 |

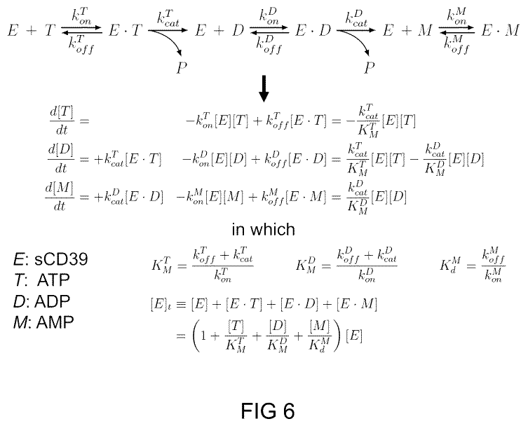

Enzymatic activity was measured by adding 500 μM ATP to the enzyme and analyzing the concentration of ATP, ADP, AMP with HPLC (method description below) over time. The resulting kinetic curves where fitted with the model in FIG. 6 to obtain the enzymatic constants. In terms of the enzymatic constant Kcat the enzymes show the following order (low activity to high activity): EP28 (wt), EP17, EP14, EP15.

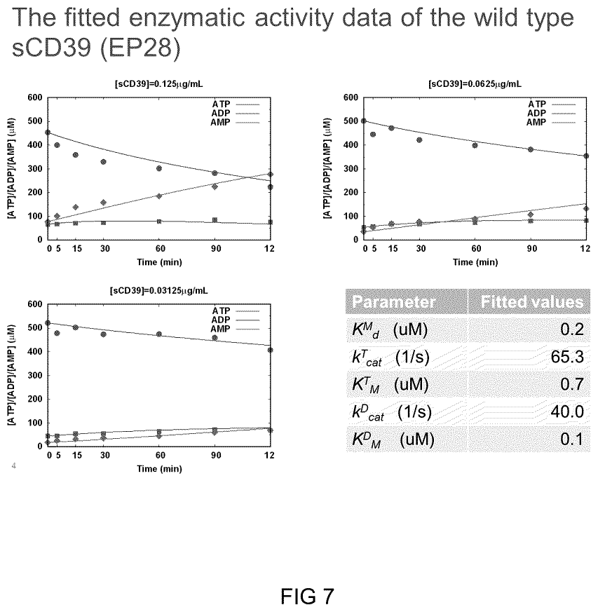

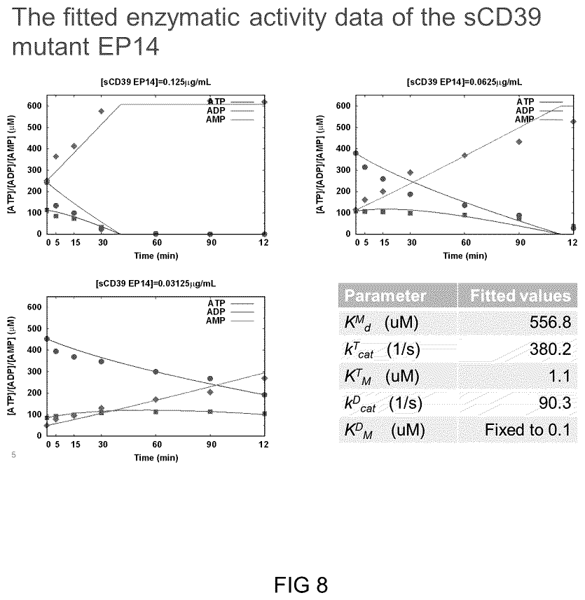

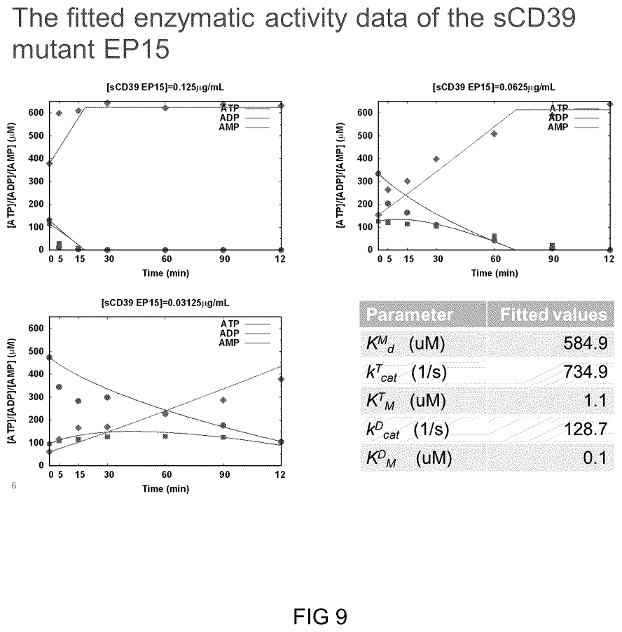

FIG. 7 shows kinetic data and model fit for EP28. FIG. 8 shows kinetic data and model fit for EP14. FIG. 9 shows kinetic data and model fit for EP15.

An overview of enzyme constants for EP28 (wt), EP14, EP15 and EP17 is shown in Table 27. Compared to the wild type (WT) the three novel variants show increased catalytic activity. Importantly, the new variants show a clear increase in the catalytic rate constant (kcat) catalytic efficiency (kcat/Km). As the reported ATP and ADP substrate concentration during tissue damage and thrombosis are above the reported Km, this increase in kcat and kcat/Km will likely translate in higher activity in vivo.

TABLE 27
Enzyme constants

| | ATP −> ADP + P | | | ADP −> AMP + P | | | inhibition |
|---|---|---|---|---|---|---|---|
| | k^{T}_{cat} | K^{T}_{M} | k^{T}_{cat}/K^{T}_{M} | k^{D}_{cat} | K^{D}_{M} | k^{D}_{cat}/K^{D}_{M} | K^{M}_{d} |
| WT | 65.3 | 0.7 | 89.7 | 40.0 | 0.1 | 400.3 | 0.2 |
| EP14 | 380.2 | 1.1 | 352.6 | 90.3 | Fixed to | 903.0 | 556.8 |
| | | | | | 0.1 | | |
| EP15 | 734.9 | 1.1 | 658.1 | 128.7 | 0.1 | 1287.1 | 584.9 |
| EP17 | 180.7 | 1.0 | 178.1 | 67.0 | 0.1 | 670.0 | 3.0 |

### (2) HPLC Validation Assay (Kinetic and Dose Response)

### (a) Materials and Methods

The candidates were tested with HPLC validation assay. 70 μl of each sample was transferred into glass vials for HPLC.

Calibration samples were prepared with Stock-Solutions 5 mM as shown in Table 28.

TABLE 28
Stock solutions

| | 5 mM | weight | H₂O | |
|---|---|---|---|---|
| Adenosine | 1336.2 μg/mL | 1.862 mg | 1394 | μL |
| Inosine | 1341.2 μg/mL | 2.668 mg | 1989 | μL |
| AMP | 1736.1 μg/mL | 2.182 mg | 1257 | μL |
| ADP | 2506.6 μg/mL | 2.500 mg | 997 | μL |
| ATP | 2755.7 μg/mL | 4.834 mg | 1754 | μL |

| | | |
|---|---|---|
| 1000 | μM | 20 μL of each stock-solutions were mixed in a HPLC vial |
| 500 | μM | 20 μL 1 mM + 20 μL H₂O |
| 100 | μM | 10 μL 1 mM + 90 μL H₂O |
| 10 | μM | 10 μL 100 μM + 90 μL H₂O |
| 1 | μM | 10 μL 10 μM + 90 μL H₂O |

HPLC Separation was done using an Agilent 1100 System with a CapPump (G1376A), Degasser (G1379A), ALS (G1329A), Thermostat (G1330B, ColComp (G1316A) and a DAD (G1315A). Solvent A: 10 mM KH₂PO₄ (04243, Riedel-de Haën)+2 mM TBA bromide, pH7.0 (86857-10G-F, Fluka) and Solvent B: 10 mM KH2PO4/ACN 1/1+2 mM TBA bromide, pH5.5. Column: Nucleodur 300-5 C18 ec, 2×150 mm, 5 μm, Macherey-Nagel 760185.20 Batch E14100258 36654055. The column temperature was 40° C., injection volume 10 μL, flow rate was 0.3 ml/min and the gradient was 0-3′: 0% B; 3-23′: 0-95% B, linear; 23-28′: 95% B, linear; 28-29′: 95-0% B, linear; 5′ Post Time “. DAD: 247 nm and 259 nm.

UPLC Separation was done using a Waters UPLC I class. Solvent A: 10 mM KH₂PO₄/10 mM K₂HPO₄ 1/1+2 mM TBA bromide, pH 7.0. Solvent B: 10 mM KH₂PO₄/ACN 1/1+2 mM TBA bromide, pH 5.5. Column: Fortis Bio C18, 2.1×50 mm, 5 μm, di2chrom BIO318-020301 SN H03161210-2. Column temperature was 40° C., injection volume was 10 μL, flow rate was 0.5 ml/min and gradient was 0-1′: 0% B; 1-8′: 0-55% B, linear; 8-10′: 55% B; 10-11′: 55-0% B, linear; 14′ Stop Time. DAD was 247 nm and 259 nm.

### (b) Results

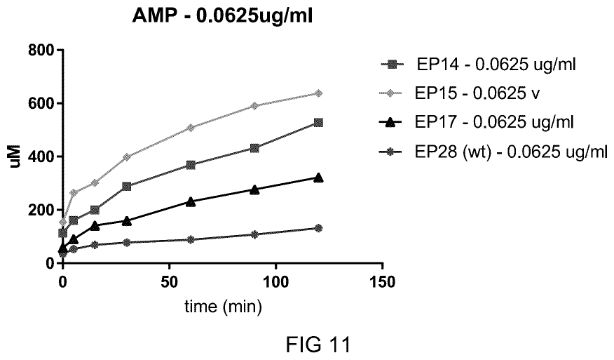

The results can be seen in FIGS. 7, 8, 9 and 11, showing kinetic data and model fit for candidates according to different embodiments.

### 10. Example 9: In Vitro Activity, Initial Screen

The CD39 versions EP1 to EP24, described in previous examples, were cloned in mammalian expression vector pRS5a_Leader_APP_His (FIG. 5), without IL2-leader and without IL2-start.

### (a) Materials and Methods

A small scale expression (20/50 ml scale) of EP-Hits in HEK293 (PEI-Transfection) for 7 days was performed, followed by IPC on APP-HPLC (as described supra).

Protein purification of 15/45 ml cell supernatant with Ni-NTA-columns (0.5 ml CV); Elution with 6 CV IMAC B buffer (20 mM NaPO4-buffer, 300 mM Imidazol, pH7.4); Concentration and rebuffering of purified protein in TBS, pH7.4;
Analysis of protein with protein gel, analytical SEC;
Delivery of all variants and three controls (parental hCD39-dMIL, or EP28, with and without IL2-start, and 8M-version without IL2-start): 90-200 μl of purified protein in TBS, pH7.4

### (b) Results and Interpretation

The results are summarized in Table 29 below.

All samples are in TBS pH7.4 and have an APP-(SEQ ID N: 247) and a His-Tag (SEQ ID NO: 249)

Only parental human CD39ΔMIL (EP28) has a 15 amino acid long IL2-start, aa1-15 (SEQ ID NO: 133).

Pi release assay BOENKTH1-0252824, double det. For 60 and 180 min. values

TABLE 29
In vitro activity
Pi release assay,
0.25 ug/ml + 500 uM ATP

| | | | | total | | | |
|---|---|---|---|---|---|---|---|
| Date of | | conc. | conc. | vol. | | mean | mean |
| purification | Name | [mg/ml] | [umolar] | [ml] | 0 min. | 60 min. | 180 min |
| 17 Jun. 2016 | EP1 | 0.19 | 3.7 | 0.11 | 10.35 | 20.90 | 45.99 |
| 14 Jun. 2016 | EP2 | 0.15 | 2.8 | 0.10 | 11.35 | 23.65 | 53.92 |
| 14 Jun. 2016 | EP3 | 0.11 | 2.2 | 0.10 | 8.92 | 19.22 | 40.76 |
| 14 Jun. 2016 | EP4 | 0.25 | 4.8 | 0.10 | 7.99 | 18.59 | 37.47 |
| 14 Jun. 2016 | EP5 | 0.11 | 2.1 | 0.10 | 17.36 | 39.59 | 72.09 |
| 17 Jun. 2016 | EP6 | 0.14 | 2.7 | 0.15 | 11.57 | 32.73 | 69.63 |
| 21 Jun. 2016 | EP7 | 0.41 | 7.8 | 0.11 | 9.47 | 21.19 | 43.68 |
| 21 Jun. 2016 | EP8 | 0.31 | 5.9 | 0.11 | 7.92 | 15.23 | 33.94 |
| 14 Jun. 2016 | EP9 | 0.15 | 2.9 | 0.10 | 5.56 | 13.39 | 31.38 |
| 21 Jun. 2016 | EP10 | 0.33 | 6.3 | 0.11 | 9.32 | 21.91 | 54.07 |
| 14 Jun. 2016 | EP11 | 0.23 | 4.4 | 0.10 | 10.24 | 21.27 | 46.21 |
| 21 Jun. 2016 | EP12 | 0.37 | 7.1 | 0.11 | 10.55 | 21.99 | 50.80 |
| 21 Jun. 2016 | EP13 | 0.13 | 2.5 | 0.11 | 4.93 | 3.13 | 3.54 |
| 21 Jun. 2016 | EP14 | 0.34 | 6.5 | 0.11 | 19.07 | 66.35 | 104.80 |
| 14 Jun. 2016 | EP15 | 0.19 | 3.7 | 0.10 | 11.11 | 29.26 | 89.92 |
| 14 Jun. 2016 | EP17 | 0.28 | 5.3 | 0.10 | 12.49 | 30.30 | 102.81 |
| 14 Jun. 2016 | EP18 | 0.15 | 2.9 | 0.10 | 3.91 | 9.70 | 23.49 |
| 17 Jun. 2016 | EP19 | 0.55 | 10.6 | 0.12 | 14.36 | 26.38 | 69.93 |
| 14 Jun. 2016 | EP20 | 0.14 | 2.7 | 0.10 | 3.36 | 2.22 | 3.60 |
| 14 Jun. 2016 | EP21 | 0.22 | 4.2 | 0.10 | 7.17 | 16.37 | 24.27 |
| 17 Jun. 2016 | EP22 | 0.17 | 3.3 | 0.11 | 8.06 | 15.33 | 37.30 |
| 21 Jun. 2016 | EP23.1 | 0.28 | 5.4 | 0.11 | 12.62 | 23.58 | 62.78 |
| 17 Jun. 2016 | EP24 | 0.10 | 2.0 | 0.19 | 3.10 | 3.45 | 4.83 |
| 14 Jun. 2016 | EP25 | 0.23 | 4.4 | 0.10 | 4.98 | 11.46 | 24.30 |
| | hCD39-dMIL | | | | | | |
| | parental | | | | | | |
| 17 Jun. 2016 | EP26 | 0.19 | 3.7 | 0.15 | 3.66 | 8.41 | 24.15 |
| | hCD39-8M | | | | | | |
| 17 Jun. 2016 | EP28-IL2start- | 2.19 | 42.0 | 0.14 | 10.25 | 23.48 | 52.73 |
| | hCD39 par | | | | | | |
| 21 Jun. 2016 | EP1repet. | 0.49 | 9.5 | 0.11 | 10.03 | 17.84 | 40.33 |
| 17 Jun. 2016 | EP2repet. | 0.55 | 10.6 | 0.12 | 12.80 | 28.22 | 66.88 |
| 17 Jun. 2016 | EP3repet. | 0.56 | 10.8 | 0.12 | 18.05 | 33.35 | 79.53 |
| 17 Jun. 2016 | EP4repet. | 0.61 | 11.7 | 0.16 | 15.98 | 29.47 | 68.39 |
| 17 Jun. 2016 | EP5repet. | 0.66 | 12.6 | 0.15 | 25.53 | 46.10 | 91.32 |
| 21 Jun. 2016 | EP23.4 | 0.29 | 5.5 | 0.11 | 10.43 | 22.45 | 56.53 |

### 11. Example 10: In Vitro Activity, Refined Screen

A subset of 12 mutants were tested a second time, but with IL-2 start allowing a larger expression scale.

### (a) Materials and Methods

The mammalian expression vector pRS5a_Leader_APP_His, with a 15 amino acid long IL2-start, aa1-15 (SEQ ID NO: 133) (FIG. 5). Small scale expression (50/100 ml scale) of EP-Hits in HEK293 (PEI-Transfection) for 7 days followed by IPC on APP-HPLC (as described supra).

Protein purification of 45/95 ml cell supernatant with Ni-NTA-columns (0.5 ml CV) Elution with 6 CV IMAC B buffer (20 mM NaPO4-buffer, 300 mM Imidazol, pH7.4) Concentration and rebuffering of purified protein in TBS, pH7.4, Analysis of protein with protein gel, analytical SEC

Delivery of all variants and control (parental hCD39-dMIL, or EP28, with IL2-start aa1-15 (SEQ ID NO: 133)):
500 μl of purified protein in TBS, pH7.4

### (b) Results and Interpretation

The results are summarized in Table 30, Table 32 and Table 33 below.

TABLE 30
Protein purifications of EP-Hits 6 Jul. 2016, part 1

| | | | total | | | |
|---|---|---|---|---|---|---|
| | conc. | conc. | vol. | yield/46 or | Yield | IPC (APP, |
| Name | [mg/ml] | [umolar] | [ml] | 16 ml [mg] | (mg/l) | mg/l) |
| EP2 | 0.64 | 12.3 | 0.5 | 0.32 | 7.13 | 6.9 |
| EP3 | 0.72 | 13.9 | 0.5 | 0.36 | 8.02 | 8.6 |
| EP4 | 0.86 | 16.4 | 0.5 | 0.43 | 9.49 | 8.6 |
| EP5 | 0.89 | 17.0 | 0.5 | 0.44 | 9.84 | 10.1 |
| EP6 | 0.40 | 7.7 | 0.5 | 0.20 | 4.46 | 4.3 |
| EP9 | 0.57 | 11.0 | 0.5 | 0.29 | 6.35 | 6.0 |
| EP11 | 0.74 | 14.2 | 0.5 | 0.37 | 8.18 | 8.3 |
| EP12 | 0.85 | 16.3 | 0.5 | 0.42 | 9.40 | 8.2 |
| EP14 | 0.82 | 15.7 | 0.5 | 0.41 | 9.09 | 7.8 |
| EP17 | 0.94 | 18.0 | 0.5 | 0.47 | 13.04 | 13.3 |
| EP18 | 0.70 | 13.4 | 0.5 | 0.35 | 7.75 | 6.4 |
| EP24 | 0.65 | 12.4 | 0.5 | 0.32 | 8.99 | 10.8 |
| EP28 with | 0.27 | 5.2 | 0.5 | 0.14 | 9.01 | 6.6 |
| IL2-start | | | | | | |

TABLE 31
Protein purifications of EP-Hits Jun. 7, 2016, part 2

| | | | | | Pi Assay: |
|---|---|---|---|---|---|
| | | | Mono- | | 1 ug/ml |
| | | Aggre- | meric | Degra- | Enzyme, |
| | | gation | peak | dation | 500 uM |
| | Expr. | (%) | (%) | (%) | ATP, |
| | vol. | (Anal. | (Anal. | (Anal. | 60 min |
| Name | (ml) | SEC) | SEC) | SEC) | (1:10) |
| EP2 | 50 | 15.7 | 69.5 | 14.9 | 53.572 |
| EP3 | 50 | 14.9 | 67.6 | 17.5 | 62.6725 |
| EP4 | 50 | 11.3 | 72.5 | 16.3 | 52.1195 |
| EP5 | 50 | 15.1 | 67.8 | 17 | 75.1995 |
| EP6 | 50 | 17.2 | 59.3 | 23.5 | 72.517 |
| EP9 | 50 | 21.7 | 57.5 | 20.8 | 63.7175 |
| EP11 | 50 | 12.3 | 72.2 | 15.6 | 59.0205 |
| EP12 | 50 | 19.9 | 63.1 | 17.1 | 48.358 |
| EP14 | 50 | 14.9 | 66.6 | 18.5 | 88.3785 |
| EP17 | 40 | 17.3 | 68.9 | 13.8 | 89.8415 |
| EP18 | 50 | 15.4 | 67.2 | 17.4 | 61.495 |
| EP24 | 40 | 8.9 | 73 | 18 | 74.713 |
| EP28 with | 20 | 17.8 | 56.4 | 25.8 | 53.6735 |
| IL2-start | | | | | |

TABLE 32
Protein purifications EP-Hits 15 Jul. 2016

| | | | | | total | | Expression |
|---|---|---|---|---|---|---|---|
| | MW | e | conc. | conc. | volume | delivered | volume |
| Name | [kD] | [M-1*cm-1] | [mg/ml] | [umolar] | [ml] | amount | (ml) |
| EP8 | 52.08 | 70875 | 0.02 | 0.5 | 0.5 | 0.01 | 50 |
| EP10 | 52.08 | 70875 | 0.76 | 14.7 | 0.5 | 0.38 | 50 |
| EP14 | 52.08 | 70875 | 0.21 | 4.0 | 0.5 | 0.10 | 50 |
| EP15 | 52.08 | 70875 | 0.81 | 15.6 | 1 | 0.81 | 100 |
| EP17 | 52.08 | 70875 | 0.22 | 4.3 | 0.5 | 0.11 | 40 |
| EP19 | 52.08 | 70875 | 1.10 | 21.2 | 0.5 | 0.55 | 50 |
| EP23 | 52.08 | 70875 | 0.46 | 8.8 | 0.5 | 0.23 | 50 |
| EP28-parental | 52.08 | 70875 | 0.77 | 14.7 | 1 | 0.77 | 100 |
| control | | | | | | | |

TABLE 33
Point mutations, in relation to the wild type
CD39 sequence according to SEQ ID NO: 1.

| | | | | | | Pi Assay: |
|---|---|---|---|---|---|---|
| | | | | | | 1 ug/ml |
| | | | | | | Enzyme, |
| | | | | | | 500 uM |
| No. | Mutation | Mutation | Mutation | Mutation | Sequence | ATP, 60 min., |
| Mutations | Pos. 1 | Pos. 2 | Pos. 3 | Pos. 4 | found | 1:10 |
| 1 | 151: V−>A | | | | 2x | 4.309 |
| 1 | 319: I−>T | | | | 2x | 54.866 |
| 1 | 365: F−>S | | | | unique | 69.0195 |
| 4 | 292: N−>K | 365: F−>S | 424: L−>P | 463: P−>S | unique | 78.81 |
| 2 | 405: K−>N | 412: Y−>F | | | unique | 89.415 |
| 2 | 424: L−>Q | 436: H−>D | | | unique | 62.912 |
| 4 | 254: L−>M | 263: W−>R | 439: F−>S | 469: S−>R | unique | 88.282 |

### 12. Example 11: In Vivo Activity, pK

### (a) Materials and Methods

For in-vivo PK 10 mg/kg compound at a final concentration of 10 mg/ml in PBS buffer was administered intravenously (1 ml/kg) via the tail vein to 4 conscious female C57BL/6 mice. Mice were obtained from WIGA and had a bodyweight of around 22 g. All in-life work was conducted under the Swiss animal welfare law.

Whole blood was collected (50 μL per time point) 0.25, 3, 8, 24 and 48 h post dose in small volume serum tubes using POCT Minivettes. Serum was separated and used for concentration determination.

The Gyrolab technology is an automated immunoassay at nanoliter scale using an affinity flow-through format which works through centrifugal forces and laser-induced fluorescence detection. Streptavidin-coated beads are pre-packed in affinity columns on a Gyrolab Bioaffy CD. Each CD contains 112 columns. The affinity-capture columns per microstructure comprise 15 nl. The injected samples enter by capillary action. The biotinylated capture reagent binds to the Streptavidin coated beads. Afterwards, the analyte solution is injected, which binds to the captured molecules. Finally, the fluorophore-labeled detection reagent is applied. In the case of CD39, two different assay read-outs were used depending on the availability of an APP tag.

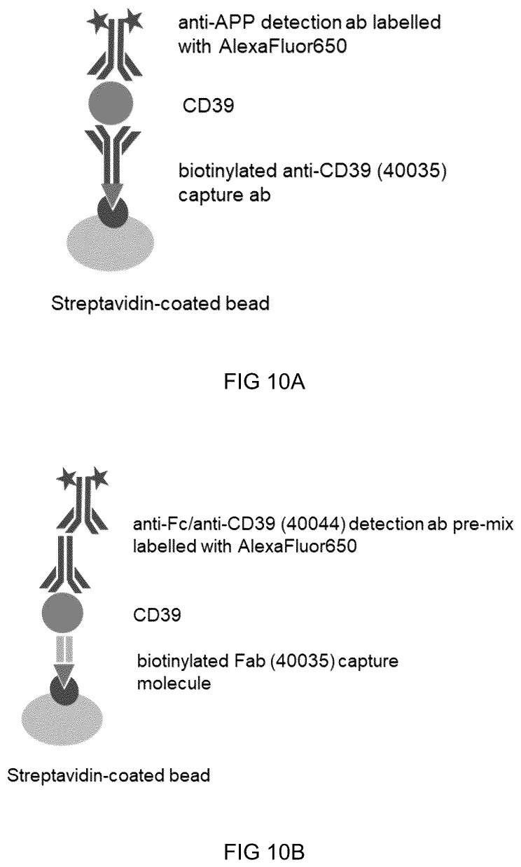

1) anti-CD39 (40035) and anti-APP (27431) is seen in FIG. 10A.

2) Fab (40035) and anti-Fc/anti-CD39 (40044) 1:1 pre-mix (all EP28aa1-16 constructs) is seen in FIG. 10B. Antibody 40044 loses activity when biotinylated by amine coupling, therefore, only antibody 40035 was biotinylated.

All standard curves for the CD39 constructs were diluted in Rexxip A containing 5% (v/v) mouse serum in a dilution series of 1:2. The applied concentration range for the APP tagged constructs was 5000 ng/ml-9.77 ng/ml and for EP28aa1-16 it was 10000 ng/ml-9.77 ng/ml. All mouse sera samples were diluted 1:100 in Rexxip A containing 5% (v/v) mouse serum. The QC samples of the CD39 constructs were diluted in Rexxip A containing 5% (v/v) mouse serum (50 and 500 ng/ml for constructs with APP tag, and 500 and 1000 ng/ml for EP28aa1-16). The final concentration for all biotinylated capture reagents was 0.1 mg/ml and the fluorescently labelled detection antibody was diluted to 10 nM in Rexxip F.

### (b) Result and Interpretation

The results are summarized in Table 34. As can be seen, all candidates show the same PK. Therefore, the selection of the candidate was not based on PK properties.

TABLE 34
pK values

| | | | | | | Mean |
|---|---|---|---|---|---|---|
| Construct | SEQ ID | Animal 1 | Animal 2 | Animal 3 | Animal 4 | t/2 (d) |
| EP28aa1-16 | SEQ ID NO: 72 | 1.1 | 1.3 | 1.0 | 1.1 | 1.1 |
| EP1xEP14aa1-6 | SEQ ID NO: 223 | 0.9 | 0.7 | 0.7 | 0.3 | 0.7 |
| EP28aa1-3 | SEQ ID NO: 58 | 0.6 | 0.7 | 0.9 | 0.8 | 0.8 |
| EP14aa1-3 | SEQ ID NO: 229 | 0.7 | 1.0 | 0.6 | 1.1 | 0.9 |
| EP14aa1-6 | SEQ ID NO: 231 | 0.7 | 0.7 | 0.7 | 0.8 | 0.7 |
| EP17aa1-3 | SEQ ID NO: 237 | 0.9 | 0.8 | 0.9 | 0.8 | 0.9 |
| EP17aa1-15 | SEQ ID NO: 241 | 0.8 | 0.9 | 0.9 | 0.6 | 0.8 |

### 13. Example 12: In Vivo Activity, AKI Model

### (a) Materials and Methods

A nephrectomy of the right kidney is performed prior to the start of I/R setting. The second kidney is removed to avoid compensatory mechanisms that change the entire dynamic of the biology. Spontaneously breathing anesthetized animals are placed on the homeothermic blanket of a homeothermic monitor system and covered by sterile gauze. The body temperature is recorded through a rectal probe and controlled in the range of 36.5-37.5° C. to avoid hypothermia. Animals are anaesthetized, shaved and disinfected (Betaseptic). Following mid-line incision/laparotomy the abdominal contents are retracted to the left and the right kidney is removed. The ureter and blood vessels are disconnected and ligated (9-0 Ethicon), the kidney is then removed.

I/R injury induction: Immediately after the nephrectomy of the right kidney abdominal contents are retracted to the right and the left renal artery is dissected free for renal ischemia induction.

Micro-aneurysm clips are used to clamp the pedicle to block the blood flow to the kidney and induce renal ischemia. The duration of the kidney ischemia starts from the time of clamping. Successful ischemia is confirmed by color change of the kidney from red to dark purple in a few seconds. After the ischemia, the micro-aneurysm clips are removed and reperfusion is indicated by a kidney color change to red.

### (b) Result and Interpretation

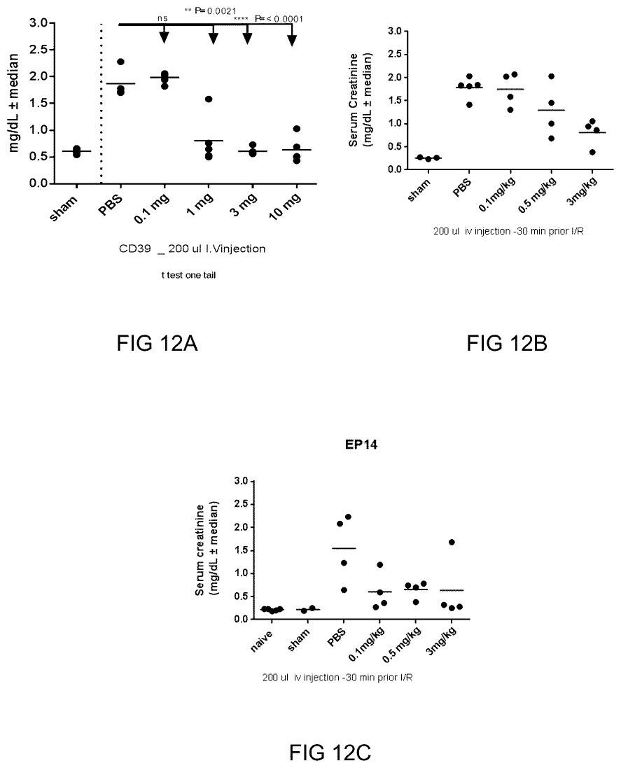

The result is seen in FIG. 12. The candidates show a dose response in vivo which correlate to their specific in vitro activity. FIG. 12A shows results for the parental EP28, FIG. 12B shows results for EP1×EP17 and FIG. 12C shows results for EP14. As can be seen, EP28 and EP1×EP17 show similar dose responses, whereas EP14 with higher in vitro activity shows full efficacy with a lower dose.

### 14. Example 13: Titer, Yield and Developability

In order to manufacture selected candidates in a commercial scale, it is important to be able to express them with relatively high yield. For therapeutic proteins, this might be less straightforward compared to therapeutic antibodies, due to format complexity in addition to lack of enrichment technology which enables selection of high producing clones.

Both candidates, EP14aa1-3 and EP28aa1-3, had comparable technical characteristics, which were challenging. Particularly, low expression titers of early expression batches (data not shown) impacts production costs or might even be even lower after upscaling because of the control of host cell proteins is not robust.

In order to try to improve protein expression by early clone selection for both candidates a tailor-made purification process required was required. To this end, pools of cells expressing the candidates EP28aa1-3 and EP14aa1-3 were generated.

A parental CHO cell line was used as host cell line for the production of the EP28aa1-16/EP14aa1-3 expressing cell line. The host cell line was derived from the CHO-K1 cell line, well known to a person skilled in the art, in a way described e.g. in the patent applications WO2015092737 and WO2015092735, both incorporated by reference in their entirety. A single vial from the CHO line was used to prepare the EP28aa1-16/EP14aa1-3 recombinant cell line.

The cells were grown in chemically defined cultivation medium. One μg of Swal linearized plasmid DNA, expression vector encoding for EP28aa1-16/EP14aa1-3, was added per transfection. The transfection reaction was performed in chemically defined cultivation medium.

Transfections were performed by electroporation using an AMAXA Gene Pulser, according to the manufactures instructions. The parental CHO cells used for transfection were in exponential growth phase with cell viabilities higher than 95%. In total, three transfections were performed with 5×10⁶ cells per transfection. Immediately after transfection, cells were transferred into Shake Flasks, containing medium chemically defined cultivation medium.

Cell pools were incubated for 48 hours at 36.5° C. and 10% CO₂ before starting the selection process. A selection procedure was carried out using the selection marker encoded in the expression vector. 48 hours after transfection and growth under low folate conditions, additional selective pressure was applied by adding 10 nM MTX to the chemically defined cultivation medium. 21 days after the start of MTX selection, pool populations consisting predominantly of MTX resistant cells have emerged. After pool recovery cells were frozen. Standard fed batches in chemically defined cultivation medium were set up for determination of concentration of the EP28aa1-16/EP14aa1-3. A reversed phase chromatography (RPC) was used to determine the product concentration. CHO cell pools producing EP28aa1-16/EP14aa1-3 were used for a FACS single cell sorting/Cell printer procedure to obtain individualized clonal cell lines.

### 15. Example 14: Therapeutic Use

Extracellular ATP, activating P2X₇R, have been clearly linked several diseases, such as enhancing graft-versus-host disease (Wilhelm et al. Graft-versus-host disease is enhanced by extracellular ATP activating P2X₇R. Nature Medicine 16:12, pages 1434-1439 (2010).)

Furthermore, both in vitro and in vivo studies indicate that CD39 represents an important apyrase in cardiovascular health by regulating levels of ADP. Apyrase is known to inhibit platelet aggregation by metabolizing extracellular ADP.

Human apyrase does not covalently bind to platelets as opposed to other therapies like clopidogrel (Plavix™), which irreversibly bind to ADP receptor on the platelet. This allows a faster disappearance of the therapeutic blockade and therefore a safer approach to patients with excessive platelet activation. This provides a safer approach to patients with excessive platelet activation.

Thus, there is a clear basis for therapeutic use of compounds which reduce levels of extracellular ATP, such as the compounds according to the invention.

Specific non-limiting examples of therapeutic uses of the compounds according to the invention are acute organ damage due to trauma and/or hypoxia, such as acute respiratory distress syndrome (ARDS), lung injury, renal failure, acute kidney injury (AKI), including acute kidney injury following coronary artery bypass graft surgery, delayed graft function after transplantation (including xenotransplantation) of kidney or other solid organs, or vascular disease, such as occlusive vascular disease, transplantation, and xenotransplantation, treatment of individuals who suffer from stroke, coronary artery disease or injury resulting from myocardial infarction, atherosclerosis, arteriosclerosis, embolism, preeclampsia, angioplasty, vessel injury, transplantation, neonatal hypoxic ischemic encephalopathy, platelet-associated ischemic disorders including lung ischemia, coronary ischemia and cerebral ischemia, ischemia-reperfusion injury (IRI) thrombotic disorders including, coronary artery thrombosis, cerebral artery thrombosis, intracardiac thrombosis, peripheral artery thrombosis, and venous thrombosis delayed graft function after transplantation (including xenotransplantation) of kidney or other solid organs. Other non-limiting examples of therapeutic uses of compounds according to the invention are treatment of burns or radiation damage, sepsis, improving wound healing, decrease bleeding or the risk of bleeding, prevention organ damage, graft-versus-host disease, or prevention of transplant rejection.

Particularly preferred therapeutic uses of the compounds according to the invention is acute kidney injury (AKI), such as acute kidney injury following coronary artery bypass graft surgery or sepsis or rhabdomyolysis. This condition increases patient mortality and there is no standard of care (SoC). The main causes of AKI in the intensive care unit are: sepsis (47.5%), major surgery (34%), cardiogenic shock (27%), hypovolemia (26%) and nephrotoxic compounds (19%). Furthermore, AKI is an independent strong risk factor for developing chronic kidney disease (CKD). 20-30% of major cardiac surgeries patients acquire acute kidney injury. Another preferred embodiment relates to the use an isolated apyrase according to the invention for the treatment of cardiac surgery associated acute kidney injury.

In another embodiment, the disclosure relates to an isolated apyrase according to the invention for use in the treatment of delayed graft function (DGF), acute respiratory distress syndrome (ARDS), acute myocardial infarction (AMI), traumatic brain injury (TBI)/acute ischemic stroke (AIS), or combinations thereof often referred to as multi-organ failures (MOF).

16. Acute kidney injury (AKI) is a common complication of sepsis. 28% of sepsis patients acquire AKI. In an additional preferred embodiment the disclosure relates to the use an isolated apyrase according to the invention for the treatment of sepsis associated acute kidney injury. Example 15: Therapeutic compositions

Therapeutic proteins are typically formulated either in aqueous form ready for administration or as lyophilisate for reconstitution with a suitable diluent prior to administration. A protein may be formulated either as a lyophilisate, or as an aqueous composition, for example in pre-filled syringes.

Suitable formulation can provide an aqueous pharmaceutical composition or a lyophilisate which can be reconstituted to give a solution with a high concentration of the therapeutic protein active ingredient and a low level of protein aggregation for delivery to a patient. High concentrations of protein are useful as they reduce the amount of material which must be delivered to a patient (the dose). Reduced dosing volumes minimize the time taken to deliver a fixed dose to the patient. The aqueous compositions of the invention with high concentration of proteins are particularly suitable for subcutaneous administration.

Thus the invention provides an aqueous pharmaceutical composition, suitable for administration in a subject, e.g., for subcutaneous administration, comprising a therapeutic protein.

The therapeutic protein may be used as a pharmaceutical composition when combined with a pharmaceutically acceptable carrier. Such a composition may contain, in addition to a therapeutic protein, carriers, various diluents, fillers, salts, buffers, stabilizers, solubilizers, and other materials well known in the art. The characteristics of the carrier will depend on the route of administration. The pharmaceutical compositions for use in the disclosed methods may also contain additional therapeutic agents for treatment of the particular targeted disorder.

### 17. Example 16: Route of Administration

Typically, the proteins according to the invention are administered by injection, for example, either intravenously, intraperitoneally, or subcutaneously. Methods to accomplish this administration are known to those of ordinary skill in the art. It may also be possible to obtain compositions that may be topically or orally administered, or which may be capable of transmission across mucous membranes. As will be appreciated by a person skilled in the art, any suitable means for administering can be used, as appropriate for a particular selected route of administration.

Examples of possible routes of administration include parenteral, (e.g., intravenous (I.V. or IV), intramuscular (IM), intradermal, subcutaneous (S.C. or SC), or infusion), oral and pulmonary (e.g., inhalation), nasal, transdermal (topical), transmucosal, intra-arterial, continuous infusion, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

An apyrase therapy can be initiated by administering a “loading dose” of the proteins according to the invention to the subject in need therapy. By “loading dose” is intended an initial dose of the proteins according to the invention that is administered to the subject, where the dose of the proteins according to the invention administered falls within the higher dosing range. The “loading dose” can be administered as a single administration, for example, a single infusion where the proteins are administered IV, or as multiple administrations, for example, multiple infusions where the proteins are administered IV, so long as the complete “loading dose” is administered within about a 24-hour period (or within the first month if multiple intravenous administration are needed, based on the severity of the disease). Following administration of the “loading dose”, the subject is then administered one or more additional therapeutically effective doses of the proteins according to the invention. Subsequent therapeutically effective doses can be administered, for example, according to a weekly dosing schedule, or once every two weeks, once every three weeks, or once every four weeks. In such embodiments, the subsequent therapeutically effective doses generally fall within the lower dosing range.

Alternatively, in some embodiments, following the “loading dose”, the subsequent therapeutically effective doses of the proteins according to the invention are administered according to a “maintenance schedule”, wherein the therapeutically effective dose of the proteins according to the invention is administered once a month, once every 6 weeks, once every two months, once every 10 weeks, once every three months, once every 14 weeks, once every four months, once every 18 weeks, once every five months, once every 22 weeks, once every six months, once every 7 months, once every 8 months, once every 9 months, once every 10 months, once every 11 months, or once every 12 months. In such embodiments, the therapeutically effective doses of the proteins according to the invention fall within the lower dosing range, particularly when the subsequent doses are administered at more frequent intervals, for example, once every two weeks to once every month, or within the higher dosing range, particularly when the subsequent doses are administered at less frequent intervals, for example, where subsequent doses are administered one month to 12 months apart.

The timing of dosing is generally measured from the day of the first dose of the active compound, which is also known as “baseline”. However, different health care providers use different naming conventions.

Notably, week zero may be referred to as week 1 by some health care providers, while day zero may be referred to as day one by some health care providers. Thus, it is possible that different physicians will designate, e.g., a dose as being given during week 3/on day 21, during week 3/on day 22, during week 4/on day 21, during week 4/on day 22, while referring to the same dosing schedule. For consistency, the first week of dosing will be referred to herein as week 0, while the first day of dosing will be referred to as day 1. However, it will be understood by a skilled artisan that this naming convention is simply used for consistency and should not be construed as limiting, i.e., weekly dosing is the provision of a weekly dose of the protein regardless of whether the physician refers to a particular week as “week 1” or “week 2”. Example of dosage regimes as noted herein are found in FIGS. 1 and 2. It will be understood that a dose need not be provided at an exact time point, e.g., a dose due approximately on day 29 could be provided, e.g., on day 24 to day 34, e.g. day 30, as long as it is provided in the appropriate week.

As used herein, the phrase “container having a sufficient amount of the protein to allow delivery of [a designated dose]” is used to mean that a given container (e.g., vial, pen, syringe) has disposed therein a volume of a protein (e.g., as part of a pharmaceutical composition) that can be used to provide a desired dose. As an example, if a desired dose is 500 mg, then a clinician may use 2 ml from a container that contains a protein formulation with a concentration of 250 mg/ml, 1 ml from a container that contains a protein formulation with a concentration of 500 mg/ml, 0.5 ml from a container contains a protein formulation with a concentration of 1000 mg/ml, etc. In each such case, these containers have a sufficient amount of the protein to allow delivery of the desired 500 mg dose.

As used herein, the phrase “formulated at a dosage to allow [route of administration] delivery of [a designated dose]” is used to mean that a given pharmaceutical composition can be used to provide a desired dose of a protein via a designated route of administration (e.g., s.c. or i.v.). As an example, if a desired subcutaneous dose is 500 mg, then a clinician may use 2 ml of a protein formulation having a concentration of 250 mg/ml, 1 ml of a protein formulation having a concentration of 500 mg/ml, 0.5 ml of a protein formulation having a concentration of 1000 mg/ml, etc. In each such case, these protein formulations are at a concentration high enough to allow subcutaneous delivery of the protein. Subcutaneous delivery typically requires delivery of volumes of less than about 2 ml, preferably a volume of about 1 ml or less. However, higher volumes may be delivered over time using, e.g., a patch/pump mechanism.

Disclosed herein is the use of a protein for the manufacture of a medicament for the treatment of tissue damage in a patient, wherein the medicament is formulated to comprise containers, each container having a sufficient amount of the protein to allow delivery of at least about 75 mg, 150 mg, 300 mg or 600 mg protein per unit dose.

Disclosed herein is the use of a protein for the manufacture of a medicament for the treatment of tissue damage in a patient, wherein the medicament is formulated at a dosage to allow systemic delivery (e.g., i.v. or s.c. delivery) 75 mg, 150 mg, 300 mg of 600 mg protein per unit dose.

### 18. Example 17: Kits

The disclosure also encompasses kits for treating a patient with tissue damage (as the case may be) with a protein. Such kits comprise a protein (e.g., in liquid or lyophilized form) or a pharmaceutical composition comprising the protein (described supra). Additionally, such kits may comprise means for administering the protein (e.g., a syringe and vial, a prefilled syringe, a prefilled pen, a patch/pump) and instructions for use. The instructions may disclose providing the protein to the patient as part of a specific dosing regimen. These kits may also contain additional therapeutic agents (described supra) for treating psoriasis, e.g., for delivery in combination with the enclosed protein.

The phrase “means for administering” is used to indicate any available implement for systemically administering a drug top a patient, including, but not limited to, a pre-filled syringe, a vial and syringe, an injection pen, an autoinjector, an i.v. drip and bag, a pump, patch/pump, etc. With such items, a patient may self-administer the drug (i.e., administer the drug on their own behalf) or a care-giver or a physician may administer the drug.

Disclosed herein are kits for the treatment of a patient having tissue damage, comprising: a) a pharmaceutical composition comprising a therapeutically effective amount of a protein; b) means for administering the protein to the patient; and c) instructions providing subcutaneously administering a protein to a patient in need thereof.

### Sequence Table

Useful amino acid and nucleotide sequences for practicing the invention are disclosed in Table 35.

TABLE 35
Sequences useful for practicing the invention.

| SEQ ID | | |
|---|---|---|
| Number | Feature | Sequence |
| Wild type | | |
| human | | |
| CD39 | | |
| SEQ ID | Amino | MEDTKESNVKTFCSKNILAILGFSSIIAVIALLAVGLTQNKALPENVKYGIVL |
| NO: 1 | acid | DAGSSHTSLYIYKWPAEKENDTGVVHQVEECRVKGPGISKFVQKVNEIGI |
| | | YLTDCMERAREVIPRSQHQETPVYLGATAGMRLLRMESEELADRVLDVV |
| | | ERSLSNYPFDFQGARIITGQEEGAYGWITINYLLGKFSQKTRWFSIVPYET |
| | | NNQETFGALDLGGASTQVTFVPQNQTIESPDNALQFRLYGKDYNVYTHS |
| | | FLCYGKDQALWQKLAKDIQVASNEILRDPCFHPGYKKVVNVSDLYKTPCT |
| | | KRFEMTLPFQQFEIQGIGNYQQCHQSILELFNTSYCPYSQCAFNGIFLPPL |
| | | QGDFGAFSAFYFVMKFLNLTSEKVSQEKVTEMMKKFCAQPWEEIKTSYA |
| | | GVKEKYLSEYCFSGTYILSLLLQGYHFTADSWEHIHFIGKIQGSDAGWTLG |
| | | YMLNLTNMIPAEQPLSTPLSHSTYVFLMVLFSLVLFTVAIIGLLIFHKPSYFW |
| | | KDMV |
| Wild type | | |
| rat CD39 | | |
| SEQ ID | Amino | MEDIKDSKVKRFCSKNILIILGFSSVLAVIALIAVGLTHNKPLPENVKYGIVLD |
| NO: 2 | acid | AGSSHTNLYIYKWPAEKENDTGVVQLLEECQVKGPGISKYAQKTDEIAAY |
| | | LAECMKMSTERIPASKQHQTPVYLGATAGMRLLRMESKQSADEVLAAVS |
| | | RSLKSYPFDFQGAKIITGQEEGAYGWITINYLLGRFTQEQSWLNFISDSQK |
| | | QATFGALDLGGSSTQVTFVPLNQTLEAPETSLQFRLYGTDYTVYTHSFLC |
| | | YGKDQALWQKLAQDIQVSSGGILKDPCFYPGYKKVVNVSELYGTPCTKR |
| | | FEKKLPFNQFQVQGTGDYEQCHQSILKFFNNSHCPYSQCAFNGVFLPPL |
| | | QGSFGAFSAFYFVMDFFKKMANDSVSSQEKMTEITKNFCSKPWEEVKAS |
| | | YPTVKEKYLSEYCFSGTYILSLLLQGYNFTGTSWDQIHFMGKIKDSNAGW |
| | | TLGYMLNLTNMIPAEQPLSPPLPHSTYISLMVLFSLVLVAMVITGLFIFSKPS |
| | | YFWKEAV |
| CD39L3 | | |
| SEQ ID | Amino | QIHKQEVLPPGLKYGIVLDAGSSRTTVYVYQWPAEKENNTGVVSQTFKCS |
| NO: 3 | acid | VKGSGISSYGNNPQDVPRAFEECMQKVKGQVPSHLHGSTPIHLGATAGM |
| | | RLLRLQNETAANEVLESIQSYFKSQPFDFRGAQIISGQEEGVYGWITANYL |
| | | MGNFLEKNLWHMWVHPHGVETTGALDLGGASTQISFVAGEKMDLNTSDI |
| | | MQVSLYGYVYTLYTHSFQCYGRNEAEKKFLAMLLQNSPTKNHLTNPCYP |
| | | RDYSISFTMGHVFDSLCTVDQRPESYNPNDVITFEGTGDPSLCKEKVASIF |
| | | DFKACHDQETCSFDGVYQPKIKGPFVAFAGFYYTASALNLSGSFSLDTFN |
| | | SSTWNFCSQNWSQLPLLLPKFDEVYARSYCFSANYIYHLFVNGYKFTEET |
| | | WPQIHFEKEVGNSSIAWSLGYMLSLTNQIPAESPLIRLPIEP |
| EP28 | | |
| SEQ ID | Amino | TQNKALPENVKYGIVLDAGSSHTSLYIYKWPAEKENDTGVVHQVEECRVK |
| NO: 4 | acid | GPGISKFVQKVNEIGIYLTDCMERAREVIPRSQHQETPVYLGATAGMRLLR |
| | | MESEELADRVLDVVERSLSNYPFDFQGARIITGQEEGAYGWITINYLLGKF |
| | | SQKNQETFGALDLGGASTQVTFVPQNQTIESPDNALQFRLYGKDYNVYT |
| | | HSFLCYGKDQALWQKLAKDIQVASNEILRDPCFHPGYKKVVNVSDLYKTP |
| | | CTKRFEMTLPFQQFEIQGIGNYQQCHQSILELFNTSYCPYSQCAFNGIFLP |
| | | PLQGDFGAFSAFYFVMKFLNLTSEKVSQEKVTEMMKKFCAQPWEEIKTS |
| | | YAGVKEKYLSEYCFSGTYILSLLLQGYHFTADSWEHIHFIGKIQGSDAGWT |
| | | LGYMLNLTNMIPAEQPLSTPLSHST |
| SEQ ID | DNA | ACCCAGAACAAGGCCCTGCCCGAGAACGTGAAGTACGGCATCGTGCT |
| NO: 5 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCAGGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGCTGGACGTGGTGGAAAGAAGCCTGAGCAACTACCCATTCGAT |
| | | TTTCAAGGCGCCAGAATCATCACCGGCCAGGAAGAAGGCGCCTACGG |
| | | CTGGATCACCATCAACTACCTGCTGGGCAAGTTCAGCCAGAAGAATCA |
| | | GGAAACCTTCGGCGCCCTGGACCTGGGCGGAGCTTCTACCCAAGTGA |
| | | CCTTCGTGCCCCAGAATCAGACCATCGAGAGCCCCGACAACGCCCTG |
| | | CAGTTCCGGCTGTACGGCAAGGACTACAATGTGTACACCCACAGCTTT |
| | | CTGTGCTACGGAAAGGACCAGGCTCTGTGGCAGAAGCTGGCCAAGGA |
| | | CATCCAGGTGGCCAGCAACGAGATCCTGCGGGACCCTTGCTTCCACC |
| | | CCGGCTACAAGAAAGTCGTGAACGTGTCCGACCTGTACAAGACCCCC |
| | | TGCACCAAGAGATTCGAGATGACCCTGCCCTTCCAGCAGTTCGAGATC |
| | | CAGGGCATCGGCAATTACCAGCAGTGCCACCAGAGCATCCTGGAACT |
| | | GTTCAACACCAGCTACTGCCCCTACAGCCAGTGCGCCTTCAACGGCA |
| | | TCTTCCTGCCACCTCTGCAGGGGGATTTCGGCGCCTTCAGCGCCTTC |
| | | TACTTCGTGATGAAGTTCCTGAACCTGACCAGCGAGAAGGTGTCCCAG |
| | | GAAAAAGTGACAGAGATGATGAAGAAGTTCTGCGCCCAGCCCTGGGA |
| | | GGAAATCAAGACCTCCTACGCTGGCGTGAAAGAGAAGTACCTGAGCG |
| | | AGTACTGCTTCAGCGGCACCTACATCCTGAGCCTGCTGCTGCAGGGC |
| | | TACCACTTCACCGCCGATAGCTGGGAGCACATCCACTTCATCGGCAA |
| | | GATTCAGGGCAGCGACGCCGGCTGGACACTGGGCTACATGCTGAATC |
| | | TGACCAACATGATCCCCGCCGAGCAGCCCCTGAGCACACCTCTGAGC |
| | | CACAGCACC |
| EP1 | | |
| SEQ ID | Amino | TQNKALPENVKYGIVLDAGSSHTSLYIYKWPAEKENDTGVVHQVEECRVK |
| NO: 6 | acid | GPGISKFVQKVNEIGIYLTDCMERAMEVIPRSQHQETPVYLGATAGMRLL |
| | | RMESEELADRVLDVVERSLSNYPFDFQGARIITGQEEGAYGWITINYLLGK |
| | | FSQKNQETFGALDLGGASTQVTFVPQNQTIESPDNALQFRLYGKDYNVY |
| | | THSFLCYGKDQALWQKLAKDIQVASNEILRDPCFHPGYKKVVNVSDLYKT |
| | | PCTKRFEMTLPFQQFEIQGIGNYQQCHQSILELFNTSYCPYSQCAFNGIFL |
| | | PPLQGDFGAFSAFYFVMKFLNLTSEKVSQEKVTEMMKKFCAQPWEEIKT |
| | | SYAGVKEKYLSEYCFSGTYILSLLLQGYHFTADSWEHIHFIGKIQGSDAGW |
| | | TLGYMLNLTNMIPAEQPLSTPLSHST |
| SEQ ID | DNA | ACCCAGAACAAGGCCCTGCCCGAGAACGTGAAGTACGGCATCGTGCT |
| NO: 7 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCATGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGCTGGACGTGGTGGAAAGAAGCCTGAGCAACTACCCATTCGAT |
| | | TTTCAAGGCGCCAGAATCATCACCGGCCAGGAAGAAGGCGCCTACGG |
| | | CTGGATCACCATCAACTACCTGCTGGGCAAGTTCAGCCAGAAGAATCA |
| | | GGAAACCTTCGGCGCCCTGGACCTGGGCGGAGCTTCTACCCAAGTGA |
| | | CCTTCGTGCCCCAGAATCAGACCATCGAGAGCCCCGACAACGCCCTG |
| | | CAGTTCCGGCTGTACGGCAAGGACTACAATGTGTACACCCACAGCTTT |
| | | CTGTGCTACGGAAAGGACCAGGCTCTGTGGCAGAAGCTGGCCAAGGA |
| | | CATCCAGGTGGCCAGCAACGAGATCCTGCGGGACCCTTGCTTCCACC |
| | | CCGGCTACAAGAAAGTCGTGAACGTGTCCGACCTGTACAAGACCCCC |
| | | TGCACCAAGAGATTCGAGATGACCCTGCCCTTCCAGCAGTTCGAGATC |
| | | CAGGGCATCGGCAATTACCAGCAGTGCCACCAGAGCATCCTGGAACT |
| | | GTTCAACACCAGCTACTGCCCCTACAGCCAGTGCGCCTTCAACGGCA |
| | | TCTTCCTGCCACCTCTGCAGGGGGATTTCGGCGCCTTCAGCGCCTTC |
| | | TACTTCGTGATGAAGTTCCTGAACCTGACCAGCGAGAAGGTGTCCCAG |
| | | GAAAAAGTGACAGAGATGATGAAGAAGTTCTGCGCCCAGCCCTGGGA |
| | | GGAAATCAAGACCTCCTACGCTGGCGTGAAAGAGAAGTACCTGAGCG |
| | | AGTACTGCTTCAGCGGCACCTACATCCTGAGCCTGCTGCTGCAGGGC |
| | | TACCACTTCACCGCCGATAGCTGGGAGCACATCCACTTCATCGGCAA |
| | | GATTCAGGGCAGCGACGCCGGCTGGACACTGGGCTACATGCTGAATC |
| | | TGACCAACATGATCCCCGCCGAGCAGCCCCTGAGCACACCTCTGAGC |
| | | CACAGCACC |
| EP2 | | |
| SEQ ID | Amino | TQNKALPENVKYGIVLDAGSSHTSLYIYKWPAEKENDTGVVHQVEECRVK |
| NO: 8 | acid | GPGISKFVQKVNEIGIYLTDCMERAMEVIPRSQHQETPVYLGATAGMRLL |
| | | RMESEELADRVMDVVERSLSNYPFDFQGARIITGQEEGAYGWITINYLLG |
| | | KFSQKNQETFGALDLGGASTQVTFVPQNQTIESPDNALQFRLYGKDYNV |
| | | YTHSFLCYGKDQALWQKLAKDIQVASNEILRDPCFHPGYKKVVNVSDLYK |
| | | TPCTKRFEMTLPFQQFEIQGIGNYQQCHQSILELFNTSYCPYSQCAFNGIF |
| | | LPPLQGDFGAFSAFYFVMKFLNLTSEKVSQEKVTEMMKKFCAQPWEEIKT |
| | | SYAGVKEKYLSEYCFSGTYILSLLLQGYHFTADSWEHIHFIGKIQGSDAGW |
| | | TLGYMLNLTNMIPAEQPLSTPLSHST |
| SEQ ID | DNA | ACCCAGAACAAGGCCCTGCCCGAGAACGTGAAGTACGGCATCGTGCT |
| NO: 9 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCATGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGATGGACGTGGTGGAAAGAAGCCTGAGCAACTACCCATTCGAT |
| | | TTTCAAGGCGCCAGAATCATCACCGGCCAGGAAGAAGGCGCCTACGG |
| | | CTGGATCACCATCAACTACCTGCTGGGCAAGTTCAGCCAGAAGAATCA |
| | | GGAAACCTTCGGCGCCCTGGACCTGGGCGGAGCTTCTACCCAAGTGA |
| | | CCTTCGTGCCCCAGAATCAGACCATCGAGAGCCCCGACAACGCCCTG |
| | | CAGTTCCGGCTGTACGGCAAGGACTACAATGTGTACACCCACAGCTTT |
| | | CTGTGCTACGGAAAGGACCAGGCTCTGTGGCAGAAGCTGGCCAAGGA |
| | | CATCCAGGTGGCCAGCAACGAGATCCTGCGGGACCCTTGCTTCCACC |
| | | CCGGCTACAAGAAAGTCGTGAACGTGTCCGACCTGTACAAGACCCCC |
| | | TGCACCAAGAGATTCGAGATGACCCTGCCCTTCCAGCAGTTCGAGATC |
| | | CAAGGCATCGGCAATTACCAGCAGTGCCACCAGAGCATCCTGGAACT |
| | | GTTCAACACCAGCTACTGCCCCTACAGCCAGTGCGCCTTCAACGGCA |
| | | TCTTCCTGCCACCTCTGCAGGGGGATTTCGGCGCCTTCAGCGCCTTC |
| | | TACTTCGTGATGAAGTTCCTGAACCTGACCAGCGAGAAGGTGTCCCAG |
| | | GAAAAAGTGACAGAGATGATGAAGAAGTTCTGCGCCCAGCCCTGGGA |
| | | GGAAATCAAGACCTCCTACGCTGGCGTGAAAGAGAAGTACCTGAGCG |
| | | AGTACTGCTTCAGCGGCACCTACATCCTGAGCCTGCTGCTGCAGGGC |
| | | TACCACTTCACCGCCGATAGCTGGGAGCACATCCACTTCATCGGCAA |
| | | GATTCAGGGCAGCGACGCCGGCTGGACACTGGGCTACATGCTGAATC |
| | | TGACCAACATGATCCCCGCCGAGCAGCCCCTGAGCACACCTCTGAGC |
| | | CACAGCACC |
| EP3 | | |
| SEQ ID | Amino | TQNKALPENVKYGIVLDAGSSHTSLYIYKWPAEKENDTGVVHQVEECRVK |
| NO: 10 | acid | GPGISKFVQKVNEIGIYLTDCMERAMEVIPRSQHQETPVYLGATAGMRLL |
| | | RMESEELADRVLDVVERSLSNYPFDFQGARIITGQEEGAYGWITINYLLGK |
| | | FSQKNQETFGALDLGGASTQVTFVPQNQTIESPDNALQFRLYGKDYNVY |
| | | THSFLCYGKDQALWQKLAKDIQVASNEILRDPCFHPGYKKVVNVSDLYKT |
| | | PCTKGFEMTLPFQQFEIQGIGNYQQCHQSILELFNTSYCPYSQCAFNGIFL |
| | | PPLQGDFGAFSAFYFVMKFLNLTSEKVSQEKVTEMMKKFCAQPWEEIKT |
| | | SYAGVKEKYLSEYCFSGTYILSLLLQGYHFTADSWEHIHFIGKIQGSDAGW |
| | | TLGYMLNLTNMIPAEQPLRTPLSHST |
| SEQ ID | DNA | ACCCAGAACAAGGCCCTGCCCGAGAACGTGAAGTACGGCATCGTGCT |
| NO: 11 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCATGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGCTGGACGTGGTGGAAAGAAGCCTGAGCAACTACCCATTCGAT |
| | | TTTCAAGGCGCCAGAATCATCACCGGCCAGGAAGAAGGCGCCTACGG |
| | | CTGGATCACCATCAACTACCTGCTGGGCAAGTTCAGCCAGAAGAATCA |
| | | GGAAACCTTCGGCGCCCTGGACCTGGGCGGAGCTTCTACCCAAGTGA |
| | | CCTTCGTGCCCCAGAATCAGACCATCGAGAGCCCCGACAACGCCCTG |
| | | CAGTTCCGGCTGTACGGCAAGGACTACAATGTGTACACCCACAGCTTT |
| | | CTGTGCTACGGAAAGGACCAGGCTCTGTGGCAGAAGCTGGCCAAGGA |
| | | CATCCAGGTGGCCAGCAACGAGATCCTGCGGGACCCTTGCTTCCACC |
| | | CCGGCTACAAGAAAGTCGTGAACGTGTCCGACCTGTACAAGACCCCC |
| | | TGCACCAAGGGATTCGAGATGACCCTGCCCTTCCAGCAGTTCGAGAT |
| | | CCAGGGCATCGGCAATTACCAGCAGTGCCACCAGAGCATCCTGGAAC |
| | | TGTTCAACACCAGCTACTGCCCCTACAGCCAGTGCGCCTTCAACGGC |
| | | ATCTTCCTGCCACCTCTGCAGGGGGATTTCGGCGCCTTCAGCGCCTT |
| | | CTACTTCGTGATGAAGTTCCTGAACCTGACCAGCGAGAAGGTGTCCCA |
| | | GGAAAAAGTGACAGAGATGATGAAGAAGTTCTGCGCCCAGCCCTGGG |
| | | AGGAAATCAAGACCTCCTACGCTGGCGTGAAAGAGAAGTACCTGAGC |
| | | GAGTACTGCTTCAGCGGCACCTACATCCTGAGCCTGCTGCTGCAGGG |
| | | CTACCACTTCACCGCCGATAGCTGGGAGCACATCCACTTCATCGGCAA |
| | | GATTCAGGGCAGCGACGCCGGCTGGACACTGGGCTACATGCTGAATC |
| | | TGACCAACATGATCCCCGCCGAGCAGCCCCTGCGCACACCTCTGAGC |
| | | CACAGCACC |
| EP4 | | |
| SEQ ID | Amino | TQNKALPENVKYGIVLDAGSSHTSLYIYKWPAEKENDTGVVHQVEECRVK |
| NO: 12 | acid | GPGISKFAQKVNEIGIYLTDCMERAMEVIPRSQHQETPVYLGATAGMRLL |
| | | RMESEELADRVLDVVERSLSNYPFDFQGARIITGQEEGAYGWITINYLLGK |
| | | FSQKNQETFGALDLGGASTQVTFVPQNQTIESPDNALQFRLYGKDYNVY |
| | | THSFLCYGKDQALWQKLAKDIQVASNEILRDPCFHPGYKKVVNVSDLYKT |
| | | PCTKRFEMTLPFQQFEIQGIGNYQQCHQSILELFNTSYCPYSQCAFNGIFL |
| | | PPLQGDFGAFSAFYFVMKFLNLTSEKVSQEKVTEMMKKFCAQPWEEIKT |
| | | SYAGVKEKYLSEYCFSGTYILSLLLQGYHFTADSWEHIHFIGKIQGSDAGW |
| | | TLGYMLNLTNMIPAEQPLRTPLSHST |
| SEQ ID | DNA | ACCCAGAACAAGGCCCTGCCCGAGAACGTGAAGTACGGCATCGTGCT |
| NO: 13 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGCGCAGAAAGTGAACGA |
| | | GATCGGCATCTACCTGACCGACTGCATGGAACGGGCCATGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGCTGGACGTGGTGGAAAGAAGCCTGAGCAACTACCCATTCGAT |
| | | TTTCAAGGCGCCAGAATCATCACCGGCCAGGAAGAAGGCGCCTACGG |
| | | CTGGATCACCATCAACTACCTGCTGGGCAAGTTCAGCCAGAAGAATCA |
| | | GGAAACCTTCGGCGCCCTGGACCTGGGCGGAGCTTCTACCCAAGTGA |
| | | CCTTCGTGCCCCAGAATCAGACCATCGAGAGCCCCGACAACGCCCTG |
| | | CAGTTCCGGCTGTACGGCAAGGACTACAATGTGTACACCCACAGCTTT |
| | | CTGTGCTACGGAAAGGACCAGGCTCTGTGGCAGAAGCTGGCCAAGGA |
| | | CATCCAGGTGGCCAGCAACGAGATCCTGCGGGACCCTTGCTTCCACC |
| | | CCGGCTACAAGAAAGTCGTGAACGTGTCCGACCTGTACAAGACCCCC |
| | | TGCACCAAGAGATTCGAGATGACCCTGCCCTTCCAGCAGTTCGAGATC |
| | | CAGGGCATCGGCAATTACCAGCAGTGCCACCAGAGCATCCTGGAACT |
| | | GTTCAACACCAGCTACTGCCCCTACAGCCAGTGCGCCTTCAACGGCA |
| | | TCTTCCTGCCACCTCTGCAGGGGGATTTCGGCGCCTTCAGCGCCTTC |
| | | TACTTCGTGATGAAGTTCCTGAACCTGACCAGCGAGAAGGTGTCCCAG |
| | | GAAAAAGTGACAGAGATGATGAAGAAGTTCTGCGCCCAGCCCTGGGA |
| | | GGAAATCAAGACCTCCTACGCTGGCGTGAAAGAGAAGTACCTGAGCG |
| | | AGTACTGCTTCAGCGGCACCTACATCCTGAGCCTGCTGCTGCAGGGC |
| | | TACCACTTCACCGCCGATAGCTGGGAGCACATCCACTTCATCGGCAA |
| | | GATTCAGGGCAGCGACGCCGGCTGGACACTGGGCTACATGCTGAATC |
| | | TGACCAACATGATCCCCGCCGAGCAGCCCCTGAGGACACCTCTGAGC |
| | | CACAGCACC |
| EP5 | | |
| SEQ ID | Amino | TQNKALPENVKYGIVLDAGSSHTSLYIYKWPAEKENDTGVVHQVEECRVK |
| NO: 14 | acid | GPGISKFVQKVNEIGIYLTDCMERAREVIPRSQHQETPVYLGATAGMRLLR |
| | | MESEELADRVMDVVERSLSNYPFDFQGARIITGQEEGAYGWITINYLLGK |
| | | FSQKNQETFGALDLGGASTQVTFVPQNQTIESPDNALQFRLYGKDYNVY |
| | | THSFLCYGKDQALWQKLAKDIQVASNEILRDPCFHPGYKKVVNVSDLYKT |
| | | PCTKRFEMTLPFQQFEIQGIGNYQQCHQSILELFNTSYCPYSQCAFNGIFL |
| | | PPLQGDFGAFSAFYFVMKFLNLTSEKVSQEKVTEMMKKFCAQPWEEIKT |
| | | SYAGVKEKYLSEYCFSGTYILSLLLQGYHFTADSWEHIHFIDKIQGSDAGW |
| | | TLGYMLNLTNMIPAEQPLSTPLSHST |
| SEQ ID | DNA | ACCCAGAACAAGGCCCTGCCCGAGAACGTGAAGTACGGCATCGTGCT |
| NO: 15 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCAGGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGATGGACGTGGTGGAAAGAAGCCTGAGCAACTACCCATTCGAT |
| | | TTTCAAGGCGCCAGAATCATCACCGGCCAGGAAGAAGGCGCCTACGG |
| | | CTGGATCACCATCAACTACCTGCTGGGCAAGTTCAGCCAGAAGAATCA |
| | | GGAAACCTTCGGCGCCCTGGACCTGGGCGGAGCTTCTACCCAAGTGA |
| | | CCTTCGTGCCCCAGAATCAGACCATCGAGAGCCCCGACAACGCCCTG |
| | | CAGTTCCGGCTGTACGGCAAGGACTACAATGTGTACACCCACAGCTTT |
| | | CTGTGCTACGGAAAGGACCAGGCTCTGTGGCAGAAGCTGGCCAAGGA |
| | | CATCCAGGTGGCCAGCAACGAGATCCTGCGGGACCCTTGCTTCCACC |
| | | CCGGCTACAAGAAAGTCGTGAACGTGTCCGACCTGTACAAGACCCCC |
| | | TGCACCAAGAGATTCGAGATGACCCTGCCCTTCCAGCAGTTCGAGATC |
| | | CAGGGCATCGGCAATTACCAGCAGTGCCACCAGAGCATCCTGGAACT |
| | | GTTCAACACCAGCTACTGCCCCTACAGCCAGTGCGCCTTCAACGGCA |
| | | TCTTCCTGCCACCTCTGCAGGGGGATTTCGGCGCCTTCAGCGCCTTC |
| | | TACTTCGTGATGAAGTTCCTGAACCTGACCAGCGAGAAGGTGTCCCAG |
| | | GAAAAAGTGACAGAGATGATGAAGAAGTTCTGCGCCCAGCCCTGGGA |
| | | GGAAATCAAGACCTCCTACGCTGGCGTGAAAGAGAAGTACCTGAGCG |
| | | AGTACTGCTTCAGCGGCACCTACATCCTGAGCCTGCTGCTGCAGGGC |
| | | TACCACTTCACCGCCGATAGCTGGGAGCACATCCACTTCATCGACAAG |
| | | ATTCAGGGCAGCGACGCCGGCTGGACACTGGGCTACATGCTGAATCT |
| | | GACCAACATGATCCCCGCCGAGCAGCCCCTGAGCACACCTCTGAGCC |
| | | ACAGCACC |
| EP6 | | |
| SEQ ID | Amino | TQNKALPENVKYGIVLDAGSSHTSLYIYKWPAEKENDTGVVHQVEECRVK |
| NO: 16 | acid | GPGISKFVQKVNEIGISLTDCMERAREVIPRSQHQETPVYLGATAGMRLLR |
| | | MESEELADRVMDVVERSLSNYPFDFQGARIITGQEEGAYGWITINYLLGK |
| | | FSQKNQETFGALDLGGASTQVTFVPQNQTIESPDNALQFRLYGKDYNVY |
| | | THSFLCYGKDQALRQKLAKDIQVASNEILRDPCFHPGYKKVVNVSDLYKT |
| | | PCTKRFEMTLPFQQFEIQGIGNYQQCHQSILELFNTSYCPYSQCAFNGIFL |
| | | PPLQGDFGAFSAFYFVMKFLNLTSEKVSQEKVTEMMKKFCAQPWEEIKT |
| | | SYAGVKEKYLSEYCFSGTYILSLLLQGYHFTADSWEHIHFIGKIQGSDAGW |
| | | TLGYMLNLTNMIPAEQPLSTPLSHST |
| SEQ ID | DNA | ACCCAGAACAAGGCCCTGCCCGAGAACGTGAAGTACGGCATCGTGCT |
| NO: 17 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTCCCTGACCGACTGCATGGAACGGGCCAGGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGATGGACGTGGTGGAAAGAAGCCTGAGCAACTACCCATTCGAT |
| | | TTTCAAGGCGCCAGAATCATCACCGGCCAGGAAGAAGGCGCCTACGG |
| | | CTGGATCACCATCAACTACCTGCTGGGCAAGTTCAGCCAGAAGAATCA |
| | | GGAAACCTTCGGCGCCCTGGACCTGGGCGGAGCTTCTACCCAAGTGA |
| | | CCTTCGTGCCCCAGAATCAGACCATCGAGAGCCCCGACAACGCCCTG |
| | | CAGTTCCGGCTGTACGGCAAGGACTACAATGTGTACACCCACAGCTTT |
| | | CTGTGCTACGGAAAGGACCAGGCTCTGCGGCAGAAGCTGGCCAAGG |
| | | ACATCCAGGTGGCCAGCAACGAGATCCTGCGGGACCCTTGCTTCCAC |
| | | CCCGGCTACAAGAAAGTCGTGAACGTGTCCGACCTGTACAAGACCCC |
| | | CTGCACCAAGAGATTCGAGATGACCCTGCCCTTCCAGCAGTTCGAGAT |
| | | CCAGGGCATCGGCAATTACCAGCAGTGCCACCAGAGCATCCTGGAAC |
| | | TGTTCAACACCAGCTACTGCCCCTACAGCCAGTGCGCCTTCAACGGC |
| | | ATCTTCCTGCCACCTCTGCAGGGGGATTTCGGCGCCTTCAGCGCCTT |
| | | CTACTTCGTGATGAAGTTCCTGAACCTGACCAGCGAGAAGGTGTCCCA |
| | | GGAAAAAGTGACAGAGATGATGAAGAAGTTCTGCGCCCAGCCCTGGG |
| | | AGGAAATCAAGACCTCCTACGCTGGCGTGAAAGAGAAGTACCTGAGC |
| | | GAGTACTGCTTCAGCGGCACCTACATCCTGAGCCTGCTGCTGCAGGG |
| | | CTACCACTTCACCGCCGATAGCTGGGAGCACATCCACTTCATCGGCAA |
| | | GATTCAGGGCAGCGACGCCGGCTGGACACTGGGCTACATGCTGAATC |
| | | TGACCAACATGATCCCCGCCGAGCAGCCCCTGAGCACACCTCTGAGC |
| | | CACAGCACC |
| EP7 | | |
| SEQ ID | Amino | TQNKALPENVKYGIVLDAGSSHTSLYIYKWPAEEENDTGVVHQVEECRVK |
| NO: 18 | acid | GPGISKFVQKVNEIGIYLSDCMERAREVIPRSQHQETPVYLGATAGMRLL |
| | | RMESEELADRVLDAVERSLSNYPFDFQGARIITGQEEGAYGWITINYLLGK |
| | | FSQKNQETFGALDLGGASTQVTFVPQNQTIESPDNALQFRLYGKDYNVY |
| | | THSFLCYGKDQALWQKLAKDIQVASNEILRDPCFHPGYKKVVNVSDLYKT |
| | | PCTKRFEMTLPFQQFEIQGTGNYQQCHQSILELFNTSYPYSQCAFNGIF |
| | | LPPLQGDFGAFSAFYFVMKFLNLTSEKVSQEKVTEMMKKFCAQPWEEIKT |
| | | SYAGVKEKYLSEYCFSGTYILSLLLQGYHFTADSWEHIHFIGKIQGSDAGW |
| | | TLGYMLNLTNMIPAEQPLSTPLSHST |
| SEQ ID | DNA | ACCCAGAACAAGGCCCTGCCCGAGAACGTGAAGTACGGCATCGTGCT |
| NO: 19 | | GGATGCCGGCAGCAGCCACACCAGCCTGTACATCTACAAGTGGCCTG |
| | | CCGAGGAAGAAAACGACACCGGCGTGGTGCATCAGGTGGAAGAGTG |
| | | CAGAGTGAAGGGCCCTGGCATCAGCAAGTTCGTGCAGAAAGTGAACG |
| | | AGATCGGCATCTACCTGTCCGACTGCATGGAACGGGCCAGGGAAGTG |
| | | ATCCCCAGAAGCCAGCACCAGGAAACCCCCGTGTATCTGGGAGCCAC |
| | | CGCCGGCATGAGACTGCTGAGAATGGAAAGCGAGGAACTGGCCGAC |
| | | CGGGTGCTGGACGCGGTGGAAAGAAGCCTGAGCAACTACCCATTCGA |
| | | TTTTCAAGGCGCCAGAATCATCACCGGCCAGGAAGAAGGCGCCTACG |
| | | GCTGGATCACCATCAACTACCTGCTGGGCAAGTTCAGCCAGAAGAATC |
| | | AGGAAACCTTCGGCGCCCTGGACCTGGGCGGAGCTTCTACCCAAGTG |
| | | ACCTTCGTGCCCCAGAATCAGACCATCGAGAGCCCCGACAACGCCCT |
| | | GCAGTTCCGGCTGTACGGCAAGGACTACAATGTGTACACCCACAGCT |
| | | TTCTGTGCTACGGAAAGGACCAGGCTCTGTGGCAGAAGCTGGCCAAG |
| | | GACATCCAGGTGGCCAGCAACGAGATCCTGCGGGACCCTTGCTTCCA |
| | | CCCCGGCTACAAGAAAGTCGTGAACGTGTCCGACCTGTACAAGACCC |
| | | CCTGCACCAAGAGATTCGAGATGACCCTGCCCTTCCAGCAGTTCGAG |
| | | ATCCAGGGCACCGGCAATTACCAGCAGTGCCACCAGAGCATCCTGGA |
| | | ACTGTTCAACACCAGCTACTGCCCCTACAGCCAGTGCGCCTTCAACG |
| | | GCATCTTCCTGCCACCTCTGCAGGGGGATTTCGGCGCCTTCAGCGCC |
| | | TTCTACTTCGTGATGAAGTTCCTGAACCTGACCAGCGAGAAGGTGTCC |
| | | CAGGAAAAAGTGACAGAGATGATGAAGAAGTTCTGCGCCCAGCCCTG |
| | | GGAGGAAATCAAGACCTCCTACGCTGGCGTGAAAGAGAAGTACCTGA |
| | | GCGAGTACTGCTTCAGCGGCACCTACATCCTGAGCCTGCTGCTGCAG |
| | | GGCTACCACTTCACCGCCGATAGCTGGGAGCACATCCACTTCATCGG |
| | | CAAGATTCAGGGCAGCGACGCCGGCTGGACACTGGGCTACATGCTGA |
| | | ATCTGACCAACATGATCCCCGCCGAGCAGCCCCTGAGCACACCTCTG |
| | | AGCCACAGCACC |
| EP8 | | |
| SEQ ID | Amino | TQNKALPENVKYGIVLDAGSSHTSLYIYKWPAEKENDTGVVHQVEECRVK |
| NO: 20 | acid | GPGISKFVQKVNEIGIYLTDCMERAREVIPRSQHQETPVYLGATAGMRLLR |
| | | MESEELADRVLDAVERSLSNYPFDFQGARIITGQEEGAYGWITINYLLGKF |
| | | SQKNQETFGALDLGGASTQVTFVPQNQTIESPDNALQFRLYGKDYNVYT |
| | | HSFLCYGKDQALWQKLAKDIQVASNEILRDPCFHPGYKKVVNVSDLYKTP |
| | | CTKRFEMTLPFQQFEIQGIGNYQQCHQSILELFNTSYCPYSQCAFNGIFLP |
| | | PLQGDFGAFSAFYFVMKFLNLTSEKVSQEKVTEMMKKFCAQPWEEIKTS |
| | | YAGVKEKYLSEYCFSGTYILSLLLQGYHFTADSWEHIHFIGKIQGSDAGVVT |
| | | LGYMLNLTNMIPAEQPLSTPLSHST |
| SEQ ID | DNA | ACCCAGAACAAGGCCCTGCCCGAGAACGTGAAGTACGGCATCGTGCT |
| NO: 21 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCAGGGAAGTGA |
| | | TTCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGCTGGACGCGGTGGAAAGAAGCCTGAGCAACTACCCATTCGAT |
| | | TTTCAAGGCGCCAGAATCATCACCGGCCAGGAAGAAGGCGCCTACGG |
| | | CTGGATCACCATCAACTACCTGCTGGGTAAGTTCAGCCAGAAGAATCA |
| | | GGAAACCTTCGGCGCCCTGGACCTGGGCGGAGCTTCTACCCAAGTGA |
| | | CCTTCGTGCCCCAGAATCAGACCATCGAGAGCCCCGACAACGCCCTG |
| | | CAGTTCCGGCTGTACGGCAAGGACTACAATGTGTACACCCACAGCTTT |
| | | CTGTGCTACGGAAAGGACCAGGCTCTGTGGCAGAAGCTGGCCAAGGA |
| | | CATCCAGGTGGCCAGCAACGAGATCCTGCGGGACCCTTGCTTCCACC |
| | | CCGGCTACAAGAAAGTAGTGAACGTGTCCGACCTGTACAAGACCCCC |
| | | TGCACCAAGAGATTCGAGATGACCCTGCCATTCCAGCAGTTCGAGATC |
| | | CAGGGCATCGGCAATTACCAGCAGTGCCACCAGAGCATCCTGGAACT |
| | | GTTCAACACCAGCTACTGCCCCTACAGCCAGTGCGCCTTCAACGGCA |
| | | TCTTCCTGCCACCTCTGCAGGGGGATTTCGGCGCCTTCAGCGCCTTC |
| | | TACTTCGTGATGAAGTTCCTGAACCTGACCAGCGAGAAGGTGTCCCAG |
| | | GAAAAAGTGACAGAGATGATGAAGAAGTTCTGCGCCCAGCCCTGGGA |
| | | GGAAATCAAGACCTCCTACGCTGGCGTGAAAGAGAAGTACCTGAGCG |
| | | AGTACTGCTTCAGCGGCACCTACATCCTGAGCCTGCTGCTGCAGGGC |
| | | TACCACTTCACCGCCGATAGCTGGGAGCACATCCACTTCATCGGCAA |
| | | GATTCAGGGCAGCGACGCCGGCTGGACACTGGGCTACATGCTGAATC |
| | | TGACCAACATGATCCCCGCCGAGCAGCCCCTGAGCACACCTCTGAGC |
| | | CACAGCACC |
| EP9 | | |
| SEQ ID | Amino | TQNKALPENVKYGIVLDAGSSHTSLYIYKWPAEKENDTGVVHQVEECRVK |
| NO: 22 | acid | GPGISKFVQKVNEIGIYLTDCMERAREVIPRSQHQETPVYLGATAGMRLLR |
| | | MESEELADRVLDVVERSLSNYPFDFQGARIITGQEEGAYGWITINYLLGKF |
| | | SQKNQETFGALDLGGASTQVTFVPQNQTIESPDNALQFRLYGKDYNVYT |
| | | HSFLCYGKDQALRQKLAKDIQVASNEILRDPCFHPGYKKVVNVSDLYKTP |
| | | CTKRFEMTLPFQQFEIQGIGNYQQCHQSILELFNTSYCPYSQCAFNGIFLP |
| | | PLQGDFGAFSAFYFVMKFLNLTSEKVSQEKVTEMMKKFCAQPWEEIKTS |
| | | YAGVKEKYLSEYCFSGTYILSLLLQGYHFTADSWEHIHFIGKIQGSDAGVVT |
| | | LGYMLNLTNMIPAEQPLSTPLSHST |
| SEQ ID | DNA | ACCCAGAACAAGGCCCTGCCCGAGAACGTGAAGTACGGCATCGTGCT |
| NO: 23 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCAGGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGCTGGACGTGGTGGAAAGAAGCCTGAGCAACTACCCATTCGAT |
| | | TTTCAAGGCGCCAGAATCATCACCGGCCAGGAAGAAGGCGCCTACGG |
| | | CTGGATCACCATCAACTACCTGCTGGGCAAGTTCAGCCAGAAGAATCA |
| | | GGAAACCTTCGGCGCCCTGGACCTGGGCGGAGCTTCTACCCAAGTGA |
| | | CCTTCGTGCCCCAGAATCAGACCATCGAGAGCCCCGACAACGCCCTG |
| | | CAGTTCCGGCTGTACGGCAAGGACTACAATGTGTACACCCACAGCTTT |
| | | CTGTGCTACGGAAAGGACCAGGCTCTGAGGCAGAAGCTGGCCAAGGA |
| | | CATCCAGGTGGCCAGCAACGAGATCCTGCGGGACCCTTGCTTCCACC |
| | | CCGGCTACAAGAAAGTCGTGAACGTGTCCGACCTGTACAAGACCCCC |
| | | TGCACCAAGAGATTCGAGATGACCCTGCCCTTCCAGCAGTTCGAGATC |
| | | CAGGGCATCGGCAATTACCAGCAGTGCCACCAGAGCATCCTGGAACT |
| | | GTTCAACACCAGCTACTGCCCCTACAGCCAGTGCGCCTTCAACGGCA |
| | | TCTTCCTGCCACCTCTGCAGGGGGATTTCGGCGCCTTCAGCGCCTTC |
| | | TACTTCGTGATGAAGTTCCTGAACCTGACCAGCGAGAAGGTGTCCCAG |
| | | GAAAAAGTGACAGAGATGATGAAGAAGTTCTGCGCCCAGCCCTGGGA |
| | | GGAAATCAAGACCTCCTACGCTGGCGTGAAAGAGAAGTACCTGAGCG |
| | | AGTACTGCTTCAGCGGCACCTACATCCTGAGCCTGCTGCTGCAGGGC |
| | | TACCACTTCACCGCCGATAGCTGGGAGCACATCCACTTCATCGGCAA |
| | | GATTCAGGGCAGCGACGCCGGCTGGACACTGGGCTACATGCTGAATC |
| | | TGACCAACATGATCCCCGCCGAGCAGCCCCTGAGCACACCTCTGAGC |
| | | CACAGCACC |
| EP10 | | |
| SEQ ID | Amino | TQNKALPENVKYGIVLDAGSSHTSLYIYKWPAEKENDTGVVHQVEECRVK |
| NO: 24 | acid | GPGISKFVQKVNEIGIYLTDCMERAREVIPRSQHQETPVYLGATAGMRLLR |
| | | MESEELADRVLDVVERSLSNYPFDFQGARIITGQEEGAYGWITINYLLGKF |
| | | SQKNQETFGALDLGGASTQVTFVPQNQTIESPDNALQFRLYGKDYNVYT |
| | | HSFLCYGKDQALWQKLAKDIQVASNEILRDPCFHPGYKKVVNVSDLYKTP |
| | | CTKRFEMTLPFQQFEIQGTGNYQQCHQSILELFNTSYCPYSQCAFNGIFL |
| | | PPLQGDFGAFSAFYFVMKFLNLTSEKVSQEKVTEMMKKFCAQPWEEIKT |
| | | SYAGVKEKYLSEYCFSGTYILSLLLQGYHFTADSWEHIHFIGKIQGSDAGW |
| | | TLGYMLNLTNMIPAEQPLSTPLSHST |
| SEQ ID | DNA | ACCCAGAACAAGGCCCTGCCCGAGAACGTGAAGTACGGCATCGTGCT |
| NO: 25 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCAGGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGCTGGACGTGGTGGAAAGAAGCCTGAGCAACTACCCATTCGAT |
| | | TTTCAAGGCGCCAGAATCATCACCGGCCAGGAAGAAGGCGCCTACGG |
| | | CTGGATCACCATCAACTACCTGCTGGGCAAGTTCAGCCAGAAGAATCA |
| | | GGAAACCTTCGGCGCCCTGGACCTGGGCGGAGCTTCTACCCAAGTGA |
| | | CCTTCGTGCCCCAGAATCAGACCATCGAGAGCCCCGACAACGCCCTG |
| | | CAGTTCCGGCTGTACGGCAAGGACTACAATGTGTACACCCACAGCTTT |
| | | CTGTGCTATGGAAAGGACCAGGCTCTGTGGCAGAAGCTGGCCAAGGA |
| | | CATCCAGGTGGCCAGCAACGAGATCCTGCGGGACCCTTGCTTCCACC |
| | | CCGGCTACAAGAAAGTCGTGAACGTGTCCGACCTGTACAAGACCCCC |
| | | GCACCAAGAGATTCGAGATGACCCTGCCCTTCCAGCAGTTCGAGATC |
| | | CAGGGCACCGGCAATTACCAGCAGTGCCACCAGAGCATCCTGGAACT |
| | | GTTCAACACCAGCTACTGCCCCTACAGCCAGTGCGCCTTCAACGGCA |
| | | TCTTCCTGCCACCTCTGCAGGGGGATTTCGGCGCCTTCAGCGCCTTC |
| | | TACTTCGTGATGAAGTTCCTGAACCTGACCAGCGAGAAGGTGTCCCAG |
| | | GAAAAAGTGACAGAGATGATGAAGAAGTTCTGCGCCCAGCCCTGGGA |
| | | GGAAATCAAGACCTCCTACGCTGGCGTGAAAGAGAAGTACCTGAGCG |
| | | AGTACTGCTTCAGCGGCACCTACATCCTGAGCCTGCTGCTGCAGGGC |
| | | TACCACTTCACCGCCGATAGCTGGGAGCACATCCACTTCATCGGCAA |
| | | GATTCAGGGCAGCGACGCCGGCTGGACACTGGGCTACATGCTGAATC |
| | | TGACCAACATGATCCCCGCCGAGCAGCCTTTGAGCACACCTCTGAGC |
| | | CACAGCACC |
| EP11 | | |
| SEQ ID | Amino | TQNKALPENVKYGIVLDAGSSHTSLYIYKWPAEKENDTGVVHQVEECRVK |
| NO: 26 | acid | GPGISKFVQKVNEIGIYLTDCMERAMEVIPRSQHQETPVYLGATAGMRLL |
| | | RMESEELADRVLDVVERSLSNYPFDFQGARIITGQEEGAYGWITINYLLGK |
| | | FSQKNQETFGALDLGGASTQVTFVPQNQTIESPDNALQFRLYGKDYNVY |
| | | THSFLCYGKDQALWQKLAKDIQVASNEILRDPCFHPGYKKVVNVSDLYKT |
| | | PCTKRFEMTLPFQQFEIQGTGNYQQCHQSILELFNTSYCPYSQCAFNGIF |
| | | LPPLQGDFGAFSAFYFVMKFLNLTSEKVSQEKVTEMMKKFCAQPWEEIKT |
| | | SYAGVKEKYLSEYCFSGTYILSLLLQGYHFTADSWEHIHFIGKIQGSDAGW |
| | | TLGYMLNLTNMIPAEQPLSTPLSHST |
| SEQ ID | DNA | ACCCAGAACAAGGCCCTGCCCGAGAACGTGAAGTACGGCATCGTGCT |
| NO: 27 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCATGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGCTGGACGTGGTGGAAAGAAGCCTGAGCAACTACCCATTCGAT |
| | | TTTCAAGGCGCCAGAATCATCACCGGCCAGGAAGAAGGCGCCTACGG |
| | | CTGGATCACCATCAACTACCTGCTGGGCAAGTTCAGCCAGAAGAATCA |
| | | GGAAACCTTCGGCGCCCTGGACCTGGGCGGAGCTTCTACCCAAGTGA |
| | | CCTTCGTGCCCCAGAATCAGACCATCGAGAGCCCCGACAACGCCCTG |
| | | CAGTTCCGGCTGTACGGCAAGGACTACAATGTGTACACCCACAGCTTT |
| | | CTGTGCTACGGAAAGGACCAGGCTCTGTGGCAGAAGCTGGCCAAGGA |
| | | CATCCAGGTGGCCAGCAACGAGATCCTGCGGGACCCTTGCTTCCACC |
| | | CCGGCTACAAGAAAGTCGTGAACGTGTCCGACCTGTACAAGACCCCC |
| | | TGCACCAAGAGATTCGAGATGACCCTGCCCTTCCAGCAGTTCGAGATC |
| | | CAGGGCACCGGCAATTACCAGCAGTGCCACCAGAGCATCCTGGAACT |
| | | GTTCAACACCAGCTACTGCCCCTACAGCCAGTGCGCCTTCAACGGCA |
| | | TCTTCCTGCCACCTCTGCAGGGGGATTTCGGCGCCTTCAGCGCCTTC |
| | | TACTTCGTGATGAAGTTCCTGAACCTGACTAGCGAGAAGGTGTCCCAG |
| | | GAAAAAGTGACAGAGATGATGAAGAAGTTCTGCGCCCAGCCCTGGGA |
| | | GGAAATCAAGACCTCCTACGCTGGCGTGAAAGAGAAGTACCTGAGCG |
| | | AGTACTGCTTCAGCGGCACCTACATCCTGAGCCTGCTGCTGCAGGGC |
| | | TACCACTTCACCGCCGATAGCTGGGAGCACATCCACTTCATCGGCAA |
| | | GATTCAGGGCAGCGACGCCGGCTGGACACTGGGCTACATGCTGAATC |
| | | TGACCAACATGATCCCCGCCGAGCAGCCCCTGAGCACACCTCTGAGC |
| | | CACAGCACC |
| EP12 | | |
| SEQ ID | Amino | TQNKALPENVKYGIVLDAGSSHTSLYIYKWPAEKENDTGVVHQVEECRVK |
| NO: 28 | acid | GPGISKFVQKVNEIGIYLTDCMERAREVIPRSQHQETPVYLGATAGMRLLR |
| | | MESEELADRVLDVVERSLSNYPFDFQGARIITGQEEGAYGWITINYLLGKF |
| | | SQKNQETFGALDLGGASTQVTFVPQNQTIESPDNALQFRLYGKDYNVYT |
| | | HSFLCYGKDQALWQKLAKDIQVASNDILRDPCFHPGYKKVVNVSDLYKTP |
| | | CTKRFEMTLPFQQFEIQGTGNYQQCHQSILELFNTSYCPYSQCAFNGIFL |
| | | PPLQGDFGAFSAFYFVMKFLNLTSEKVSQEKVTEMMKKFCAQPWEEIKT |
| | | SYAGVKEKYLSEYCFSGTYILSLLLQGYHFTADSWEHIHFIGKIQGSDAGW |
| | | TLGYMLNLTNMIPAEQPLSTPLSHST |
| SEQ ID | DNA | ACCCAGAACAAGGCCCTGCCCGAGAACGTGAAGTACGGCATCGTGCT |
| NO: 29 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCAGGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGCTGGACGTGGTGGAAAGAAGCCTGAGCAACTACCCATTCGAT |
| | | TTTCAAGGCGCCAGAATCATCACCGGCCAGGAAGAAGGCGCCTACGG |
| | | CTGGATCACCATCAACTACCTGCTGGGCAAGTTCAGCCAGAAGAATCA |
| | | GGAAACCTTCGGCGCCCTGGACCTGGGCGGAGCTTCTACCCAAGTGA |
| | | CCTTCGTGCCCCAGAATCAGACCATCGAGAGCCCCGACAACGCCCTG |
| | | CAGTTCCGGCTGTACGGCAAGGACTACAATGTGTACACCCACAGCTTT |
| | | CTGTGCTACGGAAAGGACCAGGCTCTGTGGCAGAAGCTGGCCAAGGA |
| | | CATCCAGGTGGCCAGCAACGATATCCTGCGGGACCCTTGCTTCCACC |
| | | CCGGCTACAAGAAAGTCGTGAACGTGTCCGACCTGTACAAGACCCCC |
| | | TGCACCAAGAGATTCGAGATGACCCTGCCCTTCCAGCAGTTCGAGATC |
| | | CAGGGCACCGGCAATTACCAGCAGTGCCACCAGAGCATCCTGGAACT |
| | | GTTCAACACCAGCTACTGCCCCTACAGCCAGTGCGCCTTCAACGGCA |
| | | TCTTCCTGCCACCTCTGCAGGGGGATTTCGGCGCCTTCAGCGCCTTC |
| | | TACTTCGTGATGAAGTTCCTGAACCTGACCAGCGAGAAGGTGTCCCAG |
| | | GAAAAAGTGACAGAGATGATGAAGAAGTTCTGCGCCCAGCCCTGGGA |
| | | GGAAATCAAGACCTCCTACGCTGGCGTGAAAGAGAAGTACCTGAGCG |
| | | AGTACTGCTTCAGCGGCACCTACATCCTGAGCCTGCTGCTGCAGGGC |
| | | TACCACTTCACCGCCGATAGCTGGGAGCACATCCACTTCATCGGCAA |
| | | GATTCAGGGCAGCGACGCCGGCTGGACACTGGGCTACATGCTGAATC |
| | | TGACCAACATGATCCCCGCCGAGCAGCCCCTGAGCACACCTCTGAGC |
| | | CACAGCACC |
| EP13 | | |
| SEQ ID | Amino | TQNKALPENVKYGIVLDAGSSHTSLYIYKWPAEKENDTGVVHQVEECRVK |
| NO: 30 | acid | GPGISKFVQKVNEIGIYLTDCMERAREVIPRSQHQETPVYLGATAGMRLLR |
| | | MESEELADRVLDVVERSLSNYPFDFQGARIITGQEEGAYGWITINYLLGKF |
| | | SQKNQETFGALDLGGASTQVTFVPQNQTIESPDNALQFRLYGKDYNVYT |
| | | HSFLCYGKDQALWQKLAKDIQVASNEILRDPCFHPGYKKVVNVSDLYKTP |
| | | CTKRFEMTLPFQQFEIQGIGNYQQCHQSILELFNTSYCPYSQCAFNGIFLP |
| | | PLQGDFGAFSAFYSVMKFLNLTSEKVSQEKVTEMMKKFCAQPWEEIKTS |
| | | YAGVKEKYLSEYCFSGTYILSLLPQGYHFTADSWEHIHFIGKIQGSDAGWT |
| | | LGYMLNLTNMIPAEQPLSTPLSHST |
| SEQ ID | DNA | ACCCAGAACAAGGCCCTGCCCGAGAACGTGAAGTACGGCATCGTGCT |
| NO: 31 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCAGGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGCTGGACGTGGTGGAAAGAAGCCTGAGCAACTACCCATTCGAT |
| | | TTTCAAGGCGCCAGAATCATCACCGGCCAGGAAGAAGGCGCCTACGG |
| | | CTGGATCACCATCAACTACCTGCTGGGCAAGTTCAGCCAGAAGAATCA |
| | | GGAAACCTTCGGCGCCCTGGACCTGGGCGGAGCTTCTACCCAAGTGA |
| | | CCTTCGTGCCCCAGAATCAGACCATCGAGAGCCCCGACAACGCCCTG |
| | | CAGTTCCGGCTGTACGGCAAGGACTACAATGTGTACACCCACAGCTTT |
| | | CTGTGCTACGGAAAGGACCAGGCTCTGTGGCAGAAGCTGGCCAAGGA |
| | | CATCCAGGTGGCCAGCAACGAGATCCTGCGGGACCCTTGCTTCCACC |
| | | CCGGCTACAAGAAAGTCGTGAACGTGTCCGACCTGTACAAGACCCCC |
| | | TGCACCAAGAGATTCGAGATGACCCTGCCCTTCCAGCAGTTCGAGATC |
| | | CAGGGCATCGGCAATTACCAGCAGTGCCACCAGAGCATCCTGGAACT |
| | | GTTCAACACCAGCTACTGCCCCTACAGCCAGTGCGCCTTCAACGGCA |
| | | TCTTCCTGCCACCTCTGCAGGGGGATTTCGGCGCCTTCAGCGCCTTC |
| | | TACTCCGTGATGAAGTTCCTGAACCTGACCAGCGAGAAGGTGTCCCA |
| | | GGAAAAAGTGACAGAGATGATGAAGAAGTTCTGCGCCCAGCCCTGGG |
| | | AGGAAATCAAGACCTCCTACGCTGGCGTGAAAGAGAAGTACCTGAGC |
| | | GAGTACTGCTTCAGCGGCACCTACATCCTGAGCCTGCTGCCGCAGGG |
| | | CTACCACTTCACCGCCGATAGCTGGGAGCACATCCACTTCATCGGCAA |
| | | GATTCAGGGCAGCGACGCCGGCTGGACACTGGGCTACATGCTGAATC |
| | | TGACCAACATGATCCCCGCCGAGCAGCCCCTGAGCACACCTCTGAGC |
| | | CACAGCACC |
| EP14 | | |
| SEQ ID | Amino | TQNKALPENVKYGIVLDAGSSHTSLYIYKWPAEKENDTGVVHQVEECRVK |
| NO: 32 | acid | GPGISKFVQKVNEIGIYLTDCMERAREVIPRSQHQETPVYLGATAGMRLLR |
| | | MESEELADRVLDVVERSLSNYPFDFQGARIITGQEEGAYGWITINYLLGKF |
| | | SQKNQETFGALDLGGASTQVTFVPQNQTIESPDNALQFRLYGKDYNVYT |
| | | HSFLCYGKDQALWQKLAKDIQVASNEILRDPCFHPGYKKVVNVSDLYKTP |
| | | CTKRFEMTLPFQQFEIQGIGNYQQCHQSILELFNTSYCPYSQCAFNGIFLP |
| | | PLQGDFGAFSAFYSVMKFLNLTSEKVSQEKVTEMMKKFCAQPWEEIKTS |
| | | YAGVKEKYLSEYCFSGTYILSLLLQGYHFTADSWEHIHFIGKIQGSDAGWT |
| | | LGYMLNLTNMIPAEQPLSTPLSHST |
| SEQ ID | DNA | ACCCAGAACAAGGCCCTGCCCGAGAACGTGAAGTACGGCATCGTGCT |
| NO: 33 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCAGGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGCTGGACGTGGTGGAAAGAAGCCTGAGCAACTACCCATTCGAT |
| | | TTTCAAGGCGCCAGAATCATCACCGGCCAGGAAGAAGGCGCCTACGG |
| | | CTGGATCACCATCAACTACCTGCTGGGCAAGTTCAGCCAGAAGAATCA |
| | | GGAAACCTTCGGCGCCTTGGACCTGGGCGGAGCTTCTACCCAAGTGA |
| | | CCTTCGTGCCCCAGAATCAGACCATCGAGAGCCCCGACAACGCCCTG |
| | | CAGTTCCGGCTGTACGGCAAGGACTACAATGTGTACACCCACAGCTTT |
| | | CTGTGCTACGGAAAGGACCAGGCTCTGTGGCAGAAGCTGGCCAAGGA |
| | | CATCCAGGTGGCCAGCAACGAGATCCTGCGGGACCCTTGCTTCCACC |
| | | CCGGCTACAAGAAAGTCGTGAACGTGTCCGACCTGTACAAGACCCCC |
| | | TGCACCAAGAGATTCGAGATGACCCTGCCCTTCCAGCAGTTCGAGATC |
| | | CAGGGCATCGGCAATTACCAGCAGTGCCACCAGAGCATCCTGGAACT |
| | | GTTCAACACCAGCTACTGCCCCTACAGCCAGTGCGCCTTCAACGGCA |
| | | TCTTCCTGCCACCTCTGCAGGGGGATTTCGGCGCCTTCAGCGCCTTC |
| | | TACTCCGTGATGAAGTTCCTGAACCTGACCAGCGAGAAGGTGTCCCA |
| | | GGAAAAAGTGACAGAGATGATGAAGAAGTTCTGCGCCCAGCCCTGGG |
| | | AGGAAATCAAGACCTCCTACGCTGGCGTGAAAGAGAAGTACCTGAGC |
| | | GAGTACTGCTTCAGCGGCACCTACATCCTGAGCCTGCTGCTGCAGGG |
| | | CTACCACTTCACCGCCGATAGCTGGGAGCACATCCACTTCATCGGCAA |
| | | GATTCAGGGCAGCGACGCCGGCTGGACACTGGGTTACATGCTGAATC |
| | | TGACCAACATGATCCCCGCCGAGCAGCCCCTGAGCACACCTCTGAGC |
| | | CACAGCACC |
| EP15 | | |
| SEQ ID | Amino | TQNKALPENVKYGIVLDAGSSHTSLYIYKWPAEKENDTGVVHQVEECRVK |
| NO: 34 | acid | GPGISKFVQKVNEIGIYLTDCMERAREVIPRSQHQETPVYLGATAGMRLLR |
| | | MESEELADRVLDVVERSLSNYPFDFQGARIITGQEEGAYGWITINYLLGKF |
| | | SQKNQETFGALDLGGASTQVTFVPQNQTIESPDNALQFRLYGKDYNVYT |
| | | HSFLCYGKDQALWQKLAKDIQVASNEILRDPCFHPGYKKVVKVSDLYKTP |
| | | CTKRFEMTLPFQQFEIQGIGNYQQCHQSILELFNTSYCPYSQCAFNGIFLP |
| | | PLQGDFGAFSAFYSVMKFLNLTSEKVSQEKVTEMMKKFCAQPWEEIKTS |
| | | YAGVKEKYLSEYCFSGTYILSLLPQGYHFTADSWEHIHFIGKIQGSDAGWT |
| | | LGYMLNLTNMISAEQPLSTPLSHST |
| SEQ ID | DNA | ACCCAGAACAAGGCCCTGCCCGAGAACGTGAAGTACGGCATCGTGCT |
| NO: 35 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCAGGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGCTGGACGTGGTGGAAAGAAGCCTGAGCAACTACCCATTCGAT |
| | | TTTCAAGGCGCCAGAATCATCACCGGCCAGGAAGAAGGCGCCTACGG |
| | | CTGGATCACCATCAACTACCTGCTGGGCAAGTTCAGCCAGAAGAATCA |
| | | GGAAACCTTCGGCGCCCTGGACCTGGGCGGAGCTTCTACCCAAGTGA |
| | | CCTTCGTGCCCCAGAATCAGACCATCGAGAGCCCCGACAACGCCCTG |
| | | CAGTTCCGGCTGTACGGCAAGGACTACAATGTGTACACCCACAGCTTT |
| | | CTGTGCTACGGAAAGGACCAGGCGCTGTGGCAGAAGCTGGCCAAGG |
| | | ACATCCAGGTGGCCAGCAACGAGATCCTGCGGGACCCTTGCTTCCAC |
| | | CCCGGCTACAAGAAAGTCGTGAAGGTGTCCGACCTGTACAAGACCCC |
| | | CTGCACCAAGAGATTCGAGATGACCCTGCCCTTCCAGCAGTTCGAGAT |
| | | CCAGGGCATCGGCAATTACCAGCAGTGCCACCAGAGCATCCTGGAAC |
| | | TGTTCAACACCAGCTACTGCCCATACAGCCAGTGCGCCTTCAACGGCA |
| | | TCTTCCTGCCACCTCTGCAGGGGGATTTCGGCGCCTTCAGCGCCTTC |
| | | TACTCCGTGATGAAGTTCCTGAACCTGACCAGCGAGAAGGTGTCCCA |
| | | GGAAAAAGTGACAGAGATGATGAAGAAGTTCTGCGCCCAGCCCTGGG |
| | | AGGAAATCAAGACCTCCTACGCTGGCGTGAAAGAGAAGTACCTGAGC |
| | | GAGTACTGCTTCAGCGGCACCTACATCCTGAGCCTGCTGCCGCAGGG |
| | | CTACCACTTCACCGCCGATAGCTGGGAGCACATCCACTTCATCGGCAA |
| | | GATTCAGGGCAGCGACGCCGGCTGGACACTGGGCTACATGCTGAATC |
| | | TGACCAACATGATCTCCGCCGAGCAGCCCCTGAGCACACCTCTGAGC |
| | | CACAGCACC |
| EP17 | | |
| SEQ ID | Amino | TQNKALPENVKYGIVLDAGSSHTSLYIYKWPAEKENDTGVVHQVEECRVK |
| NO: 36 | acid | GPGISKFVQKVNEIGIYLTDCMERAREVIPRSQHQETPVYLGATAGMRLLR |
| | | MESEELADRVLDVVERSLSNYPFDFQGARIITGQEEGAYGWITINYLLGKF |
| | | SQKNQETFGALDLGGASTQVTFVPQNQTIESPDNALQFRLYGKDYNVYT |
| | | HSFLCYGKDQALWQKLAKDIQVASNEILRDPCFHPGYKKVVNVSDLYKTP |
| | | CTKRFEMTLPFQQFEIQGIGNYQQCHQSILELFNTSYCPYSQCAFNGIFLP |
| | | PLQGDFGAFSAFYFVMKFLNLTSEKVSQEKVTEMMKKFCAQPWEEIKTS |
| | | YAGVNEKYLSEFCFSGTYILSLLLQGYHFTADSWEHIHFIGKIQGSDAGWT |
| | | LGYMLNLTNMIPAEQPLSTPLSHST |
| SEQ ID | DNA | ACCCAGAACAAGGCCCTGCCCGAGAACGTGAAGTACGGCATCGTGCT |
| NO: 37 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCAGGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGCTGGACGTGGTGGAAAGAAGCCTGAGCAACTACCCATTCGAT |
| | | TTTCAAGGCGCCAGAATCATCACCGGCCAGGAAGAAGGCGCCTACGG |
| | | CTGGATCACCATCAACTACCTGCTGGGCAAGTTCAGCCAGAAGAATCA |
| | | GGAAACCTTCGGCGCCCTGGACCTGGGCGGAGCTTCTACCCAAGTGA |
| | | CCTTCGTGCCCCAGAATCAGACCATCGAGAGCCCCGACAACGCCCTG |
| | | CAGTTCCGGCTGTACGGCAAGGACTACAATGTGTACACCCACAGCTTT |
| | | CTGTGCTACGGAAAGGACCAGGCTCTGTGGCAGAAGCTGGCCAAGGA |
| | | CATCCAGGTGGCCAGCAACGAGATCCTGCGGGACCCTTGCTTCCACC |
| | | CCGGCTACAAGAAAGTCGTGAACGTGTCCGACCTGTACAAGACCCCC |
| | | TGCACCAAGAGATTCGAGATGACCCTGCCCTTCCAGCAGTTCGAGATC |
| | | CAGGGCATCGGCAATTACCAGCAGTGCCACCAGAGCATCCTGGAACT |
| | | GTTCAACACCAGCTACTGCCCCTACAGCCAGTGCGCCTTCAACGGCA |
| | | TCTTCCTGCCACCTCTGCAGGGGGATTTCGGCGCCTTCAGCGCCTTC |
| | | TACTTCGTGATGAAGTTCCTGAACCTGACCAGCGAGAAGGTGTCCCAG |
| | | GAAAAAGTGACAGAGATGATGAAGAAGTTCTGCGCCCAACCCTGGGA |
| | | GGAAATCAAGACCTCCTACGCTGGCGTGAACGAGAAGTACCTGAGCG |
| | | AGTTTTGCTTCAGCGGCACCTACATCCTGAGCCTGCTGCTGCAGGGC |
| | | TACCACTTCACCGCCGATAGCTGGGAGCACATCCACTTCATCGGCAA |
| | | GATTCAGGGCAGCGACGCCGGCTGGACACTGGGCTACATGCTGAATC |
| | | TGACCAACATGATCCCCGCCGAGCAGCCCCTGAGCACACCTCTGAGC |
| | | CACAGCACC |
| EP17.1 | | |
| SEQ ID | Amino | TQNKALPENVKYGIVLDAGSSHTSLYIYKWPAEKENDTGVVHQVEECRVK |
| NO: 38 | acid | GPGISKFVQKVNEIGIYLTDCMERAREVIPRSQHQETPVYLGATAGMRLLR |
| | | MESEELADRVLDVVERSLSNYPFDFQGARIITGQEEGAYGWITINYLLGKF |
| | | SQKNQETFGALDLGGASTQVTFVPQNQTIESPDNALQFRLYGKDYNVYT |
| | | HSFLCYGKDQALWQKLAKDIQVASNEILRDPCFHPGYKKVVNVSDLYKTP |
| | | CTKRFEMTLPFQQFEIQGIGNYQQCHQSILELFNTSYCPYSQCAFNGIFLP |
| | | PLQGDFGAFSAFYFVMKFLNLTSEKVSQEKVTEMMKKFCAQPWEEIKTS |
| | | YAGVKEKYLSEFCFSGTYILSLLLQGYHFTADSWEHIHFIGKIQGSDAGWT |
| | | LGYMLNLTNMIPAEQPLSTPLSHST |
| SEQ ID | DNA | ACCCAGAACAAGGCCCTGCCCGAGAACGTGAAGTACGGCATCGTGCT |
| NO: 39 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCAGGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGCTGGACGTGGTGGAAAGAAGCCTGAGCAACTACCCATTCGAT |
| | | TTTCAAGGCGCCAGAATCATCACCGGCCAGGAAGAAGGCGCCTACGG |
| | | CTGGATCACCATCAACTACCTGCTGGGCAAGTTCAGCCAGAAGAATCA |
| | | GGAAACCTTCGGCGCCCTGGACCTGGGCGGAGCTTCTACCCAAGTGA |
| | | CCTTCGTGCCCCAGAATCAGACCATCGAGAGCCCCGACAACGCCCTG |
| | | CAGTTCCGGCTGTACGGCAAGGACTACAATGTGTACACCCACAGCTTT |
| | | CTGTGCTACGGAAAGGACCAGGCTCTGTGGCAGAAGCTGGCCAAGGA |
| | | CATCCAGGTGGCCAGCAACGAGATCCTGCGGGACCCTTGCTTCCACC |
| | | CCGGCTACAAGAAAGTCGTGAACGTGTCCGACCTGTACAAGACCCCC |
| | | TGCACCAAGAGATTCGAGATGACCCTGCCCTTCCAGCAGTTCGAGATC |
| | | CAGGGCATCGGCAATTACCAGCAGTGCCACCAGAGCATCCTGGAACT |
| | | GTTCAACACCAGCTACTGCCCCTACAGCCAGTGCGCCTTCAACGGCA |
| | | TCTTCCTGCCACCTCTGCAGGGGGATTTCGGCGCCTTCAGCGCCTTC |
| | | TACTTCGTGATGAAGTTCCTGAACCTGACCAGCGAGAAGGTGTCCCAG |
| | | GAAAAAGTGACAGAGATGATGAAGAAGTTCTGCGCCCAGCCCTGGGA |
| | | GGAAATCAAGACCTCCTACGCTGGCGTGAAAGAGAAGTACCTGAGCG |
| | | AGTTTTGCTTCAGCGGCACCTACATCCTGAGCCTGCTGCTGCAGGGC |
| | | TACCACTTCACCGCCGATAGCTGGGAGCACATCCACTTCATCGGCAA |
| | | GATTCAGGGCAGCGACGCCGGCTGGACACTGGGCTACATGCTGAATC |
| | | TGACCAACATGATCCCCGCCGAGCAGCCCCTGAGCACACCTCTGAGC |
| | | CACAGCACC |
| EP18 | | |
| SEQ ID | Amino | TQNKALPENVKYGIVLDAGSSHTSLYIYKWPAEKENDTGVVHQVEECRVK |
| NO: 40 | acid | GPGISKFVQKVNEIDIYLTDCMERAREVIPRSQHQETPVYLGATAGMRLLR |
| | | MESEELADRVLDVVERSLSNYPFDFQGARIITGQEEGAYGWITINYLLGKF |
| | | SQKNQETFGALDLGGASTQVTFVPQNQTIESPDNALQFRLYGKDYNVYT |
| | | HSFLCYGKDQALWQKLAKDIQVASNEILRDPCFHPGYKKVVNVSDLYKTP |
| | | CTKRFEMTLPFQQFEIQGIGNYQQCHQSILELFNTSYCPYSQCAFNGIFLP |
| | | PLQGDFGAFSAFYFVMKFLNLTSEKVSQEKVTEMMKKFCAQPWEEIKTS |
| | | YAGVKEKYLSEYCFSGTYILSLLQQGYHFTADSWEHIHFIGKIQGSDAGW |
| | | TLGYMLNLTNMIPAEQPLSTPLSHST |
| SEQ ID | DNA | ACCCAGAACAAGGCCCTGCCCGAGAACGTGAAGTACGGCATCGTGCT |
| NO: 41 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGACATCTACCTGACCGACTGCATGGAACGGGCCAGGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGCTGGACGTGGTGGAAAGAAGCCTGAGCAACTACCCATTCGAT |
| | | TTTCAAGGCGCCAGAATCATCACCGGCCAGGAAGAAGGCGCCTACGG |
| | | CTGGATCACCATCAACTACCTGCTGGGCAAGTTCAGCCAGAAGAATCA |
| | | GGAAACCTTCGGCGCCCTGGACCTGGGCGGAGCTTCTACCCAAGTGA |
| | | CCTTCGTGCCCCAGAATCAGACCATCGAGAGCCCCGACAACGCCCTG |
| | | CAGTTCCGGCTGTACGGCAAGGACTACAATGTGTACACCCACAGCTTT |
| | | CTGTGCTACGGAAAGGACCAGGCTCTGTGGCAGAAGCTGGCCAAGGA |
| | | CATCCAGGTGGCCAGCAACGAGATCCTGCGGGACCCTTGCTTCCACC |
| | | CCGGCTACAAGAAAGTCGTGAACGTGTCCGACCTGTACAAGACCCCC |
| | | TGCACCAAGAGATTCGAGATGACCCTGCCCTTCCAGCAGTTCGAGATC |
| | | CAGGGCATCGGCAATTACCAGCAGTGCCACCAGAGCATCCTGGAACT |
| | | GTTCAACACCAGCTACTGCCCCTACAGCCAGTGCGCCTTCAACGGCA |
| | | TCTTCCTGCCACCTCTGCAGGGGGATTTCGGCGCCTTCAGCGCCTTC |
| | | TACTTCGTGATGAAGTTCCTGAACCTGACCAGCGAGAAGGTGTCCCAG |
| | | GAAAAAGTGACAGAGATGATGAAGAAGTTCTGCGCCCAGCCCTGGGA |
| | | GGAAATCAAGACCTCCTACGCTGGCGTGAAAGAGAAGTACCTGAGCG |
| | | AGTACTGCTTCAGCGGCACCTACATCCTGAGCCTGCTGCAGCAGGGC |
| | | TACCACTTCACCGCCGATAGCTGGGAGCACATCCACTTCATCGGCAA |
| | | GATTCAGGGCAGCGACGCCGGCTGGACACTGGGCTACATGCTGAATC |
| | | TGACCAACATGATCCCCGCCGAGCAGCCCCTGAGCACACCTCTGAGC |
| | | CACAGCACC |
| EP19 | | |
| SEQ ID | Amino | TQNKALPENVKYGIVLDAGSSHTSLYIYKWPAEKENDTGVVHQVEECRVK |
| NO: 42 | acid | GPGISKFVQKVNEIGIYLTDCMERAREVIPRSQHQETPVYLGATAGMRLLR |
| | | MESEELADRVLDVVERSLSNYPFDFQGARIITGQEEGAYGWITINYLLGKF |
| | | SQKNQETFGALDLGGASTQVTFVPQNQTIESPDNALQFRLYGKDYNVYT |
| | | HSFLCYGKDQALWQKLAKDIQVASNEILRDPCFHPGYKKVVNVSDLYKTP |
| | | CTKRFEMTLPFQQFEIQGIGNYQQCHQSILELFNTSYCPYSQCAFNGIFLP |
| | | PLQGDFGAFSAFYFVMKFLNLTSEKVSQEKVTEMMKKFCAQPWEEIKTS |
| | | YAGVKEKYLSEYCFSGTYILSLLQQGYHFTADSWEDIHFIGKIQGSDAGW |
| | | TLGYMLNLTNMIPAEQPLSTPLSHST |
| SEQ ID | DNA | ACCCAGAACAAGGCCCTGCCCGAGAACGTGAAGTACGGCATCGTGCT |
| NO: 43 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCAGGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGCTGGACGTGGTGGAAAGAAGCCTGAGCAACTACCCATTCGAT |
| | | TTTCAAGGCGCCAGAATCATCACCGGCCAGGAAGAAGGCGCCTACGG |
| | | CTGGATCACCATCAACTACCTGCTGGGCAAGTTCAGCCAGAAGAATCA |
| | | GGAAACCTTCGGCGCCCTGGACCTGGGCGGAGCTTCTACCCAAGTGA |
| | | CCTTCGTGCCCCAGAATCAGACCATCGAGAGCCCCGACAACGCCCTG |
| | | CAGTTCCGGCTGTACGGCAAGGACTACAATGTGTACACCCACAGCTTT |
| | | CTGTGCTACGGAAAGGACCAGGCTCTGTGGCAGAAGCTGGCCAAGGA |
| | | CATCCAGGTGGCCAGCAACGAGATCCTGCGGGACCCTTGCTTCCACC |
| | | CCGGCTACAAGAAAGTCGTGAACGTGTCCGACCTGTACAAGACCCCC |
| | | TGCACCAAGAGATTCGAGATGACCCTGCCCTTCCAGCAGTTCGAGATC |
| | | CAGGGCATCGGCAATTACCAGCAGTGCCACCAGAGCATCCTGGAACT |
| | | GTTCAACACCAGCTACTGCCCCTACAGCCAGTGCGCCTTCAACGGCA |
| | | TCTTCCTGCCACCTCTGCAGGGGGATTTCGGCGCCTTCAGCGCCTTC |
| | | TACTTCGTGATGAAGTTCCTGAACCTGACCAGCGAGAAGGTGTCCCAG |
| | | GAAAAAGTGACAGAGATGATGAAGAAGTTCTGCGCCCAGCCCTGGGA |
| | | GGAAATCAAGACCTCCTACGCTGGCGTGAAAGAGAAGTACCTGAGCG |
| | | AGTACTGCTTCAGCGGCACCTACATCCTGAGCCTGCTGCAGCAGGGC |
| | | TACCACTTCACCGCCGATAGCTGGGAGGACATCCACTTCATCGGCAA |
| | | GATTCAGGGCAGCGACGCCGGCTGGACACTGGGCTACATGCTGAATC |
| | | TGACCAACATGATCCCCGCCGAGCAGCCCCTGAGCACACCTCTGAGC |
| | | CACAGCACC |
| EP20 | | |
| SEQ ID | Amino | TQNKALPENVKYGIVLDAGSSHTSLYIYKWPAEKENDTGVVHQVEECRVK |
| NO: 44 | acid | GPGISKFVQKVNEIGIYLTDCMERAREVIPRSQHQETPVYLGATAGMRLLR |
| | | MESEELADRVLDVVERSLSNYPFDFQGARIITGQEEGAYGWITINYLLGKF |
| | | SQKNQETFGALDLGGASTQVTFVPQNQTIESPDNALQFRLYGKDYNVYT |
| | | HSFLCYGKDQALWQKLAKDIQVASNGILRDPCFHPGYKKVVNVSDLYKTP |
| | | CTKRFEMTLPFQQFEIQGIGNYQQCHQSILELFNTSYCPYSQCAFNGIFLP |
| | | PLQGDFGAFSAFYFVMKFLNLTSEKVSQEKVTEMMKKFCAQPWEEIKTS |
| | | YAGVKEKYLSEYCFSGTYILSLLLQGYHFTADSWEHIHSIGKIQGSDAGWT |
| | | LGYMLNLTNMIPAEQPLSTPLSHST |
| SEQ ID | DNA | ACCCAGAACAAGGCCCTGCCCGAGAACGTGAAGTACGGCATCGTGCT |
| NO: 45 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCAGGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGCTGGACGTGGTGGAAAGAAGCCTGAGCAACTACCCATTCGAT |
| | | TTTCAAGGCGCCAGAATCATCACCGGCCAGGAAGAAGGCGCCTACGG |
| | | CTGGATCACCATCAACTACCTGCTGGGCAAGTTCAGCCAGAAGAATCA |
| | | GGAAACCTTCGGCGCCCTGGACCTGGGCGGAGCTTCTACCCAAGTGA |
| | | CCTTCGTGCCCCAGAATCAGACCATCGAGAGCCCCGACAACGCCCTG |
| | | CAGTTCCGGCTGTACGGCAAGGACTACAATGTGTACACCCACAGCTTT |
| | | CTGTGCTACGGAAAGGACCAGGCTCTGTGGCAGAAGCTGGCCAAGGA |
| | | CATCCAGGTGGCCAGCAACGGGATCCTGCGGGACCCTTGCTTCCACC |
| | | CCGGCTACAAGAAAGTCGTGAACGTGTCCGACCTGTACAAGACCCCC |
| | | TGCACCAAGAGATTCGAGATGACCCTGCCCTTCCAGCAGTTCGAGATC |
| | | CAGGGCATCGGCAATTACCAGCAGTGCCACCAGAGCATCCTGGAACT |
| | | GTTCAACACCAGCTACTGCCCCTACAGCCAGTGCGCCTTCAACGGCA |
| | | TCTTCCTGCCACCTCTGCAGGGGGATTTCGGCGCCTTCAGCGCCTTC |
| | | TACTTCGTGATGAAGTTCCTGAACCTGACCAGCGAGAAGGTGTCCCAG |
| | | GAAAAAGTGACAGAGATGATGAAGAAGTTCTGCGCCCAGCCCTGGGA |
| | | GGAAATCAAGACCTCCTACGCTGGCGTGAAAGAGAAGTACCTGAGCG |
| | | AGTACTGCTTCAGCGGCACCTACATCCTGAGCCTGCTGCTGCAGGGC |
| | | TACCACTTCACCGCCGATAGCTGGGAGCACATCCACTCCATCGGCAA |
| | | GATTCAGGGCAGCGACGCCGGCTGGACACTGGGCTACATGCTGAATC |
| | | TGACCAACATGATCCCCGCCGAGCAGCCCCTGAGCACACCTCTGAGC |
| | | CACAGCACC |
| EP21 | | |
| SEQ ID | Amino | TQNKALPENVKYGIVLDAGSSHTSLYIYKWPAEKENDTGVVHQVEECRVK |
| NO: 46 | acid | GPGISKFVQKVNEIGIYLTDCMERAMEVIPRSQHQETPVYLGATAGMRLL |
| | | RMESEELADRVLDVVERSLSNYPFDFQGARIITGQEEGAYGWITINYLLGK |
| | | FSQKNQETFGALDLGGASTQVTFVPQNQTIESPDNALQFRLYGKDYNVY |
| | | THSFLCYGKDQALWQKLAKDIQVASNEILRDPCFHPGYKKVVNVSDLYKT |
| | | PCTKRFEMTLPFQQFEIQGIGNYQQCHQSILELFNTSYCPYSQCAFNGIFL |
| | | PPLQGDFGAFSAFYFVMKFLNLTSEKVSQEKVTEMMKKFCAQPWEEIKT |
| | | SYAGVKEKYLSEYCFSGTYILSLLLQGYHFTADSWEHIHFIGKIQGSDAGW |
| | | TLGYMLNLTNMIPAEQPLRTPLSHST |
| SEQ ID | DNA | ACCCAGAACAAGGCCCTGCCCGAGAACGTGAAGTACGGCATCGTGCT |
| NO: 47 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCATGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGCTGGACGTGGTGGAAAGAAGCCTGAGCAACTACCCATTCGAT |
| | | TTTCAAGGCGCCAGAATCATCACCGGCCAGGAAGAAGGCGCCTACGG |
| | | CTGGATCACCATCAACTACCTGCTGGGCAAGTTCAGCCAGAAGAATCA |
| | | GGAAACCTTCGGCGCCCTGGACCTGGGCGGAGCTTCTACCCAAGTGA |
| | | CCTTCGTGCCCCAGAATCAGACCATCGAGAGCCCCGACAACGCCCTG |
| | | CAGTTCCGGCTGTACGGCAAGGACTACAATGTGTACACCCACAGCTTT |
| | | CTGTGCTACGGAAAGGACCAGGCTCTGTGGCAGAAGCTGGCCAAGGA |
| | | CATCCAGGTGGCCAGCAACGAGATCCTGCGGGACCCTTGCTTCCACC |
| | | CCGGCTACAAGAAAGTCGTGAACGTGTCCGACCTGTACAAGACCCCC |
| | | TGCACCAAGAGATTCGAGATGACCCTGCCCTTCCAGCAGTTCGAGATC |
| | | CAGGGCATCGGCAATTACCAGCAGTGCCACCAGAGCATCCTGGAACT |
| | | GTTCAACACCAGCTACTGCCCCTACAGCCAGTGCGCCTTCAACGGCA |
| | | TCTTCCTGCCACCTCTGCAGGGGGATTTCGGCGCCTTCAGCGCCTTC |
| | | TACTTCGTGATGAAGTTCCTGAACCTGACCAGCGAGAAGGTGTCCCAG |
| | | GAAAAAGTGACAGAGATGATGAAGAAGTTCTGCGCCCAGCCCTGGGA |
| | | GGAAATCAAGACCTCCTACGCTGGCGTGAAAGAGAAGTACCTGAGCG |
| | | AGTACTGCTTCAGCGGCACCTACATCCTGAGCCTGCTGCTGCAGGGC |
| | | TACCACTTCACCGCCGATAGCTGGGAGCACATCCACTTCATCGGCAA |
| | | GATTCAGGGCAGCGACGCCGGCTGGACACTGGGCTACATGCTGAATC |
| | | TGACCAACATGATCCCCGCCGAGCAGCCCCTGCGCACACCTCTGAGC |
| | | CACAGCACC |
| EP22 | | |
| SEQ ID | Amino | TQNKALPENVKYGIVLDAGSSHTSLYIYKWPAEKENDTGVVHQVEECRVK |
| NO: 48 | acid | GPGISKFVQKVNEIGIYLTDCMERAREVIPRSQHQETPVYLGATAGMRLLR |
| | | MESEELADRVLDVVERSLSNYPFDFQGARIITGQEEGAYGWITINYLLGKF |
| | | SQKNQETFGALDLGGASTQVTFVPQNQTIESPDNALQFRLYGKDYNVYT |
| | | HSFLCYGKDQALWQKLAKDIQVASNGILRDPCFHPGYKKVVNVSDLYKTP |
| | | CTKRFEMTLPFQQFEIQGIGNYQQCHQSILELFNTSYCPYSQCAFNGIFLP |
| | | PLQGDFGAFSAFYFVMKFLNLTSEKVSQEKVTEMMKKFCAQPWEEIKTS |
| | | YAGVKEKYLSEYCFSGTYILSLLLQGYHFTADSWEHIHFIGKIQGSDAGWT |
| | | LGYMLNLTNMIPAEQPLGTPLSHST |
| SEQ ID | DNA | ACCCAGAACAAGGCCCTGCCCGAGAACGTGAAGTACGGCATCGTGCT |
| NO: 49 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCAGGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGCTGGACGTGGTGGAAAGAAGCCTGAGCAACTACCCATTCGAT |
| | | TTTCAAGGCGCCAGAATCATCACCGGCCAGGAAGAAGGCGCCTACGG |
| | | CTGGATCACCATCAACTACCTGCTGGGCAAGTTCAGCCAGAAGAATCA |
| | | GGAAACCTTCGGCGCCCTGGACCTGGGCGGAGCTTCTACCCAAGTGA |
| | | CCTTCGTGCCCCAGAATCAGACCATCGAGAGCCCCGACAACGCCCTG |
| | | CAGTTCCGGCTGTACGGCAAGGACTACAATGTGTACACCCACAGCTTT |
| | | CTGTGCTACGGAAAGGACCAGGCTCTGTGGCAGAAGCTGGCCAAGGA |
| | | CATCCAGGTGGCCAGCAACGGGATCCTGCGGGACCCTTGCTTCCACC |
| | | CCGGCTACAAGAAAGTCGTGAACGTGTCCGACCTGTACAAGACCCCC |
| | | TGCACCAAGAGATTCGAGATGACCCTGCCCTTCCAGCAGTTCGAGATC |
| | | CAGGGCATCGGCAATTACCAGCAGTGCCACCAGAGCATCCTGGAACT |
| | | GTTCAACACCAGCTACTGCCCCTACAGCCAGTGCGCCTTCAACGGCA |
| | | TCTTCCTGCCACCTCTGCAGGGGGATTTCGGCGCCTTCAGCGCCTTC |
| | | TACTTCGTGATGAAGTTCCTGAACCTGACCAGCGAGAAGGTGTCCCAG |
| | | GAAAAAGTGACAGAGATGATGAAGAAGTTCTGCGCCCAGCCCTGGGA |
| | | GGAAATCAAGACCTCCTACGCTGGCGTGAAAGAGAAGTACCTGAGCG |
| | | AGTACTGCTTCAGCGGCACCTACATCCTGAGCCTGCTGCTGCAGGGC |
| | | TACCACTTCACCGCCGATAGCTGGGAGCACATCCACTTCATCGGCAA |
| | | GATTCAGGGCAGCGACGCCGGCTGGACACTGGGCTACATGTTGAATC |
| | | TGACCAACATGATCCCCGCCGAGCAGCCCCTGGGCACACCTCTGAGC |
| | | CACAGCACC |
| EP23 | | |
| SEQ ID | Amino | TQNKALPENVKYGIVLDAGSSHTSLYIYKWPAEKENDTGVVHQVEECRVK |
| NO: 50 | acid | GPGISKFVQKVNEIGIYLTDCMERAREVIPRSQHQETPVYLGATAGMRLLR |
| | | MESEELADRVLDVVERSLSNYPFDFQGARIITGQEEGAYGWITINYLLGKF |
| | | SQKNQETFGALDLGGASTQVTFVPQNQTIESPDNALQFRLYGKDYNVYT |
| | | HSFMCYGKDQALRQKLAKDIQVASNEILRDPCFHPGYKKVVNVSDLYKTP |
| | | CTKRFEMTLPFQQFEIQGIGNYQQCHQSILELFNTSYCPYSQCAFNGIFLP |
| | | PLQGDFGAFSAFYFVMKFLNLTSEKVSQEKVTEMMKKFCAQPWEEIKTS |
| | | YAGVKEKYLSEYCFSGTYILSLLLQGYHFTADSWEHIHSIGKIQGSDAGWT |
| | | LGYMLNLTNMIPAEQPLRTPLSHST |
| SEQ ID | DNA | ACCCAGAACAAGGCCCTGCCCGAGAACGTGAAGTACGGCATCGTGCT |
| NO: 51 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCAGGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGCTGGACGTGGTGGAAAGAAGCCTGAGCAACTACCCATTCGAT |
| | | TTTCAAGGCGCCAGAATCATCACCGGCCAGGAAGAAGGCGCCTACGG |
| | | CTGGATCACCATCAACTACCTGCTGGGCAAGTTCAGCCAGAAGAATCA |
| | | GGAAACCTTCGGCGCCCTGGACCTGGGCGGAGCTTCTACCCAAGTGA |
| | | CCTTCGTGCCCCAGAATCAGACCATCGAGAGCCCCGACAACGCCCTG |
| | | CAGTTCCGGCTGTACGGCAAGGACTACAATGTGTACACCCACAGCTTT |
| | | ATGTGCTACGGAAAGGACCAGGCTCTGAGGCAGAAGCTGGCCAAGGA |
| | | CATCCAGGTGGCCAGCAACGAGATCCTGCGGGACCCTTGCTTCCACC |
| | | CCGGCTACAAGAAAGTCGTGAACGTGTCCGACCTGTACAAGACCCCC |
| | | TGTACCAAGAGATTCGAGATGACCCTGCCCTTCCAGCAGTTCGAGATC |
| | | CAGGGCATCGGCAATTACCAGCAGTGCCACCAGAGCATCCTGGAACT |
| | | GTTCAACACCAGCTACTGCCCCTACAGCCAGTGCGCCTTCAACGGCA |
| | | TCTTCCTGCCACCTCTGCAGGGGGATTTCGGCGCCTTCAGCGCCTTC |
| | | TACTTCGTGATGAAGTTCCTGAACCTGACCAGCGAGAAGGTGTCCCAG |
| | | GAAAAAGTGACAGAGATGATGAAGAAGTTCTGCGCCCAGCCCTGGGA |
| | | GGAAATCAAGACCTCCTACGCTGGCGTGAAAGAGAAGTACCTGAGCG |
| | | AGTACTGCTTCAGCGGCACCTACATCCTGAGCCTGCTGCTGCAGGGC |
| | | TACCACTTCACCGCCGATAGCTGGGAGCACATCCACTCCATCGGCAA |
| | | GATTCAGGGCAGCGACGCCGGCTGGACACTGGGCTACATGCTGAATC |
| | | TGACCAACATGATCCCCGCCGAGCAGCCCCTGAGAACACCTCTGAGC |
| | | CACAGCACC |
| EP24 | | |
| SEQ ID | Amino | TQNKALPENVKYGIVLDAGSSHTSLYIYKWPAEKENDTGVVHQVEECRVK |
| NO: 52 | acid | GPGISKFVQKVNEIGIYLTDCMERAKEVIPRSQHQETPVYLGATAGMRLLR |
| | | MESEELADRVLDVVERSLSNYPFDFQGARIITGQEEGAYGWITINYLLGKF |
| | | SQKNQETFGALDLGGASTQVTFVPQNQTIESPDNALQFRLYGKDYNVYT |
| | | HSFLCYGKDQALWQKLAKDIQVASNEILRDPCFHPGYKKVVNVSDLYKTP |
| | | CTKRFEMTLPFQQFEIQGIGNYQQCHQSILELFNTSYCPYSQCAFNG IFLP |
| | | PLQGDFGAFSAFYFVMKFLNLTSEKVSQEKVTEMMKKFCAQPWEEIKTS |
| | | YAGVKEKYLSEYCFSGTYILSLLQQGYHFTADSVVEHNHFIGKIQGSDAGW |
| | | TLGYMLNLTNMIPAEQPLSTPLSHST |
| SEQ ID | DNA | ACCCAGAACAAGGCCCTGCCCGAGAACGTGAAGTACGGCATCGTGCT |
| NO: 53 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCAAGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGCTGGACGTGGTGGAAAGAAGCCTGAGCAACTACCCATTCGAT |
| | | TTTCAAGGCGCCAGAATCATCACCGGCCAGGAAGAAGGCGCCTACGG |
| | | CTGGATCACCATCAACTACCTGCTGGGCAAGTTCAGCCAGAAGAATCA |
| | | GGAAACCTTCGGCGCCCTGGACCTGGGCGGAGCTTCTACCCAAGTGA |
| | | CCTTCGTGCCCCAGAATCAGACCATCGAGAGCCCCGACAACGCCCTG |
| | | CAGTTCCGGCTGTACGGCAAGGACTACAATGTGTACACCCACAGCTTT |
| | | CTGTGCTACGGAAAGGACCAGGCTCTGTGGCAGAAGCTGGCCAAGGA |
| | | CATCCAGGTGGCCAGCAACGAGATCCTGCGGGACCCTTGCTTCCACC |
| | | CCGGCTACAAGAAAGTCGTGAACGTGTCCGACCTGTACAAGACCCCC |
| | | TGCACCAAGAGATTCGAGATGACCCTGCCCTTCCAGCAGTTCGAGATC |
| | | CAGGGCATCGGCAATTACCAGCAGTGCCACCAGAGCATCCTGGAACT |
| | | GTTCAACACCAGCTACTGCCCCTACAGCCAGTGCGCCTTCAACGGCA |
| | | TCTTCCTGCCACCTCTGCAGGGGGATTTCGGCGCCTTCAGCGCCTTC |
| | | TACTTCGTGATGAAGTTCCTGAACCTGACCAGCGAGAAGGTGTCCCAG |
| | | GAAAAAGTGACAGAGATGATGAAGAAGTTCTGCGCCCAGCCCTGGGA |
| | | GGAAATCAAGACCTCCTACGCTGGCGTGAAAGAGAAGTACCTGAGCG |
| | | AGTACTGCTTCAGCGGCACCTACATCCTGAGCCTGCTGCAGCAGGGC |
| | | TACCACTTCACCGCCGATAGCTGGGAGCACAACCACTTCATCGGCAA |
| | | GATTCAGGGCAGCGACGCCGGCTGGACACTGGGCTACATGCTGAATC |
| | | TGACCAACATGATCCCCGCCGAGCAGCCCCTGAGCACACCTCTGAGC |
| | | CACAGCACC |
| EP1xEP17_K405N | | |
| SEQ ID | Amino | TQNKALPENVKYGIVLDAGSSHTSLYIYKWPAEKENDTGVVHQVEECRVK |
| NO: 54 | acid | GPGISKFVQKVNEIGIYLTDCMERAMEVIPRSQHQETPVYLGATAGMRLL |
| | | RMESEELADRVLDVVERSLSNYPFDFQGARIITGQEEGAYGWITINYLLGK |
| | | FSQKNQETFGALDLGGASTQVTFVPQNQTIESPDNALQFRLYGKDYNVY |
| | | THSFLCYGKDQALWQKLAKDIQVASNEILRDPCFHPGYKKVVNVSDLYKT |
| | | PCTKRFEMTLPFQQFEIQGIGNYQQCHQSILELFNTSYCPYSQCAFNGIFL |
| | | PPLQGDFGAFSAFYFVMKFLNLTSEKVSQEKVTEMMKKFCAQPWEEIKT |
| | | SYAGVNEKYLSEFCFSGTYILSLLLQGYHFTADSVVEHIHFIGKIQGSDAGW |
| | | TLGYMLNLTNMIPAEQPLSTPLSHST |
| SEQ ID | DNA | ACCCAGAACAAGGCCCTGCCCGAGAACGTGAAGTACGGCATCGTGCT |
| NO: 55 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCATGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGCTGGACGTGGTGGAAAGAAGCCTGAGCAACTACCCATTCGAT |
| | | TTTCAAGGCGCCAGAATCATCACCGGCCAGGAAGAAGGCGCCTACGG |
| | | CTGGATCACCATCAACTACCTGCTGGGCAAGTTCAGCCAGAAGAATCA |
| | | GGAAACCTTCGGCGCCCTGGACCTGGGCGGAGCTTCTACCCAAGTGA |
| | | CCTTCGTGCCCCAGAATCAGACCATCGAGAGCCCCGACAACGCCCTG |
| | | CAGTTCCGGCTGTACGGCAAGGACTACAATGTGTACACCCACAGCTTT |
| | | CTGTGCTACGGAAAGGACCAGGCTCTGTGGCAGAAGCTGGCCAAGGA |
| | | CATCCAGGTGGCCAGCAACGAGATCCTGCGGGACCCTTGCTTCCACC |
| | | CCGGCTACAAGAAAGTCGTGAACGTGTCCGACCTGTACAAGACCCCC |
| | | TGCACCAAGAGATTCGAGATGACCCTGCCCTTCCAGCAGTTCGAGATC |
| | | CAGGGCATCGGCAATTACCAGCAGTGCCACCAGAGCATCCTGGAACT |
| | | GTTCAACACCAGCTACTGCCCCTACAGCCAGTGCGCCTTCAACGGCA |
| | | TCTTCCTGCCACCTCTGCAGGGGGATTTCGGCGCCTTCAGCGCCTTC |
| | | TACTTCGTGATGAAGTTCCTGAACCTGACCAGCGAGAAGGTGTCCCAG |
| | | GAAAAAGTGACAGAGATGATGAAGAAGTTCTGCGCCCAACCCTGGGA |
| | | GGAAATCAAGACCTCCTACGCTGGCGTGAACGAGAAGTACCTGAGCG |
| | | AGTTTTGCTTCAGCGGCACCTACATCCTGAGCCTGCTGCTGCAGGGC |
| | | TACCACTTCACCGCCGATAGCTGGGAGCACATCCACTTCATCGGCAA |
| | | GATTCAGGGCAGCGACGCCGGCTGGACACTGGGCTACATGCTGAATC |
| | | TGACCAACATGATCCCCGCCGAGCAGCCCCTGAGCACACCTCTGAGC |
| | | CACAGCACC |
| EP1xEP17 | | |
| SEQ ID | Amino | TQNKALPENVKYGIVLDAGSSHTSLYIYKWPAEKENDTGVVHQVEECRVK |
| NO: 56 | acid | GPGISKFVQKVNEIGIYLTDCMERAMEVIPRSQHQETPVYLGATAGMRLL |
| | | RMESEELADRVLDVVERSLSNYPFDFQGARIITGQEEGAYGWITINYLLGK |
| | | FSQKNQETFGALDLGGASTQVTFVPQNQTIESPDNALQFRLYGKDYNVY |
| | | THSFLCYGKDQALWQKLAKDIQVASNEILRDPCFHPGYKKVVNVSDLYKT |
| | | PCTKRFEMTLPFQQFEIQGIGNYQQCHQSILELFNTSYCPYSQCAFNGIFL |
| | | PPLQGDFGAFSAFYFVMKFLNLTSEKVSQEKVTEMMKKFCAQPWEEIKT |
| | | SYAGVKEKYLSEFCFSGTYILSLLLQGYHFTADSWEHIHFIGKIQGSDAGW |
| | | TLGYMLNLTNMIPAEQPLSTPLSHST |
| SEQ ID | DNA | ACCCAGAACAAGGCCCTGCCCGAGAACGTGAAGTACGGCATCGTGCT |
| NO: 57 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCATGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGCTGGACGTGGTGGAAAGAAGCCTGAGCAACTACCCATTCGAT |
| | | TTTCAAGGCGCCAGAATCATCACCGGCCAGGAAGAAGGCGCCTACGG |
| | | CTGGATCACCATCAACTACCTGCTGGGCAAGTTCAGCCAGAAGAATCA |
| | | GGAAACCTTCGGCGCCCTGGACCTGGGCGGAGCTTCTACCCAAGTGA |
| | | CCTTCGTGCCCCAGAATCAGACCATCGAGAGCCCCGACAACGCCCTG |
| | | CAGTTCCGGCTGTACGGCAAGGACTACAATGTGTACACCCACAGCTTT |
| | | CTGTGCTACGGAAAGGACCAGGCTCTGTGGCAGAAGCTGGCCAAGGA |
| | | CATCCAGGTGGCCAGCAACGAGATCCTGCGGGACCCTTGCTTCCACC |
| | | CCGGCTACAAGAAAGTCGTGAACGTGTCCGACCTGTACAAGACCCCC |
| | | TGCACCAAGAGATTCGAGATGACCCTGCCCTTCCAGCAGTTCGAGATC |
| | | CAGGGCATCGGCAATTACCAGCAGTGCCACCAGAGCATCCTGGAACT |
| | | GTTCAACACCAGCTACTGCCCCTACAGCCAGTGCGCCTTCAACGGCA |
| | | TCTTCCTGCCACCTCTGCAGGGGGATTTCGGCGCCTTCAGCGCCTTC |
| | | TACTTCGTGATGAAGTTCCTGAACCTGACCAGCGAGAAGGTGTCCCAG |
| | | GAAAAAGTGACAGAGATGATGAAGAAGTTCTGCGCCCAGCCCTGGGA |
| | | GGAAATCAAGACCTCCTACGCTGGCGTGAAAGAGAAGTACCTGAGCG |
| | | AGTTTTGCTTCAGCGGCACCTACATCCTGAGCCTGCTGCTGCAGGGC |
| | | TACCACTTCACCGCCGATAGCTGGGAGCACATCCACTTCATCGGCAA |
| | | GATTCAGGGCAGCGACGCCGGCTGGACACTGGGCTACATGCTGAATC |
| | | TGACCAACATGATCCCCGCCGAGCAGCCCCTGAGCACACCTCTGAGC |
| | | CACAGCACC |
| EP28aa1-3 | | |
| SEQ ID | Amino | APTTQNKALPENVKYGIVLDAGSSHTSLYIYKWPAEKENDTGVVHQVEEC |
| NO: 58 | acid | RVKGPGISKFVQKVNEIGIYLTDCMERAREVIPRSQHQETPVYLGATAGM |
| | | RLLRMESEELADRVLDVVERSLSNYPFDFQGARIITGQEEGAYGWITINYL |
| | | LGKFSQKNQETFGALDLGGASTQVTFVPQNQTIESPDNALQFRLYGKDY |
| | | NVYTHSFLCYGKDQALWQKLAKDIQVASNEILRDPCFHPGYKKVVNVSDL |
| | | YKTPCTKRFEMTLPFQQFEIQGIGNYQQCHQSILELFNTSYCPYSQCAFN |
| | | GIFLPPLQGDFGAFSAFYFVMKFLNLTSEKVSQEKVTEMMKKFCAQPWE |
| | | EIKTSYAGVKEKYLSEYCFSGTYILSLLLQGYHFTADSWEHIHFIGKIQGSD |
| | | AGWTLGYMLNLTNMIPAEQPLSTPLSHST |
| SEQ ID | DNA | GCCCCTACCACCCAGAACAAGGCCCTGCCCGAGAACGTGAAGTACGG |
| NO: 59 | | CATCGTGCTGGACGCCGGCTCCTCCCACACCTCCCTGTACATCTACAA |
| | | GTGGCCTGCCGAGAAAGAAAACGACACCGGCGTGGTGCACCAAGTG |
| | | GAAGAGTGCAGAGTGAAGGGCCCCGGCATCTCCAAGTTCGTGCAGAA |
| | | AGTGAACGAGATCGGCATCTACCTGACCGACTGCATGGAACGGGCCA |
| | | GAGAAGTGATCCCTCGGTCCCAGCACCAGGAAACCCCTGTCTACCTG |
| | | GGCGCCACCGCCGGCATGCGGCTGCTGCGGATGGAATCCGAGGAAC |
| | | TGGCCGACCGGGTGCTGGACGTGGTGGAACGGTCCCTGTCCAACTAC |
| | | CCATTCGATTTTCAAGGCGCCAGAATCATCACCGGCCAGGAAGAGGG |
| | | CGCCTACGGCTGGATCACCATCAACTACCTGCTGGGCAAGTTCTCCC |
| | | AGAAGAATCAGGAAACCTTCGGCGCCCTGGACCTGGGCGGAGCCAG |
| | | CACCCAAGTCACATTCGTGCCCCAGAACCAGACCATCGAGAGCCCCG |
| | | ACAACGCCCTGCAGTTCCGGCTGTACGGCAAGGACTACAACGTGTAC |
| | | ACCCACAGCTTTCTGTGCTACGGCAAGGACCAGGCCCTGTGGCAGAA |
| | | GCTGGCCAAGGACATCCAAGTGGCCTCCAACGAGATCCTGCGGGACC |
| | | CCTGCTTCCACCCCGGCTACAAGAAAGTGGTCAACGTGTCCGACCTG |
| | | TACAAGACCCCTTGCACCAAGAGATTCGAGATGACCCTGCCCTTCCAG |
| | | CAGTTCGAGATCCAGGGCATCGGCAACTACCAGCAGTGCCACCAGTC |
| | | CATCCTGGAACTGTTCAACACCTCCTACTGCCCCTACTCCCAGTGCGC |
| | | CTTCAACGGCATCTTCCTGCCTCCACTGCAGGGCGACTTCGGCGCCT |
| | | TCTCCGCCTTCTACTTCGTGATGAAGTTCCTGAACCTGACCTCCGAGA |
| | | AAGTGTCCCAGGAAAAAGTGACCGAGATGATGAAGAAGTTCTGCGCC |
| | | CAGCCCTGGGAGGAAATCAAGACCTCCTACGCTGGCGTGAAAGAGAA |
| | | GTACCTGTCCGAGTACTGCTTCTCCGGCACCTACATCCTGTCCCTGCT |
| | | GCTGCAGGGCTACCACTTCACCGCCGACAGCTGGGAGCACATCCACT |
| | | TCATCGGCAAGATCCAGGGATCCGACGCTGGCTGGACCCTGGGCTAC |
| | | ATGCTGAATCTGACCAACATGATCCCCGCCGAGCAGCCCCTGTCCAC |
| | | CCCTCTGTCTCACTCCACC |
| EP14xEP17 | | |
| SEQ ID | Amino | TQNKALPENVKYGIVLDAGSSHTSLYIYKWPAEKENDTGVVHQVEECRVK |
| NO: 60 | acid | GPGISKFVQKVNEIGIYLTDCMERAREVIPRSQHQETPVYLGATAGMRLLR |
| | | MESEELADRVLDVVERSLSNYPFDFQGARIITGQEEGAYGWITINYLLGKF |
| | | SQKNQETFGALDLGGASTQVTFVPQNQTIESPDNALQFRLYGKDYNVYT |
| | | HSFLCYGKDQALWQKLAKDIQVASNEILRDPCFHPGYKKVVNVSDLYKTP |
| | | CTKRFEMTLPFQQFEIQGIGNYQQCHQSILELFNTSYCPYSQCAFNGIFLP |
| | | PLQGDFGAFSAFYSVMKFLNLTSEKVSQEKVTEMMKKFCAQPWEEIKTS |
| | | YAGVKEKYLSEFCFSGTYILSLLLQGYHFTADSWEHIHFIGKIQGSDAGWT |
| | | LGYMLNLTNMIPAEQPLSTPLSHST |
| SEQ ID | DNA | ACCCAGAACAAGGCCCTGCCCGAGAACGTGAAGTACGGCATCGTGCT |
| NO: 61 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCAGGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGCTGGACGTGGTGGAAAGAAGCCTGAGCAACTACCCATTCGAT |
| | | TTTCAAGGCGCCAGAATCATCACCGGCCAGGAAGAAGGCGCCTACGG |
| | | CTGGATCACCATCAACTACCTGCTGGGCAAGTTCAGCCAGAAGAATCA |
| | | GGAAACCTTCGGCGCCTTGGACCTGGGCGGAGCTTCTACCCAAGTGA |
| | | CCTTCGTGCCCCAGAATCAGACCATCGAGAGCCCCGACAACGCCCTG |
| | | CAGTTCCGGCTGTACGGCAAGGACTACAATGTGTACACCCACAGCTTT |
| | | CTGTGCTACGGAAAGGACCAGGCTCTGTGGCAGAAGCTGGCCAAGGA |
| | | CATCCAGGTGGCCAGCAACGAGATCCTGCGGGACCCTTGCTTCCACC |
| | | CCGGCTACAAGAAAGTCGTGAACGTGTCCGACCTGTACAAGACCCCC |
| | | TGCACCAAGAGATTCGAGATGACCCTGCCCTTCCAGCAGTTCGAGATC |
| | | CAGGGCATCGGCAATTACCAGCAGTGCCACCAGAGCATCCTGGAACT |
| | | GTTCAACACCAGCTACTGCCCCTACAGCCAGTGCGCCTTCAACGGCA |
| | | TCTTCCTGCCACCTCTGCAGGGGGATTTCGGCGCCTTCAGCGCCTTC |
| | | TACTCCGTGATGAAGTTCCTGAACCTGACCAGCGAGAAGGTGTCCCA |
| | | GGAAAAAGTGACAGAGATGATGAAGAAGTTCTGCGCCCAGCCCTGGG |
| | | AGGAAATCAAGACCTCCTACGCTGGCGTGAAAGAGAAGTACCTGAGC |
| | | GAGTTTTGCTTCAGCGGCACCTACATCCTGAGCCTGCTGCTGCAGGG |
| | | CTACCACTTCACCGCCGATAGCTGGGAGCACATCCACTTCATCGGCAA |
| | | GATTCAGGGCAGCGACGCCGGCTGGACACTGGGTTACATGCTGAATC |
| | | TGACCAACATGATCCCCGCCGAGCAGCCCCTGAGCACACCTCTGAGC |
| | | CACAGCACC |
| EP10xEP19_H436D | | |
| SEQ ID | Amino | TQNKALPENVKYGIVLDAGSSHTSLYIYKWPAEKENDTGVVHQVEECRVK |
| NO: 62 | acid | GPGISKFVQKVNEIGIYLTDCMERAREVIPRSQHQETPVYLGATAGMRLLR |
| | | MESEELADRVLDVVERSLSNYPFDFQGARIITGQEEGAYGWITINYLLGKF |
| | | SQKNQETFGALDLGGASTQVTFVPQNQTIESPDNALQFRLYGKDYNVYT |
| | | HSFLCYGKDQALWQKLAKDIQVASNEILRDPCFHPGYKKVVNVSDLYKTP |
| | | CTKRFEMTLPFQQFEIQGTGNYQQCHQSILELFNTSYCPYSQCAFNGIFL |
| | | PPLQGDFGAFSAFYFVMKFLNLTSEKVSQEKVTEMMKKFCAQPWEEIKT |
| | | SYAGVKEKYLSEYCFSGTYILSLLQQGYHFTADSWEDIHFIGKIQGSDAG |
| | | WTLGYMLNLTNMIPAEQPLSTPLSHST |
| SEQ ID | DNA | ACCCAGAACAAGGCCCTGCCCGAGAACGTGAAGTACGGCATCGTGCT |
| NO: 63 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCAGGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGCTGGACGTGGTGGAAAGAAGCCTGAGCAACTACCCATTCGAT |
| | | TTTCAAGGCGCCAGAATCATCACCGGCCAGGAAGAAGGCGCCTACGG |
| | | CTGGATCACCATCAACTACCTGCTGGGCAAGTTCAGCCAGAAGAATCA |
| | | GGAAACCTTCGGCGCCCTGGACCTGGGCGGAGCTTCTACCCAAGTGA |
| | | CCTTCGTGCCCCAGAATCAGACCATCGAGAGCCCCGACAACGCCCTG |
| | | CAGTTCCGGCTGTACGGCAAGGACTACAATGTGTACACCCACAGCTTT |
| | | CTGTGCTACGGAAAGGACCAGGCTCTGTGGCAGAAGCTGGCCAAGGA |
| | | CATCCAGGTGGCCAGCAACGAGATCCTGCGGGACCCTTGCTTCCACC |
| | | CCGGCTACAAGAAAGTCGTGAACGTGTCCGACCTGTACAAGACCCCC |
| | | TGCACCAAGAGATTCGAGATGACCCTGCCCTTCCAGCAGTTCGAGATC |
| | | CAGGGCACCGGCAATTACCAGCAGTGCCACCAGAGCATCCTGGAACT |
| | | GTTCAACACCAGCTACTGCCCCTACAGCCAGTGCGCCTTCAACGGCA |
| | | TCTTCCTGCCACCTCTGCAGGGGGATTTCGGCGCCTTCAGCGCCTTC |
| | | TACTTCGTGATGAAGTTCCTGAACCTGACCAGCGAGAAGGTGTCCCAG |
| | | GAAAAAGTGACAGAGATGATGAAGAAGTTCTGCGCCCAGCCCTGGGA |
| | | GGAAATCAAGACCTCCTACGCTGGCGTGAAAGAGAAGTACCTGAGCG |
| | | AGTACTGCTTCAGCGGCACCTACATCCTGAGCCTGCTGCAGCAGGGC |
| | | TACCACTTCACCGCCGATAGCTGGGAGGACATCCACTTCATCGGCAA |
| | | GATTCAGGGCAGCGACGCCGGCTGGACACTGGGCTACATGCTGAATC |
| | | TGACCAACATGATCCCCGCCGAGCAGCCCCTGAGCACACCTCTGAGC |
| | | CACAGCACC |
| EP14xEP19 | | |
| SEQ ID | Amino | TQNKALPENVKYGIVLDAGSSHTSLYIYKWPAEKENDTGVVHQVEECRVK |
| NO: 64 | acid | GPGISKFVQKVNEIGIYLTDCMERAREVIPRSQHQETPVYLGATAGMRLLR |
| | | MESEELADRVLDVVERSLSNYPFDFQGARIITGQEEGAYGWITINYLLGKF |
| | | SQKNQETFGALDLGGASTQVTFVPQNQTIESPDNALQFRLYGKDYNVYT |
| | | HSFLCYGKDQALWQKLAKDIQVASNEILRDPCFHPGYKKVVNVSDLYKTP |
| | | CTKRFEMTLPFQQFEIQGIGNYQQCHQSILELFNTSYCPYSQCAFNGIFLP |
| | | PLQGDFGAFSAFYSVMKFLNLTSEKVSQEKVTEMMKKFCAQPWEEIKTS |
| | | YAGVKEKYLSEYCFSGTYILSLLQQGYHFTADSWEHIHFIGKIQGSDAGW |
| | | TLGYMLNLTNMIPAEQPLSTPLSHST |
| SEQ ID | DNA | ACCCAGAACAAGGCCCTGCCCGAGAACGTGAAGTACGGCATCGTGCT |
| NO: 65 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCAGGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGCTGGACGTGGTGGAAAGAAGCCTGAGCAACTACCCATTCGAT |
| | | TTTCAAGGCGCCAGAATCATCACCGGCCAGGAAGAAGGCGCCTACGG |
| | | CTGGATCACCATCAACTACCTGCTGGGCAAGTTCAGCCAGAAGAATCA |
| | | GGAAACCTTCGGCGCCTTGGACCTGGGCGGAGCTTCTACCCAAGTGA |
| | | CCTTCGTGCCCCAGAATCAGACCATCGAGAGCCCCGACAACGCCCTG |
| | | CAGTTCCGGCTGTACGGCAAGGACTACAATGTGTACACCCACAGCTTT |
| | | CTGTGCTACGGAAAGGACCAGGCTCTGTGGCAGAAGCTGGCCAAGGA |
| | | CATCCAGGTGGCCAGCAACGAGATCCTGCGGGACCCTTGCTTCCACC |
| | | CCGGCTACAAGAAAGTCGTGAACGTGTCCGACCTGTACAAGACCCCC |
| | | TGCACCAAGAGATTCGAGATGACCCTGCCCTTCCAGCAGTTCGAGATC |
| | | CAGGGCATCGGCAATTACCAGCAGTGCCACCAGAGCATCCTGGAACT |
| | | GTTCAACACCAGCTACTGCCCCTACAGCCAGTGCGCCTTCAACGGCA |
| | | TCTTCCTGCCACCTCTGCAGGGGGATTTCGGCGCCTTCAGCGCCTTC |
| | | TACTCCGTGATGAAGTTCCTGAACCTGACCAGCGAGAAGGTGTCCCA |
| | | GGAAAAAGTGACAGAGATGATGAAGAAGTTCTGCGCCCAGCCCTGGG |
| | | AGGAAATCAAGACCTCCTACGCTGGCGTGAAAGAGAAGTACCTGAGC |
| | | GAGTACTGCTTCAGCGGCACCTACATCCTGAGCCTGCTGCAGCAGGG |
| | | CTACCACTTCACCGCCGATAGCTGGGAGCACATCCACTTCATCGGCAA |
| | | GATTCAGGGCAGCGACGCCGGCTGGACACTGGGTTACATGCTGAATC |
| | | TGACCAACATGATCCCCGCCGAGCAGCCCCTGAGCACACCTCTGAGC |
| | | CACAGCACC |
| EP28aa1-15 | | |
| SEQ ID | Amino | APTSSSTKKTQLTSSTQNKALPENVKYGIVLDAGSSHTSLYIYKWPAEKEN |
| NO: 66 | acid | DTGVVHQVEECRVKGPGISKFVQKVNEIGIYLTDCMERAREVIPRSQHQE |
| | | TPVYLGATAGMRLLRMESEELADRVLDVVERSLSNYPFDFQGARIITGQE |
| | | EGAYGWITINYLLGKFSQKNQETFGALDLGGASTQVTFVPQNQTIESPDN |
| | | ALQFRLYGKDYNVYTHSFLCYGKDQALWQKLAKDIQVASNEILRDPCFHP |
| | | GYKKVVNVSDLYKTPCTKRFEMTLPFQQFEIQGIGNYQQCHQSILELFNT |
| | | SYCPYSQCAFNGIFLPPLQGDFGAFSAFYFVMKFLNLTSEKVSQEKVTEM |
| | | MKKFCAQPWEEIKTSYAGVKEKYLSEYCFSGTYILSLLLQGYHFTADSWE |
| | | HIHFIGKIQGSDAGVVTLGYMLNLTNMIPAEQPLSTPLSHST |
| SEQ ID | DNA | GCCCCTACCAGCAGCAGCACCAAGAAAACCCAGCTGACCAGCAGCAC |
| NO: 67 | | CCAGAACAAGGCCCTGCCCGAGAACGTGAAGTACGGCATCGTGCTGG |
| | | ATGCCGGCAGCAGCCACACCAGCCTGTACATCTACAAGTGGCCTGCC |
| | | GAGAAAGAAAACGACACCGGCGTGGTGCATCAGGTGGAAGAGTGCAG |
| | | AGTGAAGGGCCCTGGCATCAGCAAGTTCGTGCAGAAAGTGAACGAGA |
| | | TCGGCATCTACCTGACCGACTGCATGGAACGGGCCAGGGAAGTGATC |
| | | CCCAGAAGCCAGCACCAGGAAACCCCCGTGTATCTGGGAGCCACCGC |
| | | CGGCATGAGACTGCTGAGAATGGAAAGCGAGGAACTGGCCGACCGG |
| | | GTGCTGGACGTGGTGGAAAGAAGCCTGAGCAACTACCCATTCGATTTT |
| | | CAAGGCGCCAGAATCATCACCGGCCAGGAAGAAGGCGCCTACGGCT |
| | | GGATCACCATCAACTACCTGCTGGGCAAGTTCAGCCAGAAGAATCAG |
| | | GAAACCTTCGGCGCCCTGGACCTGGGCGGAGCTTCTACCCAAGTGAC |
| | | CTTCGTGCCCCAGAATCAGACCATCGAGAGCCCCGACAACGCCCTGC |
| | | AGTTCCGGCTGTACGGCAAGGACTACAATGTGTACACCCACAGCTTTC |
| | | TGTGCTACGGAAAGGACCAGGCTCTGTGGCAGAAGCTGGCCAAGGAC |
| | | ATCCAGGTGGCCAGCAACGAGATCCTGCGGGACCCTTGCTTCCACCC |
| | | CGGCTACAAGAAAGTCGTGAACGTGTCCGACCTGTACAAGACCCCCT |
| | | GCACCAAGAGATTCGAGATGACCCTGCCCTTCCAGCAGTTCGAGATC |
| | | CAGGGCATCGGCAATTACCAGCAGTGCCACCAGAGCATCCTGGAACT |
| | | GTTCAACACCAGCTACTGCCCCTACAGCCAGTGCGCCTTCAACGGCA |
| | | TCTTCCTGCCACCTCTGCAGGGGGATTTCGGCGCCTTCAGCGCCTTC |
| | | TACTTCGTGATGAAGTTCCTGAACCTGACCAGCGAGAAGGTGTCCCAG |
| | | GAAAAAGTGACAGAGATGATGAAGAAGTTCTGCGCCCAGCCCTGGGA |
| | | GGAAATCAAGACCTCCTACGCTGGCGTGAAAGAGAAGTACCTGAGCG |
| | | AGTACTGCTTCAGCGGCACCTACATCCTGAGCCTGCTGCTGCAGGGC |
| | | TACCACTTCACCGCCGATAGCTGGGAGCACATCCACTTCATCGGCAA |
| | | GATTCAGGGCAGCGACGCCGGCTGGACACTGGGCTACATGCTGAATC |
| | | TGACCAACATGATCCCCGCCGAGCAGCCCCTGAGCACACCTCTGAGC |
| | | CACAGCACC |
| EP17xE19_H436D | | |
| SEQ ID | Amino | TQNKALPENVKYGIVLDAGSSHTSLYIYKWPAEKENDTGVVHQVEECRVK |
| NO: 68 | acid | GPGISKFVQKVNEIGIYLTDCMERAREVIPRSQHQETPVYLGATAGMRLLR |
| | | MESEELADRVLDVVERSLSNYPFDFQGARIITGQEEGAYGWITINYLLGKF |
| | | SQKNQETFGALDLGGASTQVTFVPQNQTIESPDNALQFRLYGKDYNVYT |
| | | HSFLCYGKDQALWQKLAKDIQVASNEILRDPCFHPGYKKVVNVSDLYKTP |
| | | CTKRFEMTLPFQQFEIQGIGNYQQCHQSILELFNTSYCPYSQCAFNGIFLP |
| | | PLQGDFGAFSAFYFVMKFLNLTSEKVSQEKVTEMMKKFCAQPWEEIKTS |
| | | YAGVKEKYLSEFCFSGTYILSLLQQGYHFTADSWEDIHFIGKIQGSDAGWT |
| | | LGYMLNLTNMIPAEQPLSTPLSHST |
| SEQ ID | DNA | ACCCAGAACAAGGCCCTGCCCGAGAACGTGAAGTACGGCATCGTGCT |
| NO: 69 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCAGGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGCTGGACGTGGTGGAAAGAAGCCTGAGCAACTACCCATTCGAT |
| | | TTTCAAGGCGCCAGAATCATCACCGGCCAGGAAGAAGGCGCCTACGG |
| | | CTGGATCACCATCAACTACCTGCTGGGCAAGTTCAGCCAGAAGAATCA |
| | | GGAAACCTTCGGCGCCCTGGACCTGGGCGGAGCTTCTACCCAAGTGA |
| | | CCTTCGTGCCCCAGAATCAGACCATCGAGAGCCCCGACAACGCCCTG |
| | | CAGTTCCGGCTGTACGGCAAGGACTACAATGTGTACACCCACAGCTTT |
| | | CTGTGCTACGGAAAGGACCAGGCTCTGTGGCAGAAGCTGGCCAAGGA |
| | | CATCCAGGTGGCCAGCAACGAGATCCTGCGGGACCCTTGCTTCCACC |
| | | CCGGCTACAAGAAAGTCGTGAACGTGTCCGACCTGTACAAGACCCCC |
| | | TGCACCAAGAGATTCGAGATGACCCTGCCCTTCCAGCAGTTCGAGATC |
| | | CAGGGCATCGGCAATTACCAGCAGTGCCACCAGAGCATCCTGGAACT |
| | | GTTCAACACCAGCTACTGCCCCTACAGCCAGTGCGCCTTCAACGGCA |
| | | TCTTCCTGCCACCTCTGCAGGGGGATTTCGGCGCCTTCAGCGCCTTC |
| | | TACTTCGTGATGAAGTTCCTGAACCTGACCAGCGAGAAGGTGTCCCAG |
| | | GAAAAAGTGACAGAGATGATGAAGAAGTTCTGCGCCCAGCCCTGGGA |
| | | GGAAATCAAGACCTCCTACGCTGGCGTGAAAGAGAAGTACCTGAGCG |
| | | AGTTTTGCTTCAGCGGCACCTACATCCTGAGCCTGCTGCAGCAGGGC |
| | | TACCACTTCACCGCCGATAGCTGGGAGGACATCCACTTCATCGGCAA |
| | | GATTCAGGGCAGCGACGCCGGCTGGACACTGGGCTACATGCTGAATC |
| | | TGACCAACATGATCCCCGCCGAGCAGCCCCTGAGCACACCTCTGAGC |
| | | CACAGCACC |
| EP17xEP19 | | |
| SEQ ID | Amino | TQNKALPENVKYGIVLDAGSSHTSLYIYKWPAEKENDTGVVHQVEECRVK |
| NO: 70 | acid | GPGISKFVQKVNEIGIYLTDCMERAREVIPRSQHQETPVYLGATAGMRLLR |
| | | MESEELADRVLDVVERSLSNYPFDFQGARIITGQEEGAYGWITINYLLGKF |
| | | SQKNQETFGALDLGGASTQVTFVPQNQTIESPDNALQFRLYGKDYNVYT |
| | | HSFLCYGKDQALWQKLAKDIQVASNEILRDPCFHPGYKKVVNVSDLYKTP |
| | | CTKRFEMTLPFQQFEIQGIGNYQQCHQSILELFNTSYCPYSQCAFNGIFLP |
| | | PLQGDFGAFSAFYFVMKFLNLTSEKVSQEKVTEMMKKFCAQPWEEIKTS |
| | | YAGVKEKYLSEFCFSGTYILSLLQQGYHFTADSWEHIHFIGKIQGSDAGVVT |
| | | LGYMLNLTNMIPAEQPLSTPLSHST |
| SEQ ID | DNA | ACCCAGAACAAGGCCCTGCCCGAGAACGTGAAGTACGGCATCGTGCT |
| NO: 71 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCAGGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGCTGGACGTGGTGGAAAGAAGCCTGAGCAACTACCCATTCGAT |
| | | TTTCAAGGCGCCAGAATCATCACCGGCCAGGAAGAAGGCGCCTACGG |
| | | CTGGATCACCATCAACTACCTGCTGGGCAAGTTCAGCCAGAAGAATCA |
| | | GGAAACCTTCGGCGCCCTGGACCTGGGCGGAGCTTCTACCCAAGTGA |
| | | CCTTCGTGCCCCAGAATCAGACCATCGAGAGCCCCGACAACGCCCTG |
| | | CAGTTCCGGCTGTACGGCAAGGACTACAATGTGTACACCCACAGCTTT |
| | | CTGTGCTACGGAAAGGACCAGGCTCTGTGGCAGAAGCTGGCCAAGGA |
| | | CATCCAGGTGGCCAGCAACGAGATCCTGCGGGACCCTTGCTTCCACC |
| | | CCGGCTACAAGAAAGTCGTGAACGTGTCCGACCTGTACAAGACCCCC |
| | | TGCACCAAGAGATTCGAGATGACCCTGCCCTTCCAGCAGTTCGAGATC |
| | | CAGGGCATCGGCAATTACCAGCAGTGCCACCAGAGCATCCTGGAACT |
| | | GTTCAACACCAGCTACTGCCCCTACAGCCAGTGCGCCTTCAACGGCA |
| | | TCTTCCTGCCACCTCTGCAGGGGGATTTCGGCGCCTTCAGCGCCTTC |
| | | TACTTCGTGATGAAGTTCCTGAACCTGACCAGCGAGAAGGTGTCCCAG |
| | | GAAAAAGTGACAGAGATGATGAAGAAGTTCTGCGCCCAGCCCTGGGA |
| | | GGAAATCAAGACCTCCTACGCTGGCGTGAAAGAGAAGTACCTGAGCG |
| | | AGTTTTGCTTCAGCGGCACCTACATCCTGAGCCTGCTGCAGCAGGGC |
| | | TACCACTTCACCGCCGATAGCTGGGAGCACATCCACTTCATCGGCAA |
| | | GATTCAGGGCAGCGACGCCGGCTGGACACTGGGCTACATGCTGAATC |
| | | TGACCAACATGATCCCCGCCGAGCAGCCCCTGAGCACACCTCTGAGC |
| | | CACAGCACC |
| EP28aa1-16 | | |
| SEQ ID | Amino | APTSSSTKKTQLTSSGTQNKALPENVKYGIVLDAGSSHTSLYIYKWPAEKE |
| NO: 72 | acid | NDTGVVHQVEECRVKGPGISKFVQKVNEIGIYLTDCMERAREVIPRSQHQ |
| | | ETPVYLGATAGMRLLRMESEELADRVLDVVERSLSNYPFDFQGARIITGQ |
| | | EEGAYGWITINYLLGKFSQKNQETFGALDLGGASTQVTFVPQNQTIESPD |
| | | NALQFRLYGKDYNVYTHSFLCYGKDQALWQKLAKDIQVASNEILRDPCFH |
| | | PGYKKVVNVSDLYKTPCTKRFEMTLPFQQFEIQGIGNYQQCHQSILELFN |
| | | TSYCPYSQCAFNGIFLPPLQGDFGAFSAFYFVMKFLNLTSEKVSQEKVTE |
| | | MMKKFCAQPWEEIKTSYAGVKEKYLSEYCFSGTYILSLLLQGYHFTADSW |
| | | EHIHFIGKIQGSDAGVVTLGYMLNLTNMIPAEQPLSTPLSHST |
| SEQ ID | DNA | GCCCCTACCTCCTCCAGCACCAAGAAAACCCAGCTGACCTCCAGCGG |
| NO: 73 | | CACCCAGAACAAGGCCCTGCCCGAGAACGTGAAGTACGGCATCGTGC |
| | | TGGACGCCGGCTCCTCCCACACCTCCCTGTACATCTACAAGTGGCCT |
| | | GCCGAGAAAGAAAACGACACCGGCGTGGTGCACCAAGTGGAAGAGT |
| | | GCAGAGTGAAGGGCCCCGGCATCTCCAAGTTCGTGCAGAAAGTGAAC |
| | | GAGATCGGCATCTACCTGACCGACTGCATGGAACGGGCCAGAGAAGT |
| | | GATCCCTCGGTCCCAGCACCAGGAAACCCCTGTCTACCTGGGCGCCA |
| | | CCGCCGGCATGCGGCTGCTGCGGATGGAATCCGAGGAACTGGCCGA |
| | | CCGGGTGCTGGACGTGGTGGAACGGTCCCTGTCCAACTACCCATTCG |
| | | ATTTTCAAGGCGCCAGAATCATCACCGGCCAGGAAGAGGGCGCCTAC |
| | | GGCTGGATCACCATCAACTACCTGCTGGGCAAGTTCTCCCAGAAGAAT |
| | | CAGGAAACCTTCGGCGCCCTGGACCTGGGCGGAGCCAGCACCCAAG |
| | | TCACATTCGTGCCCCAGAACCAGACCATCGAGAGCCCCGACAACGCC |
| | | CTGCAGTTCCGGCTGTACGGCAAGGACTACAACGTGTACACCCACAG |
| | | CTTTCTGTGCTACGGCAAGGACCAGGCCCTGTGGCAGAAGCTGGCCA |
| | | AGGACATCCAAGTGGCCTCCAACGAGATCCTGCGGGACCCCTGCTTC |
| | | CACCCCGGCTACAAGAAAGTGGTCAACGTGTCCGACCTGTACAAGAC |
| | | CCCTTGCACCAAGAGATTCGAGATGACCCTGCCCTTCCAGCAGTTCGA |
| | | GATCCAGGGCATCGGCAACTACCAGCAGTGCCACCAGTCCATCCTGG |
| | | AACTGTTCAACACCTCCTACTGCCCCTACTCCCAGTGCGCCTTCAACG |
| | | GCATCTTCCTGCCTCCACTGCAGGGCGACTTCGGCGCCTTCTCCGCC |
| | | TTCTACTTCGTGATGAAGTTCCTGAACCTGACCTCCGAGAAAGTGTCC |
| | | CAGGAAAAAGTGACCGAGATGATGAAGAAGTTCTGCGCCCAGCCCTG |
| | | GGAGGAAATCAAGACCTCCTACGCTGGCGTGAAAGAGAAGTACCTGT |
| | | CCGAGTACTGCTTCTCCGGCACCTACATCCTGTCCCTGCTGCTGCAG |
| | | GGCTACCACTTCACCGCCGACAGCTGGGAGCACATCCACTTCATCGG |
| | | CAAGATCCAGGGATCCGACGCTGGCTGGACCCTGGGCTACATGCTGA |
| | | ATCTGACCAACATGATCCCCGCCGAGCAGCCCCTGTCCACCCCTCTG |
| | | TCTCACTCCACC |
| EP1xEP14xEP19 | | |
| SEQ ID | Amino | TQNKALPENVKYGIVLDAGSSHTSLYIYKWPAEKENDTGVVHQVEECRVK |
| NO: 74 | acid | GPGISKFVQKVNEIGIYLTDCMERAMEVIPRSQHQETPVYLGATAGMRLL |
| | | RMESEELADRVLDVVERSLSNYPFDFQGARIITGQEEGAYGWITINYLLGK |
| | | FSQKNQETFGALDLGGASTQVTFVPQNQTIESPDNALQFRLYGKDYNVY |
| | | THSFLCYGKDQALWQKLAKDIQVASNEILRDPCFHPGYKKVVNVSDLYKT |
| | | PCTKRFEMTLPFQQFEIQGIGNYQQCHQSILELFNTSYCPYSQCAFNGIFL |
| | | PPLQGDFGAFSAFYSVMKFLNLTSEKVSQEKVTEMMKKFCAQPWEEIKT |
| | | SYAGVKEKYLSEYCFSGTYILSLLQQGYHFTADSWEHIHFIGKIQGSDAG |
| | | WTLGYMLNLTNMIPAEQPLSTPLSHST |
| SEQ ID | DNA | ACCCAGAACAAGGCCCTGCCCGAGAACGTGAAGTACGGCATCGTGCT |
| NO: 75 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCATGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGCTGGACGTGGTGGAAAGAAGCCTGAGCAACTACCCATTCGAT |
| | | TTTCAAGGCGCCAGAATCATCACCGGCCAGGAAGAAGGCGCCTACGG |
| | | CTGGATCACCATCAACTACCTGCTGGGCAAGTTCAGCCAGAAGAATCA |
| | | GGAAACCTTCGGCGCCCTGGACCTGGGCGGAGCTTCTACCCAAGTGA |
| | | CCTTCGTGCCCCAGAATCAGACCATCGAGAGCCCCGACAACGCCCTG |
| | | CAGTTCCGGCTGTACGGCAAGGACTACAATGTGTACACCCACAGCTTT |
| | | CTGTGCTACGGAAAGGACCAGGCTCTGTGGCAGAAGCTGGCCAAGGA |
| | | CATCCAGGTGGCCAGCAACGAGATCCTGCGGGACCCTTGCTTCCACC |
| | | CCGGCTACAAGAAAGTCGTGAACGTGTCCGACCTGTACAAGACCCCC |
| | | TGCACCAAGAGATTCGAGATGACCCTGCCCTTCCAGCAGTTCGAGATC |
| | | CAGGGCATCGGCAATTACCAGCAGTGCCACCAGAGCATCCTGGAACT |
| | | GTTCAACACCAGCTACTGCCCCTACAGCCAGTGCGCCTTCAACGGCA |
| | | TCTTCCTGCCACCTCTGCAGGGGGATTTCGGCGCCTTCAGCGCCTTC |
| | | TACTCCGTGATGAAGTTCCTGAACCTGACCAGCGAGAAGGTGTCCCA |
| | | GGAAAAAGTGACAGAGATGATGAAGAAGTTCTGCGCCCAGCCCTGGG |
| | | AGGAAATCAAGACCTCCTACGCTGGCGTGAAAGAGAAGTACCTGAGC |
| | | GAGTACTGCTTCAGCGGCACCTACATCCTGAGCCTGCTGCAGCAGGG |
| | | CTACCACTTCACCGCCGATAGCTGGGAGCACATCCACTTCATCGGCAA |
| | | GATTCAGGGCAGCGACGCCGGCTGGACACTGGGCTACATGCTGAATC |
| | | TGACCAACATGATCCCCGCCGAGCAGCCCCTGAGCACACCTCTGAGC |
| | | CACAGCACC |
| EP1xEP17xEP19 | | |
| SEQ ID | Amino | TQNKALPENVKYGIVLDAGSSHTSLYIYKWPAEKENDTGVVHQVEECRVK |
| NO: 76 | acid | GPGISKFVQKVNEIGIYLTDCMERAMEVIPRSQHQETPVYLGATAGMRLL |
| | | RMESEELADRVLDVVERSLSNYPFDFQGARIITGQEEGAYGWITINYLLGK |
| | | FSQKNQETFGALDLGGASTQVTFVPQNQTIESPDNALQFRLYGKDYNVY |
| | | THSFLCYGKDQALWQKLAKDIQVASNEILRDPCFHPGYKKVVNVSDLYKT |
| | | PCTKRFEMTLPFQQFEIQGIGNYQQCHQSILELFNTSYCPYSQCAFNGIFL |
| | | PPLQGDFGAFSAFYFVMKFLNLTSEKVSQEKVTEMMKKFCAQPWEEIKT |
| | | SYAGVKEKYLSEFCFSGTYILSLLQQGYHFTADSWEHIHFIGKIQGSDAG |
| | | VVTLGYMLNLTNMIPAEQPLSTPLSHST |
| SEQ ID | DNA | ACCCAGAACAAGGCCCTGCCCGAGAACGTGAAGTACGGCATCGTGCT |
| NO: 77 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCATGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGCTGGACGTGGTGGAAAGAAGCCTGAGCAACTACCCATTCGAT |
| | | TTTCAAGGCGCCAGAATCATCACCGGCCAGGAAGAAGGCGCCTACGG |
| | | CTGGATCACCATCAACTACCTGCTGGGCAAGTTCAGCCAGAAGAATCA |
| | | GGAAACCTTCGGCGCCCTGGACCTGGGCGGAGCTTCTACCCAAGTGA |
| | | CCTTCGTGCCCCAGAATCAGACCATCGAGAGCCCCGACAACGCCCTG |
| | | CAGTTCCGGCTGTACGGCAAGGACTACAATGTGTACACCCACAGCTTT |
| | | CTGTGCTACGGAAAGGACCAGGCTCTGTGGCAGAAGCTGGCCAAGGA |
| | | CATCCAGGTGGCCAGCAACGAGATCCTGCGGGACCCTTGCTTCCACC |
| | | CCGGCTACAAGAAAGTCGTGAACGTGTCCGACCTGTACAAGACCCCC |
| | | TGCACCAAGAGATTCGAGATGACCCTGCCCTTCCAGCAGTTCGAGATC |
| | | CAGGGCATCGGCAATTACCAGCAGTGCCACCAGAGCATCCTGGAACT |
| | | GTTCAACACCAGCTACTGCCCCTACAGCCAGTGCGCCTTCAACGGCA |
| | | TCTTCCTGCCACCTCTGCAGGGGGATTTCGGCGCCTTCAGCGCCTTC |
| | | TACTTCGTGATGAAGTTCCTGAACCTGACCAGCGAGAAGGTGTCCCAG |
| | | GAAAAAGTGACAGAGATGATGAAGAAGTTCTGCGCCCAGCCCTGGGA |
| | | GGAAATCAAGACCTCCTACGCTGGCGTGAAAGAGAAGTACCTGAGCG |
| | | AGTTCTGCTTCAGCGGCACCTACATCCTGAGCCTGCTGCAGCAGGGC |
| | | TACCACTTCACCGCCGATAGCTGGGAGCACATCCACTTCATCGGCAA |
| | | GATTCAGGGCAGCGACGCCGGCTGGACACTGGGCTACATGCTGAATC |
| | | TGACCAACATGATCCCCGCCGAGCAGCCCCTGAGCACACCTCTGAGC |
| | | CACAGCACC |
| EP1xEP14 | | |
| SEQ ID | Amino | TQNKALPENVKYGIVLDAGSSHTSLYIYKWPAEKENDTGVVHQVEECRVK |
| NO: 78 | acid | GPGISKFVQKVNEIGIYLTDCMERAMEVIPRSQHQETPVYLGATAGMRLL |
| | | RMESEELADRVLDVVERSLSNYPFDFQGARIITGQEEGAYGWITINYLLGK |
| | | FSQKNQETFGALDLGGASTQVTFVPQNQTIESPDNALQFRLYGKDYNVY |
| | | THSFLCYGKDQALWQKLAKDIQVASNEILRDPCFHPGYKKVVNVSDLYKT |
| | | PCTKRFEMTLPFQQFEIQGIGNYQQCHQSILELFNTSYCPYSQCAFNGIFL |
| | | PPLQGDFGAFSAFYSVMKFLNLTSEKVSQEKVTEMMKKFCAQPWEEIKT |
| | | SYAGVKEKYLSEYCFSGTYILSLLLQGYHFTADSWEHIHFIGKIQGSDAGW |
| | | TLGYMLNLTNMIPAEQPLSTPLSHST |
| SEQ ID | DNA | ACCCAGAACAAGGCCCTGCCCGAGAACGTGAAGTACGGCATCGTGCT |
| NO: 79 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCATGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGCTGGACGTGGTGGAAAGAAGCCTGAGCAACTACCCATTCGAT |
| | | TTTCAAGGCGCCAGAATCATCACCGGCCAGGAAGAAGGCGCCTACGG |
| | | CTGGATCACCATCAACTACCTGCTGGGCAAGTTCAGCCAGAAGAATCA |
| | | GGAAACCTTCGGCGCCTTGGACCTGGGCGGAGCTTCTACCCAAGTGA |
| | | CCTTCGTGCCCCAGAATCAGACCATCGAGAGCCCCGACAACGCCCTG |
| | | CAGTTCCGGCTGTACGGCAAGGACTACAATGTGTACACCCACAGCTTT |
| | | CTGTGCTACGGAAAGGACCAGGCTCTGTGGCAGAAGCTGGCCAAGGA |
| | | CATCCAGGTGGCCAGCAACGAGATCCTGCGGGACCCTTGCTTCCACC |
| | | CCGGCTACAAGAAAGTCGTGAACGTGTCCGACCTGTACAAGACCCCC |
| | | TGCACCAAGAGATTCGAGATGACCCTGCCCTTCCAGCAGTTCGAGATC |
| | | CAGGGCATCGGCAATTACCAGCAGTGCCACCAGAGCATCCTGGAACT |
| | | GTTCAACACCAGCTACTGCCCCTACAGCCAGTGCGCCTTCAACGGCA |
| | | TCTTCCTGCCACCTCTGCAGGGGGATTTCGGCGCCTTCAGCGCCTTC |
| | | TACTCCGTGATGAAGTTCCTGAACCTGACCAGCGAGAAGGTGTCCCA |
| | | GGAAAAAGTGACAGAGATGATGAAGAAGTTCTGCGCCCAGCCCTGGG |
| | | AGGAAATCAAGACCTCCTACGCTGGCGTGAAAGAGAAGTACCTGAGC |
| | | GAGTACTGCTTCAGCGGCACCTACATCCTGAGCCTGCTGCTGCAGGG |
| | | CTACCACTTCACCGCCGATAGCTGGGAGCACATCCACTTCATCGGCAA |
| | | GATTCAGGGCAGCGACGCCGGCTGGACACTGGGTTACATGCTGAATC |
| | | TGACCAACATGATCCCCGCCGAGCAGCCCCTGAGCACACCTCTGAGC |
| | | CACAGCACC |
| EP28aa1-6 | | |
| SEQ ID | Amino | APTSSSTQNKALPENVKYGIVLDAGSSHTSLYIYKWPAEKENDTGVVHQV |
| NO: 80 | acid | EECRVKGPGISKFVQKVNEIGIYLTDCMERAREVIPRSQHQETPVYLGATA |
| | | GMRLLRMESEELADRVLDVVERSLSNYPFDFQGARIITGQEEGAYGWITI |
| | | NYLLGKFSQKNQETFGALDLGGASTQVTFVPQNQTIESPDNALQFRLYGK |
| | | DYNVYTHSFLCYGKDQALWQKLAKDIQVASNEILRDPCFHPGYKKVVNVS |
| | | DLYKTPCTKRFEMTLPFQQFEIQGIGNYQQCHQSILELFNTSYCPYSQCA |
| | | FNGIFLPPLQGDFGAFSAFYFVMKFLNLTSEKVSQEKVTEMMKKFCAQP |
| | | WEEIKTSYAGVKEKYLSEYCFSGTYILSLLLQGYHFTADSWEHIHFIGKIQG |
| | | SDAGWTLGYMLNLTNMIPAEQPLSTPLSHST |
| SEQ ID | DNA | GCCCCTACCAGCAGCAGCACCCAGAACAAGGCCCTGCCCGAGAACGT |
| NO: 81 | | GAAGTACGGCATCGTGCTGGATGCCGGCAGCAGCCACACCAGCCTGT |
| | | ACATCTACAAGTGGCCTGCCGAGAAAGAAAACGACACCGGCGTGGTG |
| | | CATCAGGTGGAAGAGTGCAGAGTGAAGGGCCCTGGCATCAGCAAGTT |
| | | CGTGCAGAAAGTGAACGAGATCGGCATCTACCTGACCGACTGCATGG |
| | | AACGGGCCAGGGAAGTGATCCCCAGAAGCCAGCACCAGGAAACCCC |
| | | CGTGTATCTGGGAGCCACCGCCGGCATGAGACTGCTGAGAATGGAAA |
| | | GCGAGGAACTGGCCGACCGGGTGCTGGACGTGGTGGAAAGAAGCCT |
| | | GAGCAACTACCCATTCGATTTTCAAGGCGCCAGAATCATCACCGGCCA |
| | | GGAAGAAGGCGCCTACGGCTGGATCACCATCAACTACCTGCTGGGCA |
| | | AGTTCAGCCAGAAGAATCAGGAAACCTTCGGCGCCCTGGACCTGGGC |
| | | GGAGCTTCTACCCAAGTGACCTTCGTGCCCCAGAATCAGACCATCGA |
| | | GAGCCCCGACAACGCCCTGCAGTTCCGGCTGTACGGCAAGGACTACA |
| | | ATGTGTACACCCACAGCTTTCTGTGCTACGGAAAGGACCAGGCTCTGT |
| | | GGCAGAAGCTGGCCAAGGACATCCAGGTGGCCAGCAACGAGATCCT |
| | | GCGGGACCCTTGCTTCCACCCCGGCTACAAGAAAGTCGTGAACGTGT |
| | | CCGACCTGTACAAGACCCCCTGCACCAAGAGATTCGAGATGACCCTG |
| | | CCCTTCCAGCAGTTCGAGATCCAGGGCATCGGCAATTACCAGCAGTG |
| | | CCACCAGAGCATCCTGGAACTGTTCAACACCAGCTACTGCCCCTACAG |
| | | CCAGTGCGCCTTCAACGGCATCTTCCTGCCACCTCTGCAGGGGGATT |
| | | TCGGCGCCTTCAGCGCCTTCTACTTCGTGATGAAGTTCCTGAACCTGA |
| | | CCAGCGAGAAGGTGTCCCAGGAAAAAGTGACAGAGATGATGAAGAAG |
| | | TTCTGCGCCCAGCCCTGGGAGGAAATCAAGACCTCCTACGCTGGCGT |
| | | GAAAGAGAAGTACCTGAGCGAGTACTGCTTCAGCGGCACCTACATCC |
| | | TGAGCCTGCTGCTGCAGGGCTACCACTTCACCGCCGATAGCTGGGAG |
| | | CACATCCACTTCATCGGCAAGATTCAGGGCAGCGACGCCGGCTGGAC |
| | | ACTGGGCTACATGCTGAATCTGACCAACATGATCCCCGCCGAGCAGC |
| | | CCCTGAGCACACCTCTGAGCCACAGCACC |
| EP28_E174A | | |
| SEQ ID | Amino | TQNKALPENVKYGIVLDAGSSHTSLYIYKWPAEKENDTGVVHQVEECRVK |
| NO: 82 | acid | GPGISKFVQKVNEIGIYLTDCMERAREVIPRSQHQETPVYLGATAGMRLLR |
| | | MESEELADRVLDVVERSLSNYPFDFQGARIITGQEAGAYGWITINYLLGKF |
| | | SQKNQETFGALDLGGASTQVTFVPQNQTIESPDNALQFRLYGKDYNVYT |
| | | HSFLCYGKDQALWQKLAKDIQVASNEILRDPCFHPGYKKVVNVSDLYKTP |
| | | CTKRFEMTLPFQQFEIQGIGNYQQCHQSILELFNTSYCPYSQCAFNGIFLP |
| | | PLQGDFGAFSAFYFVMKFLNLTSEKVSQEKVTEMMKKFCAQPWEEIKTS |
| | | YAGVKEKYLSEYCFSGTYILSLLLQGYHFTADSWEHIHFIGKIQGSDAGWT |
| | | LGYMLNLTNMIPAEQPLSTPLSHST |
| SEQ ID | DNA | ACCCAGAACAAGGCCCTGCCCGAGAACGTGAAGTACGGCATCGTGCT |
| NO: 83 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCAGGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGCTGGACGTGGTGGAAAGAAGCCTGAGCAACTACCCATTCGAT |
| | | TTTCAAGGCGCCAGAATCATCACCGGCCAGGAAGCCGGCGCCTACGG |
| | | CTGGATCACCATCAACTACCTGCTGGGCAAGTTCAGCCAGAAGAATCA |
| | | GGAAACCTTCGGCGCCCTGGACCTGGGCGGAGCTTCTACCCAAGTGA |
| | | CCTTCGTGCCCCAGAATCAGACCATCGAGAGCCCCGACAACGCCCTG |
| | | CAGTTCCGGCTGTACGGCAAGGACTACAATGTGTACACCCACAGCTTT |
| | | CTGTGCTACGGAAAGGACCAGGCTCTGTGGCAGAAGCTGGCCAAGGA |
| | | CATCCAGGTGGCCAGCAACGAGATCCTGCGGGACCCTTGCTTCCACC |
| | | CCGGCTACAAGAAAGTCGTGAACGTGTCCGACCTGTACAAGACCCCC |
| | | TGCACCAAGAGATTCGAGATGACCCTGCCCTTCCAGCAGTTCGAGATC |
| | | CAGGGCATCGGCAATTACCAGCAGTGCCACCAGAGCATCCTGGAACT |
| | | GTTCAACACCAGCTACTGCCCCTACAGCCAGTGCGCCTTCAACGGCA |
| | | TCTTCCTGCCACCTCTGCAGGGGGATTTCGGCGCCTTCAGCGCCTTC |
| | | TACTTCGTGATGAAGTTCCTGAACCTGACCAGCGAGAAGGTGTCCCAG |
| | | GAAAAAGTGACAGAGATGATGAAGAAGTTCTGCGCCCAGCCCTGGGA |
| | | GGAAATCAAGACCTCCTACGCTGGCGTGAAAGAGAAGTACCTGAGCG |
| | | AGTACTGCTTCAGCGGCACCTACATCCTGAGCCTGCTGCTGCAGGGC |
| | | TACCACTTCACCGCCGATAGCTGGGAGCACATCCACTTCATCGGCAA |
| | | GATTCAGGGCAGCGACGCCGGCTGGACACTGGGCTACATGCTGAATC |
| | | TGACCAACATGATCCCCGCCGAGCAGCCCCTGAGCACACCTCTGAGC |
| | | CACAGCACC |
| EP28_E174A_S218A | | |
| SEQ ID | Amino | TQNKALPENVKYGIVLDAGSSHTSLYIYKWPAEKENDTGVVHQVEECRVK |
| NO: 84 | acid | GPGISKFVQKVNEIGIYLTDCMERAREVIPRSQHQETPVYLGATAGMRLLR |
| | | MESEELADRVLDVVERSLSNYPFDFQGARIITGQEAGAYGWITINYLLGKF |
| | | SQKNQETFGALDLGGAATQVTFVPQNQTIESPDNALQFRLYGKDYNVYT |
| | | HSFLCYGKDQALWQKLAKDIQVASNEILRDPCFHPGYKKVVNVSDLYKTP |
| | | CTKRFEMTLPFQQFEIQGIGNYQQCHQSILELFNTSYCPYSQCAFNGIFLP |
| | | PLQGDFGAFSAFYFVMKFLNLTSEKVSQEKVTEMMKKFCAQPWEEIKTS |
| | | YAGVKEKYLSEYCFSGTYILSLLLQGYHFTADSWEHIHFIGKIQGSDAGWT |
| | | LGYMLNLTNMIPAEQPLSTPLSHST |
| SEQ ID | DNA | ACCCAGAACAAGGCCCTGCCCGAGAACGTGAAGTACGGCATCGTGCT |
| NO: 85 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCAGGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGCTGGACGTGGTGGAAAGAAGCCTGAGCAACTACCCATTCGAT |
| | | TTTCAAGGCGCCAGAATCATCACCGGCCAGGAAGCCGGCGCCTACGG |
| | | CTGGATCACCATCAACTACCTGCTGGGCAAGTTCAGCCAGAAGAATCA |
| | | GGAAACCTTCGGCGCCCTGGACCTGGGCGGAGCTGCTACCCAAGTG |
| | | ACCTTCGTGCCCCAGAATCAGACCATCGAGAGCCCCGACAACGCCCT |
| | | GCAGTTCCGGCTGTACGGCAAGGACTACAATGTGTACACCCACAGCT |
| | | TTCTGTGCTACGGAAAGGACCAGGCTCTGTGGCAGAAGCTGGCCAAG |
| | | GACATCCAGGTGGCCAGCAACGAGATCCTGCGGGACCCTTGCTTCCA |
| | | CCCCGGCTACAAGAAAGTCGTGAACGTGTCCGACCTGTACAAGACCC |
| | | CCTGCACCAAGAGATTCGAGATGACCCTGCCCTTCCAGCAGTTCGAG |
| | | ATCCAGGGCATCGGCAATTACCAGCAGTGCCACCAGAGCATCCTGGA |
| | | ACTGTTCAACACCAGCTACTGCCCCTACAGCCAGTGCGCCTTCAACG |
| | | GCATCTTCCTGCCACCTCTGCAGGGGGATTTCGGCGCCTTCAGCGCC |
| | | TTCTACTTCGTGATGAAGTTCCTGAACCTGACCAGCGAGAAGGTGTCC |
| | | CAGGAAAAAGTGACAGAGATGATGAAGAAGTTCTGCGCCCAGCCCTG |
| | | GGAGGAAATCAAGACCTCCTACGCTGGCGTGAAAGAGAAGTACCTGA |
| | | GCGAGTACTGCTTCAGCGGCACCTACATCCTGAGCCTGCTGCTGCAG |
| | | GGCTACCACTTCACCGCCGATAGCTGGGAGCACATCCACTTCATCGG |
| | | CAAGATTCAGGGCAGCGACGCCGGCTGGACACTGGGCTACATGCTGA |
| | | ATCTGACCAACATGATCCCCGCCGAGCAGCCCCTGAGCACACCTCTG |
| | | AGCCACAGCACC |
| EP14_N73Q | | |
| SEQ ID | Amino | TQNKALPENVKYGIVLDAGSSHTSLYIYKWPAEKEQDTGVVHQVEECRVK |
| NO: 86 | acid | GPGISKFVQKVNEIGIYLTDCMERAREVIPRSQHQETPVYLGATAGMRLLR |
| | | MESEELADRVLDVVERSLSNYPFDFQGARIITGQEEGAYGWITINYLLGKF |
| | | SQKNQETFGALDLGGASTQVTFVPQNQTIESPDNALQFRLYGKDYNVYT |
| | | HSFLCYGKDQALWQKLAKDIQVASNEILRDPCFHPGYKKVVNVSDLYKTP |
| | | CTKRFEMTLPFQQFEIQGIGNYQQCHQSILELFNTSYCPYSQCAFNGIFLP |
| | | PLQGDFGAFSAFYSVMKFLNLTSEKVSQEKVTEMMKKFCAQPWEEIKTS |
| | | YAGVKEKYLSEYCFSGTYILSLLLQGYHFTADSWEHIHFIGKIQGSDAGVVT |
| | | LGYMLNLTNMIPAEQPLSTPLSHST |
| SEQ ID | DNA | ACCCAGAACAAGGCCCTGCCCGAGAACGTGAAGTACGGCATCGTGCT |
| NO: 87 | | GGATGCCGGCAGCAGCCACACCAGCCTGTACATCTACAAGTGGCCTG |
| | | CCGAGAAAGAACAGGACACCGGCGTGGTGCATCAGGTGGAAGAGTG |
| | | CAGAGTGAAGGGCCCTGGCATCAGCAAGTTCGTGCAGAAAGTGAACG |
| | | AGATCGGCATCTACCTGACCGACTGCATGGAACGGGCCAGGGAAGTG |
| | | ATCCCCAGAAGCCAGCACCAGGAAACCCCCGTGTATCTGGGAGCCAC |
| | | CGCCGGCATGAGACTGCTGAGAATGGAAAGCGAGGAACTGGCCGAC |
| | | CGGGTGCTGGACGTGGTGGAAAGAAGCCTGAGCAACTACCCATTCGA |
| | | TTTTCAAGGCGCCAGAATCATCACCGGCCAGGAAGAAGGCGCCTACG |
| | | GCTGGATCACCATCAACTACCTGCTGGGCAAGTTCAGCCAGAAGAATC |
| | | AGGAAACCTTCGGCGCCCTGGACCTGGGCGGAGCTTCTACCCAAGTG |
| | | ACCTTCGTGCCCCAGAATCAGACCATCGAGAGCCCCGACAACGCCCT |
| | | GCAGTTCCGGCTGTACGGCAAGGACTACAATGTGTACACCCACAGCT |
| | | TTCTGTGCTACGGAAAGGACCAGGCTCTGTGGCAGAAGCTGGCCAAG |
| | | GACATCCAGGTGGCCAGCAACGAGATCCTGCGGGACCCTTGCTTCCA |
| | | CCCCGGCTACAAGAAAGTCGTGAACGTGTCCGACCTGTACAAGACCC |
| | | CCTGCACCAAGAGATTCGAGATGACCCTGCCCTTCCAGCAGTTCGAG |
| | | ATCCAGGGCATCGGCAATTACCAGCAGTGCCACCAGAGCATCCTGGA |
| | | ACTGTTCAACACCAGCTACTGCCCCTACAGCCAGTGCGCCTTCAACG |
| | | GCATCTTCCTGCCACCTCTGCAGGGGGATTTCGGCGCCTTCAGCGCC |
| | | TTCTACTCCGTGATGAAGTTCCTGAACCTGACCAGCGAGAAGGTGTCC |
| | | CAGGAAAAAGTGACAGAGATGATGAAGAAGTTCTGCGCCCAGCCCTG |
| | | GGAGGAAATCAAGACCTCCTACGCTGGCGTGAAAGAGAAGTACCTGA |
| | | GCGAGTACTGCTTCAGCGGCACCTACATCCTGAGCCTGCTGCTGCAG |
| | | GGCTACCACTTCACCGCCGATAGCTGGGAGCACATCCACTTCATCGG |
| | | CAAGATTCAGGGCAGCGACGCCGGCTGGACACTGGGCTACATGCTGA |
| | | ATCTGACCAACATGATCCCCGCCGAGCAGCCCCTGAGCACACCTCTG |
| | | AGCCACAGCACC |
| EP14_T229A | | |
| SEQ ID | Amino | TQNKALPENVKYGIVLDAGSSHTSLYIYKWPAEKENDTGVVHQVEECRVK |
| NO: 88 | acid | GPGISKFVQKVNEIGIYLTDCMERAREVIPRSQHQETPVYLGATAGMRLLR |
| | | MESEELADRVLDVVERSLSNYPFDFQGARIITGQEEGAYGWITINYLLGKF |
| | | SQKNQETFGALDLGGASTQVTFVPQNQAIESPDNALQFRLYGKDYNVYT |
| | | HSFLCYGKDQALWQKLAKDIQVASNEILRDPCFHPGYKKVVNVSDLYKTP |
| | | CTKRFEMTLPFQQFEIQGIGNYQQCHQSILELFNTSYCPYSQCAFNGIFLP |
| | | PLQGDFGAFSAFYSVMKFLNLTSEKVSQEKVTEMMKKFCAQPWEEIKTS |
| | | YAGVKEKYLSEYCFSGTYILSLLLQGYHFTADSWEHIHFIGKIQGSDAGWT |
| | | LGYMLNLTNMIPAEQPLSTPLSHST |
| SEQ ID | DNA | ACCCAGAACAAGGCCCTGCCCGAGAACGTGAAGTACGGCATCGTGCT |
| NO: 89 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCAGGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGCTGGACGTGGTGGAAAGAAGCCTGAGCAACTACCCATTCGAT |
| | | TTTCAAGGCGCCAGAATCATCACCGGCCAGGAAGAAGGCGCCTACGG |
| | | CTGGATCACCATCAACTACCTGCTGGGCAAGTTCAGCCAGAAGAATCA |
| | | GGAAACCTTCGGCGCCCTGGACCTGGGCGGAGCTTCTACCCAAGTGA |
| | | CCTTCGTGCCCCAGAATCAGGCCATCGAGAGCCCCGACAACGCCCTG |
| | | CAGTTCCGGCTGTACGGCAAGGACTACAATGTGTACACCCACAGCTTT |
| | | CTGTGCTACGGAAAGGACCAGGCTCTGTGGCAGAAGCTGGCCAAGGA |
| | | CATCCAGGTGGCCAGCAACGAGATCCTGCGGGACCCTTGCTTCCACC |
| | | CCGGCTACAAGAAAGTCGTGAACGTGTCCGACCTGTACAAGACCCCC |
| | | TGCACCAAGAGATTCGAGATGACCCTGCCCTTCCAGCAGTTCGAGATC |
| | | CAGGGCATCGGCAATTACCAGCAGTGCCACCAGAGCATCCTGGAACT |
| | | GTTCAACACCAGCTACTGCCCCTACAGCCAGTGCGCCTTCAACGGCA |
| | | TCTTCCTGCCACCTCTGCAGGGGGATTTCGGCGCCTTCAGCGCCTTC |
| | | TACTCCGTGATGAAGTTCCTGAACCTGACCAGCGAGAAGGTGTCCCA |
| | | GGAAAAAGTGACAGAGATGATGAAGAAGTTCTGCGCCCAGCCCTGGG |
| | | AGGAAATCAAGACCTCCTACGCTGGCGTGAAAGAGAAGTACCTGAGC |
| | | GAGTACTGCTTCAGCGGCACCTACATCCTGAGCCTGCTGCTGCAGGG |
| | | CTACCACTTCACCGCCGATAGCTGGGAGCACATCCACTTCATCGGCAA |
| | | GATTCAGGGCAGCGACGCCGGCTGGACACTGGGCTACATGCTGAATC |
| | | TGACCAACATGATCCCCGCCGAGCAGCCCCTGAGCACACCTCTGAGC |
| | | CACAGCACC |
| EP14_N292Q | | |
| SEQ ID | Amino | TQNKALPENVKYGIVLDAGSSHTSLYIYKWPAEKENDTGVVHQVEECRVK |
| NO: 90 | acid | GPGISKFVQKVNEIGIYLTDCMERAREVIPRSQHQETPVYLGATAGMRLLR |
| | | MESEELADRVLDVVERSLSNYPFDFQGARIITGQEEGAYGWITINYLLGKF |
| | | SQKNQETFGALDLGGASTQVTFVPQNQTIESPDNALQFRLYGKDYNVYT |
| | | HSFLCYGKDQALWQKLAKDIQVASNEILRDPCFHPGYKKVVQVSDLYKTP |
| | | CTKRFEMTLPFQQFEIQGIGNYQQCHQSILELFNTSYCPYSQCAFNGIFLP |
| | | PLQGDFGAFSAFYSVMKFLNLTSEKVSQEKVTEMMKKFCAQPWEEIKTS |
| | | YAGVKEKYLSEYCFSGTYILSLLLQGYHFTADSWEHIHFIGKIQGSDAGWT |
| | | LGYMLNLTNMIPAEQPLSTPLSHST |
| SEQ ID | DNA | ACCCAGAACAAGGCCCTGCCCGAGAACGTGAAGTACGGCATCGTGCT |
| NO: 91 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCAGGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGCTGGACGTGGTGGAAAGAAGCCTGAGCAACTACCCATTCGAT |
| | | TTTCAAGGCGCCAGAATCATCACCGGCCAGGAAGAAGGCGCCTACGG |
| | | CTGGATCACCATCAACTACCTGCTGGGCAAGTTCAGCCAGAAGAATCA |
| | | GGAAACCTTCGGCGCCCTGGACCTGGGCGGAGCTTCTACCCAAGTGA |
| | | CCTTCGTGCCCCAGAATCAGACCATCGAGAGCCCCGACAACGCCCTG |
| | | CAGTTCCGGCTGTACGGCAAGGACTACAATGTGTACACCCACAGCTTT |
| | | CTGTGCTACGGAAAGGACCAGGCTCTGTGGCAGAAGCTGGCCAAGGA |
| | | CATCCAGGTGGCCAGCAACGAGATCCTGCGGGACCCTTGCTTCCACC |
| | | CCGGCTACAAGAAAGTCGTGCAGGTGTCCGACCTGTACAAGACCCCC |
| | | TGCACCAAGAGATTCGAGATGACCCTGCCCTTCCAGCAGTTCGAGATC |
| | | CAGGGCATCGGCAATTACCAGCAGTGCCACCAGAGCATCCTGGAACT |
| | | GTTCAACACCAGCTACTGCCCCTACAGCCAGTGCGCCTTCAACGGCA |
| | | TCTTCCTGCCACCTCTGCAGGGGGATTTCGGCGCCTTCAGCGCCTTC |
| | | TACTCCGTGATGAAGTTCCTGAACCTGACCAGCGAGAAGGTGTCCCA |
| | | GGAAAAAGTGACAGAGATGATGAAGAAGTTCTGCGCCCAGCCCTGGG |
| | | AGGAAATCAAGACCTCCTACGCTGGCGTGAAAGAGAAGTACCTGAGC |
| | | GAGTACTGCTTCAGCGGCACCTACATCCTGAGCCTGCTGCTGCAGGG |
| | | CTACCACTTCACCGCCGATAGCTGGGAGCACATCCACTTCATCGGCAA |
| | | GATTCAGGGCAGCGACGCCGGCTGGACACTGGGCTACATGCTGAATC |
| | | TGACCAACATGATCCCCGCCGAGCAGCCCCTGAGCACACCTCTGAGC |
| | | CACAGCACC |
| EP14_N327Q | | |
| SEQ ID | Amino | TQNKALPENVKYGIVLDAGSSHTSLYIYKWPAEKENDTGVVHQVEECRVK |
| NO: 92 | acid | GPGISKFVQKVNEIGIYLTDCMERAREVIPRSQHQETPVYLGATAGMRLLR |
| | | MESEELADRVLDVVERSLSNYPFDFQGARIITGQEEGAYGWITINYLLGKF |
| | | SQKNQETFGALDLGGASTQVTFVPQNQTIESPDNALQFRLYGKDYNVYT |
| | | HSFLCYGKDQALWQKLAKDIQVASNEILRDPCFHPGYKKVVNVSDLYKTP |
| | | CTKRFEMTLPFQQFEIQGIGNYQQCHQSILELFQTSYCPYSQCAFNGIFLP |
| | | PLQGDFGAFSAFYSVMKFLNLTSEKVSQEKVTEMMKKFCAQPWEEIKTS |
| | | YAGVKEKYLSEYCFSGTYILSLLLQGYHFTADSWEHIHFIGKIQGSDAGWT |
| | | LGYMLNLTNMIPAEQPLSTPLSHST |
| SEQ ID | DNA | ACCCAGAACAAGGCCCTGCCCGAGAACGTGAAGTACGGCATCGTGCT |
| NO: 93 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCAGGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGCTGGACGTGGTGGAAAGAAGCCTGAGCAACTACCCATTCGAT |
| | | TTTCAAGGCGCCAGAATCATCACCGGCCAGGAAGAAGGCGCCTACGG |
| | | CTGGATCACCATCAACTACCTGCTGGGCAAGTTCAGCCAGAAGAATCA |
| | | GGAAACCTTCGGCGCCCTGGACCTGGGCGGAGCTTCTACCCAAGTGA |
| | | CCTTCGTGCCCCAGAATCAGACCATCGAGAGCCCCGACAACGCCCTG |
| | | CAGTTCCGGCTGTACGGCAAGGACTACAATGTGTACACCCACAGCTTT |
| | | CTGTGCTACGGAAAGGACCAGGCTCTGTGGCAGAAGCTGGCCAAGGA |
| | | CATCCAGGTGGCCAGCAACGAGATCCTGCGGGACCCTTGCTTCCACC |
| | | CCGGCTACAAGAAAGTCGTGAACGTGTCCGACCTGTACAAGACCCCC |
| | | TGCACCAAGAGATTCGAGATGACCCTGCCCTTCCAGCAGTTCGAGATC |
| | | CAGGGCATCGGCAATTACCAGCAGTGCCACCAGAGCATCCTGGAACT |
| | | GTTCCAGACCAGCTACTGCCCCTACAGCCAGTGCGCCTTCAACGGCA |
| | | TCTTCCTGCCACCTCTGCAGGGGGATTTCGGCGCCTTCAGCGCCTTC |
| | | TACTCCGTGATGAAGTTCCTGAACCTGACCAGCGAGAAGGTGTCCCA |
| | | GGAAAAAGTGACAGAGATGATGAAGAAGTTCTGCGCCCAGCCCTGGG |
| | | AGGAAATCAAGACCTCCTACGCTGGCGTGAAAGAGAAGTACCTGAGC |
| | | GAGTACTGCTTCAGCGGCACCTACATCCTGAGCCTGCTGCTGCAGGG |
| | | CTACCACTTCACCGCCGATAGCTGGGAGCACATCCACTTCATCGGCAA |
| | | GATTCAGGGCAGCGACGCCGGCTGGACACTGGGCTACATGCTGAATC |
| | | TGACCAACATGATCCCCGCCGAGCAGCCCCTGAGCACACCTCTGAGC |
| | | CACAGCACC |
| EP14_N371Q | | |
| SEQ ID | Amino | TQNKALPENVKYGIVLDAGSSHTSLYIYKWPAEKENDTGVVHQVEECRVK |
| NO: 94 | acid | GPGISKFVQKVNEIGIYLTDCMERAREVIPRSQHQETPVYLGATAGMRLLR |
| | | MESEELADRVLDVVERSLSNYPFDFQGARIITGQEEGAYGWITINYLLGKF |
| | | SQKNQETFGALDLGGASTQVTFVPQNQTIESPDNALQFRLYGKDYNVYT |
| | | HSFLCYGKDQALWQKLAKDIQVASNEILRDPCFHPGYKKVVNVSDLYKTP |
| | | CTKRFEMTLPFQQFEIQGIGNYQQCHQSILELFNTSYCPYSQCAFNGIFLP |
| | | PLQGDFGAFSAFYSVMKFLQLTSEKVSQEKVTEMMKKFCAQPWEEIKTS |
| | | YAGVKEKYLSEYCFSGTYILSLLLQGYHFTADSWEHIHFIGKIQGSDAGWT |
| | | LGYMLNLTNMIPAEQPLSTPLSHST |
| SEQ ID | DNA | ACCCAGAACAAGGCCCTGCCCGAGAACGTGAAGTACGGCATCGTGCT |
| NO: 95 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCAGGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGCTGGACGTGGTGGAAAGAAGCCTGAGCAACTACCCATTCGAT |
| | | TTTCAAGGCGCCAGAATCATCACCGGCCAGGAAGAAGGCGCCTACGG |
| | | CTGGATCACCATCAACTACCTGCTGGGCAAGTTCAGCCAGAAGAATCA |
| | | GGAAACCTTCGGCGCCCTGGACCTGGGCGGAGCTTCTACCCAAGTGA |
| | | CCTTCGTGCCCCAGAATCAGACCATCGAGAGCCCCGACAACGCCCTG |
| | | CAGTTCCGGCTGTACGGCAAGGACTACAATGTGTACACCCACAGCTTT |
| | | CTGTGCTACGGAAAGGACCAGGCTCTGTGGCAGAAGCTGGCCAAGGA |
| | | CATCCAGGTGGCCAGCAACGAGATCCTGCGGGACCCTTGCTTCCACC |
| | | CCGGCTACAAGAAAGTCGTGAACGTGTCCGACCTGTACAAGACCCCC |
| | | TGCACCAAGAGATTCGAGATGACCCTGCCCTTCCAGCAGTTCGAGATC |
| | | CAGGGCATCGGCAATTACCAGCAGTGCCACCAGAGCATCCTGGAACT |
| | | GTTCAACACCAGCTACTGCCCCTACAGCCAGTGCGCCTTCAACGGCA |
| | | TCTTCCTGCCACCTCTGCAGGGGGATTTCGGCGCCTTCAGCGCCTTC |
| | | TACTCCGTGATGAAGTTCCTGCAGCTGACCAGCGAGAAGGTGTCCCA |
| | | GGAAAAAGTGACAGAGATGATGAAGAAGTTCTGCGCCCAGCCCTGGG |
| | | AGGAAATCAAGACCTCCTACGCTGGCGTGAAAGAGAAGTACCTGAGC |
| | | GAGTACTGCTTCAGCGGCACCTACATCCTGAGCCTGCTGCTGCAGGG |
| | | CTACCACTTCACCGCCGATAGCTGGGAGCACATCCACTTCATCGGCAA |
| | | GATTCAGGGCAGCGACGCCGGCTGGACACTGGGCTACATGCTGAATC |
| | | TGACCAACATGATCCCCGCCGAGCAGCCCCTGAGCACACCTCTGAGC |
| | | CACAGCACC |
| EP14_N457Q | | |
| SEQ ID | Amino | TQNKALPENVKYGIVLDAGSSHTSLYIYKWPAEKENDTGVVHQVEECRVK |
| NO: 96 | acid | GPGISKFVQKVNEIGIYLTDCMERAREVIPRSQHQETPVYLGATAGMRLLR |
| | | MESEELADRVLDVVERSLSNYPFDFQGARIITGQEEGAYGWITINYLLGKF |
| | | SQKNQETFGALDLGGASTQVTFVPQNQTIESPDNALQFRLYGKDYNVYT |
| | | HSFLCYGKDQALWQKLAKDIQVASNEILRDPCFHPGYKKVVNVSDLYKTP |
| | | CTKRFEMTLPFQQFEIQGIGNYQQCHQSILELFNTSYCPYSQCAFNGIFLP |
| | | PLQGDFGAFSAFYSVMKFLNLTSEKVSQEKVTEMMKKFCAQPWEEIKTS |
| | | YAGVKEKYLSEYCFSGTYILSLLLQGYHFTADSWEHIHFIGKIQGSDAGWT |
| | | LGYMLQLTNMIPAEQPLSTPLSHST |
| SEQ ID | DNA | ACCCAGAACAAGGCCCTGCCCGAGAACGTGAAGTACGGCATCGTGCT |
| NO: 97 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCAGGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGCTGGACGTGGTGGAAAGAAGCCTGAGCAACTACCCATTCGAT |
| | | TTTCAAGGCGCCAGAATCATCACCGGCCAGGAAGAAGGCGCCTACGG |
| | | CTGGATCACCATCAACTACCTGCTGGGCAAGTTCAGCCAGAAGAATCA |
| | | GGAAACCTTCGGCGCCCTGGACCTGGGCGGAGCTTCTACCCAAGTGA |
| | | CCTTCGTGCCCCAGAATCAGACCATCGAGAGCCCCGACAACGCCCTG |
| | | CAGTTCCGGCTGTACGGCAAGGACTACAATGTGTACACCCACAGCTTT |
| | | CTGTGCTACGGAAAGGACCAGGCTCTGTGGCAGAAGCTGGCCAAGGA |
| | | CATCCAGGTGGCCAGCAACGAGATCCTGCGGGACCCTTGCTTCCACC |
| | | CCGGCTACAAGAAAGTCGTGAACGTGTCCGACCTGTACAAGACCCCC |
| | | TGCACCAAGAGATTCGAGATGACCCTGCCCTTCCAGCAGTTCGAGATC |
| | | CAGGGCATCGGCAATTACCAGCAGTGCCACCAGAGCATCCTGGAACT |
| | | GTTCAACACCAGCTACTGCCCCTACAGCCAGTGCGCCTTCAACGGCA |
| | | TCTTCCTGCCACCTCTGCAGGGGGATTTCGGCGCCTTCAGCGCCTTC |
| | | TACTCCGTGATGAAGTTCCTGAACCTGACCAGCGAGAAGGTGTCCCA |
| | | GGAAAAAGTGACAGAGATGATGAAGAAGTTCTGCGCCCAGCCCTGGG |
| | | AGGAAATCAAGACCTCCTACGCTGGCGTGAAAGAGAAGTACCTGAGC |
| | | GAGTACTGCTTCAGCGGCACCTACATCCTGAGCCTGCTGCTGCAGGG |
| | | CTACCACTTCACCGCCGATAGCTGGGAGCACATCCACTTCATCGGCAA |
| | | GATTCAGGGCAGCGACGCCGGCTGGACACTGGGCTACATGCTGCAG |
| | | CTGACCAACATGATCCCCGCCGAGCAGCCCCTGAGCACACCTCTGAG |
| | | CCACAGCACC |
| SEQ ID | plusMILrevsense | TGCCCTACGAGACAAACAATCAGGAAACCTTCGGCGCCCTGGACCTG |
| NO: 98 | primer | GGCGGAGCTTCTACCCAAGTGA |
| CD39(aa39-469) | | |
| SEQ ID | Amino | QNKALPENVKYGIVLDAGSSHTSLYIYKWPAEKENDTGVVHQVEECRVK |
| NO: 99 | acid | GPGISKFVQKVNEIGIYLTDCMERAREVIPRSQHQETPVYLGATAGMRLLR |
| | | MESEELADRVLDVVERSLSNYPFDFQGARIITGQEEGAYGWITINYLLGKF |
| | | SQKTRWFSIVPYETNNQETFGALDLGGASTQVTFVPQNQTIESPDNALQF |
| | | RLYGKDYNVYTHSFLCYGKDQALWQKLAKDIQVASNEILRDPCFHPGYKK |
| | | VVNVSDLYKTPCTKRFEMTLPFQQFEIQGIGNYQQCHQSILELFNTSYCPY |
| | | SQCAFNGIFLPPLQGDFGAFSAFYFVMKFLNLTSEKVSQEKVTEMMKKFC |
| | | AQPVVEEIKTSYAGVKEKYLSEYCFSGTYILSLLLQGYHFTADSVVEHIHFIG |
| | | KIQGSDAGVVTLGYMLNLTNMIPAEQPLS |
| SEQ ID | DNA | CAGAACAAAGCCCTGCCCGAGAACGTGAAGTACGGCATCGTGCTGGA |
| NO: 100 | | TGCCGGCAGCAGCCACACCAGCCTGTACATCTACAAGTGGCCTGCCG |
| | | AGAAAGAAAACGATACCGGTGTCGTGCACCAGGTGGAAGAGTGCAGA |
| | | GTGAAGGGCCCTGGCATCAGCAAGTTCGTGCAGAAAGTGAACGAGAT |
| | | CGGCATCTACCTGACCGACTGCATGGAACGGGCCAGAGAAGTGATCC |
| | | CCAGAAGCCAGCACCAGGAAACCCCCGTGTACCTGGGAGCCACAGC |
| | | CGGCATGAGACTGCTGCGGATGGAAAGCGAGGAACTGGCCGACAGA |
| | | GTGCTGGACGTGGTGGAAAGAAGCCTGAGCAACTACCCATTCGATTTT |
| | | CAAGGGGCCAGAATCATCACCGGCCAGGAAGAGGGCGCTTACGGCT |
| | | GGATCACCATCAACTACCTGCTGGGCAAGTTCAGCCAGAAAACCCGG |
| | | TGGTTCAGCATCGTGCCCTACGAGACAAACAATCAGGAAACCTTCGGA |
| | | GCCCTGGACCTGGGCGGAGCCTCTACCCAAGTGACCTTCGTGCCCCA |
| | | GAATCAGACCATCGAGAGCCCCGACAACGCCCTGCAGTTCCGGCTGT |
| | | ACGGCAAGGACTACAATGTGTACACCCACAGCTTTCTGTGCTACGGAA |
| | | AGGACCAGGCCCTGTGGCAGAAGCTGGCCAAGGACATCCAGGTGGC |
| | | CAGCAACGAGATCCTGCGGGACCCTTGCTTCCACCCCGGCTACAAGA |
| | | AAGTCGTGAACGTGTCCGACCTGTACAAGACCCCCTGCACCAAGAGA |
| | | TTCGAGATGACCCTGCCCTTCCAGCAGTTCGAGATCCAGGGCATCGG |
| | | CAACTACCAGCAGTGCCACCAGAGCATCCTGGAACTGTTCAACACCA |
| | | GCTACTGCCCCTACAGCCAGTGCGCCTTCAACGGCATCTTCCTGCCA |
| | | CCTCTGCAGGGGGACTTCGGCGCTTTCAGCGCCTTCTACTTCGTGAT |
| | | GAAGTTCCTGAACCTGACCAGCGAGAAGGTGTCCCAGGAAAAAGTGA |
| | | CAGAGATGATGAAGAAGTTCTGCGCCCAGCCCTGGGAGGAAATCAAG |
| | | ACCTCCTACGCTGGCGTGAAAGAGAAGTACCTGAGCGAGTACTGCTT |
| | | CAGCGGTACCTACATCCTGAGCCTGCTGCTGCAGGGCTACCACTTCA |
| | | CCGCCGATAGCTGGGAGCACATCCACTTCATCGGCAAGATTCAGGGC |
| | | AGCGACGCCGGCTGGACACTGGGCTACATGCTGAATCTGACCAACAT |
| | | GATCCCCGCCGAGCAGCCCCTGAGC |
| CD39(aa46-476) | | |
| SEQ ID | Amino | NVKYGIVLDAGSSHTSLYIYKWPAEKENDTGVVHQVEECRVKGPGISKFV |
| NO: 101 | acid | QKVNEIGIYLTDCMERAREVIPRSQHQETPVYLGATAGMRLLRMESEELA |
| | | DRVLDVVERSLSNYPFDFQGARIITGQEEGAYGWITINYLLGKFSQKTRW |
| | | FSNPYETNNQETFGALDLGGASTQVTFVPQNQTESPDNALQFRLYGKD |
| | | YNVYTHSFLCYGKDQALWQKLAKDIQVASNEILRDPCFHPGYKKVVNVSD |
| | | LYKTPCTKRFEMTLPFQQFEIQGIGNYQQCHQSILELFNTSYCPYSQCAF |
| | | NGIFLPPLQGDFGAFSAFYFVMKFLNLTSEKVSQEKVTEMMKKFCAQPW |
| | | EEIKTSYAGVKEKYLSEYCFSGTYILSLLLQGYHFTADSWEHIHFIGKIQGS |
| | | DAGVVTLGYMLNLTNMIPAEQPLSTPLSHST |
| SEQ ID | DNA | AACGTGAAGTACGGCATCGTGCTGGATGCCGGCAGCAGCCACACCAG |
| NO: 102 | | CCTGTACATCTACAAGTGGCCTGCCGAGAAAGAAAACGATACCGGTGT |
| | | CGTGCACCAGGTGGAAGAGTGCAGAGTGAAGGGCCCTGGCATCAGC |
| | | AAGTTCGTGCAGAAAGTGAACGAGATCGGCATCTACCTGACCGACTG |
| | | CATGGAACGGGCCAGAGAAGTGATCCCCAGAAGCCAGCACCAGGAAA |
| | | CCCCCGTGTACCTGGGAGCCACAGCCGGCATGAGACTGCTGCGGAT |
| | | GGAAAGCGAGGAACTGGCCGACAGAGTGCTGGACGTGGTGGAAAGA |
| | | AGCCTGAGCAACTACCCATTCGATTTTCAAGGGGCCAGAATCATCACC |
| | | GGCCAGGAAGAGGGCGCTTACGGCTGGATCACCATCAACTACCTGCT |
| | | GGGCAAGTTCAGCCAGAAAACCCGGTGGTTCAGCATCGTGCCCTACG |
| | | AGACAAACAATCAGGAAACCTTCGGAGCCCTGGACCTGGGCGGAGCC |
| | | TCTACCCAAGTGACCTTCGTGCCCCAGAATCAGACCATCGAGAGCCC |
| | | CGACAACGCCCTGCAGTTCCGGCTGTACGGCAAGGACTACAATGTGT |
| | | ACACCCACAGCTTTCTGTGCTACGGAAAGGACCAGGCCCTGTGGCAG |
| | | AAGCTGGCCAAGGACATCCAGGTGGCCAGCAACGAGATCCTGCGGG |
| | | ACCCTTGCTTCCACCCCGGCTACAAGAAAGTCGTGAACGTGTCCGAC |
| | | CTGTACAAGACCCCCTGCACCAAGAGATTCGAGATGACCCTGCCCTTC |
| | | CAGCAGTTCGAGATCCAGGGCATCGGCAACTACCAGCAGTGCCACCA |
| | | GAGCATCCTGGAACTGTTCAACACCAGCTACTGCCCCTACAGCCAGT |
| | | GCGCCTTCAACGGCATCTTCCTGCCACCTCTGCAGGGGGACTTCGGC |
| | | GCTTTCAGCGCCTTCTACTTCGTGATGAAGTTCCTGAACCTGACCAGC |
| | | GAGAAGGTGTCCCAGGAAAAAGTGACAGAGATGATGAAGAAGTTCTG |
| | | CGCCCAGCCCTGGGAGGAAATCAAGACCTCCTACGCTGGCGTGAAAG |
| | | AGAAGTACCTGAGCGAGTACTGCTTCAGCGGTACCTACATCCTGAGC |
| | | CTGCTGCTGCAGGGCTACCACTTCACCGCCGATAGCTGGGAGCACAT |
| | | CCACTTCATCGGCAAGATTCAGGGCAGCGACGCCGGCTGGACACTGG |
| | | GCTACATGCTGAATCTGACCAACATGATCCCCGCCGAGCAGCCCCTG |
| | | AGCACACCTCTGTCTCACAGCACC |
| CD39(aa46-461) | | |
| SEQ ID | Amino | NVKYGIVLDAGSSHTSLYIYKVVPAEKENDTGVVHQVEECRVKGPGISKFV |
| NO: 103 | acid | QKVNEIGIYLTDCMERAREVIPRSQHQETPVYLGATAGMRLLRMESEELA |
| | | DRVLDVVERSLSNYPFDFQGARIITGQEEGAYGWITINYLLGKFSQKTRW |
| | | FSNPYETNNQETFGALDLGGASTQVTFVPQNQTESPDNALQFRLYGKD |
| | | YNVYTHSFLCYGKDQALWQKLAKDIQVASNEILRDPCFHPGYKKVVNVSD |
| | | LYKTPCTKRFEMTLPFQQFEIQGIGNYQQCHQSILELFNTSYCPYSQCAF |
| | | NGIFLPPLQGDFGAFSAFYFVMKFLNLTSEKVSQEKVTEMMKKFCAQPW |
| | | EEIKTSYAGVKEKYLSEYCFSGTYILSLLLQGYHFTADSWEHIHFIGKIQGS |
| | | DAGWTLGYMLNLTNM |
| SEQ ID | DNA | AACGTGAAGTACGGCATCGTGCTGGATGCCGGCAGCAGCCACACCAG |
| NO: 104 | | CCTGTACATCTACAAGTGGCCTGCCGAGAAAGAAAACGATACCGGTGT |
| | | CGTGCACCAGGTGGAAGAGTGCAGAGTGAAGGGCCCTGGCATCAGC |
| | | AAGTTCGTGCAGAAAGTGAACGAGATCGGCATCTACCTGACCGACTG |
| | | CATGGAACGGGCCAGAGAAGTGATCCCCAGAAGCCAGCACCAGGAAA |
| | | CCCCCGTGTACCTGGGAGCCACAGCCGGCATGAGACTGCTGCGGAT |
| | | GGAAAGCGAGGAACTGGCCGACAGAGTGCTGGACGTGGTGGAAAGA |
| | | AGCCTGAGCAACTACCCATTCGATTTTCAAGGGGCCAGAATCATCACC |
| | | GGCCAGGAAGAGGGCGCTTACGGCTGGATCACCATCAACTACCTGCT |
| | | GGGCAAGTTCAGCCAGAAAACCCGGTGGTTCAGCATCGTGCCCTACG |
| | | AGACAAACAATCAGGAAACCTTCGGAGCCCTGGACCTGGGCGGAGCC |
| | | TCTACCCAAGTGACCTTCGTGCCCCAGAATCAGACCATCGAGAGCCC |
| | | CGACAACGCCCTGCAGTTCCGGCTGTACGGCAAGGACTACAATGTGT |
| | | ACACCCACAGCTTTCTGTGCTACGGAAAGGACCAGGCCCTGTGGCAG |
| | | AAGCTGGCCAAGGACATCCAGGTGGCCAGCAACGAGATCCTGCGGG |
| | | ACCCTTGCTTCCACCCCGGCTACAAGAAAGTCGTGAACGTGTCCGAC |
| | | CTGTACAAGACCCCCTGCACCAAGAGATTCGAGATGACCCTGCCCTTC |
| | | CAGCAGTTCGAGATCCAGGGCATCGGCAACTACCAGCAGTGCCACCA |
| | | GAGCATCCTGGAACTGTTCAACACCAGCTACTGCCCCTACAGCCAGT |
| | | GCGCCTTCAACGGCATCTTCCTGCCACCTCTGCAGGGGGACTTCGGC |
| | | GCTTTCAGCGCCTTCTACTTCGTGATGAAGTTCCTGAACCTGACCAGC |
| | | GAGAAGGTGTCCCAGGAAAAAGTGACAGAGATGATGAAGAAGTTCTG |
| | | CGCCCAGCCCTGGGAGGAAATCAAGACCTCCTACGCTGGCGTGAAAG |
| | | AGAAGTACCTGAGCGAGTACTGCTTCAGCGGTACCTACATCCTGAGC |
| | | CTGCTGCTGCAGGGCTACCACTTCACCGCCGATAGCTGGGAGCACAT |
| | | CCACTTCATCGGCAAGATTCAGGGCAGCGACGCCGGCTGGACACTGG |
| | | GCTACATGCTGAATCTGACCAACATG |
| CD39(aa46- | | |
| 461)_dMIL(193-204) | | |
| SEQ ID | Amino | NVKYGIVLDAGSSHTSLYIYKVVPAEKENDTGVVHQVEECRVKGPGISKFV |
| NO: 105 | acid | QKVNEIGIYLTDCMERAREVIPRSQHQETPVYLGATAGMRLLRMESEELA |
| | | DRVLDVVERSLSNYPFDFQGARMTGQEEGAYGWITINYLLGKFSQKNQET |
| | | FGALDLGGASTQVTFVPQNQTESPDNALQFRLYGKDYNVYTHSFLCYGK |
| | | DQALWQKLAKDIQVASNEILRDPCFHPGYKKVVNVSDLYKTPCTKRFEMT |
| | | LPFQQFEIQGIGNYQQCHQSILELFNTSYCPYSQCAFNGIFLPPLQGDFGA |
| | | FSAFYFVMKFLNLTSEKVSQEKVTEMMKKFCAQPWEEIKTSYAGVKEKYL |
| | | SEYCFSGTYILSLLLQGYHFTADSWEHIHFIGKIQGSDAGVVTLGYMLNLTN |
| | | M |
| SEQ ID | DNA | AACGTGAAGTACGGCATCGTGCTGGATGCCGGCAGCAGCCACACCAG |
| NO: 106 | | CCTGTACATCTACAAGTGGCCTGCCGAGAAAGAAAACGATACCGGTGT |
| | | CGTGCACCAGGTGGAAGAGTGCAGAGTGAAGGGCCCTGGCATCAGC |
| | | AAGTTCGTGCAGAAAGTGAACGAGATCGGCATCTACCTGACCGACTG |
| | | CATGGAACGGGCCAGAGAAGTGATCCCCAGAAGCCAGCACCAGGAAA |
| | | CCCCCGTGTACCTGGGAGCCACAGCCGGCATGAGACTGCTGCGGAT |
| | | GGAAAGCGAGGAACTGGCCGACAGAGTGCTGGACGTGGTGGAAAGA |
| | | AGCCTGAGCAACTACCCATTCGATTTTCAAGGGGCCAGAATCATCACC |
| | | GGCCAGGAAGAGGGCGCTTACGGCTGGATCACCATCAACTACCTGCT |
| | | GGGCAAGTTCAGCCAGAAAAATCAGGAAACCTTCGGAGCCCTGGACC |
| | | TGGGCGGAGCCTCTACCCAAGTGACCTTCGTGCCCCAGAATCAGACC |
| | | ATCGAGAGCCCCGACAACGCCCTGCAGTTCCGGCTGTACGGCAAGGA |
| | | CTACAATGTGTACACCCACAGCTTTCTGTGCTACGGAAAGGACCAGGC |
| | | CCTGTGGCAGAAGCTGGCCAAGGACATCCAGGTGGCCAGCAACGAG |
| | | ATCCTGCGGGACCCTTGCTTCCACCCCGGCTACAAGAAAGTCGTGAA |
| | | CGTGTCCGACCTGTACAAGACCCCCTGCACCAAGAGATTCGAGATGA |
| | | CCCTGCCCTTCCAGCAGTTCGAGATCCAGGGCATCGGCAACTACCAG |
| | | CAGTGCCACCAGAGCATCCTGGAACTGTTCAACACCAGCTACTGCCC |
| | | CTACAGCCAGTGCGCCTTCAACGGCATCTTCCTGCCACCTCTGCAGG |
| | | GGGACTTCGGCGCTTTCAGCGCCTTCTACTTCGTGATGAAGTTCCTGA |
| | | ACCTGACCAGCGAGAAGGTGTCCCAGGAAAAAGTGACAGAGATGATG |
| | | AAGAAGTTCTGCGCCCAGCCCTGGGAGGAAATCAAGACCTCCTACGC |
| | | TGGCGTGAAAGAGAAGTACCTGAGCGAGTACTGCTTCAGCGGTACCT |
| | | ACATCCTGAGCCTGCTGCTGCAGGGCTACCACTTCACCGCCGATAGC |
| | | TGGGAGCACATCCACTTCATCGGCAAGATTCAGGGCAGCGACGCCGG |
| | | CTGGACACTGGGCTACATGCTGAATCTGACCAACATG |
| CD39(aa46- | | |
| 461)_delta | | |
| cys1 | | |
| SEQ ID | Amino | NVKYGIVLDAGSSHTSLYIYKVVPAEKENDTGVVHQVEEARVKGPGISKFV |
| NO: 107 | acid | QKVNEIGIYLTDAMERAREVIPRSQHQETPVYLGATAGMRLLRMESEELA |
| | | DRVLDVVERSLSNYPFDFQGARIITGQEEGAYGWITINYLLGKFSQKTRW |
| | | FSNPYETNNQETFGALDLGGASTQVTFVPQNQTESPDNALQFRLYGKD |
| | | YNVYTHSFLCYGKDQALWQKLAKDIQVASNEILRDPCFHPGYKKVVNVSD |
| | | LYKTPCTKRFEMTLPFQQFEIQGIGNYQQCHQSILELFNTSYCPYSQCAF |
| | | NGIFLPPLQGDFGAFSAFYFVMKFLNLTSEKVSQEKVTEMMKKFCAQPW |
| | | EEIKTSYAGVKEKYLSEYCFSGTYILSLLLQGYHFTADSWEHIHFIGKIQGS |
| | | DAGWTLGYMLNLTNM |
| SEQ ID | DNA | AACGTGAAGTACGGCATCGTGCTGGATGCCGGCAGCAGCCACACCAG |
| NO: 108 | | CCTGTACATCTACAAGTGGCCTGCCGAGAAAGAAAACGATACCGGTGT |
| | | CGTGCACCAGGTGGAAGAGGCCAGAGTGAAGGGCCCTGGCATCAGC |
| | | AAGTTCGTGCAGAAAGTGAACGAGATCGGCATCTACCTGACCGACGC |
| | | CATGGAACGGGCCAGAGAAGTGATCCCCAGAAGCCAGCACCAGGAAA |
| | | CCCCCGTGTACCTGGGAGCCACAGCCGGCATGAGACTGCTGCGGAT |
| | | GGAAAGCGAGGAACTGGCCGACAGAGTGCTGGACGTGGTGGAAAGA |
| | | AGCCTGAGCAACTACCCATTCGATTTTCAAGGGGCCAGAATCATCACC |
| | | GGCCAGGAAGAGGGCGCTTACGGCTGGATCACCATCAACTACCTGCT |
| | | GGGCAAGTTCAGCCAGAAAACCCGGTGGTTCAGCATCGTGCCCTACG |
| | | AGACAAACAATCAGGAAACCTTCGGAGCCCTGGACCTGGGCGGAGCC |
| | | TCTACCCAAGTGACCTTCGTGCCCCAGAATCAGACCATCGAGAGCCC |
| | | CGACAACGCCCTGCAGTTCCGGCTGTACGGCAAGGACTACAATGTGT |
| | | ACACCCACAGCTTTCTGTGCTACGGAAAGGACCAGGCCCTGTGGCAG |
| | | AAGCTGGCCAAGGACATCCAGGTGGCCAGCAACGAGATCCTGCGGG |
| | | ACCCTTGCTTCCACCCCGGCTACAAGAAAGTCGTGAACGTGTCCGAC |
| | | CTGTACAAGACCCCCTGCACCAAGAGATTCGAGATGACCCTGCCCTTC |
| | | CAGCAGTTCGAGATCCAGGGCATCGGCAACTACCAGCAGTGCCACCA |
| | | GAGCATCCTGGAACTGTTCAACACCAGCTACTGCCCCTACAGCCAGT |
| | | GCGCCTTCAACGGCATCTTCCTGCCACCTCTGCAGGGGGACTTCGGC |
| | | GCTTTCAGCGCCTTCTACTTCGTGATGAAGTTCCTGAACCTGACCAGC |
| | | GAGAAGGTGTCCCAGGAAAAAGTGACAGAGATGATGAAGAAGTTCTG |
| | | CGCCCAGCCCTGGGAGGAAATCAAGACCTCCTACGCTGGCGTGAAAG |
| | | AGAAGTACCTGAGCGAGTACTGCTTCAGCGGTACCTACATCCTGAGC |
| | | CTGCTGCTGCAGGGCTACCACTTCACCGCCGATAGCTGGGAGCACAT |
| | | CCACTTCATCGGCAAGATTCAGGGCAGCGACGCCGGCTGGACACTGG |
| | | GCTACATGCTGAATCTGACCAACATG |
| CD39(aa46- | | |
| 461)_delta | | |
| cys2 | | |
| SEQ ID | Amino | NVKYGIVLDAGSSHTSLYIYKVVPAEKENDTGVVHQVEECRVKGPGISKFV |
| NO: 109 | acid | QKVNEIGIYLTDCMERAREVIPRSQHQETPVYLGATAGMRLLRMESEELA |
| | | DRVLDVVERSLSNYPFDFQGARIITGQEEGAYGWITINYLLGKFSQKTRW |
| | | FSIVPYETNNQETFGALDLGGASTQVTFVPQNQTESPDNALQFRLYGKD |
| | | YNVYTHSFLCYGKDQALWQKLAKDIQVASNEILRDPAFHPGYKKVVNVSD |
| | | LYKTPCTKRFEMTLPFQQFEIQGIGNYQQAHQSILELFNTSYCPYSQCAFN |
| | | GIFLPPLQGDFGAFSAFYFVMKFLNLTSEKVSQEKVTEMMKKFCAQPWE |
| | | EIKTSYAGVKEKYLSEYCFSGTYILSLLLQGYHFTADSWEHIHFIGKIQGSD |
| | | AGWTLGYMLNLTNM |
| SEQ ID | DNA | AACGTGAAGTACGGCATCGTGCTGGATGCCGGCAGCAGCCACACCAG |
| NO: 110 | | CCTGTACATCTACAAGTGGCCTGCCGAGAAAGAAAACGATACCGGTGT |
| | | CGTGCACCAGGTGGAAGAGTGCAGAGTGAAGGGCCCTGGCATCAGC |
| | | AAGTTCGTGCAGAAAGTGAACGAGATCGGCATCTACCTGACCGACTG |
| | | CATGGAACGGGCCAGAGAAGTGATCCCCAGAAGCCAGCACCAGGAAA |
| | | CCCCCGTGTACCTGGGAGCCACAGCCGGCATGAGACTGCTGCGGAT |
| | | GGAAAGCGAGGAACTGGCCGACAGAGTGCTGGACGTGGTGGAAAGA |
| | | AGCCTGAGCAACTACCCATTCGATTTTCAAGGGGCCAGAATCATCACC |
| | | GGCCAGGAAGAGGGCGCTTACGGCTGGATCACCATCAACTACCTGCT |
| | | GGGCAAGTTCAGCCAGAAAACCCGGTGGTTCAGCATCGTGCCCTACG |
| | | AGACAAACAATCAGGAAACCTTCGGAGCCCTGGACCTGGGCGGAGCC |
| | | TCTACCCAAGTGACCTTCGTGCCCCAGAATCAGACCATCGAGAGCCC |
| | | CGACAACGCCCTGCAGTTCCGGCTGTACGGCAAGGACTACAATGTGT |
| | | ACACCCACAGCTTTCTGTGCTACGGAAAGGACCAGGCCCTGTGGCAG |
| | | AAGCTGGCCAAGGACATCCAGGTGGCCAGCAACGAGATCCTGCGGG |
| | | ACCCTGCCTTCCACCCCGGCTACAAGAAAGTCGTGAACGTGTCCGAC |
| | | CTGTACAAGACCCCCTGCACCAAGAGATTCGAGATGACCCTGCCCTTC |
| | | CAGCAGTTCGAGATCCAGGGCATCGGCAACTACCAGCAGGCCCACCA |
| | | GAGCATCCTGGAACTGTTCAACACCAGCTACTGCCCCTACAGCCAGT |
| | | GCGCCTTCAACGGCATCTTCCTGCCACCTCTGCAGGGGGACTTCGGC |
| | | GCTTTCAGCGCCTTCTACTTCGTGATGAAGTTCCTGAACCTGACCAGC |
| | | GAGAAGGTGTCCCAGGAAAAAGTGACAGAGATGATGAAGAAGTTCTG |
| | | CGCCCAGCCCTGGGAGGAAATCAAGACCTCCTACGCTGGCGTGAAAG |
| | | AGAAGTACCTGAGCGAGTACTGCTTCAGCGGTACCTACATCCTGAGC |
| | | CTGCTGCTGCAGGGCTACCACTTCACCGCCGATAGCTGGGAGCACAT |
| | | CCACTTCATCGGCAAGATTCAGGGCAGCGACGCCGGCTGGACACTGG |
| | | GCTACATGCTGAATCTGACCAACATG |
| CD39(aa46- | | |
| 461)_delta | | |
| cys3 | | |
| SEQ ID | Amino | NVKYGIVLDAGSSHTSLYIYKWPAEKENDTGVVHQVEECRVKGPGISKFV |
| NO: 111 | acid | QKVNEIGIYLTDCMERAREVIPRSQHQETPVYLGATAGMRLLRMESEELA |
| | | DRVLDVVERSLSNYPFDFQGARIITGQEEGAYGWITINYLLGKFSQKTRW |
| | | FSIVPYETNNQETFGALDLGGASTQVTFVPQNQTIESPDNALQFRLYGKD |
| | | YNVYTHSFLAYGKDQALWQKLAKDIQVASNEILRDPCFHPGYKKVVNVSD |
| | | LYKTPATKRFEMTLPFQQFEIQGIGNYQQCHQSILELFNTSYCPYSQCAFN |
| | | GIFLPPLQGDFGAFSAFYFVMKFLNLTSEKVSQEKVTEMMKKFCAQPWE |
| | | EIKTSYAGVKEKYLSEYCFSGTYILSLLLQGYHFTADSWEHIHFIGKIQGSD |
| | | AGWTLGYMLNLTNM |
| SEQ ID | DNA | AACGTGAAGTACGGCATCGTGCTGGATGCCGGCAGCAGCCACACCAG |
| NO: 112 | | CCTGTACATCTACAAGTGGCCTGCCGAGAAAGAAAACGATACCGGTGT |
| | | CGTGCACCAGGTGGAAGAGTGCAGAGTGAAGGGCCCTGGCATCAGC |
| | | AAGTTCGTGCAGAAAGTGAACGAGATCGGCATCTACCTGACCGACTG |
| | | CATGGAACGGGCCAGAGAAGTGATCCCCAGAAGCCAGCACCAGGAAA |
| | | CCCCCGTGTACCTGGGAGCCACAGCCGGCATGAGACTGCTGCGGAT |
| | | GGAAAGCGAGGAACTGGCCGACAGAGTGCTGGACGTGGTGGAAAGA |
| | | AGCCTGAGCAACTACCCATTCGATTTTCAAGGGGCCAGAATCATCACC |
| | | GGCCAGGAAGAGGGCGCTTACGGCTGGATCACCATCAACTACCTGCT |
| | | GGGCAAGTTCAGCCAGAAAACCCGGTGGTTCAGCATCGTGCCCTACG |
| | | AGACAAACAATCAGGAAACCTTCGGAGCCCTGGACCTGGGCGGAGCC |
| | | TCTACCCAAGTGACCTTCGTGCCCCAGAATCAGACCATCGAGAGCCC |
| | | CGACAACGCCCTGCAGTTCCGGCTGTACGGCAAGGACTACAATGTGT |
| | | ACACCCACAGCTTTCTGGCCTACGGAAAGGACCAGGCCCTGTGGCAG |
| | | AAGCTGGCCAAGGACATCCAGGTGGCCAGCAACGAGATCCTGCGGG |
| | | ACCCTTGCTTCCACCCCGGCTACAAGAAAGTCGTGAACGTGTCCGAC |
| | | CTGTACAAGACCCCCGCCACCAAGAGATTCGAGATGACCCTGCCCTT |
| | | CCAGCAGTTCGAGATCCAGGGCATCGGCAACTACCAGCAGTGCCACC |
| | | AGAGCATCCTGGAACTGTTCAACACCAGCTACTGCCCCTACAGCCAGT |
| | | GCGCCTTCAACGGCATCTTCCTGCCACCTCTGCAGGGGGACTTCGGC |
| | | GCTTTCAGCGCCTTCTACTTCGTGATGAAGTTCCTGAACCTGACCAGC |
| | | GAGAAGGTGTCCCAGGAAAAAGTGACAGAGATGATGAAGAAGTTCTG |
| | | CGCCCAGCCCTGGGAGGAAATCAAGACCTCCTACGCTGGCGTGAAAG |
| | | AGAAGTACCTGAGCGAGTACTGCTTCAGCGGTACCTACATCCTGAGC |
| | | CTGCTGCTGCAGGGCTACCACTTCACCGCCGATAGCTGGGAGCACAT |
| | | CCACTTCATCGGCAAGATTCAGGGCAGCGACGCCGGCTGGACACTGG |
| | | GCTACATGCTGAATCTGACCAACATG |
| CD39(aa46- | | |
| 461)_delta | | |
| cys4 | | |
| SEQ ID | Amino | NVKYGIVLDAGSSHTSLYIYKVVPAEKENDTGVVHQVEECRVKGPGISKFV |
| NO: 113 | acid | QKVNEIGIYLTDCMERAREVIPRSQHQETPVYLGATAGMRLLRMESEELA |
| | | DRVLDVVERSLSNYPFDFQGARIITGQEEGAYGWITINYLLGKFSQKTRW |
| | | FSIVPYETNNQETFGALDLGGASTQVTFVPQNQTIESPDNALQFRLYGKD |
| | | YNVYTHSFLCYGKDQALWQKLAKDIQVASNEILRDPCFHPGYKKVVNVSD |
| | | LYKTPCTKRFEMTLPFQQFEIQGIGNYQQCHQSILELFNTSYAPYSQAAFN |
| | | GIFLPPLQGDFGAFSAFYFVMKFLNLTSEKVSQEKVTEMMKKFCAQPWE |
| | | EIKTSYAGVKEKYLSEYCFSGTYILSLLLQGYHFTADSWEHIHFIGKIQGSD |
| | | AGWTLGYMLNLTNM |
| SEQ ID | DNA | AACGTGAAGTACGGCATCGTGCTGGATGCCGGCAGCAGCCACACCAG |
| NO: 114 | | CCTGTACATCTACAAGTGGCCTGCCGAGAAAGAAAACGATACCGGTGT |
| | | CGTGCACCAGGTGGAAGAGTGCAGAGTGAAGGGCCCTGGCATCAGC |
| | | AAGTTCGTGCAGAAAGTGAACGAGATCGGCATCTACCTGACCGACTG |
| | | CATGGAACGGGCCAGAGAAGTGATCCCCAGAAGCCAGCACCAGGAAA |
| | | CCCCCGTGTACCTGGGAGCCACAGCCGGCATGAGACTGCTGCGGAT |
| | | GGAAAGCGAGGAACTGGCCGACAGAGTGCTGGACGTGGTGGAAAGA |
| | | AGCCTGAGCAACTACCCATTCGATTTTCAAGGGGCCAGAATCATCACC |
| | | GGCCAGGAAGAGGGCGCTTACGGCTGGATCACCATCAACTACCTGCT |
| | | GGGCAAGTTCAGCCAGAAAACCCGGTGGTTCAGCATCGTGCCCTACG |
| | | AGACAAACAATCAGGAAACCTTCGGAGCCCTGGACCTGGGCGGAGCC |
| | | TCTACCCAAGTGACCTTCGTGCCCCAGAATCAGACCATCGAGAGCCC |
| | | CGACAACGCCCTGCAGTTCCGGCTGTACGGCAAGGACTACAATGTGT |
| | | ACACCCACAGCTTTCTGTGCTACGGAAAGGACCAGGCCCTGTGGCAG |
| | | AAGCTGGCCAAGGACATCCAGGTGGCCAGCAACGAGATCCTGCGGG |
| | | ACCCTTGCTTCCACCCCGGCTACAAGAAAGTCGTGAACGTGTCCGAC |
| | | CTGTACAAGACCCCCTGCACCAAGAGATTCGAGATGACCCTGCCCTTC |
| | | CAGCAGTTCGAGATCCAGGGCATCGGCAACTACCAGCAGTGCCACCA |
| | | GAGCATCCTGGAACTGTTCAACACCAGCTACGCCCCCTACAGCCAGG |
| | | CCGCCTTCAACGGCATCTTCCTGCCACCTCTGCAGGGGGACTTCGGC |
| | | GCTTTCAGCGCCTTCTACTTCGTGATGAAGTTCCTGAACCTGACCAGC |
| | | GAGAAGGTGTCCCAGGAAAAAGTGACAGAGATGATGAAGAAGTTCTG |
| | | CGCCCAGCCCTGGGAGGAAATCAAGACCTCCTACGCTGGCGTGAAAG |
| | | AGAAGTACCTGAGCGAGTACTGCTTCAGCGGTACCTACATCCTGAGC |
| | | CTGCTGCTGCAGGGCTACCACTTCACCGCCGATAGCTGGGAGCACAT |
| | | CCACTTCATCGGCAAGATTCAGGGCAGCGACGCCGGCTGGACACTGG |
| | | GCTACATGCTGAATCTGACCAACATG |
| CD39(aa46- | | |
| 461)_delta | | |
| cys5 | | |
| SEQ ID | Amino | NVKYGIVLDAGSSHTSLYIYKVVPAEKENDTGVVHQVEECRVKGPGISKFV |
| NO: 115 | acid | QKVNEIGIYLTDCMERAREVIPRSQHQETPVYLGATAGMRLLRMESEELA |
| | | DRVLDVVERSLSNYPFDFQGARIITGQEEGAYGWITINYLLGKFSQKTRW |
| | | FSIVPYETNNQETFGALDLGGASTQVTFVPQNQTIESPDNALQFRLYGKD |
| | | YNVYTHSFLCYGKDQALWQKLAKDIQVASNEILRDPCFHPGYKKVVNVSD |
| | | LYKTPCTKRFEMTLPFQQFEIQGIGNYQQCHQSILELFNTSYCPYSQCAF |
| | | NGIFLPPLQGDFGAFSAFYFVMKFLNLTSEKVSQEKVTEMMKKFAAQPW |
| | | EEIKTSYAGVKEKYLSEYAFSGTYILSLLLQGYHFTADSWEHIHFIGKIQGS |
| | | DAGWTLGYMLNLTNM |
| SEQ ID | DNA | AACGTGAAGTACGGCATCGTGCTGGATGCCGGCAGCAGCCACACCAG |
| NO: 116 | | CCTGTACATCTACAAGTGGCCTGCCGAGAAAGAAAACGATACCGGTGT |
| | | CGTGCACCAGGTGGAAGAGTGCAGAGTGAAGGGCCCTGGCATCAGC |
| | | AAGTTCGTGCAGAAAGTGAACGAGATCGGCATCTACCTGACCGACTG |
| | | CATGGAACGGGCCAGAGAAGTGATCCCCAGAAGCCAGCACCAGGAAA |
| | | CCCCCGTGTACCTGGGAGCCACAGCCGGCATGAGACTGCTGCGGAT |
| | | GGAAAGCGAGGAACTGGCCGACAGAGTGCTGGACGTGGTGGAAAGA |
| | | AGCCTGAGCAACTACCCATTCGATTTTCAAGGGGCCAGAATCATCACC |
| | | GGCCAGGAAGAGGGCGCTTACGGCTGGATCACCATCAACTACCTGCT |
| | | GGGCAAGTTCAGCCAGAAAACCCGGTGGTTCAGCATCGTGCCCTACG |
| | | AGACAAACAATCAGGAAACCTTCGGAGCCCTGGACCTGGGCGGAGCC |
| | | TCTACCCAAGTGACCTTCGTGCCCCAGAATCAGACCATCGAGAGCCC |
| | | CGACAACGCCCTGCAGTTCCGGCTGTACGGCAAGGACTACAATGTGT |
| | | ACACCCACAGCTTTCTGTGCTACGGAAAGGACCAGGCCCTGTGGCAG |
| | | AAGCTGGCCAAGGACATCCAGGTGGCCAGCAACGAGATCCTGCGGG |
| | | ACCCTTGCTTCCACCCCGGCTACAAGAAAGTCGTGAACGTGTCCGAC |
| | | CTGTACAAGACCCCCTGCACCAAGAGATTCGAGATGACCCTGCCCTTC |
| | | CAGCAGTTCGAGATCCAGGGCATCGGCAACTACCAGCAGTGCCACCA |
| | | GAGCATCCTGGAACTGTTCAACACCAGCTACTGCCCCTACAGCCAGT |
| | | GCGCCTTCAACGGCATCTTCCTGCCACCTCTGCAGGGGGACTTCGGC |
| | | GCTTTCAGCGCCTTCTACTTCGTGATGAAGTTCCTGAACCTGACCAGC |
| | | GAGAAGGTGTCCCAGGAAAAAGTGACAGAGATGATGAAGAAGTTCGC |
| | | CGCCCAGCCCTGGGAGGAAATCAAGACCTCCTACGCTGGCGTGAAAG |
| | | AGAAGTACCTGAGCGAGTACGCCTTCAGCGGTACCTACATCCTGAGC |
| | | CTGCTGCTGCAGGGCTACCACTTCACCGCCGATAGCTGGGAGCACAT |
| | | CCACTTCATCGGCAAGATTCAGGGCAGCGACGCCGGCTGGACACTGG |
| | | GCTACATGCTGAATCTGACCAACATG |
| CD39(aa46- | | |
| 461)_dMIL(193- | | |
| 204)_delta | | |
| cys1 | | |
| SEQ ID | Amino | NVKYGIVLDAGSSHTSLYIYKVVPAEKENDTGVVHQVEEARVKGPGISKFV |
| NO: 117 | acid | QKVNEIGIYLTDAMERAREVIPRSQHQETPVYLGATAGMRLLRMESEELA |
| | | DRVLDVVERSLSNYPFDFQGARIITGQEEGAYGWITINYLLGKFSQKNQET |
| | | FGALDLGGASTQVTFVPQNQTIESPDNALQFRLYGKDYNVYTHSFLCYGK |
| | | DQALWQKLAKDIQVASNEILRDPCFHPGYKKVVNVSDLYKTPCTKRFEMT |
| | | LPFQQFEIQGIGNYQQCHQSILELFNTSYCPYSQCAFNGIFLPPLQGDFGA |
| | | FSAFYFVMKFLNLTSEKVSQEKVTEMMKKFCAQPWEEIKTSYAGVKEKYL |
| | | SEYCFSGTYILSLLLQGYHFTADSWEHIHFIGKIQGSDAGWTLGYMLNLTN |
| | | M |
| SEQ ID | DNA | AACGTGAAGTACGGCATCGTGCTGGATGCCGGCAGCAGCCACACCAG |
| NO: 118 | | CCTGTACATCTACAAGTGGCCTGCCGAGAAAGAAAACGATACCGGTGT |
| | | CGTGCACCAGGTGGAAGAGGCCAGAGTGAAGGGCCCTGGCATCAGC |
| | | AAGTTCGTGCAGAAAGTGAACGAGATCGGCATCTACCTGACCGACGC |
| | | CATGGAACGGGCCAGAGAAGTGATCCCCAGAAGCCAGCACCAGGAAA |
| | | CCCCCGTGTACCTGGGAGCCACAGCCGGCATGAGACTGCTGCGGAT |
| | | GGAAAGCGAGGAACTGGCCGACAGAGTGCTGGACGTGGTGGAAAGA |
| | | AGCCTGAGCAACTACCCATTCGATTTTCAAGGGGCCAGAATCATCACC |
| | | GGCCAGGAAGAGGGCGCTTACGGCTGGATCACCATCAACTACCTGCT |
| | | GGGCAAGTTCAGCCAGAAAAATCAGGAAACCTTCGGAGCCCTGGACC |
| | | TGGGCGGAGCCTCTACCCAAGTGACCTTCGTGCCCCAGAATCAGACC |
| | | ATCGAGAGCCCCGACAACGCCCTGCAGTTCCGGCTGTACGGCAAGGA |
| | | CTACAATGTGTACACCCACAGCTTTCTGTGCTACGGAAAGGACCAGGC |
| | | CCTGTGGCAGAAGCTGGCCAAGGACATCCAGGTGGCCAGCAACGAG |
| | | ATCCTGCGGGACCCTTGCTTCCACCCCGGCTACAAGAAAGTCGTGAA |
| | | CGTGTCCGACCTGTACAAGACCCCCTGCACCAAGAGATTCGAGATGA |
| | | CCCTGCCCTTCCAGCAGTTCGAGATCCAGGGCATCGGCAACTACCAG |
| | | CAGTGCCACCAGAGCATCCTGGAACTGTTCAACACCAGCTACTGCCC |
| | | CTACAGCCAGTGCGCCTTCAACGGCATCTTCCTGCCACCTCTGCAGG |
| | | GGGACTTCGGCGCTTTCAGCGCCTTCTACTTCGTGATGAAGTTCCTGA |
| | | ACCTGACCAGCGAGAAGGTGTCCCAGGAAAAAGTGACAGAGATGATG |
| | | AAGAAGTTCTGCGCCCAGCCCTGGGAGGAAATCAAGACCTCCTACGC |
| | | TGGCGTGAAAGAGAAGTACCTGAGCGAGTACTGCTTCAGCGGTACCT |
| | | ACATCCTGAGCCTGCTGCTGCAGGGCTACCACTTCACCGCCGATAGC |
| | | TGGGAGCACATCCACTTCATCGGCAAGATTCAGGGCAGCGACGCCGG |
| | | CTGGACACTGGGCTACATGCTGAATCTGACCAACATG |
| CD39(aa46- | | |
| 461)_dMIL(193- | | |
| 204)_delta | | |
| cys2 | | |
| SEQ ID | Amino | NVKYGIVLDAGSSHTSLYIYKVVPAEKENDTGVVHQVEECRVKGPGISKFV |
| NO: 119 | acid | QKVNEIGIYLTDCMERAREVIPRSQHQETPVYLGATAGMRLLRMESEELA |
| | | DRVLDVVERSLSNYPFDFQGARIITGQEEGAYGWITINYLLGKFSQKNQET |
| | | FGALDLGGASTQVTFVPQNQTIESPDNALQFRLYGKDYNVYTHSFLCYGK |
| | | DQALWQKLAKDIQVASNEILRDPAFHPGYKKVVNVSDLYKTPCTKRFEMT |
| | | LPFQQFEIQGIGNYQQAHQSILELFNTSYCPYSQCAFNGIFLPPLQGDFGA |
| | | FSAFYFVMKFLNLTSEKVSQEKVTEMMKKFCAQPWEEIKTSYAGVKEKYL |
| | | SEYCFSGTYILSLLLQGYHFTADSWEHIHFIGKIQGSDAGVVTLGYMLNLTN |
| | | M |
| SEQ ID | DNA | AACGTGAAGTACGGCATCGTGCTGGATGCCGGCAGCAGCCACACCAG |
| NO: 120 | | CCTGTACATCTACAAGTGGCCTGCCGAGAAAGAAAACGATACCGGTGT |
| | | CGTGCACCAGGTGGAAGAGTGCAGAGTGAAGGGCCCTGGCATCAGC |
| | | AAGTTCGTGCAGAAAGTGAACGAGATCGGCATCTACCTGACCGACTG |
| | | CATGGAACGGGCCAGAGAAGTGATCCCCAGAAGCCAGCACCAGGAAA |
| | | CCCCCGTGTACCTGGGAGCCACAGCCGGCATGAGACTGCTGCGGAT |
| | | GGAAAGCGAGGAACTGGCCGACAGAGTGCTGGACGTGGTGGAAAGA |
| | | AGCCTGAGCAACTACCCATTCGATTTTCAAGGGGCCAGAATCATCACC |
| | | GGCCAGGAAGAGGGCGCTTACGGCTGGATCACCATCAACTACCTGCT |
| | | GGGCAAGTTCAGCCAGAAAAATCAGGAAACCTTCGGAGCCCTGGACC |
| | | TGGGCGGAGCCTCTACCCAAGTGACCTTCGTGCCCCAGAATCAGACC |
| | | ATCGAGAGCCCCGACAACGCCCTGCAGTTCCGGCTGTACGGCAAGGA |
| | | CTACAATGTGTACACCCACAGCTTTCTGTGCTACGGAAAGGACCAGGC |
| | | CCTGTGGCAGAAGCTGGCCAAGGACATCCAGGTGGCCAGCAACGAG |
| | | ATCCTGCGGGACCCTGCCTTCCACCCCGGCTACAAGAAAGTCGTGAA |
| | | CGTGTCCGACCTGTACAAGACCCCCTGCACCAAGAGATTCGAGATGA |
| | | CCCTGCCCTTCCAGCAGTTCGAGATCCAGGGCATCGGCAACTACCAG |
| | | CAGGCCCACCAGAGCATCCTGGAACTGTTCAACACCAGCTACTGCCC |
| | | CTACAGCCAGTGCGCCTTCAACGGCATCTTCCTGCCACCTCTGCAGG |
| | | GGGACTTCGGCGCTTTCAGCGCCTTCTACTTCGTGATGAAGTTCCTGA |
| | | ACCTGACCAGCGAGAAGGTGTCCCAGGAAAAAGTGACAGAGATGATG |
| | | AAGAAGTTCTGCGCCCAGCCCTGGGAGGAAATCAAGACCTCCTACGC |
| | | TGGCGTGAAAGAGAAGTACCTGAGCGAGTACTGCTTCAGCGGTACCT |
| | | ACATCCTGAGCCTGCTGCTGCAGGGCTACCACTTCACCGCCGATAGC |
| | | TGGGAGCACATCCACTTCATCGGCAAGATTCAGGGCAGCGACGCCGG |
| | | CTGGACACTGGGCTACATGCTGAATCTGACCAACATG |
| CD39(aa46- | | |
| 461)_dMIL(193- | | |
| 204)_delta | | |
| cys3 | | |
| SEQ ID | Amino | NVKYGIVLDAGSSHTSLYIYKVVPAEKENDTGVVHQVEECRVKGPGISKFV |
| NO: 121 | acid | QKVNEIGIYLTDCMERAREVIPRSQHQETPVYLGATAGMRLLRMESEELA |
| | | DRVLDVVERSLSNYPFDFQGARMTGQEEGAYGWITINYLLGKFSQKNQET |
| | | FGALDLGGASTQVTFVPQNQTESPDNALQFRLYGKDYNVYTHSFLAYGK |
| | | DQALWQKLAKDIQVASNEILRDPCFHPGYKKVVNVSDLYKTPATKRFEMT |
| | | LPFQQFEIQGIGNYQQCHQSILELFNTSYCPYSQCAFNGIFLPPLQGDFGA |
| | | FSAFYFVMKFLNLTSEKVSQEKVTEMMKKFCAQPWEEIKTSYAGVKEKYL |
| | | SEYCFSGTYILSLLLQGYHFTADSWEHIHFIGKIQGSDAGVVTLGYMLNLTN |
| | | M |
| SEQ ID | DNA | AACGTGAAGTACGGCATCGTGCTGGATGCCGGCAGCAGCCACACCAG |
| NO: 122 | | CCTGTACATCTACAAGTGGCCTGCCGAGAAAGAAAACGATACCGGTGT |
| | | CGTGCACCAGGTGGAAGAGTGCAGAGTGAAGGGCCCTGGCATCAGC |
| | | AAGTTCGTGCAGAAAGTGAACGAGATCGGCATCTACCTGACCGACTG |
| | | CATGGAACGGGCCAGAGAAGTGATCCCCAGAAGCCAGCACCAGGAAA |
| | | CCCCCGTGTACCTGGGAGCCACAGCCGGCATGAGACTGCTGCGGAT |
| | | GGAAAGCGAGGAACTGGCCGACAGAGTGCTGGACGTGGTGGAAAGA |
| | | AGCCTGAGCAACTACCCATTCGATTTTCAAGGGGCCAGAATCATCACC |
| | | GGCCAGGAAGAGGGCGCTTACGGCTGGATCACCATCAACTACCTGCT |
| | | GGGCAAGTTCAGCCAGAAAAATCAGGAAACCTTCGGAGCCCTGGACC |
| | | TGGGCGGAGCCTCTACCCAAGTGACCTTCGTGCCCCAGAATCAGACC |
| | | ATCGAGAGCCCCGACAACGCCCTGCAGTTCCGGCTGTACGGCAAGGA |
| | | CTACAATGTGTACACCCACAGCTTTCTGGCCTACGGAAAGGACCAGG |
| | | CCCTGTGGCAGAAGCTGGCCAAGGACATCCAGGTGGCCAGCAACGA |
| | | GATCCTGCGGGACCCTTGCTTCCACCCCGGCTACAAGAAAGTCGTGA |
| | | ACGTGTCCGACCTGTACAAGACCCCCGCCACCAAGAGATTCGAGATG |
| | | ACCCTGCCCTTCCAGCAGTTCGAGATCCAGGGCATCGGCAACTACCA |
| | | GCAGTGCCACCAGAGCATCCTGGAACTGTTCAACACCAGCTACTGCC |
| | | CCTACAGCCAGTGCGCCTTCAACGGCATCTTCCTGCCACCTCTGCAG |
| | | GGGGACTTCGGCGCTTTCAGCGCCTTCTACTTCGTGATGAAGTTCCTG |
| | | AACCTGACCAGCGAGAAGGTGTCCCAGGAAAAAGTGACAGAGATGAT |
| | | GAAGAAGTTCTGCGCCCAGCCCTGGGAGGAAATCAAGACCTCCTACG |
| | | CTGGCGTGAAAGAGAAGTACCTGAGCGAGTACTGCTTCAGCGGTACC |
| | | TACATCCTGAGCCTGCTGCTGCAGGGCTACCACTTCACCGCCGATAG |
| | | CTGGGAGCACATCCACTTCATCGGCAAGATTCAGGGCAGCGACGCCG |
| | | GCTGGACACTGGGCTACATGCTGAATCTGACCAACATG |
| CD39(aa46- | | |
| 461)_dMIL(193- | | |
| 204)_delta | | |
| cys4 | | |
| SEQ ID | Amino | NVKYGIVLDAGSSHTSLYIYKWPAEKENDTGVVHQVEECRVKGPGISKFV |
| NO: 123 | acid | QKVNEIGIYLTDCMERAREVIPRSQHQETPVYLGATAGMRLLRMESEELA |
| | | DRVLDVVERSLSNYPFDFQGARIITGQEEGAYGWITINYLLGKFSQKNQET |
| | | FGALDLGGASTQVTFVPQNQTESPDNALQFRLYGKDYNVYTHSFLCYGK |
| | | DQALWQKLAKDIQVASNEILRDPCFHPGYKKVVNVSDLYKTPCTKRFEMT |
| | | LPFQQFEIQGIGNYQQCHQSILELFNTSYAPYSQAAFNGIFLPPLQGDFGA |
| | | FSAFYFVMKFLNLTSEKVSQEKVTEMMKKFCAQPWEEIKTSYAGVKEKYL |
| | | SEYCFSGTYILSLLLQGYHFTADSWEHIHFIGKIQGSDAGWTLGYMLNLTN |
| | | M |
| SEQ ID | DNA | AACGTGAAGTACGGCATCGTGCTGGATGCCGGCAGCAGCCACACCAG |
| NO: 124 | | CCTGTACATCTACAAGTGGCCTGCCGAGAAAGAAAACGATACCGGTGT |
| | | CGTGCACCAGGTGGAAGAGTGCAGAGTGAAGGGCCCTGGCATCAGC |
| | | AAGTTCGTGCAGAAAGTGAACGAGATCGGCATCTACCTGACCGACTG |
| | | CATGGAACGGGCCAGAGAAGTGATCCCCAGAAGCCAGCACCAGGAAA |
| | | CCCCCGTGTACCTGGGAGCCACAGCCGGCATGAGACTGCTGCGGAT |
| | | GGAAAGCGAGGAACTGGCCGACAGAGTGCTGGACGTGGTGGAAAGA |
| | | AGCCTGAGCAACTACCCATTCGATTTTCAAGGGGCCAGAATCATCACC |
| | | GGCCAGGAAGAGGGCGCTTACGGCTGGATCACCATCAACTACCTGCT |
| | | GGGCAAGTTCAGCCAGAAAAATCAGGAAACCTTCGGAGCCCTGGACC |
| | | TGGGCGGAGCCTCTACCCAAGTGACCTTCGTGCCCCAGAATCAGACC |
| | | ATCGAGAGCCCCGACAACGCCCTGCAGTTCCGGCTGTACGGCAAGGA |
| | | CTACAATGTGTACACCCACAGCTTTCTGTGCTACGGAAAGGACCAGGC |
| | | CCTGTGGCAGAAGCTGGCCAAGGACATCCAGGTGGCCAGCAACGAG |
| | | ATCCTGCGGGACCCTTGCTTCCACCCCGGCTACAAGAAAGTCGTGAA |
| | | CGTGTCCGACCTGTACAAGACCCCCTGCACCAAGAGATTCGAGATGA |
| | | CCCTGCCCTTCCAGCAGTTCGAGATCCAGGGCATCGGCAACTACCAG |
| | | CAGTGCCACCAGAGCATCCTGGAACTGTTCAACACCAGCTACGCCCC |
| | | CTACAGCCAGGCCGCCTTCAACGGCATCTTCCTGCCACCTCTGCAGG |
| | | GGGACTTCGGCGCTTTCAGCGCCTTCTACTTCGTGATGAAGTTCCTGA |
| | | ACCTGACCAGCGAGAAGGTGTCCCAGGAAAAAGTGACAGAGATGATG |
| | | AAGAAGTTCTGCGCCCAGCCCTGGGAGGAAATCAAGACCTCCTACGC |
| | | TGGCGTGAAAGAGAAGTACCTGAGCGAGTACTGCTTCAGCGGTACCT |
| | | ACATCCTGAGCCTGCTGCTGCAGGGCTACCACTTCACCGCCGATAGC |
| | | TGGGAGCACATCCACTTCATCGGCAAGATTCAGGGCAGCGACGCCGG |
| | | CTGGACACTGGGCTACATGCTGAATCTGACCAACATG |
| CD39(aa46- | | |
| 461)_dMIL(193- | | |
| 204)_delta | | |
| cys5 | | |
| SEQ ID | Amino | NVKYGIVLDAGSSHTSLYIYKWPAEKENDTGVVHQVEECRVKGPGISKFV |
| NO: 125 | acid | QKVNEIGIYLTDCMERAREVIPRSQHQETPVYLGATAGMRLLRMESEELA |
| | | DRVLDVVERSLSNYPFDFQGARIITGQEEGAYGWITINYLLGKFSQKNQET |
| | | FGALDLGGASTQVTFVPQNQTESPDNALQFRLYGKDYNVYTHSFLCYGK |
| | | DQALWQKLAKDIQVASNEILRDPCFHPGYKKVVNVSDLYKTPCTKRFEMT |
| | | LPFQQFEIQGIGNYQQCHQSILELFNTSYCPYSQCAFNGIFLPPLQGDFGA |
| | | FSAFYFVMKFLNLTSEKVSQEKVTEMMKKFAAQPWEEIKTSYAGVKEKYL |
| | | SEYAFSGTYILSLLLQGYHFTADSWEHIHFIGKIQGSDAGWTLGYMLNLTN |
| | | M |
| SEQ ID | DNA | AACGTGAAGTACGGCATCGTGCTGGATGCCGGCAGCAGCCACACCAG |
| NO: 126 | | CCTGTACATCTACAAGTGGCCTGCCGAGAAAGAAAACGATACCGGTGT |
| | | CGTGCACCAGGTGGAAGAGTGCAGAGTGAAGGGCCCTGGCATCAGC |
| | | AAGTTCGTGCAGAAAGTGAACGAGATCGGCATCTACCTGACCGACTG |
| | | CATGGAACGGGCCAGAGAAGTGATCCCCAGAAGCCAGCACCAGGAAA |
| | | CCCCCGTGTACCTGGGAGCCACAGCCGGCATGAGACTGCTGCGGAT |
| | | GGAAAGCGAGGAACTGGCCGACAGAGTGCTGGACGTGGTGGAAAGA |
| | | AGCCTGAGCAACTACCCATTCGATTTTCAAGGGGCCAGAATCATCACC |
| | | GGCCAGGAAGAGGGCGCTTACGGCTGGATCACCATCAACTACCTGCT |
| | | GGGCAAGTTCAGCCAGAAAAATCAGGAAACCTTCGGAGCCCTGGACC |
| | | TGGGCGGAGCCTCTACCCAAGTGACCTTCGTGCCCCAGAATCAGACC |
| | | ATCGAGAGCCCCGACAACGCCCTGCAGTTCCGGCTGTACGGCAAGGA |
| | | CTACAATGTGTACACCCACAGCTTTCTGTGCTACGGAAAGGACCAGGC |
| | | CCTGTGGCAGAAGCTGGCCAAGGACATCCAGGTGGCCAGCAACGAG |
| | | ATCCTGCGGGACCCTTGCTTCCACCCCGGCTACAAGAAAGTCGTGAA |
| | | CGTGTCCGACCTGTACAAGACCCCCTGCACCAAGAGATTCGAGATGA |
| | | CCCTGCCCTTCCAGCAGTTCGAGATCCAGGGCATCGGCAACTACCAG |
| | | CAGTGCCACCAGAGCATCCTGGAACTGTTCAACACCAGCTACTGCCC |
| | | CTACAGCCAGTGCGCCTTCAACGGCATCTTCCTGCCACCTCTGCAGG |
| | | GGGACTTCGGCGCTTTCAGCGCCTTCTACTTCGTGATGAAGTTCCTGA |
| | | ACCTGACCAGCGAGAAGGTGTCCCAGGAAAAAGTGACAGAGATGATG |
| | | AAGAAGTTCGCCGCCCAGCCCTGGGAGGAAATCAAGACCTCCTACGC |
| | | TGGCGTGAAAGAGAAGTACCTGAGCGAGTACGCCTTCAGCGGTACCT |
| | | ACATCCTGAGCCTGCTGCTGCAGGGCTACCACTTCACCGCCGATAGC |
| | | TGGGAGCACATCCACTTCATCGGCAAGATTCAGGGCAGCGACGCCGG |
| | | CTGGACACTGGGCTACATGCTGAATCTGACCAACATG |
| CD39(aa38- | | |
| 476)_delta337-344 | | |
| SEQ ID | Amino | TQNKALPENVKYGIVLDAGSSHTSLYIYKWPAEKENDTGVVHQVEECRVK |
| NO: 127 | acid | GPGISKFVQKVNEIGIYLTDCMERAREVIPRSQHQETPVYLGATAGMRLLR |
| | | MESEELADRVLDVVERSLSNYPFDFQGARIITGQEEGAYGWITINYLLGKF |
| | | SQKTRWFSIVPYETNNQETFGALDLGGASTQVTFVPQNQTIESPDNALQF |
| | | RLYGKDYNVYTHSFLCYGKDQALWQKLAKDIQVASNEILRDPCFHPGYKK |
| | | VVNVSDLYKTPCTKRFEMTLPFQQFEIQGIGNYQQCHQSILELFNTSFNGI |
| | | FLPPLQGDFGAFSAFYFVMKFLNLTSEKVSQEKVTEMMKKFCAQPWEEIK |
| | | TSYAGVKEKYLSEYCFSGTYILSLLLQGYHFTADSWEHIHFIGKIQGSDAG |
| | | VVTLGYMLNLTNMIPAEQPLSTPLSHST |
| SEQ ID | DNA | ACACAGAACAAAGCCCTGCCCGAGAACGTGAAGTACGGCATCGTGCT |
| NO: 128 | | GGATGCCGGCAGCAGCCACACCAGCCTGTACATCTACAAGTGGCCTG |
| | | CCGAGAAAGAAAACGATACCGGTGTCGTGCACCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCAGAGAAGTGA |
| | | TCCCCAGAAGCCAGCACCAGGAAACCCCCGTGTACCTGGGAGCCACA |
| | | GCCGGCATGAGACTGCTGCGGATGGAAAGCGAGGAACTGGCCGACA |
| | | GAGTGCTGGACGTGGTGGAAAGAAGCCTGAGCAACTACCCATTCGAT |
| | | TTTCAAGGGGCCAGAATCATCACCGGCCAGGAAGAGGGCGCTTACGG |
| | | CTGGATCACCATCAACTACCTGCTGGGCAAGTTCAGCCAGAAAACCC |
| | | GGTGGTTCAGCATCGTGCCCTACGAGACAAACAATCAGGAAACCTTC |
| | | GGAGCCCTGGACCTGGGCGGAGCCTCTACCCAAGTGACCTTCGTGCC |
| | | CCAGAATCAGACCATCGAGAGCCCCGACAACGCCCTGCAGTTCCGGC |
| | | TGTACGGCAAGGACTACAATGTGTACACCCACAGCTTTCTGTGCTACG |
| | | GAAAGGACCAGGCCCTGTGGCAGAAGCTGGCCAAGGACATCCAGGT |
| | | GGCCAGCAACGAGATCCTGCGGGACCCTTGCTTCCACCCCGGCTACA |
| | | AGAAAGTCGTGAACGTGTCCGACCTGTACAAGACCCCCTGCACCAAG |
| | | AGATTCGAGATGACCCTGCCCTTCCAGCAGTTCGAGATCCAGGGCAT |
| | | CGGCAACTACCAGCAGTGCCACCAGAGCATCCTGGAACTGTTCAACA |
| | | CCAGCTTCAACGGCATCTTCCTGCCACCTCTGCAGGGGGACTTCGGC |
| | | GCTTTCAGCGCCTTCTACTTCGTGATGAAGTTCCTGAACCTGACCAGC |
| | | GAGAAGGTGTCCCAGGAAAAAGTGACAGAGATGATGAAGAAGTTCTG |
| | | CGCCCAGCCCTGGGAGGAAATCAAGACCTCCTACGCTGGCGTGAAAG |
| | | AGAAGTACCTGAGCGAGTACTGCTTCAGCGGTACCTACATCCTGAGC |
| | | CTGCTGCTGCAGGGCTACCACTTCACCGCCGATAGCTGGGAGCACAT |
| | | CCACTTCATCGGCAAGATTCAGGGCAGCGACGCCGGCTGGACACTGG |
| | | GCTACATGCTGAATCTGACCAACATGATCCCCGCCGAGCAGCCCCTG |
| | | AGCACACCTCTGTCTCACAGCACC |
| CD39(aa38- | | |
| 476)_C338A_C343A | | |
| SEQ ID | Amino | TQNKALPENVKYGIVLDAGSSHTSLYIYKWPAEKENDTGVVHQVEECRVK |
| NO: 129 | acid | GPGISKFVQKVNEIGIYLTDCMERAREVIPRSQHQETPVYLGATAGMRLLR |
| | | MESEELADRVLDVVERSLSNYPFDFQGARIITGQEEGAYGWITINYLLGKF |
| | | SQKTRWFSIVPYETNNQETFGALDLGGASTQVTFVPQNQTIESPDNALQF |
| | | RLYGKDYNVYTHSFLCYGKDQALWQKLAKDIQVASNEILRDPCFHPGYKK |
| | | VVNVSDLYKTPCTKRFEMTLPFQQFEIQGIGNYQQCHQSILELFNTSYAPY |
| | | SQAAFNGIFLPPLQGDFGAFSAFYFVMKFLNLTSEKVSQEKVTEMMKKFC |
| | | AQPWEEIKTSYAGVKEKYLSEYCFSGTYILSLLLQGYHFTADSWEHIHFIG |
| | | KIQGSDAGWTLGYMLNLTNMIPAEQPLSTPLSHST |
| SEQ ID | DNA | ACACAGAACAAAGCCCTGCCCGAGAACGTGAAGTACGGCATCGTGCT |
| NO: 130 | | GGATGCCGGCAGCAGCCACACCAGCCTGTACATCTACAAGTGGCCTG |
| | | CCGAGAAAGAAAACGATACCGGTGTCGTGCACCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCAGAGAAGTGA |
| | | TCCCCAGAAGCCAGCACCAGGAAACCCCCGTGTACCTGGGAGCCACA |
| | | GCCGGCATGAGACTGCTGCGGATGGAAAGCGAGGAACTGGCCGACA |
| | | GAGTGCTGGACGTGGTGGAAAGAAGCCTGAGCAACTACCCATTCGAT |
| | | TTTCAAGGGGCCAGAATCATCACCGGCCAGGAAGAGGGCGCTTACGG |
| | | CTGGATCACCATCAACTACCTGCTGGGCAAGTTCAGCCAGAAAACCC |
| | | GGTGGTTCAGCATCGTGCCCTACGAGACAAACAATCAGGAAACCTTC |
| | | GGAGCCCTGGACCTGGGCGGAGCCTCTACCCAAGTGACCTTCGTGCC |
| | | CCAGAATCAGACCATCGAGAGCCCCGACAACGCCCTGCAGTTCCGGC |
| | | TGTACGGCAAGGACTACAATGTGTACACCCACAGCTTTCTGTGCTACG |
| | | GAAAGGACCAGGCCCTGTGGCAGAAGCTGGCCAAGGACATCCAGGT |
| | | GGCCAGCAACGAGATCCTGCGGGACCCTTGCTTCCACCCCGGCTACA |
| | | AGAAAGTCGTGAACGTGTCCGACCTGTACAAGACCCCCTGCACCAAG |
| | | AGATTCGAGATGACCCTGCCCTTCCAGCAGTTCGAGATCCAGGGCAT |
| | | CGGCAACTACCAGCAGTGCCACCAGAGCATCCTGGAACTGTTCAACA |
| | | CCAGCTACGCCCCCTACAGCCAGGCCGCCTTCAACGGCATCTTCCTG |
| | | CCACCTCTGCAGGGGGACTTCGGCGCTTTCAGCGCCTTCTACTTCGT |
| | | GATGAAGTTCCTGAACCTGACCAGCGAGAAGGTGTCCCAGGAAAAAG |
| | | TGACAGAGATGATGAAGAAGTTCTGCGCCCAGCCCTGGGAGGAAATC |
| | | AAGACCTCCTACGCTGGCGTGAAAGAGAAGTACCTGAGCGAGTACTG |
| | | CTTCAGCGGTACCTACATCCTGAGCCTGCTGCTGCAGGGCTACCACTT |
| | | CACCGCCGATAGCTGGGAGCACATCCACTTCATCGGCAAGATTCAGG |
| | | GCAGCGACGCCGGCTGGACACTGGGCTACATGCTGAATCTGACCAAC |
| | | ATGATCCCCGCCGAGCAGCCCCTGAGCACACCTCTGTCTCACAGCAC |
| | | C |
| Expression tag | | |
| aa1-16 | | |
| SEQ ID | Amino | APTSSSTKKTQLTSSG |
| NO: 131 | acid | |
| SEQ ID | DNA | GCCCCCACCAGCAGCAGCACCAAGAAGACCCAGCTGACCAGCAGCG |
| NO: 132 | | GC |
| Expression tag | | |
| aa1-15 | | |
| SEQ ID | Amino | APTSSSTKKTQLTSS |
| NO: 133 | acid | |
| SEQ ID | DNA | GCCCCTACCAGCAGCAGCACCAAGAAAACCCAGCTGACCAGCAGC |
| NO: 134 | | |
| Expression tag | | |
| aa1-6 | | |
| SEQ ID | Amino | APTSSS |
| NO: 135 | acid | |
| SEQ ID | DNA | GCCCCTACCAGCAGCAGC |
| NO: 136 | | |
| Expression tag | | |
| aa1-3 | | |
| SEQ ID | Amino | APT |
| NO: 137 | acid | |
| SEQ ID | DNA | GCCCCTACC |
| NO: 138 | | |
| Expression tag | | |
| aa1-9 | | |
| SEQ ID | Amino | APTSSSTKK |
| NO: 139 | acid | |
| SEQ ID | DNA | GCCCCTACCAGCAGCAGCACCAAGAAA |
| NO: 140 | | |
| Expression tag | | |
| aa1-12 | | |
| SEQ ID | Amino | APTSSSTKKTQL |
| NO: 141 | acid | |
| SEQ ID | DNA | GCCCCTACCAGCAGCAGCACCAAGAAAACCCAGCTG |
| NO: 142 | | |
| Expression tag | | |
| aa4-12 | | |
| SEQ ID | Amino | SSSTKKTQL |
| NO: 143 | acid | |
| SEQ ID | DNA | AGCAGCAGCACCAAGAAAACCCAGCTG |
| NO: 144 | | |
| EP28_8M | | |
| SEQ ID | Amino | TQNKALPENVKYGIVLDAGSSHTSLYIYKWPAEKENDTGVVHQVEECRVK |
| NO: 145 | acid | GPGISKFVQKVNEIGIYLTDCMERAREVIPRSQHQETPVYLGATAGMRLLR |
| | | MESEELADRVLDVVERSLSNYPFDFQGARIITGQDEGAYGWITINYLLGKF |
| | | SQKNQETFGALDLGGASTQVTFVPQNQTIESPDNALQFRLYGKDYNVYT |
| | | HSFLCYGRDQALWQKLAKDIQVASNEILRDPCFHPGYKKVVNVSDLYKTP |
| | | CTKRFEMTLPFQQFEIQGIGNYQQCHQSILELFNTSYCPYSQCAFNGIFLP |
| | | PLQGDFGAFSNFYYVMKFLNLTSEKVSQEKVTEMMKKFCAQPWEEIKTS |
| | | YAGQERWLRDYCFSGTYILSLLLQGYHFTADSWEHIHFIGKIQGSDAGWT |
| | | LGYMLNLTNMIPAEQPLSTPLSHST |
| SEQ ID | DNA | ACCCAGAACAAGGCCCTGCCCGAGAACGTGAAGTACGGCATCGTGCT |
| NO: 146 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCAGAGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGCTGGACGTGGTGGAAAGAAGCCTGAGCAACTACCCATTCGAT |
| | | TTTCAAGGCGCCAGAATCATCACCGGCCAGGACGAGGGCGCCTACGG |
| | | CTGGATCACCATCAACTACCTGCTGGGCAAGTTCAGCCAGAAGAATCA |
| | | GGAAACCTTCGGCGCCCTGGACCTGGGCGGAGCTTCTACCCAAGTGA |
| | | CCTTCGTGCCCCAGAATCAGACCATCGAGAGCCCCGACAACGCCCTG |
| | | CAGTTCCGGCTGTACGGCAAGGACTACAATGTGTACACCCACAGCTTT |
| | | CTGTGCTACGGCCGGGACCAGGCTCTGTGGCAGAAGCTGGCCAAGG |
| | | ACATCCAGGTGGCCAGCAACGAGATCCTGCGGGACCCTTGCTTCCAC |
| | | CCCGGCTACAAGAAAGTCGTGAACGTGTCCGACCTGTACAAGACCCC |
| | | CTGCACCAAGAGATTCGAGATGACCCTGCCCTTCCAGCAGTTCGAGAT |
| | | CCAGGGCATCGGCAACTACCAGCAGTGCCACCAGAGCATCCTGGAAC |
| | | TGTTCAACACCAGCTACTGCCCCTACAGCCAGTGCGCCTTCAACGGC |
| | | ATCTTCCTGCCACCTCTGCAGGGGGATTTCGGCGCCTTCAGCAACTTC |
| | | TACTACGTGATGAAGTTCCTGAACCTGACCAGCGAGAAGGTGTCCCA |
| | | GGAAAAAGTGACAGAGATGATGAAGAAGTTCTGCGCCCAGCCCTGGG |
| | | AGGAAATCAAGACCTCTTACGCCGGACAGGAACGGTGGCTGCGGGAC |
| | | TACTGTTTCAGCGGCACCTACATCCTGTCCCTGCTGCTGCAGGGCTAC |
| | | CACTTCACCGCCGATAGCTGGGAGCACATCCACTTCATCGGCAAGATT |
| | | CAGGGCAGCGACGCCGGCTGGACACTGGGCTACATGCTGAATCTGAC |
| | | CAACATGATCCCCGCCGAGCAGCCCCTGAGCACACCTCTGTCTCACA |
| | | GCACC |
| CD39(aa38- | | |
| 476)_dMIL(193- | | |
| 204)_CKAPPA | | |
| SEQ ID | Amino | TVAAPSVFIFPPSDEQLKSGTASVVCLLNNFYPREAKVQWKVDNALQSGN |
| NO: 147 | acid | SQESVTEQDSKDSTYSLSSTLTLSKADYEKHKVYACEVTHQGLSSPVTKS |
| | | FNRGEGGGGSTQNKALPENVKYGIVLDAGSSHTSLYIYKWPAEKENDTG |
| | | VVHQVEECRVKGPGISKFVQKVNEIGIYLTDCMERAREVIPRSQHQETPV |
| | | YLGATAGMRLLRMESEELADRVLDVVERSLSNYPFDFQGARIITGQEEGA |
| | | YGWITINYLLGKFSQKNQETFGALDLGGASTQVTFVPQNQTIESPDNALQ |
| | | FRLYGKDYNVYTHSFLCYGKDQALWQKLAKDIQVASNEILRDPCFHPGYK |
| | | KVVNVSDLYKTPCTKRFEMTLPFQQFEIQGIGNYQQCHQSILELFNTSYCP |
| | | YSQCAFNGIFLPPLQGDFGAFSAFYFVMKFLNLTSEKVSQEKVTEMMKKF |
| | | CAQPWEEIKTSYAGVKEKYLSEYCFSGTYILSLLLQGYHFTADSVVEHIHFI |
| | | GKIQGSDAGVVTLGYMLNLTNMIPAEQPLSTPLSHST |
| SEQ ID | DNA | ACGGTGGCCGCTCCCAGCGTGTTCATCTTCCCCCCCAGCGACGAGCA |
| NO: 148 | | GCTGAAGAGCGGCACCGCCAGCGTGGTGTGCCTGCTGAACAACTTCT |
| | | ACCCCCGGGAGGCCAAGGTGCAGTGGAAGGTGGACAACGCCCTGCA |
| | | GAGCGGCAACAGCCAGGAAAGCGTCACCGAGCAGGACAGCAAGGAC |
| | | TCCACCTACAGCCTGAGCAGCACCCTGACCCTGAGCAAGGCCGACTA |
| | | CGAGAAGCACAAGGTGTACGCCTGCGAGGTGACCCACCAGGGCCTG |
| | | TCCAGCCCCGTGACCAAGAGCTTCAACCGGGGCGAGGGAGGCGGAG |
| | | GATCTACCCAGAACAAGGCCCTGCCCGAGAACGTGAAGTACGGCATC |
| | | GTGCTGGATGCCGGCAGCAGCCACACCAGCCTGTACATCTACAAGTG |
| | | GCCTGCCGAGAAAGAAAACGACACCGGCGTGGTGCATCAGGTGGAA |
| | | GAGTGCAGAGTGAAGGGCCCTGGCATCAGCAAGTTCGTGCAGAAAGT |
| | | GAACGAGATCGGCATCTACCTGACCGACTGCATGGAACGGGCCAGGG |
| | | AAGTGATCCCCAGAAGCCAGCACCAGGAAACCCCCGTGTATCTGGGA |
| | | GCCACCGCCGGCATGAGACTGCTGAGAATGGAAAGCGAGGAACTGG |
| | | CCGACCGGGTGCTGGACGTGGTGGAAAGAAGCCTGAGCAACTACCCA |
| | | TTCGATTTTCAAGGCGCCAGAATCATCACCGGCCAGGAAGAAGGCGC |
| | | CTACGGCTGGATCACCATCAACTACCTGCTGGGCAAGTTCAGCCAGA |
| | | AGAATCAGGAAACCTTCGGCGCCCTGGACCTGGGCGGAGCTTCTACC |
| | | CAAGTGACCTTCGTGCCCCAGAATCAGACCATCGAGAGCCCCGACAA |
| | | CGCCCTGCAGTTCCGGCTGTACGGCAAGGACTACAATGTGTACACCC |
| | | ACAGCTTTCTGTGCTACGGAAAGGACCAGGCTCTGTGGCAGAAGCTG |
| | | GCCAAGGACATCCAGGTGGCCAGCAACGAGATCCTGCGGGACCCTTG |
| | | CTTCCACCCCGGCTACAAGAAAGTCGTGAACGTGTCCGACCTGTACAA |
| | | GACCCCCTGCACCAAGAGATTCGAGATGACCCTGCCCTTCCAGCAGT |
| | | TCGAGATCCAGGGCATCGGCAATTACCAGCAGTGCCACCAGAGCATC |
| | | CTGGAACTGTTCAACACCAGCTACTGCCCCTACAGCCAGTGCGCCTTC |
| | | AACGGCATCTTCCTGCCACCTCTGCAGGGGGATTTCGGCGCCTTCAG |
| | | CGCCTTCTACTTCGTGATGAAGTTCCTGAACCTGACCAGCGAGAAGGT |
| | | GTCCCAGGAAAAAGTGACAGAGATGATGAAGAAGTTCTGCGCCCAGC |
| | | CCTGGGAGGAAATCAAGACCTCCTACGCTGGCGTGAAAGAGAAGTAC |
| | | CTGAGCGAGTACTGCTTCAGCGGCACCTACATCCTGAGCCTGCTGCT |
| | | GCAGGGCTACCACTTCACCGCCGATAGCTGGGAGCACATCCACTTCA |
| | | TCGGCAAGATTCAGGGCAGCGACGCCGGCTGGACACTGGGCTACAT |
| | | GCTGAATCTGACCAACATGATCCCCGCCGAGCAGCCCCTGAGCACAC |
| | | CTCTGAGCCACAGCACC |
| CD39(aa38- | | |
| 476)_dMIL(193- | | |
| 204)_UBI | | |
| SEQ ID | Amino | MQIFVKTLTGKTITLEVEPSDTIENVKAKIQDKEGIPPDQQRLIFAGKQLED |
| NO: 149 | acid | GRTLSDYNIQKESTLHLVLRLRGGGGGGSTQNKALPENVKYGIVLDAGSS |
| | | HTSLYIYKWPAEKENDTGVVHQVEECRVKGPGISKFVQKVNEIGIYLTDC |
| | | MERAREVIPRSQHQETPVYLGATAGMRLLRMESEELADRVLDVVERSLS |
| | | NYPFDFQGARIITGQEEGAYGWITINYLLGKFSQKNQETFGALDLGGAST |
| | | QVTFVPQNQTESPDNALQFRLYGKDYNVYTHSFLCYGKDQALWQKLAK |
| | | DIQVASNEILRDPCFHPGYKKVVNVSDLYKTPCTKRFEMTLPFQQFEIQGI |
| | | GNYQQCHQSILELFNTSYCPYSQCAFNGIFLPPLQGDFGAFSAFYFVMKF |
| | | LNLTSEKVSQEKVTEMMKKFCAQPWEEIKTSYAGVKEKYLSEYCFSGTYI |
| | | LSLLLQGYHFTADSWEHIHFIGKIQGSDAGWTLGYMLNLTNMIPAEQPLST |
| | | PLSHST |
| SEQ ID | DNA | ATGCAAATCTTCGTGAAGACCCTGACTGGTAAGACCATCACCCTCGAG |
| NO: 150 | | GTGGAGCCCAGTGACACCATCGAGAATGTCAAGGCAAAGATCCAAGA |
| | | TAAGGAAGGCATCCCTCCTGATCAGCAGAGGTTGATCTTTGCTGGGAA |
| | | ACAGCTGGAAGATGGACGCACCCTGTCTGACTACAACATCCAGAAAG |
| | | AGTCCACTCTGCACTTGGTCCTGCGCTTGAGGGGGGGTGGAGGCGG |
| | | AGGATCTACCCAGAACAAGGCCCTGCCCGAGAACGTGAAGTACGGCA |
| | | TCGTGCTGGATGCCGGCAGCAGCCACACCAGCCTGTACATCTACAAG |
| | | TGGCCTGCCGAGAAAGAAAACGACACCGGCGTGGTGCATCAGGTGGA |
| | | AGAGTGCAGAGTGAAGGGCCCTGGCATCAGCAAGTTCGTGCAGAAAG |
| | | TGAACGAGATCGGCATCTACCTGACCGACTGCATGGAACGGGCCAGG |
| | | GAAGTGATCCCCAGAAGCCAGCACCAGGAAACCCCCGTGTATCTGGG |
| | | AGCCACCGCCGGCATGAGACTGCTGAGAATGGAAAGCGAGGAACTG |
| | | GCCGACCGGGTGCTGGACGTGGTGGAAAGAAGCCTGAGCAACTACC |
| | | CATTCGATTTTCAAGGCGCCAGAATCATCACCGGCCAGGAAGAAGGC |
| | | GCCTACGGCTGGATCACCATCAACTACCTGCTGGGCAAGTTCAGCCA |
| | | GAAGAATCAGGAAACCTTCGGCGCCCTGGACCTGGGCGGAGCTTCTA |
| | | CCCAAGTGACCTTCGTGCCCCAGAATCAGACCATCGAGAGCCCCGAC |
| | | AACGCCCTGCAGTTCCGGCTGTACGGCAAGGACTACAATGTGTACAC |
| | | CCACAGCTTTCTGTGCTACGGAAAGGACCAGGCTCTGTGGCAGAAGC |
| | | TGGCCAAGGACATCCAGGTGGCCAGCAACGAGATCCTGCGGGACCCT |
| | | TGCTTCCACCCCGGCTACAAGAAAGTCGTGAACGTGTCCGACCTGTA |
| | | CAAGACCCCCTGCACCAAGAGATTCGAGATGACCCTGCCCTTCCAGC |
| | | AGTTCGAGATCCAGGGCATCGGCAATTACCAGCAGTGCCACCAGAGC |
| | | ATCCTGGAACTGTTCAACACCAGCTACTGCCCCTACAGCCAGTGCGC |
| | | CTTCAACGGCATCTTCCTGCCACCTCTGCAGGGGGATTTCGGCGCCT |
| | | TCAGCGCCTTCTACTTCGTGATGAAGTTCCTGAACCTGACCAGCGAGA |
| | | AGGTGTCCCAGGAAAAAGTGACAGAGATGATGAAGAAGTTCTGCGCC |
| | | CAGCCCTGGGAGGAAATCAAGACCTCCTACGCTGGCGTGAAAGAGAA |
| | | GTACCTGAGCGAGTACTGCTTCAGCGGCACCTACATCCTGAGCCTGC |
| | | TGCTGCAGGGCTACCACTTCACCGCCGATAGCTGGGAGCACATCCAC |
| | | TTCATCGGCAAGATTCAGGGCAGCGACGCCGGCTGGACACTGGGCTA |
| | | CATGCTGAATCTGACCAACATGATCCCCGCCGAGCAGCCCCTGAGCA |
| | | CACCTCTGAGCCACAGCACC |
| CD39(aa38- | HSA Domain | |
| 476_dMIL(193- | I-G4S- | |
| 204_HSAI | CD39-dMIL | |
| SEQ ID | Amino | DAHKSEVAHRFKDLGEENFKALVLIAFAQYLQQSPFEDHVKLVNEVTEFA |
| NO: 151 | acid | KTCVADESAENCDKSLHTLFGDKLCTVATLRETYGEMADCCAKQEPERN |
| | | ECFLQHKDDNPNLPRLVRPEVDVMCTAFHDNEETFLKKYLYEIARRHPYF |
| | | YAPELLFFAKRYKAAFTECCQAADKAACLLPKLDELRGGGGSTQNKALPE |
| | | NVKYGIVLDAGSSHTSLYIYKWPAEKENDTGVVHQVEECRVKGPGISKFV |
| | | QKVNEIGIYLTDCMERAREVIPRSQHQETPVYLGATAGMRLLRMESEELA |
| | | DRVLDVVERSLSNYPFDFQGARIITGQEEGAYGWITINYLLGKFSQKNQET |
| | | FGALDLGGASTQVTFVPQNQTIESPDNALQFRLYGKDYNVYTHSFLCYGK |
| | | DQALWQKLAKDIQVASNEILRDPCFHPGYKKVVNVSDLYKTPCTKRFEMT |
| | | LPFQQFEIQGIGNYQQCHQSILELFNTSYCPYSQCAFNGIFLPPLQGDFGA |
| | | FSAFYFVMKFLNLTSEKVSQEKVTEMMKKFCAQPWEEIKTSYAGVKEKYL |
| | | SEYCFSGTYILSLLLQGYHFTADSWEHIHFIGKIQGSDAGVVTLGYMLNLTN |
| | | MIPAEQPLSTPLSHST |
| SEQ ID | DNA | GACGCCCACAAGAGCGAGGTGGCCCACCGGTTCAAGGACCTGGGCG |
| NO: 152 | | AGGAAAACTTCAAGGCCCTGGTGCTGATCGCCTTCGCCCAGTACCTG |
| | | CAGCAGAGCCCCTTCGAAGATCACGTAAAGTTAGTCAACGAGGTTACG |
| | | GAATTCGCAAAGACATGCGTTGCTGACGAATCCGCTGAGAATTGTGAC |
| | | AAGAGTTTGCACACTTTATTCGGAGATAAGTTGTGTACTGTAGCTACTT |
| | | TGAGAGAGACTTACGGTGAAATGGCTGACTGCTGTGCAAAACAGGAA |
| | | CCAGAACGTAACGAATGTTTCCTTCAGCATAAGGATGATAACCCTAAC |
| | | CTTCCAAGGCTTGTTAGGCCAGAAGTCGACGTGATGTGCACCGCCTT |
| | | CCATGATAATGAAGAGACTTTTCTTAAAAAGTACCTATACGAGATTGCA |
| | | AGGCGTCATCCATATTTTTACGCCCCAGAGCTGTTGTTTTTCGCAAAG |
| | | AGATACAAAGCTGCATTTACTGAGTGTTGCCAAGCTGCCGACAAGGCC |
| | | GCTTGTTTGCTACCAAAGTTGGACGAATTGAGAGGAGGCGGAGGATC |
| | | TACCCAGAACAAGGCCCTGCCCGAGAACGTGAAGTACGGCATCGTGC |
| | | TGGATGCCGGCAGCAGCCACACCAGCCTGTACATCTACAAGTGGCCT |
| | | GCCGAGAAAGAAAACGACACCGGCGTGGTGCATCAGGTGGAAGAGT |
| | | GCAGAGTGAAGGGCCCTGGCATCAGCAAGTTCGTGCAGAAAGTGAAC |
| | | GAGATCGGCATCTACCTGACCGACTGCATGGAACGGGCCAGGGAAGT |
| | | GATCCCCAGAAGCCAGCACCAGGAAACCCCCGTGTATCTGGGAGCCA |
| | | CCGCCGGCATGAGACTGCTGAGAATGGAAAGCGAGGAACTGGCCGA |
| | | CCGGGTGCTGGACGTGGTGGAAAGAAGCCTGAGCAACTACCCATTCG |
| | | ATTTTCAAGGCGCCAGAATCATCACCGGCCAGGAAGAAGGCGCCTAC |
| | | GGCTGGATCACCATCAACTACCTGCTGGGCAAGTTCAGCCAGAAGAA |
| | | TCAGGAAACCTTCGGCGCCCTGGACCTGGGCGGAGCTTCTACCCAAG |
| | | TGACCTTCGTGCCCCAGAATCAGACCATCGAGAGCCCCGACAACGCC |
| | | CTGCAGTTCCGGCTGTACGGCAAGGACTACAATGTGTACACCCACAG |
| | | CTTTCTGTGCTACGGAAAGGACCAGGCTCTGTGGCAGAAGCTGGCCA |
| | | AGGACATCCAGGTGGCCAGCAACGAGATCCTGCGGGACCCTTGCTTC |
| | | CACCCCGGCTACAAGAAAGTCGTGAACGTGTCCGACCTGTACAAGAC |
| | | CCCCTGCACCAAGAGATTCGAGATGACCCTGCCCTTCCAGCAGTTCG |
| | | AGATCCAGGGCATCGGCAATTACCAGCAGTGCCACCAGAGCATCCTG |
| | | GAACTGTTCAACACCAGCTACTGCCCCTACAGCCAGTGCGCCTTCAAC |
| | | GGCATCTTCCTGCCACCTCTGCAGGGGGATTTCGGCGCCTTCAGCGC |
| | | CTTCTACTTCGTGATGAAGTTCCTGAACCTGACCAGCGAGAAGGTGTC |
| | | CCAGGAAAAAGTGACAGAGATGATGAAGAAGTTCTGCGCCCAGCCCT |
| | | GGGAGGAAATCAAGACCTCCTACGCTGGCGTGAAAGAGAAGTACCTG |
| | | AGCGAGTACTGCTTCAGCGGCACCTACATCCTGAGCCTGCTGCTGCA |
| | | GGGCTACCACTTCACCGCCGATAGCTGGGAGCACATCCACTTCATCG |
| | | GCAAGATTCAGGGCAGCGACGCCGGCTGGACACTGGGCTACATGCT |
| | | GAATCTGACCAACATGATCCCCGCCGAGCAGCCCCTGAGCACACCTC |
| | | TGAGCCACAGCACC |
| CD39(aa38- | | |
| 476)_dMIL(193- | | |
| 204)_HSAII | | |
| SEQ ID | Amino | DEGKASSAKQRLKCASLQKFGERAFKAWAVARLSQRFPKAEFAEVSKLV |
| NO: 153 | acid | TDLTKVHTECCHGDLLECADDRADLAKYICENQDSISSKLKECCEKPLLEK |
| | | SHCIAEVENDEMPADLPSLAADFVESKDVCKNYAEAKDVFLGMFLYEYAR |
| | | RHPDYSVVLLLRLAKTYETTLEKCCAAADPHECYAKVFDEFKPGGGGST |
| | | QNKALPENVKYGIVLDAGSSHTSLYIYKWPAEKENDTGVVHQVEECRVK |
| | | GPGISKFVQKVNEIGIYLTDCMERAREVIPRSQHQETPVYLGATAGMRLLR |
| | | MESEELADRVLDVVERSLSNYPFDFQGARIITGQEEGAYGWITINYLLGKF |
| | | SQKNQETFGALDLGGASTQVTFVPQNQTIESPDNALQFRLYGKDYNVYT |
| | | HSFLCYGKDQALWQKLAKDIQVASNEILRDPCFHPGYKKVVNVSDLYKTP |
| | | CTKRFEMTLPFQQFEIQGIGNYQQCHQSILELFNTSYCPYSQCAFNGIFLP |
| | | PLQGDFGAFSAFYFVMKFLNLTSEKVSQEKVTEMMKKFCAQPWEEIKTS |
| | | YAGVKEKYLSEYCFSGTYILSLLLQGYHFTADSWEHIHFIGKIQGSDAGWT |
| | | LGYMLNLTNMIPAEQPLSTPLSHST |
| SEQ ID | DNA | GACGAGGGTAAGGCATCATCTGCCAAGCAGAGATTAAAATGTGCATCT |
| NO: 154 | | TTGCAAAAATTTGGAGAGAGAGCTTTTAAGGCATGGGCTGTTGCCCGA |
| | | CTAAGCCAAAGATTCCCAAAAGCCGAATTTGCTGAAGTATCCAAGCTG |
| | | GTGACTGATTTGACTAAAGTACATACAGAATGTTGCCATGGCGACCTT |
| | | TTAGAATGTGCTGATGACAGAGCAGATTTGGCTAAGTATATCTGCGAA |
| | | AATCAAGATTCAATCAGCTCTAAGCTGAAGGAATGTTGCGAGAAACCA |
| | | CTGTTAGAAAAATCGCATTGTATTGCTGAAGTTGAAAATGATGAGATGC |
| | | CTGCTGACTTGCCTTCTCTTGCCGCTGATTTTGTTGAGTCGAAGGATG |
| | | TCTGTAAGAATTATGCTGAAGCTAAAGACGTTTTCCTGGGTATGTTCTT |
| | | ATATGAGTACGCAAGACGTCACCCAGATTACTCTGTGGTTCTGCTACT |
| | | GAGATTGGCTAAAACATACGAGACAACGCTGGAGAAGTGCTGTGCTG |
| | | CCGCTGACCCTCATGAGTGCTATGCAAAGGTTTTTGATGAATTCAAAC |
| | | CAGGAGGCGGAGGATCTACCCAGAACAAGGCCCTGCCCGAGAACGT |
| | | GAAGTACGGCATCGTGCTGGATGCCGGCAGCAGCCACACCAGCCTGT |
| | | ACATCTACAAGTGGCCTGCCGAGAAAGAAAACGACACCGGCGTGGTG |
| | | CATCAGGTGGAAGAGTGCAGAGTGAAGGGCCCTGGCATCAGCAAGTT |
| | | CGTGCAGAAAGTGAACGAGATCGGCATCTACCTGACCGACTGCATGG |
| | | AACGGGCCAGGGAAGTGATCCCCAGAAGCCAGCACCAGGAAACCCC |
| | | CGTGTATCTGGGAGCCACCGCCGGCATGAGACTGCTGAGAATGGAAA |
| | | GCGAGGAACTGGCCGACCGGGTGCTGGACGTGGTGGAAAGAAGCCT |
| | | GAGCAACTACCCATTCGATTTTCAAGGCGCCAGAATCATCACCGGCCA |
| | | GGAAGAAGGCGCCTACGGCTGGATCACCATCAACTACCTGCTGGGCA |
| | | AGTTCAGCCAGAAGAATCAGGAAACCTTCGGCGCCCTGGACCTGGGC |
| | | GGAGCTTCTACCCAAGTGACCTTCGTGCCCCAGAATCAGACCATCGA |
| | | GAGCCCCGACAACGCCCTGCAGTTCCGGCTGTACGGCAAGGACTACA |
| | | ATGTGTACACCCACAGCTTTCTGTGCTACGGAAAGGACCAGGCTCTGT |
| | | GGCAGAAGCTGGCCAAGGACATCCAGGTGGCCAGCAACGAGATCCT |
| | | GCGGGACCCTTGCTTCCACCCCGGCTACAAGAAAGTCGTGAACGTGT |
| | | CCGACCTGTACAAGACCCCCTGCACCAAGAGATTCGAGATGACCCTG |
| | | CCCTTCCAGCAGTTCGAGATCCAGGGCATCGGCAATTACCAGCAGTG |
| | | CCACCAGAGCATCCTGGAACTGTTCAACACCAGCTACTGCCCCTACAG |
| | | CCAGTGCGCCTTCAACGGCATCTTCCTGCCACCTCTGCAGGGGGATT |
| | | TCGGCGCCTTCAGCGCCTTCTACTTCGTGATGAAGTTCCTGAACCTGA |
| | | CCAGCGAGAAGGTGTCCCAGGAAAAAGTGACAGAGATGATGAAGAAG |
| | | TTCTGCGCCCAGCCCTGGGAGGAAATCAAGACCTCCTACGCTGGCGT |
| | | GAAAGAGAAGTACCTGAGCGAGTACTGCTTCAGCGGCACCTACATCC |
| | | TGAGCCTGCTGCTGCAGGGCTACCACTTCACCGCCGATAGCTGGGAG |
| | | CACATCCACTTCATCGGCAAGATTCAGGGCAGCGACGCCGGCTGGAC |
| | | ACTGGGCTACATGCTGAATCTGACCAACATGATCCCCGCCGAGCAGC |
| | | CCCTGAGCACACCTCTGAGCCACAGCACC |
| EP14_plusMIL | | |
| SEQ ID | Amino | TQNKALPENVKYGIVLDAGSSHTSLYIYKWPAEKENDTGVVHQVEECRVK |
| NO: 155 | acid | GPGISKFVQKVNEIGIYLTDCMERAREVIPRSQHQETPVYLGATAGMRLLR |
| | | MESEELADRVLDVVERSLSNYPFDFQGARIITGQEEGAYGWITINYLLGKF |
| | | SQKTRWFSIVPYETNNQETFGALDLGGASTQVTFVPQNQTIESPDNALQF |
| | | RLYGKDYNVYTHSFLCYGKDQALWQKLAKDIQVASNEILRDPCFHPGYKK |
| | | VVNVSDLYKTPCTKRFEMTLPFQQFEIQGIGNYQQCHQSILELFNTSYCPY |
| | | SQCAFNGIFLPPLQGDFGAFSAFYSVMKFLNLTSEKVSQEKVTEMMKKFC |
| | | AQPWEEIKTSYAGVKEKYLSEYCFSGTYILSLLLQGYHFTADSWEHIHFIG |
| | | KIQGSDAGWTLGYMLNLTNMIPAEQPLSTPLSHST |
| SEQ ID | DNA | ACCCAGAACAAGGCCCTGCCCGAGAACGTGAAGTACGGCATCGTGCT |
| NO: 156 | | GGATGCCGGCAGCAGCCACACCAGCCTGTACATCTACAAGTGGCCTG |
| | | CCGAGAAAGAAAACGACACCGGCGTGGTGCATCAGGTGGAAGAGTGC |
| | | AGAGTGAAGGGCCCTGGCATCAGCAAGTTCGTGCAGAAAGTGAACGA |
| | | GATCGGCATCTACCTGACCGACTGCATGGAACGGGCCAGGGAAGTGA |
| | | TCCCCAGAAGCCAGCACCAGGAAACCCCCGTGTATCTGGGAGCCACC |
| | | GCCGGCATGAGACTGCTGAGAATGGAAAGCGAGGAACTGGCCGACC |
| | | GGGTGCTGGACGTGGTGGAAAGAAGCCTGAGCAACTACCCATTCGAT |
| | | TTTCAAGGCGCCAGAATCATCACCGGCCAGGAAGAAGGCGCCTACGG |
| | | CTGGATCACCATCAACTACCTGCTGGGCAAGTTCAGCCAGAAGACCA |
| | | GATGGTTCAGCATCGTGCCCTACGAGACAAACAATCAGGAAACCTTCG |
| | | GCGCCCTGGACCTGGGCGGAGCTTCTACCCAAGTGACCTTCGTGCCC |
| | | CAGAATCAGACCATCGAGAGCCCCGACAACGCCCTGCAGTTCCGGCT |
| | | GTACGGCAAGGACTACAATGTGTACACCCACAGCTTTCTGTGCTACGG |
| | | AAAGGACCAGGCTCTGTGGCAGAAGCTGGCCAAGGACATCCAGGTGG |
| | | CCAGCAACGAGATCCTGCGGGACCCTTGCTTCCACCCCGGCTACAAG |
| | | AAAGTCGTGAACGTGTCCGACCTGTACAAGACCCCCTGCACCAAGAG |
| | | ATTCGAGATGACCCTGCCCTTCCAGCAGTTCGAGATCCAGGGCATCG |
| | | GCAATTACCAGCAGTGCCACCAGAGCATCCTGGAACTGTTCAACACCA |
| | | GCTACTGCCCCTACAGCCAGTGCGCCTTCAACGGCATCTTCCTGCCA |
| | | CCTCTGCAGGGGGATTTCGGCGCCTTCAGCGCCTTCTACTCCGTGAT |
| | | GAAGTTCCTGAACCTGACCAGCGAGAAGGTGTCCCAGGAAAAAGTGA |
| | | CAGAGATGATGAAGAAGTTCTGCGCCCAGCCCTGGGAGGAAATCAAG |
| | | ACCTCCTACGCTGGCGTGAAAGAGAAGTACCTGAGCGAGTACTGCTT |
| | | CAGCGGCACCTACATCCTGAGCCTGCTGCTGCAGGGCTACCACTTCA |
| | | CCGCCGATAGCTGGGAGCACATCCACTTCATCGGCAAGATTCAGGGC |
| | | AGCGACGCCGGCTGGACACTGGGCTACATGCTGAATCTGACCAACAT |
| | | GATCCCCGCCGAGCAGCCCCTGAGCACACCTCTGAGCCACAGCACC |
| SEQ ID | Forward | TCGCGATCCTGGAAGGCGTGCACTGCGCCCCTACCAGCAGCAGCACC |
| NO: 157 | primer | AAGAAAACCCAGCTGACCAGCAGCACCCAGAACAAGGCCCTGC |
| SEQ ID | Reverse | TAGAAGGCACAGTCGAGG |
| NO: 158 | primer | |
| SEQ ID | Forward | CTGGTCGCGATCCTGGAAGGCGTGCACTGCGCCCCTACCAGCAGCA |
| NO: 159 | primer | GCACCCAGAACAAGGCCCTG |
| SEQ ID | Forward | CTGGTCGCGATCCTGGAAGGCGTGCACTGCGCCCCTACCAGCAGCA |
| NO: 160 | primer | GCACCCAGAACAAGGCCCTG |
| SEQ ID | Forward | CTGGTCGCGATCCTGGAAGGCGTGCACTGCGCCCCTACCACCCAGAA |
| NO: 161 | primer | CAAGGCCCTG |
| SEQ ID | Forward | CTGGTCGCGATCCTGGAAGGCGTGCACTGCGCCCCTACCAGCAGCA |
| NO: 162 | primer | GCACCAAGAAAACCCAGAACAAGGCCCTG |
| SEQ ID | Forward | CTGGTCGCGATCCTGGAAGGCGTGCACTGCGCCCCTACCAGCAGCA |
| NO: 163 | primer | GCACCAAGAAAACCCAGCTGACCCAGAACAAGGCCCTG |
| SEQ ID | Forward | CTGGTCGCGATCCTGGAAGGCGTGCACTGCAGCAGCAGCACCAAGAA |
| NO: 164 | primer | AACCCAGCTGACCCAGAACAAGGCCCTG |
| SEQ ID | Primer | CATACGATTTAGGTGA |
| NO: 165 | P270 | |
| SEQ ID | Primer | TAGAAGGCACAGTCGAGG |
| NO: 166 | P271 | |
| SEQ ID | Ubiquitin | MQIFVKTLTGKTITLEVEPSDTIENVKAKIQDKEGIPPDQQRLIFAGKQLED |
| NO: 167 | tag | GRTLSDYNIQKESTLHLVLRLRGG |
| SEQ ID | CKappa | TVAAPSVFIFPPSDEQLKSGTASVVCLLNNFYPREAKVQWKVDNALQSGN |
| NO: 168 | tag | SQESVTEQDSKDSTYSLSSTLTLSKADYEKHKVYACEVTHQGLSSPVTKS |
| | | FNRGE |
| SEQ ID | HSA | DAHKSEVAHRFKDLGEENFKALVLIAFAQYLQQSPFEDHVKLVNEVTEFA |
| NO: 169 | Domain | KTCVADESAENCDKSLHTLFGDKLCTVATLRETYGEMADCCAKQEPERN |
| | I tag | ECFLQHKDDNPNLPRLVRPEVDVMCTAFHDNEETFLKKYLYEIARRHPYF |
| | | YAPELLFFAKRYKAAFTECCQAADKAACLLPKLDELR |
| SEQ ID | HSA | DEGKASSAKQRLKCASLQKFGERAFKAWAVARLSQRFPKAEFAEVSKLV |
| NO: 170 | Domain | TDLTKVHTECCHGDLLECADDRADLAKYICENQDSISSKLKECCEKPLLEK |
| | II tag | SHCIAEVENDEMPADLPSLAADFVESKDVCKNYAEAKDVFLGMFLYEYAR |
| | | RHPDYSVVLLLRLAKTYETTLEKCCAAADPHECYAKVFDEFKP |
| SEQ ID | P928 | CTGCCGAGAAAGAACAGGACACCGGCGTGG |
| NO: 171 | primer | |
| SEQ ID | P929 | CCACGCCGGTGTCCTGTTCTTTCTCGGCAG |
| NO: 172 | primer | |
| SEQ ID | P930 | CGTGCCCCAGAATCAGGCCATCGAGAGCC |
| NO: 173 | primer | |
| SEQ ID | P931 | GGCTCTCGATGGCCTGATTCTGGGGCACG |
| NO: 174 | primer | |
| SEQ ID | P932 | GGCTACAAGAAAGTCGTGCAGGTGTCCGACCTGTACAAGAC |
| NO: 175 | primer | |
| SEQ ID | P933 | GTCTTGTACAGGTCGGACACCTGCACGACTTTCTTGTAGCC |
| NO: 176 | primer | |
| SEQ ID | P934 | GCATCCTGGAACTGTTCCAGACCAGCTACTGCCCC |
| NO: 177 | primer | |
| SEQ ID | P935 | GGGGCAGTAGCTGGTCTGGAACAGTTCCAGGATGC |
| NO: 178 | primer | |
| SEQ ID | P936 | CCGTGATGAAGTTCCTGCAGCTGACCAGCGAGAAG |
| NO: 179 | primer | |
| SEQ ID | P937 | CTTCTCGCTGGTCAGCTGCAGGAACTTCATCACGG |
| NO: 180 | primer | |
| SEQ ID | P938 | CACTGGGCTACATGCTGCAGCTGACCAACATGATCC |
| NO: 181 | primer | |
| SEQ ID | P939 | GGATCATGTTGGTCAGCTGCAGCATGTAGCCCAGTG |
| NO: 182 | primer | |
| SEQ ID | P878 | CTACATCCTGAGCCTGCTGCAGCAGGGCTACCACTTCAC |
| NO: 183 | primer | |
| SEQ ID | P879 | GTGAAGTGGTAGCCCTGCTGCAGCAGGCTCAGGATGTAG |
| NO: 184 | primer | |
| SEQ ID | P880 | GAGAAGTACCTGAGCGAGTTTTGCTTCAGCGGCACCTACATCC |
| NO: 185 | primer | |
| SEQ ID | P881 | GGATGTAGGTGCCGCTGAAGCAAAACTCGCTCAGGTACTTCTC |
| NO: 186 | primer | |
| SEQ ID | P882 | GTTCGAGATCCAGGGCACCGGCAATTACCAGCAGTG |
| NO: 187 | primer | |
| SEQ ID | P883 | CACTGCTGGTAATTGCCGGTGCCCTGGATCTCGAAC |
| NO: 188 | primer | |
| SEQ ID | P884 | CGCCGATAGCTGGGAGCACATCCACTTCATCGGCAAG |
| NO: 189 | primer | |
| SEQ ID | P885 | CTTGCCGATGAAGTGGATGTGCTCCCAGCTATCGGCG |
| NO: 190 | primer | |
| SEQ ID | R113Mt | GAGATCGGCATCTACCTGACCGACTGCATGGAACGGGCCATGGAAGT |
| NO: 191 | empl | GATCCCCAGAAGCCAGCACCAGGAAACCCCCGTGTATCTGGGAGCCA |
| | | CCGCCGGCATGAGACTGCTGAGAATG |
| SEQ ID | R113MFW | GAGATCGGCATCTACCTGACCGACT |
| NO: 192 | | |
| SEQ ID | R113M | CATTCTCAGCAGTCTCAT |
| NO: 193 | Rev | |
| SEQ ID | F330Ste | CTTCAGCGCCTTCTACTcCGTGATGAAGTTCCTGAACCTGACCAGCGA |
| NO: 194 | mpl | GAAGGTGTCCCAGGAAAAAGTGACAGAGATGATGAAGAAGTTCTGCG |
| | | CCCAGCCCTGGGAGGAAATCAAGAC |
| SEQ ID | F330S | CTTCAGCGCCTTCTACTCC |
| NO: 195 | FW | |
| SEQ ID | F3305 | GGTCTTGATTTCCTCCCAG |
| NO: 196 | Rev | |
| SEQ ID | Y377F | CTGGGAGGAAATCAAGACCTCCTACGCTGGCGTGAAAGAGAAGTACC |
| NO: 197 | templ | TGAGCGAGTtCTGCTTCAGCGGCACCTACATCCTGAGCCTGCTGCTGC |
| | | AGGGCTACCACTTCACCGCCGATA |
| SEQ ID | L389C | CTGGGAGGAAATCAAGACCTCCTACGCTGGCGTGAAAGAGAAGTACC |
| NO: 198 | templ | TGAGCGAGTACTGCTTCAGCGGCACCTACATCCTGAGCCTGCTGCaG |
| | | CAGGGCTACCACTTCACCGCCGATA |
| SEQ ID | Y377F/L | CTGGGAGGAAATCAAGACCTCCTACGCTGGCGTGAAAGAGAAGTACC |
| NO: 199 | 389Q | TGAGCGAGTTCTGCTTCAGCGGCACCTACATCCTGAGCCTGCTGCaG |
| | templ | CAGGGCTACCACTTCACCGCCGATA |
| SEQ ID | FW | CTGGGAGGAAATCAAGACC |
| NO: 200 | primer | |
| SEQ ID | Rev | TATCGGCGGTGAAGTGGTA |
| NO: 201 | primer | |
| SEQ ID | WT | CTGGGAGGAAATCAAGACCTCCTACGCTGGCGTGAAAGAGAAGTACC |
| NO: 202 | primer | TGAGCGAGTACTGCTTCAGCGGCACCTACATCCTGAGCCTGCTGCTG |
| | | CAGGGCTACCACTTCACCGCCGATA |
| SEQ ID | fwMut174 | CACCGGCCAGGAAGCCGGCGCCTACG |
| NO: 203 | | |
| SEQ ID | revMut174 | CGTAGGCGCCGGCTTCCTGGCCGGTG |
| NO: 204 | | |
| SEQ ID | fwMut218 | GGACCTGGGCGGAGCTGCTACCCAAGTGACCTTC |
| NO: 205 | | |
| SEQ ID | revMut218 | GAAGGTCACTTGGGTAGCAGCTCCGCCCAGGTCC |
| NO: 206 | | |
| SEQ ID | plusMILfw | CTGGATCACCATCAACTACCTGCTGGGCAAGTTCAGCCAGAAGACCA |
| NO: 207 | | GATGGTTCAGCATCGTGCCCTACGAGACAAACA |
| SEQ ID | plusMILrev | TCACTTGGGTAGAAGCTCCGCCCAGGTCCAGGGCGCCGAAGGTTTCC |
| NO: 208 | | TGATTGTTTGTCTCGTAGGGCA |
| EP1aa1-3 | | |
| SEQ ID | Amino | APTTQNKALPENVKYGIVLDAGSSHTSLYIYKWPAEKENDTGVVHQVEEC |
| NO: 209 | acid | RVKGPGISKFVQKVNEIGIYLTDCMERAMEVIPRSQHQETPVYLGATAGM |
| | | RLLRMESEELADRVLDVVERSLSNYPFDFQGARIITGQEEGAYGWITINYL |
| | | LGKFSQKNQETFGALDLGGASTQVTFVPQNQTIESPDNALQFRLYGKDY |
| | | NVYTHSFLCYGKDQALWQKLAKDIQVASNEILRDPCFHPGYKKVVNVSDL |
| | | YKTPCTKRFEMTLPFQQFEIQGIGNYQQCHQSILELFNTSYCPYSQCAFN |
| | | GIFLPPLQGDFGAFSAFYFVMKFLNLTSEKVSQEKVTEMMKKFCAQPWE |
| | | EIKTSYAGVKEKYLSEYCFSGTYILSLLLQGYHFTADSWEHIHFIGKIQGSD |
| | | AGWTLGYMLNLTNMIPAEQPLSTPLSHST |
| SEQ ID | DNA | GCCCCTACCACCCAGAACAAGGCCCTGCCCGAGAACGTGAAGTACGG |
| NO: 210 | | CATCGTGCTGGATGCCGGCAGCAGCCACACCAGCCTGTACATCTACA |
| | | AGTGGCCTGCCGAGAAAGAAAACGACACCGGCGTGGTGCATCAGGTG |
| | | GAAGAGTGCAGAGTGAAGGGCCCTGGCATCAGCAAGTTCGTGCAGAA |
| | | AGTGAACGAGATCGGCATCTACCTGACCGACTGCATGGAACGGGCCA |
| | | TGGAAGTGATCCCCAGAAGCCAGCACCAGGAAACCCCCGTGTATCTG |
| | | GGAGCCACCGCCGGCATGAGACTGCTGAGAATGGAAAGCGAGGAAC |
| | | TGGCCGACCGGGTGCTGGACGTGGTGGAAAGAAGCCTGAGCAACTA |
| | | CCCATTCGATTTTCAAGGCGCCAGAATCATCACCGGCCAGGAAGAAG |
| | | GCGCCTACGGCTGGATCACCATCAACTACCTGCTGGGCAAGTTCAGC |
| | | CAGAAGAATCAGGAAACCTTCGGCGCCCTGGACCTGGGCGGAGCTTC |
| | | TACCCAAGTGACCTTCGTGCCCCAGAATCAGACCATCGAGAGCCCCG |
| | | ACAACGCCCTGCAGTTCCGGCTGTACGGCAAGGACTACAATGTGTAC |
| | | ACCCACAGCTTTCTGTGCTACGGAAAGGACCAGGCTCTGTGGCAGAA |
| | | GCTGGCCAAGGACATCCAGGTGGCCAGCAACGAGATCCTGCGGGAC |
| | | CCTTGCTTCCACCCCGGCTACAAGAAAGTCGTGAACGTGTCCGACCT |
| | | GTACAAGACCCCCTGCACCAAGAGATTCGAGATGACCCTGCCCTTCC |
| | | AGCAGTTCGAGATCCAGGGCATCGGCAATTACCAGCAGTGCCACCAG |
| | | AGCATCCTGGAACTGTTCAACACCAGCTACTGCCCCTACAGCCAGTGC |
| | | GCCTTCAACGGCATCTTCCTGCCACCTCTGCAGGGGGATTTCGGCGC |
| | | CTTCAGCGCCTTCTACTTCGTGATGAAGTTCCTGAACCTGACCAGCGA |
| | | GAAGGTGTCCCAGGAAAAAGTGACAGAGATGATGAAGAAGTTCTGCG |
| | | CCCAGCCCTGGGAGGAAATCAAGACCTCCTACGCTGGCGTGAAAGAG |
| | | AAGTACCTGAGCGAGTACTGCTTCAGCGGCACCTACATCCTGAGCCT |
| | | GCTGCTGCAGGGCTACCACTTCACCGCCGATAGCTGGGAGCACATCC |
| | | ACTTCATCGGCAAGATTCAGGGCAGCGACGCCGGCTGGACACTGGGC |
| | | TACATGCTGAATCTGACCAACATGATCCCCGCCGAGCAGCCCCTGAG |
| | | CACACCTCTGAGCCACAGCACC |
| EP1xEP17aa1-3 | | |
| SEQ ID | Amino | APTTQNKALPENVKYGIVLDAGSSHTSLYIYKWPAEKENDTGVVHQVEEC |
| NO: 211 | acid | RVKGPGISKFVQKVNEIGIYLTDCMERAMEVIPRSQHQETPVYLGATAGM |
| | | RLLRMESEELADRVLDVVERSLSNYPFDFQGARIITGQEEGAYGWITINYL |
| | | LGKFSQKNQETFGALDLGGASTQVTFVPQNQTIESPDNALQFRLYGKDY |
| | | NVYTHSFLCYGKDQALWQKLAKDIQVASNEILRDPCFHPGYKKVVNVSDL |
| | | YKTPCTKRFEMTLPFQQFEIQGIGNYQQCHQSILELFNTSYCPYSQCAFN |
| | | GIFLPPLQGDFGAFSAFYFVMKFLNLTSEKVSQEKVTEMMKKFCAQPWE |
| | | EIKTSYAGVKEKYLSEFCFSGTYILSLLLQGYHFTADSWEHIHFIGKIQGSD |
| | | AGWTLGYMLNLTNMIPAEQPLSTPLSHST |
| SEQ ID | DNA | GCCCCTACCACCCAGAACAAGGCCCTGCCCGAGAACGTGAAGTACGG |
| NO: 212 | | CATCGTGCTGGATGCCGGCAGCAGCCACACCAGCCTGTACATCTACA |
| | | AGTGGCCTGCCGAGAAAGAAAACGACACCGGCGTGGTGCATCAGGTG |
| | | GAAGAGTGCAGAGTGAAGGGCCCTGGCATCAGCAAGTTCGTGCAGAA |
| | | AGTGAACGAGATCGGCATCTACCTGACCGACTGCATGGAACGGGCCA |
| | | TGGAAGTGATCCCCAGAAGCCAGCACCAGGAAACCCCCGTGTATCTG |
| | | GGAGCCACCGCCGGCATGAGACTGCTGAGAATGGAAAGCGAGGAAC |
| | | TGGCCGACCGGGTGCTGGACGTGGTGGAAAGAAGCCTGAGCAACTA |
| | | CCCATTCGATTTTCAAGGCGCCAGAATCATCACCGGCCAGGAAGAAG |
| | | GCGCCTACGGCTGGATCACCATCAACTACCTGCTGGGCAAGTTCAGC |
| | | CAGAAGAATCAGGAAACCTTCGGCGCCCTGGACCTGGGCGGAGCTTC |
| | | TACCCAAGTGACCTTCGTGCCCCAGAATCAGACCATCGAGAGCCCCG |
| | | ACAACGCCCTGCAGTTCCGGCTGTACGGCAAGGACTACAATGTGTAC |
| | | ACCCACAGCTTTCTGTGCTACGGAAAGGACCAGGCTCTGTGGCAGAA |
| | | GCTGGCCAAGGACATCCAGGTGGCCAGCAACGAGATCCTGCGGGAC |
| | | CCTTGCTTCCACCCCGGCTACAAGAAAGTCGTGAACGTGTCCGACCT |
| | | GTACAAGACCCCCTGCACCAAGAGATTCGAGATGACCCTGCCCTTCC |
| | | AGCAGTTCGAGATCCAGGGCATCGGCAATTACCAGCAGTGCCACCAG |
| | | AGCATCCTGGAACTGTTCAACACCAGCTACTGCCCCTACAGCCAGTGC |
| | | GCCTTCAACGGCATCTTCCTGCCACCTCTGCAGGGGGATTTCGGCGC |
| | | CTTCAGCGCCTTCTACTTCGTGATGAAGTTCCTGAACCTGACCAGCGA |
| | | GAAGGTGTCCCAGGAAAAAGTGACAGAGATGATGAAGAAGTTCTGCG |
| | | CCCAGCCCTGGGAGGAAATCAAGACCTCCTACGCTGGCGTGAAAGAG |
| | | AAGTACCTGAGCGAGTTTTGCTTCAGCGGCACCTACATCCTGAGCCTG |
| | | CTGCTGCAGGGCTACCACTTCACCGCCGATAGCTGGGAGCACATCCA |
| | | CTTCATCGGCAAGATTCAGGGCAGCGACGCCGGCTGGACACTGGGCT |
| | | ACATGCTGAATCTGACCAACATGATCCCCGCCGAGCAGCCCCTGAGC |
| | | ACACCTCTGAGCCACAGCACC |
| EP1xEP17aa1-6 | | |
| SEQ ID | Amino | APTSSSTQNKALPENVKYGIVLDAGSSHTSLYIYKWPAEKENDTGVVHQV |
| NO: 213 | acid | EECRVKGPGISKFVQKVNEIGIYLTDCMERAMEVIPRSQHQETPVYLGAT |
| | | AGMRLLRMESEELADRVLDVVERSLSNYPFDFQGARIITGQEEGAYGWIT |
| | | INYLLGKFSQKNQETFGALDLGGASTQVTFVPQNQTIESPDNALQFRLYG |
| | | KDYNVYTHSFLCYGKDQALWQKLAKDIQVASNEILRDPCFHPGYKKVVNV |
| | | SDLYKTPCTKRFEMTLPFQQFEIQGIGNYQQCHQSILELFNTSYCPYSQC |
| | | AFNGIFLPPLQGDFGAFSAFYFVMKFLNLTSEKVSQEKVTEMMKKFCAQP |
| | | WEEIKTSYAGVKEKYLSEFCFSGTYILSLLLQGYHFTADSWEHIHFIGKIQG |
| | | SDAGWTLGYMLNLTNMIPAEQPLSTPLSHST |
| SEQ ID | DNA | GCCCCTACCAGCAGCAGCACCCAGAACAAGGCCCTGCCCGAGAACGT |
| NO: 214 | | GAAGTACGGCATCGTGCTGGATGCCGGCAGCAGCCACACCAGCCTGT |
| | | ACATCTACAAGTGGCCTGCCGAGAAAGAAAACGACACCGGCGTGGTG |
| | | CATCAGGTGGAAGAGTGCAGAGTGAAGGGCCCTGGCATCAGCAAGTT |
| | | CGTGCAGAAAGTGAACGAGATCGGCATCTACCTGACCGACTGCATGG |
| | | AACGGGCCATGGAAGTGATCCCCAGAAGCCAGCACCAGGAAACCCCC |
| | | GTGTATCTGGGAGCCACCGCCGGCATGAGACTGCTGAGAATGGAAAG |
| | | CGAGGAACTGGCCGACCGGGTGCTGGACGTGGTGGAAAGAAGCCTG |
| | | AGCAACTACCCATTCGATTTTCAAGGCGCCAGAATCATCACCGGCCAG |
| | | GAAGAAGGCGCCTACGGCTGGATCACCATCAACTACCTGCTGGGCAA |
| | | GTTCAGCCAGAAGAATCAGGAAACCTTCGGCGCCCTGGACCTGGGCG |
| | | GAGCTTCTACCCAAGTGACCTTCGTGCCCCAGAATCAGACCATCGAGA |
| | | GCCCCGACAACGCCCTGCAGTTCCGGCTGTACGGCAAGGACTACAAT |
| | | GTGTACACCCACAGCTTTCTGTGCTACGGAAAGGACCAGGCTCTGTG |
| | | GCAGAAGCTGGCCAAGGACATCCAGGTGGCCAGCAACGAGATCCTGC |
| | | GGGACCCTTGCTTCCACCCCGGCTACAAGAAAGTCGTGAACGTGTCC |
| | | GACCTGTACAAGACCCCCTGCACCAAGAGATTCGAGATGACCCTGCC |
| | | CTTCCAGCAGTTCGAGATCCAGGGCATCGGCAATTACCAGCAGTGCC |
| | | ACCAGAGCATCCTGGAACTGTTCAACACCAGCTACTGCCCCTACAGCC |
| | | AGTGCGCCTTCAACGGCATCTTCCTGCCACCTCTGCAGGGGGATTTC |
| | | GGCGCCTTCAGCGCCTTCTACTTCGTGATGAAGTTCCTGAACCTGACC |
| | | AGCGAGAAGGTGTCCCAGGAAAAAGTGACAGAGATGATGAAGAAGTT |
| | | CTGCGCCCAGCCCTGGGAGGAAATCAAGACCTCCTACGCTGGCGTGA |
| | | AAGAGAAGTACCTGAGCGAGTTCTGCTTCAGCGGCACCTACATCCTGA |
| | | GCCTGCTGCTGCAGGGCTACCACTTCACCGCCGATAGCTGGGAGCAC |
| | | ATCCACTTCATCGGCAAGATTCAGGGCAGCGACGCCGGCTGGACACT |
| | | GGGCTACATGCTGAATCTGACCAACATGATCCCCGCCGAGCAGCCCC |
| | | TGAGCACACCTCTGAGCCACAGCACC |
| EP1xEP17_K405Naa1-15 | | |
| SEQ ID | Amino | APTSSSTKKTQLTSSTQNKALPENVKYGIVLDAGSSHTSLYIYKWPAEKEN |
| NO: 215 | acid | DTGVVHQVEECRVKGPGISKFVQKVNEIGIYLTDCMERAMEVIPRSQHQE |
| | | TPVYLGATAGMRLLRMESEELADRVLDVVERSLSNYPFDFQGARIITGQE |
| | | EGAYGWITINYLLGKFSQKNQETFGALDLGGASTQVTFVPQNQTIESPDN |
| | | ALQFRLYGKDYNVYTHSFLCYGKDQALWQKLAKDIQVASNEILRDPCFHP |
| | | GYKKVVNVSDLYKTPCTKRFEMTLPFQQFEIQGIGNYQQCHQSILELFNT |
| | | SYCPYSQCAFNGIFLPPLQGDFGAFSAFYFVMKFLNLTSEKVSQEKVTEM |
| | | MKKFCAQPWEEIKTSYAGVNEKYLSEFCFSGTYILSLLLQGYHFTADSWE |
| | | HIHFIGKIQGSDAGWTLGYMLNLTNMIPAEQPLSTPLSHST |
| SEQ ID | DNA | GCCCCTACCAGCAGCAGCACCAAGAAAACCCAGCTGACCAGCAGCAC |
| NO: 216 | | CCAGAACAAGGCCCTGCCCGAGAACGTGAAGTACGGCATCGTGCTGG |
| | | ATGCCGGCAGCAGCCACACCAGCCTGTACATCTACAAGTGGCCTGCC |
| | | GAGAAAGAAAACGACACCGGCGTGGTGCATCAGGTGGAAGAGTGCAG |
| | | AGTGAAGGGCCCTGGCATCAGCAAGTTCGTGCAGAAAGTGAACGAGA |
| | | TCGGCATCTACCTGACCGACTGCATGGAACGGGCCATGGAAGTGATC |
| | | CCCAGAAGCCAGCACCAGGAAACCCCCGTGTATCTGGGAGCCACCGC |
| | | CGGCATGAGACTGCTGAGAATGGAAAGCGAGGAACTGGCCGACCGG |
| | | GTGCTGGACGTGGTGGAAAGAAGCCTGAGCAACTACCCATTCGATTTT |
| | | CAAGGCGCCAGAATCATCACCGGCCAGGAAGAAGGCGCCTACGGCT |
| | | GGATCACCATCAACTACCTGCTGGGCAAGTTCAGCCAGAAGAATCAG |
| | | GAAACCTTCGGCGCCCTGGACCTGGGCGGAGCTTCTACCCAAGTGAC |
| | | CTTCGTGCCCCAGAATCAGACCATCGAGAGCCCCGACAACGCCCTGC |
| | | AGTTCCGGCTGTACGGCAAGGACTACAATGTGTACACCCACAGCTTTC |
| | | TGTGCTACGGAAAGGACCAGGCTCTGTGGCAGAAGCTGGCCAAGGAC |
| | | ATCCAGGTGGCCAGCAACGAGATCCTGCGGGACCCTTGCTTCCACCC |
| | | CGGCTACAAGAAAGTCGTGAACGTGTCCGACCTGTACAAGACCCCCT |
| | | GCACCAAGAGATTCGAGATGACCCTGCCCTTCCAGCAGTTCGAGATC |
| | | CAGGGCATCGGCAATTACCAGCAGTGCCACCAGAGCATCCTGGAACT |
| | | GTTCAACACCAGCTACTGCCCCTACAGCCAGTGCGCCTTCAACGGCA |
| | | TCTTCCTGCCACCTCTGCAGGGGGATTTCGGCGCCTTCAGCGCCTTC |
| | | TACTTCGTGATGAAGTTCCTGAACCTGACCAGCGAGAAGGTGTCCCAG |
| | | GAAAAAGTGACAGAGATGATGAAGAAGTTCTGCGCCCAACCCTGGGA |
| | | GGAAATCAAGACCTCCTACGCTGGCGTGAACGAGAAGTACCTGAGCG |
| | | AGTTTTGCTTCAGCGGCACCTACATCCTGAGCCTGCTGCTGCAGGGC |
| | | TACCACTTCACCGCCGATAGCTGGGAGCACATCCACTTCATCGGCAA |
| | | GATTCAGGGCAGCGACGCCGGCTGGACACTGGGCTACATGCTGAATC |
| | | TGACCAACATGATCCCCGCCGAGCAGCCCCTGAGCACACCTCTGAGC |
| | | CACAGCACC |
| EP1xEP17xEP19aa1-3 | | |
| SEQ ID | Amino | APTTQNKALPENVKYGIVLDAGSSHTSLYIYKWPAEKENDTGVVHQVEEC |
| NO: 217 | acid | RVKGPGISKFVQKVNEIGIYLTDCMERAMEVIPRSQHQETPVYLGATAGM |
| | | RLLRMESEELADRVLDVVERSLSNYPFDFQGARITGQEEGAYGWITINYL |
| | | LGKFSQKNQETFGALDLGGASTQVTFVPQNQTIESPDNALQFRLYGKDY |
| | | NVYTHSFLCYGKDQALWQKLAKDIQVASNEILRDPCFHPGYKKVVNVSDL |
| | | YKTPCTKRFEMTLPFQQFEIQGIGNYQQCHQSILELFNTSYCPYSQCAFN |
| | | GIFLPPLQGDFGAFSAFYFVMKFLNLTSEKVSQEKVTEMMKKFCAQPWE |
| | | EIKTSYAGVKEKYLSEFCFSGTYILSLLQQGYHFTADSWEHIHFIGKIQGSD |
| | | AGWTLGYMLNLTNMIPAEQPLSTPLSHST |
| SEQ ID | DNA | GCCCCTACCACCCAGAACAAGGCCCTGCCCGAGAACGTGAAGTACGG |
| NO: 218 | | CATCGTGCTGGATGCCGGCAGCAGCCACACCAGCCTGTACATCTACA |
| | | AGTGGCCTGCCGAGAAAGAAAACGACACCGGCGTGGTGCATCAGGTG |
| | | GAAGAGTGCAGAGTGAAGGGCCCTGGCATCAGCAAGTTCGTGCAGAA |
| | | AGTGAACGAGATCGGCATCTACCTGACCGACTGCATGGAACGGGCCA |
| | | TGGAAGTGATCCCCAGAAGCCAGCACCAGGAAACCCCCGTGTATCTG |
| | | GGAGCCACCGCCGGCATGAGACTGCTGAGAATGGAAAGCGAGGAAC |
| | | TGGCCGACCGGGTGCTGGACGTGGTGGAAAGAAGCCTGAGCAACTA |
| | | CCCATTCGATTTTCAAGGCGCCAGAATCATCACCGGCCAGGAAGAAG |
| | | GCGCCTACGGCTGGATCACCATCAACTACCTGCTGGGCAAGTTCAGC |
| | | CAGAAGAATCAGGAAACCTTCGGCGCCCTGGACCTGGGCGGAGCTTC |
| | | TACCCAAGTGACCTTCGTGCCCCAGAATCAGACCATCGAGAGCCCCG |
| | | ACAACGCCCTGCAGTTCCGGCTGTACGGCAAGGACTACAATGTGTAC |
| | | ACCCACAGCTTTCTGTGCTACGGAAAGGACCAGGCTCTGTGGCAGAA |
| | | GCTGGCCAAGGACATCCAGGTGGCCAGCAACGAGATCCTGCGGGAC |
| | | CCTTGCTTCCACCCCGGCTACAAGAAAGTCGTGAACGTGTCCGACCT |
| | | GTACAAGACCCCCTGCACCAAGAGATTCGAGATGACCCTGCCCTTCC |
| | | AGCAGTTCGAGATCCAGGGCATCGGCAATTACCAGCAGTGCCACCAG |
| | | AGCATCCTGGAACTGTTCAACACCAGCTACTGCCCCTACAGCCAGTGC |
| | | GCCTTCAACGGCATCTTCCTGCCACCTCTGCAGGGGGATTTCGGCGC |
| | | CTTCAGCGCCTTCTACTTCGTGATGAAGTTCCTGAACCTGACCAGCGA |
| | | GAAGGTGTCCCAGGAAAAAGTGACAGAGATGATGAAGAAGTTCTGCG |
| | | CCCAGCCCTGGGAGGAAATCAAGACCTCCTACGCTGGCGTGAAAGAG |
| | | AAGTACCTGAGCGAGTTCTGCTTCAGCGGCACCTACATCCTGAGCCT |
| | | GCTGCAGCAGGGCTACCACTTCACCGCCGATAGCTGGGAGCACATCC |
| | | ACTTCATCGGCAAGATTCAGGGCAGCGACGCCGGCTGGACACTGGGC |
| | | TACATGCTGAATCTGACCAACATGATCCCCGCCGAGCAGCCCCTGAG |
| | | CACACCTCTGAGCCACAGCACC |
| EP1xEP17xEP19aa1-6 | | |
| SEQ ID | Amino | APTSSSTQNKALPENVKYGIVLDAGSSHTSLYIYKWPAEKENDTGVVHQV |
| NO: 219 | acid | EECRVKGPGISKFVQKVNEIGIYLTDCMERAMEVIPRSQHQETPVYLGAT |
| | | AGMRLLRMESEELADRVLDVVERSLSNYPFDFQGARIITGQEEGAYGWIT |
| | | INYLLGKFSQKNQETFGALDLGGASTQVTFVPQNQTIESPDNALQFRLYG |
| | | KDYNVYTHSFLCYGKDQALWQKLAKDIQVASNEILRDPCFHPGYKKVVNV |
| | | SDLYKTPCTKRFEMTLPFQQFEIQGIGNYQQCHQSILELFNTSYCPYSQC |
| | | AFNGIFLPPLQGDFGAFSAFYFVMKFLNLTSEKVSQEKVTEMMKKFCAQP |
| | | WEEIKTSYAGVKEKYLSEFCFSGTYILSLLQQGYHFTADSWEHIHFIGKIQ |
| | | GSDAGWTLGYMLNLTNMIPAEQPLSTPLSHST |
| SEQ ID | DNA | GCCCCTACCAGCAGCAGCACCCAGAACAAGGCCCTGCCCGAGAACGT |
| NO: 220 | | GAAGTACGGCATCGTGCTGGATGCCGGCAGCAGCCACACCAGCCTGT |
| | | ACATCTACAAGTGGCCTGCCGAGAAAGAAAACGACACCGGCGTGGTG |
| | | CATCAGGTGGAAGAGTGCAGAGTGAAGGGCCCTGGCATCAGCAAGTT |
| | | CGTGCAGAAAGTGAACGAGATCGGCATCTACCTGACCGACTGCATGG |
| | | AACGGGCCATGGAAGTGATCCCCAGAAGCCAGCACCAGGAAACCCCC |
| | | GTGTATCTGGGAGCCACCGCCGGCATGAGACTGCTGAGAATGGAAAG |
| | | CGAGGAACTGGCCGACCGGGTGCTGGACGTGGTGGAAAGAAGCCTG |
| | | AGCAACTACCCATTCGATTTTCAAGGCGCCAGAATCATCACCGGCCAG |
| | | GAAGAAGGCGCCTACGGCTGGATCACCATCAACTACCTGCTGGGCAA |
| | | GTTCAGCCAGAAGAATCAGGAAACCTTCGGCGCCCTGGACCTGGGCG |
| | | GAGCTTCTACCCAAGTGACCTTCGTGCCCCAGAATCAGACCATCGAGA |
| | | GCCCCGACAACGCCCTGCAGTTCCGGCTGTACGGCAAGGACTACAAT |
| | | GTGTACACCCACAGCTTTCTGTGCTACGGAAAGGACCAGGCTCTGTG |
| | | GCAGAAGCTGGCCAAGGACATCCAGGTGGCCAGCAACGAGATCCTGC |
| | | GGGACCCTTGCTTCCACCCCGGCTACAAGAAAGTCGTGAACGTGTCC |
| | | GACCTGTACAAGACCCCCTGCACCAAGAGATTCGAGATGACCCTGCC |
| | | CTTCCAGCAGTTCGAGATCCAGGGCATCGGCAATTACCAGCAGTGCC |
| | | ACCAGAGCATCCTGGAACTGTTCAACACCAGCTACTGCCCCTACAGCC |
| | | AGTGCGCCTTCAACGGCATCTTCCTGCCACCTCTGCAGGGGGATTTC |
| | | GGCGCCTTCAGCGCCTTCTACTTCGTGATGAAGTTCCTGAACCTGACC |
| | | AGCGAGAAGGTGTCCCAGGAAAAAGTGACAGAGATGATGAAGAAGTT |
| | | CTGCGCCCAGCCCTGGGAGGAAATCAAGACCTCCTACGCTGGCGTGA |
| | | AAGAGAAGTACCTGAGCGAGTTCTGCTTCAGCGGCACCTACATCCTGA |
| | | GCCTGCTGCAGCAGGGCTACCACTTCACCGCCGATAGCTGGGAGCAC |
| | | ATCCACTTCATCGGCAAGATTCAGGGCAGCGACGCCGGCTGGACACT |
| | | GGGCTACATGCTGAATCTGACCAACATGATCCCCGCCGAGCAGCCCC |
| | | TGAGCACACCTCTGAGCCACAGCACC |
| EP1xEP14aa1-3 | | |
| SEQ ID | Amino | APTTQNKALPENVKYGIVLDAGSSHTSLYIYKWPAEKENDTGVVHQVEEC |
| NO: 221 | acid | RVKGPGISKFVQKVNEIGIYLTDCMERAMEVIPRSQHQETPVYLGATAGM |
| | | RLLRMESEELADRVLDVVERSLSNYPFDFQGARIITGQEEGAYGWITINYL |
| | | LGKFSQKNQETFGALDLGGASTQVTFVPQNQTIESPDNALQFRLYGKDY |
| | | NVYTHSFLCYGKDQALWQKLAKDIQVASNEILRDPCFHPGYKKVVNVSDL |
| | | YKTPCTKRFEMTLPFQQFEIQGIGNYQQCHQSILELFNTSYCPYSQCAFN |
| | | GIFLPPLQGDFGAFSAFYSVMKFLNLTSEKVSQEKVTEMMKKFCAQPVVE |
| | | EIKTSYAGVKEKYLSEYCFSGTYILSLLLQGYHFTADSWEHIHFIGKIQGSD |
| | | AGWTLGYMLNLTNMIPAEQPLSTPLSHST |
| SEQ ID | DNA | GCCCCTACCACCCAGAACAAGGCCCTGCCCGAGAACGTGAAGTACGG |
| NO: 222 | | CATCGTGCTGGATGCCGGCAGCAGCCACACCAGCCTGTACATCTACA |
| | | AGTGGCCTGCCGAGAAAGAAAACGACACCGGCGTGGTGCATCAGGTG |
| | | GAAGAGTGCAGAGTGAAGGGCCCTGGCATCAGCAAGTTCGTGCAGAA |
| | | AGTGAACGAGATCGGCATCTACCTGACCGACTGCATGGAACGGGCCA |
| | | TGGAAGTGATCCCCAGAAGCCAGCACCAGGAAACCCCCGTGTATCTG |
| | | GGAGCCACCGCCGGCATGAGACTGCTGAGAATGGAAAGCGAGGAAC |
| | | TGGCCGACCGGGTGCTGGACGTGGTGGAAAGAAGCCTGAGCAACTA |
| | | CCCATTCGATTTTCAAGGCGCCAGAATCATCACCGGCCAGGAAGAAG |
| | | GCGCCTACGGCTGGATCACCATCAACTACCTGCTGGGCAAGTTCAGC |
| | | CAGAAGAATCAGGAAACCTTCGGCGCCTTGGACCTGGGCGGAGCTTC |
| | | TACCCAAGTGACCTTCGTGCCCCAGAATCAGACCATCGAGAGCCCCG |
| | | ACAACGCCCTGCAGTTCCGGCTGTACGGCAAGGACTACAATGTGTAC |
| | | ACCCACAGCTTTCTGTGCTACGGAAAGGACCAGGCTCTGTGGCAGAA |
| | | GCTGGCCAAGGACATCCAGGTGGCCAGCAACGAGATCCTGCGGGAC |
| | | CCTTGCTTCCACCCCGGCTACAAGAAAGTCGTGAACGTGTCCGACCT |
| | | GTACAAGACCCCCTGCACCAAGAGATTCGAGATGACCCTGCCCTTCC |
| | | AGCAGTTCGAGATCCAGGGCATCGGCAATTACCAGCAGTGCCACCAG |
| | | AGCATCCTGGAACTGTTCAACACCAGCTACTGCCCCTACAGCCAGTGC |
| | | GCCTTCAACGGCATCTTCCTGCCACCTCTGCAGGGGGATTTCGGCGC |
| | | CTTCAGCGCCTTCTACTCCGTGATGAAGTTCCTGAACCTGACCAGCGA |
| | | GAAGGTGTCCCAGGAAAAAGTGACAGAGATGATGAAGAAGTTCTGCG |
| | | CCCAGCCCTGGGAGGAAATCAAGACCTCCTACGCTGGCGTGAAAGAG |
| | | AAGTACCTGAGCGAGTACTGCTTCAGCGGCACCTACATCCTGAGCCT |
| | | GCTGCTGCAGGGCTACCACTTCACCGCCGATAGCTGGGAGCACATCC |
| | | ACTTCATCGGCAAGATTCAGGGCAGCGACGCCGGCTGGACACTGGGT |
| | | TACATGCTGAATCTGACCAACATGATCCCCGCCGAGCAGCCCCTGAG |
| | | CACACCTCTGAGCCACAGCACC |
| EP1xEP14aa1-6 | | |
| SEQ ID | Amino | APTSSSTQNKALPENVKYGIVLDAGSSHTSLYIYKWPAEKENDTGVVHQV |
| NO: 223 | acid | EECRVKGPGISKFVQKVNEIGIYLTDCMERAMEVIPRSQHQETPVYLGAT |
| | | AGMRLLRMESEELADRVLDVVERSLSNYPFDFQGARIITGQEEGAYGWIT |
| | | INYLLGKFSQKNQETFGALDLGGASTQVTFVPQNQTIESPDNALQFRLYG |
| | | KDYNVYTHSFLCYGKDQALWQKLAKDIQVASNEILRDPCFHPGYKKVVNV |
| | | SDLYKTPCTKRFEMTLPFQQFEIQGIGNYQQCHQSILELFNTSYCPYSQC |
| | | AFNGIFLPPLQGDFGAFSAFYSVMKFLNLTSEKVSQEKVTEMMKKFCAQP |
| | | WEEIKTSYAGVKEKYLSEYCFSGTYILSLLLQGYHFTADSWEHIHFIGKIQG |
| | | SDAGWTLGYMLNLTNMIPAEQPLSTPLSHST |
| SEQ ID | DNA | GCCCCTACCAGCAGCAGCACCCAGAACAAGGCCCTGCCCGAGAACGT |
| NO: 224 | | GAAGTACGGCATCGTGCTGGATGCCGGCAGCAGCCACACCAGCCTGT |
| | | ACATCTACAAGTGGCCTGCCGAGAAAGAAAACGACACCGGCGTGGTG |
| | | CATCAGGTGGAAGAGTGCAGAGTGAAGGGCCCTGGCATCAGCAAGTT |
| | | CGTGCAGAAAGTGAACGAGATCGGCATCTACCTGACCGACTGCATGG |
| | | AACGGGCCATGGAAGTGATCCCCAGAAGCCAGCACCAGGAAACCCCC |
| | | GTGTATCTGGGAGCCACCGCCGGCATGAGACTGCTGAGAATGGAAAG |
| | | CGAGGAACTGGCCGACCGGGTGCTGGACGTGGTGGAAAGAAGCCTG |
| | | AGCAACTACCCATTCGATTTTCAAGGCGCCAGAATCATCACCGGCCAG |
| | | GAAGAAGGCGCCTACGGCTGGATCACCATCAACTACCTGCTGGGCAA |
| | | GTTCAGCCAGAAGAATCAGGAAACCTTCGGCGCCTTGGACCTGGGCG |
| | | GAGCTTCTACCCAAGTGACCTTCGTGCCCCAGAATCAGACCATCGAGA |
| | | GCCCCGACAACGCCCTGCAGTTCCGGCTGTACGGCAAGGACTACAAT |
| | | GTGTACACCCACAGCTTTCTGTGCTACGGAAAGGACCAGGCTCTGTG |
| | | GCAGAAGCTGGCCAAGGACATCCAGGTGGCCAGCAACGAGATCCTGC |
| | | GGGACCCTTGCTTCCACCCCGGCTACAAGAAAGTCGTGAACGTGTCC |
| | | GACCTGTACAAGACCCCCTGCACCAAGAGATTCGAGATGACCCTGCC |
| | | CTTCCAGCAGTTCGAGATCCAGGGCATCGGCAATTACCAGCAGTGCC |
| | | ACCAGAGCATCCTGGAACTGTTCAACACCAGCTACTGCCCCTACAGCC |
| | | AGTGCGCCTTCAACGGCATCTTCCTGCCACCTCTGCAGGGGGATTTC |
| | | GGCGCCTTCAGCGCCTTCTACTCCGTGATGAAGTTCCTGAACCTGACC |
| | | AGCGAGAAGGTGTCCCAGGAAAAAGTGACAGAGATGATGAAGAAGTT |
| | | CTGCGCCCAGCCCTGGGAGGAAATCAAGACCTCCTACGCTGGCGTGA |
| | | AAGAGAAGTACCTGAGCGAGTACTGCTTCAGCGGCACCTACATCCTG |
| | | AGCCTGCTGCTGCAGGGCTACCACTTCACCGCCGATAGCTGGGAGCA |
| | | CATCCACTTCATCGGCAAGATTCAGGGCAGCGACGCCGGCTGGACAC |
| | | TGGGTTACATGCTGAATCTGACCAACATGATCCCCGCCGAGCAGCCC |
| | | CTGAGCACACCTCTGAGCCACAGCACC |
| EP17xEP19aa1-3 | | |
| SEQ ID | Amino | APTTQNKALPENVKYGIVLDAGSSHTSLYIYKWPAEKENDTGVVHQVEEC |
| NO: 225 | acid | RVKGPGISKFVQKVNEIGIYLTDCMERAREVIPRSQHQETPVYLGATAGM |
| | | RLLRMESEELADRVLDVVERSLSNYPFDFQGARIITGQEEGAYGWITINYL |
| | | LGKFSQKNQETFGALDLGGASTQVTFVPQNQTIESPDNALQFRLYGKDY |
| | | NVYTHSFLCYGKDQALWQKLAKDIQVASNEILRDPCFHPGYKKVVNVSDL |
| | | YKTPCTKRFEMTLPFQQFEIQGIGNYQQCHQSILELFNTSYCPYSQCAFN |
| | | GIFLPPLQGDFGAFSAFYFVMKFLNLTSEKVSQEKVTEMMKKFCAQPWE |
| | | EIKTSYAGVKEKYLSEFCFSGTYILSLLQQGYHFTADSWEHIHFIGKIQGSD |
| | | AGWTLGYMLNLTNMIPAEQPLSTPLSHST |
| SEQ ID | DNA | GCCCCTACCACCCAGAACAAGGCCCTGCCCGAGAACGTGAAGTACGG |
| NO: 226 | | CATCGTGCTGGATGCCGGCAGCAGCCACACCAGCCTGTACATCTACA |
| | | AGTGGCCTGCCGAGAAAGAAAACGACACCGGCGTGGTGCATCAGGTG |
| | | GAAGAGTGCAGAGTGAAGGGCCCTGGCATCAGCAAGTTCGTGCAGAA |
| | | AGTGAACGAGATCGGCATCTACCTGACCGACTGCATGGAACGGGCCA |
| | | GGGAAGTGATCCCCAGAAGCCAGCACCAGGAAACCCCCGTGTATCTG |
| | | GGAGCCACCGCCGGCATGAGACTGCTGAGAATGGAAAGCGAGGAAC |
| | | TGGCCGACCGGGTGCTGGACGTGGTGGAAAGAAGCCTGAGCAACTA |
| | | CCCATTCGATTTTCAAGGCGCCAGAATCATCACCGGCCAGGAAGAAG |
| | | GCGCCTACGGCTGGATCACCATCAACTACCTGCTGGGCAAGTTCAGC |
| | | CAGAAGAATCAGGAAACCTTCGGCGCCCTGGACCTGGGCGGAGCTTC |
| | | TACCCAAGTGACCTTCGTGCCCCAGAATCAGACCATCGAGAGCCCCG |
| | | ACAACGCCCTGCAGTTCCGGCTGTACGGCAAGGACTACAATGTGTAC |
| | | ACCCACAGCTTTCTGTGCTACGGAAAGGACCAGGCTCTGTGGCAGAA |
| | | GCTGGCCAAGGACATCCAGGTGGCCAGCAACGAGATCCTGCGGGAC |
| | | CCTTGCTTCCACCCCGGCTACAAGAAAGTCGTGAACGTGTCCGACCT |
| | | GTACAAGACCCCCTGCACCAAGAGATTCGAGATGACCCTGCCCTTCC |
| | | AGCAGTTCGAGATCCAGGGCATCGGCAATTACCAGCAGTGCCACCAG |
| | | AGCATCCTGGAACTGTTCAACACCAGCTACTGCCCCTACAGCCAGTGC |
| | | GCCTTCAACGGCATCTTCCTGCCACCTCTGCAGGGGGATTTCGGCGC |
| | | CTTCAGCGCCTTCTACTTCGTGATGAAGTTCCTGAACCTGACCAGCGA |
| | | GAAGGTGTCCCAGGAAAAAGTGACAGAGATGATGAAGAAGTTCTGCG |
| | | CCCAGCCCTGGGAGGAAATCAAGACCTCCTACGCTGGCGTGAAAGAG |
| | | AAGTACCTGAGCGAGTTTTGCTTCAGCGGCACCTACATCCTGAGCCTG |
| | | CTGCAGCAGGGCTACCACTTCACCGCCGATAGCTGGGAGCACATCCA |
| | | CTTCATCGGCAAGATTCAGGGCAGCGACGCCGGCTGGACACTGGGCT |
| | | ACATGCTGAATCTGACCAACATGATCCCCGCCGAGCAGCCCCTGAGC |
| | | ACACCTCTGAGCCACAGCACC |
| EP17xEP19aa1-6 | | |
| SEQ ID | Amino | APTSSSTQNKALPENVKYGIVLDAGSSHTSLYIYKWPAEKENDTGVVHQV |
| NO: 227 | acid | EECRVKGPGISKFVQKVNEIGIYLTDCMERAREVIPRSQHQETPVYLGATA |
| | | GMRLLRMESEELADRVLDVVERSLSNYPFDFQGARIITGQEEGAYGWITI |
| | | NYLLGKFSQKNQETFGALDLGGASTQVTFVPQNQTIESPDNALQFRLYGK |
| | | DYNVYTHSFLCYGKDQALWQKLAKDIQVASNEILRDPCFHPGYKKVVNVS |
| | | DLYKTPCTKRFEMTLPFQQFEIQGIGNYQQCHQSILELFNTSYCPYSQCA |
| | | FNGIFLPPLQGDFGAFSAFYFVMKFLNLTSEKVSQEKVTEMMKKFCAQP |
| | | WEEIKTSYAGVKEKYLSEFCFSGTYILSLLQQGYHFTADSWEHIHFIGKIQ |
| | | GSDAGVVTLGYMLNLTNMIPAEQPLSTPLSHST |
| SEQ ID | DNA | GCCCCTACCAGCAGCAGCACCCAGAACAAGGCCCTGCCCGAGAACGT |
| NO: 228 | | GAAGTACGGCATCGTGCTGGATGCCGGCAGCAGCCACACCAGCCTGT |
| | | ACATCTACAAGTGGCCTGCCGAGAAAGAAAACGACACCGGCGTGGTG |
| | | CATCAGGTGGAAGAGTGCAGAGTGAAGGGCCCTGGCATCAGCAAGTT |
| | | CGTGCAGAAAGTGAACGAGATCGGCATCTACCTGACCGACTGCATGG |
| | | AACGGGCCAGGGAAGTGATCCCCAGAAGCCAGCACCAGGAAACCCC |
| | | CGTGTATCTGGGAGCCACCGCCGGCATGAGACTGCTGAGAATGGAAA |
| | | GCGAGGAACTGGCCGACCGGGTGCTGGACGTGGTGGAAAGAAGCCT |
| | | GAGCAACTACCCATTCGATTTTCAAGGCGCCAGAATCATCACCGGCCA |
| | | GGAAGAAGGCGCCTACGGCTGGATCACCATCAACTACCTGCTGGGCA |
| | | AGTTCAGCCAGAAGAATCAGGAAACCTTCGGCGCCCTGGACCTGGGC |
| | | GGAGCTTCTACCCAAGTGACCTTCGTGCCCCAGAATCAGACCATCGA |
| | | GAGCCCCGACAACGCCCTGCAGTTCCGGCTGTACGGCAAGGACTACA |
| | | ATGTGTACACCCACAGCTTTCTGTGCTACGGAAAGGACCAGGCTCTGT |
| | | GGCAGAAGCTGGCCAAGGACATCCAGGTGGCCAGCAACGAGATCCT |
| | | GCGGGACCCTTGCTTCCACCCCGGCTACAAGAAAGTCGTGAACGTGT |
| | | CCGACCTGTACAAGACCCCCTGCACCAAGAGATTCGAGATGACCCTG |
| | | CCCTTCCAGCAGTTCGAGATCCAGGGCATCGGCAATTACCAGCAGTG |
| | | CCACCAGAGCATCCTGGAACTGTTCAACACCAGCTACTGCCCCTACAG |
| | | CCAGTGCGCCTTCAACGGCATCTTCCTGCCACCTCTGCAGGGGGATT |
| | | TCGGCGCCTTCAGCGCCTTCTACTTCGTGATGAAGTTCCTGAACCTGA |
| | | CCAGCGAGAAGGTGTCCCAGGAAAAAGTGACAGAGATGATGAAGAAG |
| | | TTCTGCGCCCAGCCCTGGGAGGAAATCAAGACCTCCTACGCTGGCGT |
| | | GAAAGAGAAGTACCTGAGCGAGTTTTGCTTCAGCGGCACCTACATCCT |
| | | GAGCCTGCTGCAGCAGGGCTACCACTTCACCGCCGATAGCTGGGAGC |
| | | ACATCCACTTCATCGGCAAGATTCAGGGCAGCGACGCCGGCTGGACA |
| | | CTGGGCTACATGCTGAATCTGACCAACATGATCCCCGCCGAGCAGCC |
| | | CCTGAGCACACCTCTGAGCCACAGCACC |
| EP14aa1-3 | | |
| SEQ ID | Amino | APTTQNKALPENVKYGIVLDAGSSHTSLYIYKWPAEKENDTGVVHQVEEC |
| NO: 229 | acid | RVKGPGISKFVQKVNEIGIYLTDCMERAREVIPRSQHQETPVYLGATAGM |
| | | RLLRMESEELADRVLDVVERSLSNYPFDFQGARIITGQEEGAYGWITINYL |
| | | LGKFSQKNQETFGALDLGGASTQVTFVPQNQTIESPDNALQFRLYGKDY |
| | | NVYTHSFLCYGKDQALWQKLAKDIQVASNEILRDPCFHPGYKKVVNVSDL |
| | | YKTPCTKRFEMTLPFQQFEIQGIGNYQQCHQSILELFNTSYCPYSQCAFN |
| | | GIFLPPLQGDFGAFSAFYSVMKFLNLTSEKVSQEKVTEMMKKFCAQPWE |
| | | EIKTSYAGVKEKYLSEYCFSGTYILSLLLQGYHFTADSWEHIHFIGKIQGSD |
| | | AGWTLGYMLNLTNMIPAEQPLSTPLSHST |
| SEQ ID | DNA | GCCCCTACCACCCAGAACAAGGCCCTGCCCGAGAACGTGAAGTACGG |
| NO: 230 | | CATCGTGCTGGACGCCGGCTCCTCCCACACCTCCCTGTACATCTACAA |
| | | GTGGCCTGCCGAGAAAGAAAACGACACCGGCGTGGTGCACCAAGTG |
| | | GAAGAGTGCAGAGTGAAGGGCCCCGGCATCTCCAAGTTCGTGCAGAA |
| | | AGTGAACGAGATCGGCATCTACCTGACCGACTGCATGGAACGGGCCA |
| | | GAGAAGTGATCCCTCGGTCCCAGCACCAGGAAACCCCTGTCTACCTG |
| | | GGCGCCACCGCCGGCATGCGGCTGCTGCGGATGGAATCCGAGGAAC |
| | | TGGCCGACCGGGTGCTGGACGTGGTGGAACGGTCCCTGTCCAACTAC |
| | | CCATTCGATTTTCAAGGCGCCAGAATCATCACCGGCCAGGAAGAGGG |
| | | CGCCTACGGCTGGATCACCATCAACTACCTGCTGGGCAAGTTCTCCC |
| | | AGAAGAATCAGGAAACCTTCGGCGCCCTGGACCTGGGCGGAGCCAG |
| | | CACCCAAGTCACATTCGTGCCCCAGAACCAGACCATCGAGAGCCCCG |
| | | ACAACGCCCTGCAGTTCCGGCTGTACGGCAAGGACTACAACGTGTAC |
| | | ACCCACAGCTTTCTGTGCTACGGCAAGGACCAGGCCCTGTGGCAGAA |
| | | GCTGGCCAAGGACATCCAAGTGGCCTCCAACGAGATCCTGCGGGACC |
| | | CCTGCTTCCACCCCGGCTACAAGAAAGTGGTCAACGTGTCCGACCTG |
| | | TACAAGACCCCTTGCACCAAGAGATTCGAGATGACCCTGCCCTTCCAG |
| | | CAGTTCGAGATCCAGGGCATCGGCAACTACCAGCAGTGCCACCAGTC |
| | | CATCCTGGAACTGTTCAACACCTCCTACTGCCCCTACTCCCAGTGCGC |
| | | CTTCAACGGCATCTTCCTGCCTCCACTGCAGGGCGACTTCGGCGCCT |
| | | TCTCCGCCTTCTACTCCGTGATGAAGTTCCTGAACCTGACCTCCGAGA |
| | | AAGTGTCCCAGGAAAAAGTGACCGAGATGATGAAGAAGTTCTGCGCC |
| | | CAGCCCTGGGAGGAAATCAAGACCTCCTACGCTGGCGTGAAAGAGAA |
| | | GTACCTGTCCGAGTACTGCTTCTCCGGCACCTACATCCTGTCCCTGCT |
| | | GCTGCAGGGCTACCACTTCACCGCCGACAGCTGGGAGCACATCCACT |
| | | TCATCGGCAAGATCCAGGGATCCGACGCTGGCTGGACCCTGGGCTAC |
| | | ATGCTGAATCTGACCAACATGATCCCCGCCGAGCAGCCCCTGTCCAC |
| | | CCCTCTGTCTCACTCCACC |
| EP14aa1-6 | | |
| SEQ ID | Amino | APTSSSTQNKALPENVKYGIVLDAGSSHTSLYIYKWPAEKENDTGVVHQV |
| NO: 231 | acid | EECRVKGPGISKFVQKVNEIGIYLTDCMERAREVIPRSQHQETPVYLGATA |
| | | GMRLLRMESEELADRVLDVVERSLSNYPFDFQGARIITGQEEGAYGWITI |
| | | NYLLGKFSQKNQETFGALDLGGASTQVTFVPQNQTIESPDNALQFRLYGK |
| | | DYNVYTHSFLCYGKDQALWQKLAKDIQVASNEILRDPCFHPGYKKVVNVS |
| | | DLYKTPCTKRFEMTLPFQQFEIQGIGNYQQCHQSILELFNTSYCPYSQCA |
| | | FNGIFLPPLQGDFGAFSAFYSVMKFLNLTSEKVSQEKVTEMMKKFCAQP |
| | | WEEIKTSYAGVKEKYLSEYCFSGTYILSLLLQGYHFTADSWEHIHFIGKIQG |
| | | SDAGWTLGYMLNLTNMIPAEQPLSTPLSHST |
| SEQ ID | DNA | GCCCCTACCAGCAGCAGCACCCAGAACAAGGCCCTGCCCGAGAACGT |
| NO: 232 | | GAAGTACGGCATCGTGCTGGATGCCGGCAGCAGCCACACCAGCCTGT |
| | | ACATCTACAAGTGGCCTGCCGAGAAAGAAAACGACACCGGCGTGGTG |
| | | CATCAGGTGGAAGAGTGCAGAGTGAAGGGCCCTGGCATCAGCAAGTT |
| | | CGTGCAGAAAGTGAACGAGATCGGCATCTACCTGACCGACTGCATGG |
| | | AACGGGCCAGGGAAGTGATCCCCAGAAGCCAGCACCAGGAAACCCC |
| | | CGTGTATCTGGGAGCCACCGCCGGCATGAGACTGCTGAGAATGGAAA |
| | | GCGAGGAACTGGCCGACCGGGTGCTGGACGTGGTGGAAAGAAGCCT |
| | | GAGCAACTACCCATTCGATTTTCAAGGCGCCAGAATCATCACCGGCCA |
| | | GGAAGAAGGCGCCTACGGCTGGATCACCATCAACTACCTGCTGGGCA |
| | | AGTTCAGCCAGAAGAATCAGGAAACCTTCGGCGCCTTGGACCTGGGC |
| | | GGAGCTTCTACCCAAGTGACCTTCGTGCCCCAGAATCAGACCATCGA |
| | | GAGCCCCGACAACGCCCTGCAGTTCCGGCTGTACGGCAAGGACTACA |
| | | ATGTGTACACCCACAGCTTTCTGTGCTACGGAAAGGACCAGGCTCTGT |
| | | GGCAGAAGCTGGCCAAGGACATCCAGGTGGCCAGCAACGAGATCCT |
| | | GCGGGACCCTTGCTTCCACCCCGGCTACAAGAAAGTCGTGAACGTGT |
| | | CCGACCTGTACAAGACCCCCTGCACCAAGAGATTCGAGATGACCCTG |
| | | CCCTTCCAGCAGTTCGAGATCCAGGGCATCGGCAATTACCAGCAGTG |
| | | CCACCAGAGCATCCTGGAACTGTTCAACACCAGCTACTGCCCCTACAG |
| | | CCAGTGCGCCTTCAACGGCATCTTCCTGCCACCTCTGCAGGGGGATT |
| | | TCGGCGCCTTCAGCGCCTTCTACTCCGTGATGAAGTTCCTGAACCTGA |
| | | CCAGCGAGAAGGTGTCCCAGGAAAAAGTGACAGAGATGATGAAGAAG |
| | | TTCTGCGCCCAGCCCTGGGAGGAAATCAAGACCTCCTACGCTGGCGT |
| | | GAAAGAGAAGTACCTGAGCGAGTACTGCTTCAGCGGCACCTACATCC |
| | | TGAGCCTGCTGCTGCAGGGCTACCACTTCACCGCCGATAGCTGGGAG |
| | | CACATCCACTTCATCGGCAAGATTCAGGGCAGCGACGCCGGCTGGAC |
| | | ACTGGGTTACATGCTGAATCTGACCAACATGATCCCCGCCGAGCAGC |
| | | CCCTGAGCACACCTCTGAGCCACAGCACC |
| EP1xEP14xEP19aa1-3 | | |
| SEQ ID | Amino | APTTQNKALPENVKYGIVLDAGSSHTSLYIYKWPAEKENDTGVVHQVEEC |
| NO: 233 | acid | RVKGPGISKFVQKVNEIGIYLTDCMERAMEVIPRSQHQETPVYLGATAGM |
| | | RLLRMESEELADRVLDVVERSLSNYPFDFQGARIITGQEEGAYGWITINYL |
| | | LGKFSQKNQETFGALDLGGASTQVTFVPQNQTIESPDNALQFRLYGKDY |
| | | NVYTHSFLCYGKDQALWQKLAKDIQVASNEILRDPCFHPGYKKVVNVSDL |
| | | YKTPCTKRFEMTLPFQQFEIQGIGNYQQCHQSILELFNTSYCPYSQCAFN |
| | | GIFLPPLQGDFGAFSAFYSVMKFLNLTSEKVSQEKVTEMMKKFCAQPWE |
| | | EIKTSYAGVKEKYLSEYCFSGTYILSLLQQGYHFTADSWEHIHFIGKIQGSD |
| | | AGWTLGYMLNLTNMIPAEQPLSTPLSHST |
| SEQ ID | DNA | GCCCCTACCACCCAGAACAAGGCCCTGCCCGAGAACGTGAAGTACGG |
| NO: 234 | | CATCGTGCTGGATGCCGGCAGCAGCCACACCAGCCTGTACATCTACA |
| | | AGTGGCCTGCCGAGAAAGAAAACGACACCGGCGTGGTGCATCAGGTG |
| | | GAAGAGTGCAGAGTGAAGGGCCCTGGCATCAGCAAGTTCGTGCAGAA |
| | | AGTGAACGAGATCGGCATCTACCTGACCGACTGCATGGAACGGGCCA |
| | | TGGAAGTGATCCCCAGAAGCCAGCACCAGGAAACCCCCGTGTATCTG |
| | | GGAGCCACCGCCGGCATGAGACTGCTGAGAATGGAAAGCGAGGAAC |
| | | TGGCCGACCGGGTGCTGGACGTGGTGGAAAGAAGCCTGAGCAACTA |
| | | CCCATTCGATTTTCAAGGCGCCAGAATCATCACCGGCCAGGAAGAAG |
| | | GCGCCTACGGCTGGATCACCATCAACTACCTGCTGGGCAAGTTCAGC |
| | | CAGAAGAATCAGGAAACCTTCGGCGCCCTGGACCTGGGCGGAGCTTC |
| | | TACCCAAGTGACCTTCGTGCCCCAGAATCAGACCATCGAGAGCCCCG |
| | | ACAACGCCCTGCAGTTCCGGCTGTACGGCAAGGACTACAATGTGTAC |
| | | ACCCACAGCTTTCTGTGCTACGGAAAGGACCAGGCTCTGTGGCAGAA |
| | | GCTGGCCAAGGACATCCAGGTGGCCAGCAACGAGATCCTGCGGGAC |
| | | CCTTGCTTCCACCCCGGCTACAAGAAAGTCGTGAACGTGTCCGACCT |
| | | GTACAAGACCCCCTGCACCAAGAGATTCGAGATGACCCTGCCCTTCC |
| | | AGCAGTTCGAGATCCAGGGCATCGGCAATTACCAGCAGTGCCACCAG |
| | | AGCATCCTGGAACTGTTCAACACCAGCTACTGCCCCTACAGCCAGTGC |
| | | GCCTTCAACGGCATCTTCCTGCCACCTCTGCAGGGGGATTTCGGCGC |
| | | CTTCAGCGCCTTCTACTCCGTGATGAAGTTCCTGAACCTGACCAGCGA |
| | | GAAGGTGTCCCAGGAAAAAGTGACAGAGATGATGAAGAAGTTCTGCG |
| | | CCCAGCCCTGGGAGGAAATCAAGACCTCCTACGCTGGCGTGAAAGAG |
| | | AAGTACCTGAGCGAGTACTGCTTCAGCGGCACCTACATCCTGAGCCT |
| | | GCTGCAGCAGGGCTACCACTTCACCGCCGATAGCTGGGAGCACATCC |
| | | ACTTCATCGGCAAGATTCAGGGCAGCGACGCCGGCTGGACACTGGGC |
| | | TACATGCTGAATCTGACCAACATGATCCCCGCCGAGCAGCCCCTGAG |
| | | CACACCTCTGAGCCACAGCACC |
| EP1xEP14xEP19aa1-6 | | |
| SEQ ID | Amino | APTSSSTQNKALPENVKYGIVLDAGSSHTSLYIYKWPAEKENDTGVVHQV |
| NO: 235 | acid | EECRVKGPGISKFVQKVNEIGIYLTDCMERAMEVIPRSQHQETPVYLGAT |
| | | AGMRLLRMESEELADRVLDVVERSLSNYPFDFQGARIITGQEEGAYGWIT |
| | | INYLLGKFSQKNQETFGALDLGGASTQVTFVPQNQTIESPDNALQFRLYG |
| | | KDYNVYTHSFLCYGKDQALWQKLAKDIQVASNEILRDPCFHPGYKKVVNV |
| | | SDLYKTPCTKRFEMTLPFQQFEIQGIGNYQQCHQSILELFNTSYCPYSQC |
| | | AFNGIFLPPLQGDFGAFSAFYSVMKFLNLTSEKVSQEKVTEMMKKFCAQP |
| | | WEEIKTSYAGVKEKYLSEYCFSGTYILSLLQQGYHFTADSWEHIHFIGKIQ |
| | | GSDAGWTLGYMLNLTNMIPAEQPLSTPLSHST |
| SEQ ID | DNA | GCCCCTACCAGCAGCAGCACCCAGAACAAGGCCCTGCCCGAGAACGT |
| NO: 236 | | GAAGTACGGCATCGTGCTGGATGCCGGCAGCAGCCACACCAGCCTGT |
| | | ACATCTACAAGTGGCCTGCCGAGAAAGAAAACGACACCGGCGTGGTG |
| | | CATCAGGTGGAAGAGTGCAGAGTGAAGGGCCCTGGCATCAGCAAGTT |
| | | CGTGCAGAAAGTGAACGAGATCGGCATCTACCTGACCGACTGCATGG |
| | | AACGGGCCATGGAAGTGATCCCCAGAAGCCAGCACCAGGAAACCCCC |
| | | GTGTATCTGGGAGCCACCGCCGGCATGAGACTGCTGAGAATGGAAAG |
| | | CGAGGAACTGGCCGACCGGGTGCTGGACGTGGTGGAAAGAAGCCTG |
| | | AGCAACTACCCATTCGATTTTCAAGGCGCCAGAATCATCACCGGCCAG |
| | | GAAGAAGGCGCCTACGGCTGGATCACCATCAACTACCTGCTGGGCAA |
| | | GTTCAGCCAGAAGAATCAGGAAACCTTCGGCGCCCTGGACCTGGGCG |
| | | GAGCTTCTACCCAAGTGACCTTCGTGCCCCAGAATCAGACCATCGAGA |
| | | GCCCCGACAACGCCCTGCAGTTCCGGCTGTACGGCAAGGACTACAAT |
| | | GTGTACACCCACAGCTTTCTGTGCTACGGAAAGGACCAGGCTCTGTG |
| | | GCAGAAGCTGGCCAAGGACATCCAGGTGGCCAGCAACGAGATCCTGC |
| | | GGGACCCTTGCTTCCACCCCGGCTACAAGAAAGTCGTGAACGTGTCC |
| | | GACCTGTACAAGACCCCCTGCACCAAGAGATTCGAGATGACCCTGCC |
| | | CTTCCAGCAGTTCGAGATCCAGGGCATCGGCAATTACCAGCAGTGCC |
| | | ACCAGAGCATCCTGGAACTGTTCAACACCAGCTACTGCCCCTACAGCC |
| | | AGTGCGCCTTCAACGGCATCTTCCTGCCACCTCTGCAGGGGGATTTC |
| | | GGCGCCTTCAGCGCCTTCTACTCCGTGATGAAGTTCCTGAACCTGACC |
| | | AGCGAGAAGGTGTCCCAGGAAAAAGTGACAGAGATGATGAAGAAGTT |
| | | CTGCGCCCAGCCCTGGGAGGAAATCAAGACCTCCTACGCTGGCGTGA |
| | | AAGAGAAGTACCTGAGCGAGTACTGCTTCAGCGGCACCTACATCCTG |
| | | AGCCTGCTGCAGCAGGGCTACCACTTCACCGCCGATAGCTGGGAGCA |
| | | CATCCACTTCATCGGCAAGATTCAGGGCAGCGACGCCGGCTGGACAC |
| | | TGGGCTACATGCTGAATCTGACCAACATGATCCCCGCCGAGCAGCCC |
| | | CTGAGCACACCTCTGAGCCACAGCACC |
| EP17aa1-3 | | |
| SEQ ID | Amino | APTTQNKALPENVKYGIVLDAGSSHTSLYIYKWPAEKENDTGVVHQVEEC |
| NO: 237 | acid | RVKGPGISKFVQKVNEIGIYLTDCMERAREVIPRSQHQETPVYLGATAGM |
| | | RLLRMESEELADRVLDVVERSLSNYPFDFQGARIITGQEEGAYGWITINYL |
| | | LGKFSQKNQETFGALDLGGASTQVTFVPQNQTIESPDNALQFRLYGKDY |
| | | NVYTHSFLCYGKDQALWQKLAKDIQVASNEILRDPCFHPGYKKVVNVSDL |
| | | YKTPCTKRFEMTLPFQQFEIQGIGNYQQCHQSILELFNTSYCPYSQCAFN |
| | | GIFLPPLQGDFGAFSAFYFVMKFLNLTSEKVSQEKVTEMMKKFCAQPWE |
| | | EIKTSYAGVNEKYLSEFCFSGTYILSLLLQGYHFTADSWEHIHFIGKIQGSD |
| | | AGWTLGYMLNLTNMIPAEQPLSTPLSHST |
| SEQ ID | DNA | GCCCCTACCACCCAGAACAAGGCCCTGCCCGAGAACGTGAAGTACGG |
| NO: 238 | | CATCGTGCTGGATGCCGGCAGCAGCCACACCAGCCTGTACATCTACA |
| | | AGTGGCCTGCCGAGAAAGAAAACGACACCGGCGTGGTGCATCAGGTG |
| | | GAAGAGTGCAGAGTGAAGGGCCCTGGCATCAGCAAGTTCGTGCAGAA |
| | | AGTGAACGAGATCGGCATCTACCTGACCGACTGCATGGAACGGGCCA |
| | | GGGAAGTGATCCCCAGAAGCCAGCACCAGGAAACCCCCGTGTATCTG |
| | | GGAGCCACCGCCGGCATGAGACTGCTGAGAATGGAAAGCGAGGAAC |
| | | TGGCCGACCGGGTGCTGGACGTGGTGGAAAGAAGCCTGAGCAACTA |
| | | CCCATTCGATTTTCAAGGCGCCAGAATCATCACCGGCCAGGAAGAAG |
| | | GCGCCTACGGCTGGATCACCATCAACTACCTGCTGGGCAAGTTCAGC |
| | | CAGAAGAATCAGGAAACCTTCGGCGCCCTGGACCTGGGCGGAGCTTC |
| | | TACCCAAGTGACCTTCGTGCCCCAGAATCAGACCATCGAGAGCCCCG |
| | | ACAACGCCCTGCAGTTCCGGCTGTACGGCAAGGACTACAATGTGTAC |
| | | ACCCACAGCTTTCTGTGCTACGGAAAGGACCAGGCTCTGTGGCAGAA |
| | | GCTGGCCAAGGACATCCAGGTGGCCAGCAACGAGATCCTGCGGGAC |
| | | CCTTGCTTCCACCCCGGCTACAAGAAAGTCGTGAACGTGTCCGACCT |
| | | GTACAAGACCCCCTGCACCAAGAGATTCGAGATGACCCTGCCCTTCC |
| | | AGCAGTTCGAGATCCAGGGCATCGGCAATTACCAGCAGTGCCACCAG |
| | | AGCATCCTGGAACTGTTCAACACCAGCTACTGCCCCTACAGCCAGTGC |
| | | GCCTTCAACGGCATCTTCCTGCCACCTCTGCAGGGGGATTTCGGCGC |
| | | CTTCAGCGCCTTCTACTTCGTGATGAAGTTCCTGAACCTGACCAGCGA |
| | | GAAGGTGTCCCAGGAAAAAGTGACAGAGATGATGAAGAAGTTCTGCG |
| | | CCCAACCCTGGGAGGAAATCAAGACCTCCTACGCTGGCGTGAACGAG |
| | | AAGTACCTGAGCGAGTTTTGCTTCAGCGGCACCTACATCCTGAGCCTG |
| | | CTGCTGCAGGGCTACCACTTCACCGCCGATAGCTGGGAGCACATCCA |
| | | CTTCATCGGCAAGATTCAGGGCAGCGACGCCGGCTGGACACTGGGCT |
| | | ACATGCTGAATCTGACCAACATGATCCCCGCCGAGCAGCCCCTGAGC |
| | | ACACCTCTGAGCCACAGCACC |
| EP17aa1-6 | | |
| SEQ ID | Amino | APTSSSTQNKALPENVKYGIVLDAGSSHTSLYIYKWPAEKENDTGVVHQV |
| NO: 239 | acid | EECRVKGPGISKFVQKVNEIGIYLTDCMERAREVIPRSQHQETPVYLGATA |
| | | GMRLLRMESEELADRVLDVVERSLSNYPFDFQGARIITGQEEGAYGWITI |
| | | NYLLGKFSQKNQETFGALDLGGASTQVTFVPQNQTIESPDNALQFRLYGK |
| | | DYNVYTHSFLCYGKDQALWQKLAKDIQVASNEILRDPCFHPGYKKVVNVS |
| | | DLYKTPCTKRFEMTLPFQQFEIQGIGNYQQCHQSILELFNTSYCPYSQCA |
| | | FNGIFLPPLQGDFGAFSAFYFVMKFLNLTSEKVSQEKVTEMMKKFCAQP |
| | | WEEIKTSYAGVNEKYLSEFCFSGTYILSLLLQGYHFTADSWEHIHFIGKIQG |
| | | SDAGWTLGYMLNLTNMIPAEQPLSTPLSHST |
| SEQ ID | DNA | GCCCCTACCAGCAGCAGCACCCAGAACAAGGCCCTGCCCGAGAACGT |
| NO: 240 | | GAAGTACGGCATCGTGCTGGATGCCGGCAGCAGCCACACCAGCCTGT |
| | | ACATCTACAAGTGGCCTGCCGAGAAAGAAAACGACACCGGCGTGGTG |
| | | CATCAGGTGGAAGAGTGCAGAGTGAAGGGCCCTGGCATCAGCAAGTT |
| | | CGTGCAGAAAGTGAACGAGATCGGCATCTACCTGACCGACTGCATGG |
| | | AACGGGCCAGGGAAGTGATCCCCAGAAGCCAGCACCAGGAAACCCC |
| | | CGTGTATCTGGGAGCCACCGCCGGCATGAGACTGCTGAGAATGGAAA |
| | | GCGAGGAACTGGCCGACCGGGTGCTGGACGTGGTGGAAAGAAGCCT |
| | | GAGCAACTACCCATTCGATTTTCAAGGCGCCAGAATCATCACCGGCCA |
| | | GGAAGAAGGCGCCTACGGCTGGATCACCATCAACTACCTGCTGGGCA |
| | | AGTTCAGCCAGAAGAATCAGGAAACCTTCGGCGCCCTGGACCTGGGC |
| | | GGAGCTTCTACCCAAGTGACCTTCGTGCCCCAGAATCAGACCATCGA |
| | | GAGCCCCGACAACGCCCTGCAGTTCCGGCTGTACGGCAAGGACTACA |
| | | ATGTGTACACCCACAGCTTTCTGTGCTACGGAAAGGACCAGGCTCTGT |
| | | GGCAGAAGCTGGCCAAGGACATCCAGGTGGCCAGCAACGAGATCCT |
| | | GCGGGACCCTTGCTTCCACCCCGGCTACAAGAAAGTCGTGAACGTGT |
| | | CCGACCTGTACAAGACCCCCTGCACCAAGAGATTCGAGATGACCCTG |
| | | CCCTTCCAGCAGTTCGAGATCCAGGGCATCGGCAATTACCAGCAGTG |
| | | CCACCAGAGCATCCTGGAACTGTTCAACACCAGCTACTGCCCCTACAG |
| | | CCAGTGCGCCTTCAACGGCATCTTCCTGCCACCTCTGCAGGGGGATT |
| | | TCGGCGCCTTCAGCGCCTTCTACTTCGTGATGAAGTTCCTGAACCTGA |
| | | CCAGCGAGAAGGTGTCCCAGGAAAAAGTGACAGAGATGATGAAGAAG |
| | | TTCTGCGCCCAACCCTGGGAGGAAATCAAGACCTCCTACGCTGGCGT |
| | | GAACGAGAAGTACCTGAGCGAGTTTTGCTTCAGCGGCACCTACATCCT |
| | | GAGCCTGCTGCTGCAGGGCTACCACTTCACCGCCGATAGCTGGGAGC |
| | | ACATCCACTTCATCGGCAAGATTCAGGGCAGCGACGCCGGCTGGACA |
| | | CTGGGCTACATGCTGAATCTGACCAACATGATCCCCGCCGAGCAGCC |
| | | CCTGAGCACACCTCTGAGCCACAGCACC |
| EP17aa1-15 | | |
| SEQ ID | Amino | APTSSSTKKTQLTSSTQNKALPENVKYGIVLDAGSSHTSLYIYKWPAEKEN |
| NO: 241 | acid | DTGVVHQVEECRVKGPGISKFVQKVNEIGIYLTDCMERAREVIPRSQHQE |
| | | TPVYLGATAGMRLLRMESEELADRVLDVVERSLSNYPFDFQGARIITGQE |
| | | EGAYGWITINYLLGKFSQKNQETFGALDLGGASTQVTFVPQNQTIESPDN |
| | | ALQFRLYGKDYNVYTHSFLCYGKDQALWQKLAKDIQVASNEILRDPCFHP |
| | | GYKKVVNVSDLYKTPCTKRFEMTLPFQQFEIQGIGNYQQCHQSILELFNT |
| | | SYCPYSQCAFNGIFLPPLQGDFGAFSAFYFVMKFLNLTSEKVSQEKVTEM |
| | | MKKFCAQPWEEIKTSYAGVNEKYLSEFCFSGTYILSLLLQGYHFTADSWE |
| | | HIHFIGKIQGSDAGWTLGYMLNLTNMIPAEQPLSTPLSHST |
| SEQ ID | DNA | GCCCCTACCAGCAGCAGCACCAAGAAAACCCAGCTGACCAGCAGCAC |
| NO: 242 | | CCAGAACAAGGCCCTGCCCGAGAACGTGAAGTACGGCATCGTGCTGG |
| | | ATGCCGGCAGCAGCCACACCAGCCTGTACATCTACAAGTGGCCTGCC |
| | | GAGAAAGAAAACGACACCGGCGTGGTGCATCAGGTGGAAGAGTGCAG |
| | | AGTGAAGGGCCCTGGCATCAGCAAGTTCGTGCAGAAAGTGAACGAGA |
| | | TCGGCATCTACCTGACCGACTGCATGGAACGGGCCAGGGAAGTGATC |
| | | CCCAGAAGCCAGCACCAGGAAACCCCCGTGTATCTGGGAGCCACCGC |
| | | CGGCATGAGACTGCTGAGAATGGAAAGCGAGGAACTGGCCGACCGG |
| | | GTGCTGGACGTGGTGGAAAGAAGCCTGAGCAACTACCCATTCGATTTT |
| | | CAAGGCGCCAGAATCATCACCGGCCAGGAAGAAGGCGCCTACGGCT |
| | | GGATCACCATCAACTACCTGCTGGGCAAGTTCAGCCAGAAGAATCAG |
| | | GAAACCTTCGGCGCCCTGGACCTGGGCGGAGCTTCTACCCAAGTGAC |
| | | CTTCGTGCCCCAGAATCAGACCATCGAGAGCCCCGACAACGCCCTGC |
| | | AGTTCCGGCTGTACGGCAAGGACTACAATGTGTACACCCACAGCTTTC |
| | | TGTGCTACGGAAAGGACCAGGCTCTGTGGCAGAAGCTGGCCAAGGAC |
| | | ATCCAGGTGGCCAGCAACGAGATCCTGCGGGACCCTTGCTTCCACCC |
| | | CGGCTACAAGAAAGTCGTGAACGTGTCCGACCTGTACAAGACCCCCT |
| | | GCACCAAGAGATTCGAGATGACCCTGCCCTTCCAGCAGTTCGAGATC |
| | | CAGGGCATCGGCAATTACCAGCAGTGCCACCAGAGCATCCTGGAACT |
| | | GTTCAACACCAGCTACTGCCCCTACAGCCAGTGCGCCTTCAACGGCA |
| | | TCTTCCTGCCACCTCTGCAGGGGGATTTCGGCGCCTTCAGCGCCTTC |
| | | TACTTCGTGATGAAGTTCCTGAACCTGACCAGCGAGAAGGTGTCCCAG |
| | | GAAAAAGTGACAGAGATGATGAAGAAGTTCTGCGCCCAACCCTGGGA |
| | | GGAAATCAAGACCTCCTACGCTGGCGTGAACGAGAAGTACCTGAGCG |
| | | AGTTTTGCTTCAGCGGCACCTACATCCTGAGCCTGCTGCTGCAGGGC |
| | | TACCACTTCACCGCCGATAGCTGGGAGCACATCCACTTCATCGGCAA |
| | | GATTCAGGGCAGCGACGCCGGCTGGACACTGGGCTACATGCTGAATC |
| | | TGACCAACATGATCCCCGCCGAGCAGCCCCTGAGCACACCTCTGAGC |
| | | CACAGCACC |
| EP19aa1-3 | | |
| SEQ ID | Amino | APTTQNKALPENVKYGIVLDAGSSHTSLYIYKWPAEKENDTGVVHQVEEC |
| NO: 243 | acid | RVKGPGISKFVQKVNEIGIYLTDCMERAREVIPRSQHQETPVYLGATAGM |
| | | RLLRMESEELADRVLDVVERSLSNYPFDFQGARIITGQEEGAYGWITINYL |
| | | LGKFSQKNQETFGALDLGGASTQVTFVPQNQTIESPDNALQFRLYGKDY |
| | | NVYTHSFLCYGKDQALWQKLAKDIQVASNEILRDPCFHPGYKKVVNVSDL |
| | | YKTPCTKRFEMTLPFQQFEIQGIGNYQQCHQSILELFNTSYCPYSQCAFN |
| | | GIFLPPLQGDFGAFSAFYFVMKFLNLTSEKVSQEKVTEMMKKFCAQPWE |
| | | EIKTSYAGVKEKYLSEYCFSGTYILSLLQQGYHFTADSWEDIHFIGKIQGSD |
| | | AGWTLGYMLNLTNMIPAEQPLSTPLSHST |
| SEQ ID | DNA | GCCCCTACCACCCAGAACAAGGCCCTGCCCGAGAACGTGAAGTACGG |
| NO: 244 | | CATCGTGCTGGATGCCGGCAGCAGCCACACCAGCCTGTACATCTACA |
| | | AGTGGCCTGCCGAGAAAGAAAACGACACCGGCGTGGTGCATCAGGTG |
| | | GAAGAGTGCAGAGTGAAGGGCCCTGGCATCAGCAAGTTCGTGCAGAA |
| | | AGTGAACGAGATCGGCATCTACCTGACCGACTGCATGGAACGGGCCA |
| | | GGGAAGTGATCCCCAGAAGCCAGCACCAGGAAACCCCCGTGTATCTG |
| | | GGAGCCACCGCCGGCATGAGACTGCTGAGAATGGAAAGCGAGGAAC |
| | | TGGCCGACCGGGTGCTGGACGTGGTGGAAAGAAGCCTGAGCAACTA |
| | | CCCATTCGATTTTCAAGGCGCCAGAATCATCACCGGCCAGGAAGAAG |
| | | GCGCCTACGGCTGGATCACCATCAACTACCTGCTGGGCAAGTTCAGC |
| | | CAGAAGAATCAGGAAACCTTCGGCGCCCTGGACCTGGGCGGAGCTTC |
| | | TACCCAAGTGACCTTCGTGCCCCAGAATCAGACCATCGAGAGCCCCG |
| | | ACAACGCCCTGCAGTTCCGGCTGTACGGCAAGGACTACAATGTGTAC |
| | | ACCCACAGCTTTCTGTGCTACGGAAAGGACCAGGCTCTGTGGCAGAA |
| | | GCTGGCCAAGGACATCCAGGTGGCCAGCAACGAGATCCTGCGGGAC |
| | | CCTTGCTTCCACCCCGGCTACAAGAAAGTCGTGAACGTGTCCGACCT |
| | | GTACAAGACCCCCTGCACCAAGAGATTCGAGATGACCCTGCCCTTCC |
| | | AGCAGTTCGAGATCCAGGGCATCGGCAATTACCAGCAGTGCCACCAG |
| | | AGCATCCTGGAACTGTTCAACACCAGCTACTGCCCCTACAGCCAGTGC |
| | | GCCTTCAACGGCATCTTCCTGCCACCTCTGCAGGGGGATTTCGGCGC |
| | | CTTCAGCGCCTTCTACTTCGTGATGAAGTTCCTGAACCTGACCAGCGA |
| | | GAAGGTGTCCCAGGAAAAAGTGACAGAGATGATGAAGAAGTTCTGCG |
| | | CCCAGCCCTGGGAGGAAATCAAGACCTCCTACGCTGGCGTGAAAGAG |
| | | AAGTACCTGAGCGAGTACTGCTTCAGCGGCACCTACATCCTGAGCCT |
| | | GCTGCAGCAGGGCTACCACTTCACCGCCGATAGCTGGGAGGACATCC |
| | | ACTTCATCGGCAAGATTCAGGGCAGCGACGCCGGCTGGACACTGGGC |
| | | TACATGCTGAATCTGACCAACATGATCCCCGCCGAGCAGCCCCTGAG |
| | | CACACCTCTGAGCCACAGCACC |
| EP19aa1-6 | | |
| SEQ ID | Amino | APTSSSTQNKALPENVKYGIVLDAGSSHTSLYIYKWPAEKENDTGVVHQV |
| NO: 245 | acid | EECRVKGPGISKFVQKVNEIGIYLTDCMERAREVIPRSQHQETPVYLGATA |
| | | GMRLLRMESEELADRVLDVVERSLSNYPFDFQGARIITGQEEGAYGWITI |
| | | NYLLGKFSQKNQETFGALDLGGASTQVTFVPQNQTIESPDNALQFRLYGK |
| | | DYNVYTHSFLCYGKDQALWQKLAKDIQVASNEILRDPCFHPGYKKVVNVS |
| | | DLYKTPCTKRFEMTLPFQQFEIQGIGNYQQCHQSILELFNTSYCPYSQCA |
| | | FNGIFLPPLQGDFGAFSAFYFVMKFLNLTSEKVSQEKVTEMMKKFCAQP |
| | | WEEIKTSYAGVKEKYLSEYCFSGTYILSLLQQGYHFTADSWEDIHFIGKIQ |
| | | GSDAGWTLGYMLNLTNMIPAEQPLSTPLSHST |
| SEQ ID | DNA | GCCCCTACCAGCAGCAGCACCCAGAACAAGGCCCTGCCCGAGAACGT |
| NO: 246 | | GAAGTACGGCATCGTGCTGGATGCCGGCAGCAGCCACACCAGCCTGT |
| | | ACATCTACAAGTGGCCTGCCGAGAAAGAAAACGACACCGGCGTGGTG |
| | | CATCAGGTGGAAGAGTGCAGAGTGAAGGGCCCTGGCATCAGCAAGTT |
| | | CGTGCAGAAAGTGAACGAGATCGGCATCTACCTGACCGACTGCATGG |
| | | AACGGGCCAGGGAAGTGATCCCCAGAAGCCAGCACCAGGAAACCCC |
| | | CGTGTATCTGGGAGCCACCGCCGGCATGAGACTGCTGAGAATGGAAA |
| | | GCGAGGAACTGGCCGACCGGGTGCTGGACGTGGTGGAAAGAAGCCT |
| | | GAGCAACTACCCATTCGATTTTCAAGGCGCCAGAATCATCACCGGCCA |
| | | GGAAGAAGGCGCCTACGGCTGGATCACCATCAACTACCTGCTGGGCA |
| | | AGTTCAGCCAGAAGAATCAGGAAACCTTCGGCGCCCTGGACCTGGGC |
| | | GGAGCTTCTACCCAAGTGACCTTCGTGCCCCAGAATCAGACCATCGA |
| | | GAGCCCCGACAACGCCCTGCAGTTCCGGCTGTACGGCAAGGACTACA |
| | | ATGTGTACACCCACAGCTTTCTGTGCTACGGAAAGGACCAGGCTCTGT |
| | | GGCAGAAGCTGGCCAAGGACATCCAGGTGGCCAGCAACGAGATCCT |
| | | GCGGGACCCTTGCTTCCACCCCGGCTACAAGAAAGTCGTGAACGTGT |
| | | CCGACCTGTACAAGACCCCCTGCACCAAGAGATTCGAGATGACCCTG |
| | | CCCTTCCAGCAGTTCGAGATCCAGGGCATCGGCAATTACCAGCAGTG |
| | | CCACCAGAGCATCCTGGAACTGTTCAACACCAGCTACTGCCCCTACAG |
| | | CCAGTGCGCCTTCAACGGCATCTTCCTGCCACCTCTGCAGGGGGATT |
| | | TCGGCGCCTTCAGCGCCTTCTACTTCGTGATGAAGTTCCTGAACCTGA |
| | | CCAGCGAGAAGGTGTCCCAGGAAAAAGTGACAGAGATGATGAAGAAG |
| | | TTCTGCGCCCAGCCCTGGGAGGAAATCAAGACCTCCTACGCTGGCGT |
| | | GAAAGAGAAGTACCTGAGCGAGTACTGCTTCAGCGGCACCTACATCC |
| | | TGAGCCTGCTGCAGCAGGGCTACCACTTCACCGCCGATAGCTGGGAG |
| | | GACATCCACTTCATCGGCAAGATTCAGGGCAGCGACGCCGGCTGGAC |
| | | ACTGGGCTACATGCTGAATCTGACCAACATGATCCCCGCCGAGCAGC |
| | | CCCTGAGCACACCTCTGAGCCACAGCACC |
| SEQ ID | Amino | EFRHDS |
| NO: 247 | acid | |
| SEQ ID | DNA | GAATTCCGGCACGACAGC |
| NO: 248 | | |
| SEQ ID | Amino | HHHHHH |
| NO: 249 | acid | |
| SEQ ID | DNA | CATCATCATCATCATCAC |
| NO: 250 | | |

1.-18. (canceled)

19. A method of treating tissue damage in a human subject, comprising administering a therapeutically effective dose of solubilized human apyrase to said subject.

20. The method of claim 19, wherein the solubilized human apyrase is an apyrase with at least two modifications selected from the list consisting of: N terminal deletion, C terminal deletion and central modification.

21. The method of claim 20, wherein the tissue damage is acute brain injury (stroke); acute multi-organ failure; delayed graft function after transplantation of kidney or other solid organs; burn damage; radiation damage; acute damage due to trauma and/or hypoxia, such as acute respiratory distress syndrome (ARDS) or lung injury; acute kidney injury, such as acute kidney injury secondary to thoracic surgery (e.g. aortic valve replacement, coronary artery bypass surgery) or sepsis or rhabdomyolysis or toxic effects of antibiotics or other medication; acute myocardial injury.

22. A method of treating cardiac surgery associated acute kidney injury comprising administering a therapeutically effective dose of solubilized human apyrase to a subject in need of such treatment.

23. A method of treating delayed graft function (DGF), acute respiratory distress syndrome (ARDS), acute myocardial infarction (AMI), traumatic brain injury (TBI)/acute ischemic stroke (AIS) ischemia-reperfusion injury (IRI), or combinations thereof often referred to as multi-organ failures (MOF) comprising administering a therapeutically effective dose of solubilized human apyrase to a subject in need of such treatment.

24. A method of treating sepsis associated acute kidney injury comprising administering a therapeutically effective dose of solubilized human apyrase to a subject in need of such treatment.

25. An isolated nucleic acid molecule encoding a solubilized human apyrase with at least two modifications selected from the list consisting of: N terminal deletion, C terminal deletion and central modification.

26. A cloning or expression vector comprising one or more nucleic acid sequences according to claim 25, wherein the vector is suitable for the recombinant production a solubilized human apyrase with at least two modifications selected from the list consisting of: N terminal deletion, C terminal deletion and central modification.

27. A host cell comprising one or more cloning or expression vectors according to claim 26.

28. A process for the production of a solubilized human apyrase with at least two modifications selected from the list consisting of: N terminal deletion, C terminal deletion and central modification, comprising culturing a host cell according to claim 27, purifying and recovering said apyrase.

29. The method of claim 20, wherein the solubilized human apyrase comprises the N terminal deletion, C terminal deletion and central modification.

30. The method of claim 29, wherein the the N-terminal deletion is between 30 and 50 amino acids long, the C-terminal deletion is between 20 and 40 amino acids long, and the central modification comprises a deletion and/or point mutation.

31. The method of claim 30, wherein the central modification comprises a deletion of 10 to 15 consecutive amino acids.

32. The method of claim 31, wherein the central deletion is a deletion of amino acids number 193 to 204 in relation to the wild-type CD39 sequence according to SEQ ID NO: 1.

33. The method of claim 20, wherein the central modification comprises a point mutation comprising one, two, three, four or five point mutations in relation to the wild-type CD39 sequence according to SEQ ID NO: 1, selected group consisting of K71E, N73Q, V95A, G102D, Y104S, T106S, R113M, L149M, V151A, E173D, T229A, L254M, K258R, W263R, E276D, N292Q, R304G, 1319T, N327Q, A362N, F365S, N371Q, K405N, Y412F, L424Q, H436D, 1437N, F439S, G441D, N457Q, P463S, and S469R.

34. The method of claim 20, wherein the solubilized human apyrase comprises a sequence selected from the group consisting of SEQ ID NO: 4, SEQ ID NO: 6, SEQ ID NO: 32, SEQ ID NO: 54, SEQ ID NO: 56, SEQ ID NO: 70, SEQ ID NO: 76, and SEQ ID NO: 78.

35. The method of claim 20, wherein the solubilized human apyrase comprises a sequence selected from the group consisting of SEQ ID NO: 131, SEQ ID NO: 133, SEQ ID NO: 135, SEQ ID NO: 137, SEQ ID NO: 139 and SEQ ID NO: 141.

36. The method of claim 35, wherein the solubilized human apyrase comprises a sequence selected from the group consisting of SEQ ID NO: 213, SEQ ID NO: 227, SEQ ID NO: 219, SEQ ID NO: 225, SEQ ID NO: 217, SEQ ID NO: 209, SEQ ID NO: 221, SEQ ID NO: 72, SEQ ID NO: 215, SEQ ID NO: 223, SEQ ID NO: 211, SEQ ID NO: 58 and SEQ ID NO: 229.

37. The method of claim 36, wherein the solubilized human apyrase comprises a sequence selected from the group consisting of SEQ ID NO: 58, SEQ ID NO: 72 and SEQ ID NO: 229.
